(19) United States
(12) Patent Application Publication
(10) Pub. No.: US 2026/0308557 A1
(43) Pub. Date: Oct. 8, 2026

(54) FUSION PROTEINS COMPRISING CAS ENZYMES AND DNA-BINDING PROTEINS

(71) Applicant: SAREPTA THERAPEUTICS, INC., Cambridge, MA (US)

(72) Inventors: Eric WALTON, Cambridge, MA (US); Ami M. KABADI, Cambridge, MA (US)

(73) Assignee: SAREPTA THERAPEUTICS, INC., Cambridge, MA (US)

(21) Appl. No.: 19/484,810

(22) PCT Filed: May 15, 2024
(86) PCT No.: PCT/US2024/029359

§ 371 Date: Nov. 14, 2025

Related U.S. Application Data
(60) Provisional application No. 63/502,537, filed on May 16, 2023.

(51) Int. Cl. C12N 9/22; A61K 38/46; C07K 14/00; C12N 15/11; C12N 15/85

(52) U.S. Cl. CPC C12N 9/226; A61K 38/465; C07K 14/001; C12N 15/11; C12N 15/85; C07K 2319/81; C12N 2310/20

(57) ABSTRACT

Disclosed herein are fusion protein comprising a Cas enzyme (e.g., a type II or a type IV Cas enzyme) or its fragment and a DNA-binding protein (e.g., a 7 kDa DNA-binding protein) or a DNA-binding protein fragment thereof, as well as the use of this fusion protein in order to enhance CRISPR-Cas systems genome-editing efficiency.

### CROSS REFERENCE TO RELATED APPLICATIONS

This application claims priority under 35 U.S.C. § 119(e) to U.S. Provisional Application No. 63/502,537, filed May 16, 2023, the disclosure of which is incorporated herein by reference in its entirety.

### RELATED INFORMATION

The contents of any patents, patent applications, and references cited throughout this specification are hereby incorporated by reference in their entireties.

### SEQUENCE LISTING

The instant application contains a Sequence Listing which has been submitted electronically in XML format and is hereby incorporated by reference in its entirety. Said XML copy, created on May 10, 2024, is named 4140_0650001_SequenceListing_ST26.xml and is 253,041 bytes in size.

### FIELD OF DISCLOSURE

The present disclosure pertains to the medical field including gene therapy, for example, to the treatment of genetic diseases by gene therapy.

### BACKGROUND

The CRISPR-Cas (clustered regularly interspaced short palindromic repeats-CRISPR-associated proteins) modules are adaptive immunity systems that are encoded by most archaea and many bacteria and that act against invading genetic element, such as viruses and plasmids (Deveau, H., Garneau, J. E. & Moineau, S. CRISPR/Cas system and its role in phage-bacteria interactions. Annu. Rev. Microbiol. 64, 475-493 (2010); Horvath, P. & Barrangou, R. CRISPR/Cas, the immune system of bacteria and archaea. Science 327, 167-170 (2010); Karginov, F. V. & Hannon, G. J. The CRISPR system: small RNA-guided defense in bacteria and archaea. Mol. Cell 37, 7-19 (2010); Koonin, E. V. & Makarova, K. S. CRISPR-Cas: an adaptive immunity system in prokaryotes. F1000 Biol. Rep. 1, 95 (2009); Sorek, R., Kunin, V. & Hugenholtz, P. CRISPR—a widespread system that provides acquired resistance against phages in bacteria and archaea. Nature Rev. Microbiol. 6, 181-186 (2008); van der Oost, J., Jore, M. M., Westra, E. R., Lundgren, M. & Brouns, S. J. CRISPR-based adaptive and heritable immunity in prokaryotes. Trends Biochem. Sci. 34, 401-407 (2009).

CRISPR-Cas systems can be classified into 2 classes (Class 1 and Class 2), 6 types (I to VI) and several subtypes, with multi-Cas protein effector complexes in Class 1 systems (Type I, III, and IV) and a single effector protein in Class 2 systems (Type II, V, and VI) (Koonin E V, Makarova K S, Zhang F. Diversity, classification and evolution of CRISPR-Cas systems. Curr Opin Microbiol (2017); 37:67-78; Jiang F, Doudna J A. CRISPR-Cas9 structures and mechanisms. Annu Rev Biophys 2017; 46:505-29).

The CRISPR-Cas system uses RNA-guided nucleases to cleave genetic elements, and CRISPR systems have been identified across a wide range of bacterial and archaeal hosts. Each system comprises a cluster of CRISPR-associated (Cas) genes, and a distinctive array of repetitive elements (direct repeats) interspaced by short variable sequences (spacers) derived from exogenous DNA targets (protospacers), and together they constitute the CRISPR RNA (crRNA) array. Within the DNA target, each protospacer can be associated with a protospacer adjacent motif (PAM), which can vary depending on the specific CRISPR system. Not all CRISPR-Cas systems require a PAM sequence to be present in the target DNA, for example in type I and type II CRISPR-Cas systems, but not in type III systems, the selection of proto-spacers in invading nucleic acid depends on the proto-spacer-adjacent motif (PAM) (Mojica, F. J., Diez-Villasenor, C., Garcia-Martinez, J. & Almendros, C. Short motif sequences determine the targets of the prokaryotic CRISPR defence system. Microbiology 155, 733-740 (2009); Marraffini, L. A. & Sontheimer, E. J. Self versus nonself discrimination during CRISPR RNA-directed immunity. Nature 463, 568-571 (2010); Marraffini, L. A. & Sontheimer, E. J. CRISPR interference limits horizontal gene transfer in staphylococci by targeting DNA. Science 322, 1843-1845 (2008)).

The CRISPR locus is transcribed in a long primary CRISPR transcript (pre-crRNA), which is then cleaved resulting in the crRNAs, each crRNA unit contains a guide nucleotide sequence and a partial direct repeat, where the former directs the Cas enzyme (or its fragment) to a target DNA sequence via Watson-Crick base pairing. Most of the type II systems and some CRSPR systems of other types (e.g., Cas12b, Cas12c, and Cas12f1) additionally use a trans-encoded small RNA (transcrRNA or tracrRNA), a small RNA molecule that is required for the maturation of the pre-crRNA and interference.

Different Cas enzymes can cleave double-stranded DNA and single-stranded DNA or RNA. Cas enzyme (or its fragment) can, for example, cleave dsDNA and generate sequence-specific nuclease-induced DNA nicking or double-strand breaks (DSBs). Upon cleavage by Cas enzymes, the target locus typically undergoes one of two major pathways for DNA damage repair: the error-prone nonhomologous end-joining (NHEJ) or the high-fidelity homology-directed repair (HDR) pathway, both of which can be used to achieve a desired editing outcome. In the absence of a repair template, DSBs are re-ligated through the NHEJ process, which leaves scars in the form of insertion/deletion (indel) mutations. NHEJ can be harnessed to mediate gene knockouts, as indels occurring within a coding exon can lead to frameshift mutations and premature stop codons. Multiple DSBs can additionally be exploited to mediate larger deletions in the genome. HDR is an alternative major DNA repair pathway. Although HDR typically occurs at lower and substantially more variable frequencies than NHEJ, it can be leveraged to generate precise, defined modifications at a target locus in the presence of an exogenously introduced repair template. The repair template can either be in the form of conventional double-stranded DNA targeting constructs with homology arms flanking the insertion sequence, or single-stranded DNA oligonucleotides (ssODNs). The latter provides an effective and simple method for making small edits in the genome, such as the introduction of single-nucleotide mutations for probing causal genetic variation. Unlike NHEJ, HDR is generally active only in dividing cells, and its efficiency can vary widely depending on the cell type and state, as well as the genomic locus and repair template.

Cas enzymes comprise at least one nuclease domain having endonuclease activity. For example, a Cas9 nuclease comprises a HNH domain, which cleaves the guide RNA complementary strand, and a RuvC domain, which cleaves the non-complementary strand, a Cas12a nuclease comprises a RuvC domain and a NUC domain, and a Cas13a nuclease comprises two HNEPN domains. In some embodiments, both nuclease domains are active and the CRISPR nuclease has double-stranded cleavage activity (i.e., cleaves both strands of a double-stranded nucleic acid sequence). Alternatively, one of the nuclease domains is inactivated by one or more mutations and/or deletions, and the CRISPR variant is a nickase that cleaves one strand of a double-stranded nucleic acid sequence. For example, one or more mutations in the RuvC domain of Cas9 protein (e.g., D10A, D8A, E762A, and/or D986A) results in an HNH nickase that nicks the guide RNA complementary strand; and one or more mutations in the HNH domain of Cas9 protein (e.g., H840A, H559A, N854A, N856A, and/or N863A) results in a RuvC nickase that nicks the guide RNA non-complementary strand. Comparable mutations can convert Cas12a and Cas13a nucleases to nickases.

The RNA-guided nuclease function of CRISPR-Cas can be reconstituted in mammalian cells through the heterologous expression of human codon-optimized Cas enzymes and the requisite RNA components. Furthermore, the crRNA and tracrRNA can be fused together to create a chimeric, single-guide RNA (sgRNA). Cas enzymes can thus be re-directed toward almost any target of interest (in immediate vicinity of a PAM sequence for certain CRISPR-Cas systems) by altering the nucleotide guide sequence within the sgRNA. Thus, CRISPR-Cas can be used as a programmable nuclease-based genome editing technology. Despite the huge potential of CRISPR-Cas as a programmable nuclease-based genome editing technology, CRISPR-Cas systems have a major limitation—the efficiency of CRISPR-Cas-mediated genome editing. Editing rates reported in the literature vary widely, being highly dependent on CRISPR-system utilized, experimental conditions, and genomic targets. For example, Cas9 derived from Streptococcus pyogenes (spCas9), has been reported to produce a relatively higher editing rates and is generally accepted as one of the most active Cas systems currently utilized for gene editing applications. Despite its high activity, spCas9 has the challenge of being large and therefore difficult to deliver via traditional gene therapy methods. Thus, in one aspect, the disclosure provides development of small Cas gene editing systems that permit delivery in the viral vectors. For example, while the Cas12 system possesses the advantage of being small enough to be packed in a viral vector (e.g., AAV) that allows efficient delivery to cells, it has a low genome editing.

Therefore, to fully exploit the huge potential of the CRISPR-Cas systems as programmable nuclease-based genome editing tools, more efficient CRISPR-Cas systems are needed.

### BRIEF SUMMARY

In some aspects, provided herein are fusion proteins, comprising a Cas enzyme (or its fragment) and a DNA-binding protein or a DNA-binding fragment thereof. In some aspects, the DNA-binding protein or DNA-binding fragment thereof is linked to the Cas enzyme (or its fragment) via one or more chemical bonds, by a linker, or a combination thereof. In some aspects, the chemical bond is a covalent chemical bond. In some aspects, the linker is a flexible linker, a rigid linker, or any combination thereof. In some aspects, the linker is selected from the group consisting of (G)ₙ, (G₄S)ₙ (SEQ ID NO: 125), (GSG)ₙ, (GGGGS)ₙ (SEQ ID NO: 58), (EAAK)ₙ (SEQ ID NO: 59), or (XP)ₙ, wherein n is an integer in the range of 1-35 and X is any amino acid.

In some aspects, the DNA-binding protein or DNA-binding fragment thereof is located upstream of the Cas enzyme, downstream of the Cas enzyme, or a combination thereof. In some aspects, the DNA-binding protein or DNA-binding fragment thereof is linked to the Cas enzyme (or its fragment) at the N-terminus, C-terminus, or a combination thereof.

In some aspects, the fusion protein further comprises at least a nuclear localization signal (NLS), at least a cell penetrating peptide (CPP), or a combination thereof. In some aspects, the fusion protein further comprises a single NLS or multiple NLS. In some aspects, the NLS or the CPP is linked to the fusion protein via one or more chemical bonds, by a linker, or a combination thereof. In some aspects, the chemical bond is a covalent chemical bond. In some aspects, the linker is a flexible linker, a rigid linker, or any combination thereof. In some aspects, the linker is selected from the group consisting of (G)ₙ, (G₄S)ₙ (SEQ ID NO: 125), (GSG)ₙ, (GGGGS)ₙ (SEQ ID NO: 58), (EAAK)ₙ (SEQ ID NO: 59), or (XP)ₙ, wherein n is an integer between 1 and 35 and X is any amino acid. In some aspects, the NLS or the CPP is linked to the N-terminus of the Cas enzyme, the C-terminus of the Cas enzyme, the N-terminus of the DNA-binding protein or DNA-binding fragment thereof, the C-terminus of the DNA-binding protein or DNA-binding fragment thereof, or a combination thereof. In some aspects, the NLS is selected from the group consisting of SEQ ID NOs: 34-52.

In some aspects, the DNA-binding protein or a DNA-binding fragment thereof comprises a 7 kDa DNA-binding protein, a zinc finger (ZN) protein, or a transcription activator-like effector protein (TALE) or a DNA-binding fragment thereof.

In some aspects, the DNA-binding protein or a DNA-binding fragment thereof comprises a 7 kDa DNA-binding protein, a zinc finger (ZN) protein, or a transcription activator-like effector protein (TALE), or a DNA-binding fragment thereof. In some aspects, the 7 kDa DNA-binding protein, or DNA-binding fragment thereof, comprises Sac7d, Sac7e, Mse7, Mcu7, Aho7a, Aho7b, Aho7c, Sto7, Ssh7b, Sis7a, Sis7b, Ssh7a, or Sso7d. In some aspects, the 7 kDa DNA-binding protein or DNA-binding fragment thereof, is Sso7d or Aho7c. In some aspects, the 7 kDa DNA-binding protein comprises an amino acid sequence at least 70%, at least 80%, at least 90%, at least 95%, or 100% identical to an amino acid sequence of SEQ ID NO: 31 or 33.

In some aspects, the fusion protein has an editing efficiency that is increased compared to a same Cas enzyme (or its fragment) lacking the DNA-binding protein or DNA-binding fragment thereof. In some aspects, the editing efficiency of the fusion protein is increased by at least 1-fold, 2-fold, 3-fold, 4-fold, 5-fold, 6-fold, 7-fold, 8-fold, 9-fold, or 10-fold.

In some aspects, the Cas enzyme is a type II Cas enzyme or a type V Cas enzyme. In some aspects, the Cas enzyme is a Cas9, or a Cas12 enzyme. In some aspects, the Cas enzyme is selected from the group consisting of SpCas9, SaCas9, NmeCas9, Cas12f1, AsCas12f1, CasMINI, and SpCas12f1.

In some aspects, the Cas enzyme is SpCas9 or SaCas9. In some aspects, the Cas enzyme comprises an amino acid sequence at least 70%, at least 80%, at least 90%, at least 95%, or 100% identical to an amino acid sequence of SEQ ID NO: 25, 27, 110, or 111.

In other aspects, the Cas enzyme is Cas12f1, AsCas12f1 and CasMINI, or SpCas12f1. In these aspects, the Cas enzyme comprises an amino acid sequence at least 70%, at least 80%, at least 90%, at least 95%, or 100% identical to an amino acid sequence of SEQ ID NO: 29 or 112.

In some aspects, provided herein are expression cassettes, comprising a polynucleotide comprising a nucleotide sequence encoding a fusion protein, wherein the fusion protein comprises a Cas enzyme (or its fragment) and a DNA-binding protein or a DNA-binding fragment thereof. In some aspects, the nucleotide sequence comprises a first portion encoding the Cas enzyme (or its fragment) and a second portion encoding the DNA-binding protein or DNA-binding fragment thereof.

In some aspects, the first nucleotide sequence portion is located upstream of the second nucleotide sequence portion. In these aspects, the DNA-binding protein or DNA-binding fragment thereof is located downstream of the Cas enzyme. In some aspects, the DNA-binding protein or DNA-binding fragment thereof is linked to the Cas enzyme (or its fragment) at the C-terminus.

In other aspects, the first nucleotide sequence portion is localized downstream of the second nucleotide sequence portion. In some aspects, the DNA-binding protein or DNA-binding fragment thereof is located upstream of the Cas enzyme. In some aspects, the DNA-binding protein or DNA-binding fragment thereof is linked to the Cas enzyme (or its fragment) at the N-terminus.

In some aspects, the DNA-binding protein or DNA-binding fragment thereof is linked to the Cas enzyme (or its fragment) via one or more chemical bonds, by a linker, or a combination thereof. In some aspects, the chemical bond is a covalent chemical bond. In some aspects, the DNA-binding protein or DNA-binding fragment thereof is linked to the Cas enzyme (or its fragment) by a linker. In some aspects, the linker is a flexible linker, a rigid linker, or any combination thereof. In some aspects, the linker is selected from the group consisting of (G)ₙ, (G₄S)ₙ (SEQ ID NO: 125), (GSG)ₙ, (GGGGS)ₙ (SEQ ID NO: 58), (EAAK)ₙ (SEQ ID NO: 59), or (XP)ₙ, wherein n is an integer between 1 and 35 and X is any amino acid.

In some aspects, the polynucleotide further comprises a nucleotide sequence encoding at least a nuclear localization signal (NLS), at least a cell penetrating peptide (CPP), or a combination thereof. In some aspects, the polynucleotide comprises a single nucleotide sequence encoding a single NLS or multiple nucleotide sequences encoding a multiple NLS. In some aspects, the NLS is selected from the group consisting of SEQ ID NOs: 34-52. In some aspects, the NLS or the cell penetrating peptide is linked to the fusion protein via one or more chemical bonds, by a linker, or a combination thereof. In some aspects, the chemical bond is a covalent chemical bond. In some aspects, the linker is a flexible linker, a rigid linker, or any combination thereof. In some aspects, the linker is selected from the group consisting of (G)ₙ, (G₄S)ₙ (SEQ ID NO: 125), (GSG)ₙ, (GGGGS)ₙ (SEQ ID NO: 58), (EAAK)ₙ (SEQ ID NO: 59), or (XP)ₙ, wherein n is an integer between 1 and 35 and X is any amino acid.

In some aspects, the nucleotide sequence encoding the NLS or the CPP is located downstream of the Cas enzyme, the upstream of the Cas enzyme, the downstream of the DNA-binding protein or DNA-binding fragment thereof, the upstream of the DNA-binding protein or DNA-binding fragment thereof, or a combination thereof. In some aspects, the NLS or the CPP is linked to the N-terminus of the Cas enzyme, the C-terminus of the Cas enzyme, the N-terminus of the DNA-binding protein or DNA-binding fragment thereof, the C-terminus of the DNA-binding protein or DNA-binding fragment thereof, or a combination thereof.

In some aspects, the polynucleotide further comprises a nucleotide sequence encoding a molecular tag, a selectable marker, a self-cleaving peptide or any combination thereof.

In some aspects, the nucleotide sequence encoding the self-cleaving peptide is located upstream of the nucleotide sequence encoding the Cas enzyme, upstream of the nucleotide sequence encoding the DNA-binding protein or DNA-binding fragment thereof, upstream of the nucleotide sequence encoding the molecular tag, and downstream of the nucleotide sequence encoding the selectable marker.

In other aspects, the nucleotide sequence encoding the self-cleaving peptide is located downstream of the nucleotide sequence encoding the Cas enzyme, downstream of the nucleotide sequence encoding the DNA-binding protein or DNA-binding fragment thereof, downstream of the nucleotide sequence encoding the molecular tag, and upstream of the nucleotide sequence encoding the selectable marker.

In some aspects, the molecular tag is selected from the group consisting of CBP, FLAG, GST, HA, HBH, MBP, Myc, polyHis, S-tag, SUMO, TAP, TRX, or V5. In some aspects, the selectable marker is an ampicillin, a streptomycin, a gentamycin, a kanamycin, a hygromycin, a bialaphos herbicide, or a sulfonamide resistance conferring selectable marker. In some aspects, the self-cleaving peptide is selected from the group consisting of P2A, E2A, F2A, T2A, or furin cleavage site.

In some aspects, the polynucleotide is translated into a polypeptide comprising the Cas enzyme, the DNA-binding protein or DNA-binding fragment thereof, the self-cleaving peptide, the selectable marker, and optionally the NLS, the CPP, the linker, and the molecular tag. In some aspects, the self-cleaving peptide undergoes cleavage upon translation. In some aspects, the cleavage of the self-cleaving peptide produces a first product polypeptide comprising the Cas enzyme, the DNA-binding protein or DNA-binding fragment thereof, and, optionally, portion of the self-cleaving peptide, the NLS, the CPP, the linker, and the molecular tag, and a second product polypeptide comprising the selectable marker and optionally portion of the self-cleaving peptide.

In some aspects, the DNA-binding protein or a DNA-binding fragment thereof comprises a 7 kDa DNA-binding protein, a zinc finger (ZN) protein, or a transcription activator-like effector protein (TALE) or a DNA-binding fragment thereof.

In some aspects, the DNA-binding protein or a DNA-binding fragment thereof is a 7 kDa DNA-binding protein, a zinc finger protein, or a transcription activator-like effector protein, or a DNA-binding fragment thereof. In some aspects, the 7 kDa DNA-binding protein, or DNA-binding fragment thereof, is selected from the group consisting of Sac7d, Sac7e, Mse7, Mcu7, Aho7a, Aho7b, Aho7c, Sto7, Ssh7b, Sis7a, Sis7b, Ssh7a, Sso7d. In some aspects, 7 kDa DNA-binding protein, or DNA-binding fragment thereof, is Sso7d or Aho7c. In some aspects, the 7 kDa DNA-binding protein comprises an amino acid sequence at least 70%, at least 80%, at least 90%, at least 95%, or 100% identical to an amino acid sequence of SEQ ID NO: 31 or 33.

In some aspects, the fusion protein has an editing efficiency that is increased compared to a same Cas enzyme (or its fragment) lacking the DNA-binding protein or a DNA-binding fragment thereof. In some aspects, the editing efficiency of the fusion protein is increased by at least 1-fold, 2-fold, 3-fold, 4-fold, 5-fold, 6-fold, 7-fold, 8-fold, 9-fold, or 10-fold.

In some aspects, the polynucleotide further comprises a nucleotide sequence encoding a promoter, an enhancer, a silencer, a poly A tail, a poly adenylation signal, a 3′-UTR, a 5′-UTR, an intronic sequence, or any combination thereof. In some aspects, the promoter is a constitutive promoter, a regulatable promoter, an ubiquitous promoter, or a tissue-specific promoter. In some aspects, the promoter is an UbC promoter or a CBh promoter. In some aspects, the promoter comprises a nucleotide sequence at least 70%, at least 80%, at least 90%, at least 95%, or 100% identical to an amino acid sequence of SEQ ID NO: 53 or 54.

In some aspects, the nucleotide sequence of the polynucleotide is codon optimized for translation in a mammalian cell.

In some aspects, the Cas enzyme is a type II Cas enzyme or a type V Cas enzyme. In some aspects, the Cas enzyme is a Cas9, or a Cas12 enzyme. In some aspects, the Cas enzyme is selected from the group consisting of SpCas9, SaCas9, NmeCas9, Cas12f1, AsCas12f1, CasMINI, and SpCas12f1.

In some aspects, the Cas enzyme is SpCas9 or SaCas9. In some aspects, the Cas enzyme comprises an amino acid sequence at least 70%, at least 80%, at least 90%, at least 95%, or 100% identical to an amino acid sequence of SEQ ID NO: 25, 27, 110, or 111. In some aspects, the polynucleotide comprises a nucleotide sequence least 70%, at least 80%, at least 90%, at least 95%, or 100% identical to a nucleotide sequence of SEQ ID NO: 24, 26, 113, or 114. In some aspects, the expression cassette encodes an amino acid sequence at least 70%, at least 80%, at least 90%, at least 95%, or 100% identical to an amino acid sequence of SEQ ID NO: 16, 18, 119, or 120. In some aspects, the expression cassette comprises a nucleotide sequence at least 70%, at least 80%, at least 90%, at least 95%, or 100% identical to a nucleotide sequence of SEQ ID NO: 15, 17, 120, 122.

In other aspects, the Cas enzyme is Cas12f1 or SpCas12f1. In some aspects, the Cas enzyme comprises an amino acid sequence at least 70%, at least 80%, at least 90%, at least 95%, or 100% identical to an amino acid sequence of SEQ ID NO: 29 or 112. In some aspects, the polynucleotide comprises a nucleotide sequence least 70%, at least 80%, at least 90%, at least 95%, or 100% identical to a nucleotide sequence of SEQ ID NO: 28 or 115. In some aspects, the expression cassette encodes an amino acid sequence at least 70%, at least 80%, at least 90%, at least 95%, or 100% identical to an amino acid sequence of SEQ ID NO: 11, 20, 117, or 123. In some aspects, the expression cassette comprises a nucleotide sequence at least 70%, at least 80%, at least 90%, at least 95%, or 100% identical to a nucleotide sequence of SEQ ID NO: 10, 19, 118, or 124.

In some aspects, provided herein are vectors, comprising an expression cassettes disclosed herein. In some aspects, the vector further comprises a backbone nucleotide sequence. In some aspects, the backbone nucleotide sequence encodes a prokaryotic origin of replication, a multiple cloning site, a resistance conferring selectable marker, a promoter, an enhancer, a silencer, a polyA tail, a poly adenylation signal, a 3′-UTR, a 5′-UTR, an intronic sequence, or any combination thereof. In some aspects, the vector comprises an expression cassette disclosed herein comprising a nucleotide sequence encoding a Cas9 enzyme. In other aspects, the vector comprises an expression cassette disclosed herein comprising a nucleotide sequence encoding a Cas12 enzyme. In some aspects, the vector comprises a nucleotide sequence at least 70%, at least 80%, at least 90%, at least 95%, or 100% identical to a nucleotide sequence of SEQ ID NO: 12, 14, or 116.

In some aspects, provided herein are compositions for cleaving a target nucleic acid sequence in a cell comprising a fusion protein of claims, an expression cassette disclosed herein, or a vector disclosed herein and at least one guide RNA, at least a vector encoding the at least one guide RNA, or any combination thereof. In some aspects, the Cas enzyme, and the guide RNA are capable of forming a Cas enzyme/RNA complex in the cell that introduces a double stranded cleavage at the target nucleic acid sequence. In some aspects, the double stranded cleavage is repaired by a cell DNA damage repair machinery. In some aspects, the cell DNA damage repair machinery repairs the double stranded cleavage via nonhomologous end-joining (NHEJ). In some aspects, the composition, further comprises a repair template, and, optionally, the cell DNA damage repair machinery repairs the double stranded cleavage via high-fidelity homology-directed repair (HDR). In some aspects, the repair template is a double-stranded DNA targeting constructs, a single-stranded DNA oligonucleotides (ssODNs), or a combination thereof, and comprises homology arms flanking the double stranded cleavage.

In some aspects, the guide RNA is chemically synthesized, is transcribed from the vector encoding the guide RNA in vitro before introduction into the cell, or is transcribed from the vector encoding the guide RNA in vivo in the cell, wherein the vector encoding the guide RNA and the vector of comprising the expression cassette of the disclosure, are the same or are different.

In some aspects, the guide RNA comprises a crRNA portion and a tracrRNA portion. In some aspects, the crRNA portion and the tracrRNA portion are fused. In some aspects, the target nucleic acid sequence comprises a region that is at least 80%, at least 90%, at least 95%, or 100% complementary to the crRNA portion of the guide RNA. In some aspects, the target nucleic acid sequence further comprises a protospacer adjacent motif (PAM). In some aspects, the PAM comprises a nucleotide sequence of 5′-NGG, 5′-NRN, 5-NYN, 5′-NNGRRT, 5′-NNNRRT, 5′-NGGNG, 5′-NNAGAAW, or 5′-ACAY.

In other aspects, the guide RNA comprises a crRNA and does not comprise a tracrRNA. In some aspects, the target nucleic acid sequence comprises a region that is at least 80%, at least 90%, at least 95%, or 100% complementary to the crRNA. In some aspects, the target nucleic acid sequence further comprises a protospacer adjacent motif (PAM). In some aspects, the PAM is a 5′ T-rich sequence. In some aspects, the PAM comprises a nucleotide sequence of 5′-TTTR, 5′-TTTV, 5′-NTTC, 5′-GTTT, 5′-GTTC, 5′-AATC, 5′-ACTC, 5′-GGTC, or 5′-GCTC.

In some aspects, provided herein are methods of modifying the gene expression in a cell, comprising contacting the cell with the compositions disclosed herein. In some embodiments, the modification of gene expression comprises editing a genome, Prime gene editing, and epigenetic gene regulation. In some aspects, contacting the cell with the composition results in editing the target nuclei acid sequence. In some aspects, contacting the cell with the composition results in expression of fusion proteins. In some aspects, the composition comprises a recombinant viral vector (e.g., AAV), Lipid nanoparticle (LNP), or ribonucleoprotein (RNP). In some aspects, editing the target nuclei acid region comprises introducing an indel or a large deletion in the target nucleic acid sequence. In some aspects, editing the target nucleic acid region comprises introducing an exogenous sequence comprised in a repair template in the target nucleic acid sequence.

In some aspects, the genome of the cell is edited with an efficiency which is increased compared to the efficiency at which the genome of the cell is edited when the cell is contacted with a composition comprising a same Cas enzyme, a same guide RNA, and, optionally, a same repair template, but not comprising a DNA-binding protein or a DNA-binding fragment thereof. In some aspects, the efficiency is increased of at least 1-fold, 2-fold, 3-fold, 4-fold, 5-fold, 6-fold, 7-fold, 8-fold, 9-fold, or 10-fold.

In some aspects, the cell is comprised in a subject. In some aspects, the subject is human. In some aspects, the subject suffers from a genetic disease. In some aspects, the genetic disease is selected from the group consisting of achondroplasia, hereditary hemochromatosis, Down Syndrome, hereditary spherocytosis, Tay-Sachs Disease, Usher syndrome, hereditary fructose intolerance, hemophilia, muscular dystrophy (e.g., Duchenne muscular dystrophy or DMD, and Myotonic Dystrophy, including DM1 and DM2), Dilated Cardiomyopathy (e.g., TTN-DCM), Huntington's disease, polygenic disorders, breast cancer, ovarian cancer, Parkinson's disease, Bardet-Biedl syndrome, Prader-Willi syndrome, diabetes, heart disease, arthritis, motor neuron disease, albinism, Cri-du-Chat syndrome, cystic fibrosis, fragile X syndrome, galactosemia, Huntington's disease, Jackson-Weiss syndrome, Klinefelter syndrome, Krabbe disease, Langer-Giedion syndrome, Lesch-Nyhan syndrome, Marfan syndrome, myotonic dystrophy, Nail-Patella syndrome, neurofibromatosis, Noonan syndrome, triple X syndrome, osteogenesis imperfecta, Patau syndrome, phenylketonuria, porphyria, retinoblastoma, Rett syndrome, sickle cell disease, Turner syndrome, Usher syndrome, Von Hippel-Lindau syndrome, Waardenburg syndrome, Wilson's disease, xeroderma pigmentosum, XXXX syndrome, or YY syndrome can be characterized by an impaired production of a protein, or by a defective splicing. A hereditary disease such as for example: achondroplasia, hereditary hemochromatosis, Down Syndrome, hereditary spherocytosis, Tay-Sachs Disease, Usher syndrome, hereditary fructose intolerance, hemophilia, muscular dystrophy, Duchenne muscular dystrophy or DMD, polygenic disorders, breast cancer, ovarian cancer, Parkinson's disease, Bardet-Biedl syndrome, Prader-Willi syndrome, diabetes, heart disease, arthritis, motor neuron disease, albinism, Cri-du-Chat syndrome, cystic fibrosis, fragile X syndrome, galactosemia, Huntington's disease, Jackson-Weiss syndrome, Klinefelter syndrome, Krabbe disease, Langer-Giedion syndrome. Lesch-Nyhan syndrome, Marfan syndrome, myotonic dystrophy, Nail-Patella syndrome, neurofibromatosis, Noonan syndrome, triple X syndrome, osteogenesis imperfecta, Patau syndrome, phenylketonuria, porphyria, retinoblastoma, Rett syndrome, sickle cell disease, Turner syndrome, Usher syndrome, Von Hippel-Lindau syndrome, Waardenburg syndrome, Wilson's disease, xeroderma pigmentosum, XXXX syndrome, and YY syndrome. In some aspects, the genetic disease is a muscular dystrophy. In some aspects, the muscular dystrophy is Duchenne muscular dystrophy.

In some aspects, provide herein are pharmaceutical formulations, comprising a composition described herein or a pharmaceutically acceptable salt thereof, and at least one pharmaceutically acceptable excipient or adjuvant.

In some aspects, provided herein are methods of treating a subject, comprising administering to the subject a composition disclosed herein, a pharmaceutically acceptable salt thereof, or a pharmaceutical formulation disclosed herein. In some aspects, the subject is a human. In some aspects, the subject suffers from a genetic disease. In some aspects, the genetic disease is selected from the group consisting of achondroplasia, hereditary hemochromatosis, Down Syndrome, hereditary spherocytosis, Tay-Sachs Disease, Usher syndrome, hereditary fructose intolerance, hemophilia, muscular dystrophy (e.g., Duchenne muscular dystrophy or DMD, or Myotonic Dystrophy, including DM1 and DM2), Dilated Cardiomyopathy (e.g., TTN-DCM), Huntington's disease, polygenic disorders, breast cancer, ovarian cancer, Parkinson's disease, Bardet-Biedl syndrome, Prader-Willi syndrome, diabetes, heart disease, arthritis, motor neuron disease, albinism, Cri-du-Chat syndrome, cystic fibrosis, fragile X syndrome, galactosemia, Huntington's disease, Jackson-Weiss syndrome, Klinefelter syndrome, Krabbe disease, Langer-Giedion syndrome, Lesch-Nyhan syndrome, Marfan syndrome, myotonic dystrophy, Nail-Patella syndrome, neurofibromatosis, Noonan syndrome, triple X syndrome, osteogenesis imperfecta, Patau syndrome, phenylketonuria, porphyria, retinoblastoma, Rett syndrome, sickle cell disease, Turner syndrome, Usher syndrome, Von Hippel-Lindau syndrome, Waardenburg syndrome, Wilson's disease, xeroderma pigmentosum, XXXX syndrome, or YY syndrome can be characterized by an impaired production of a protein, or by a defective splicing. A hereditary disease such as for example: achondroplasia, hereditary hemochromatosis, Down Syndrome, hereditary spherocytosis, Tay-Sachs Disease, Usher syndrome, hereditary fructose intolerance, hemophilia, muscular dystrophy, Duchenne muscular dystrophy or DMD, polygenic disorders, breast cancer, ovarian cancer, Parkinson's disease, Bardet-Biedl syndrome, Prader-Willi syndrome, diabetes, heart disease, arthritis, motor neuron disease, albinism, Cri-du-Chat syndrome, cystic fibrosis, fragile X syndrome, galactosemia, Huntington's disease, Jackson-Weiss syndrome, Klinefelter syndrome, Krabbe disease, Langer-Giedion syndrome. Lesch-Nyhan syndrome, Marfan syndrome, myotonic dystrophy, Nail-Patella syndrome, neurofibromatosis, Noonan syndrome, triple X syndrome, osteogenesis imperfecta, Patau syndrome, phenylketonuria, porphyria, retinoblastoma, Rett syndrome, sickle cell disease, Turner syndrome, Usher syndrome, Von Hippel-Lindau syndrome, Waardenburg syndrome, Wilson's disease, xeroderma pigmentosum, XXXX syndrome, and YY syndrome. In some aspects, the genetic disease is a muscular dystrophy. In some aspects, the muscular dystrophy is Duchenne muscular dystrophy.

### BRIEF DESCRIPTION OF DRAWINGS

FIG. 1 shows a schematic of an open reading frame including the Cas12f1-Sso7d fusion protein.

FIG. 2A shows the average editing efficiency across all targets, represented by pooling all replicates of all samples for a given Cas12f1 variant. Variations include the promoter driving expression (UbC or CBh) and presence or absence of the Sso7d fusion. Comparisons are made only between variants under control of the same promoter.

FIGS. 2B-C show the fold change in editing efficiency between the Sso7d fusion and wild type Cas12f1 under control of the UbC promoter (FIG. 2B) or under the control of the CBh promoter (FIG. 2C).

FIGS. 3A-B show the editing efficiency correlation between wild type Cas12f1 and the Sso7d fusion across all targets with the Cas12f1 and the Sso7d fusion under the control of the UbC promoter (FIG. 3A) or under the control of the CBh promoter (FIG. 3B).

FIGS. 3C-D show the degree of editing efficiency enhancement between wild type Cas12f1 and the Sso7d fusion across all targets with the Cas12f1 and the Sso7d fusion under the control of the UbC promoter (FIG. 3C) or under the control of the CBh promoter (FIG. 3D.)

FIG. 4A shows tape station analysis of deletion-spanning PCR products from unedited and edited genomic DNA.

FIG. 4B shows the quantification of deletion percent from the tape station analysis.

FIGS. 5A-B show average editing efficiency for “Target A” and “Target B” for native Cas12f1, Cas12f1-Sso7d, and Cas12f1-Aho7c.

### DETAILED DESCRIPTION OF THE DISCLOSURE

### 1. Definitions

In order that the present disclosure can be more readily understood, certain terms are first defined. As used in this application, except as otherwise expressly provided herein, each of the following terms shall have the meaning set forth below. Additional definitions are set forth throughout the application.

It is to be noted that, as used herein, the indefinite articles “a” or “an” should be understood to refer to “one or more” of any recited or enumerated component; for example, “a nucleic acid sequence,” is understood to represent one or more nucleic acid sequences, unless stated otherwise. As such, the terms “a” (or “an”), “one or more,” and “at least one” can be used interchangeably herein. The use of the alternative (e.g., “or”) should be understood to mean either one, both, or any combination thereof of the alternatives.

Furthermore, “and/or,” where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

As used herein, the term “about” refers to a value that is within 10% above or below the value being described.

The term “at least” prior to a value or series of values is understood to include the values adjacent to the term “at least,” and all subsequent values (numbers, integers, or fractions) that could logically be included, as clear from context. For example, the number of nucleotides in a nucleic acid molecule must be an integer, e.g., “at least 18 nucleotides of a 21-nucleotide nucleic acid molecule” means that 18, 19, 20, or 21 nucleotides have the indicated property. When “at least” is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

As used herein, “no more than” or “less than” is understood as the value adjacent to the phrase and logical lower values (numbers, integers, or fractions), as logical from context, to zero. When “no more than” is present before a series of values or a range, it is understood that “no more than” can modify each of the value in the series or range.

As described herein, any concentration range, percentage range, ratio range or integer range is to be understood to include the value of any integer within the recited range and, when appropriate, fractions thereof (such as one-tenth and one-hundredth of an integer), unless otherwise indicated.

The term “derived from,” as used herein, refers to a component that is isolated from or made using a specified molecule or organism, or information (e.g., amino acid or nucleic acid sequence) from the specified molecule or organism.

A “chemical bond” is a link between atoms, groups of atoms, or ions. Chemical bonds can be “covalent bonds” or “noncovalent bonds.” A covalent bond refers to the mutual sharing of one or more pairs of electrons between two atoms. A noncovalent bond refers to a relatively weak bond/interaction formed between molecules without sharing electrons. Exemplary noncovalent bonds are ionic interactions, hydrophobic interactions, and hydrogen bonds.

“Nucleic acid,” “polynucleotide,” and “oligonucleotide,” are used interchangeably in the present application. These terms refer only to the primary structure of the molecule. Thus, these terms include double- and single-stranded DNA, as well as double- and single-stranded RNA (e.g., messenger RNAs (mRNAs), guide RNAs (gRNAs), genomic DNAs, plasmid DNAs (pDNAs), or complementary DNAs (cDNAs)). The terms “nucleic acid,” “polynucleotide,” and “oligonucleotide,” as used herein, are defined as it is generally understood by the person skilled in the art as a molecule comprising two or more covalently linked nucleosides. Such covalently bound nucleosides can also be referred to as nucleic acid molecules or oligomers. Polynucleotides can be made recombinantly, enzymatically, or synthetically, e.g., by solid-phase chemical synthesis followed by purification. When referring to a sequence of the polynucleotide or nucleic acid, reference is made to the sequence or order of nucleobase moieties, or modifications thereof, of the covalently linked nucleotides or nucleosides.

By “isolated” nucleic acid or polynucleotide is intended a nucleic acid molecule, DNA or RNA, which is not from its native environment. An isolated polynucleotide includes polynucleotides produced and maintained (e.g., upon purification) in vitro, or recombinant polynucleotides maintained in host cells or purified (partially or substantially) from the host cell and maintained in solution. Non-limiting examples of isolated polynucleotides or nucleic acids include molecules produced synthetically (i.e., by chemical synthesis), by PCR, by enzymatic digestion (e.g., produced by digestion with restriction enzymes), by ligation, by molecular cloning of a nucleic acid sequences in a vector (e.g., a viral or a non-viral vector), or, in the case of RNA, transcripts produced by in vitro transcription or transcripts produced in host cells (e.g., a heterologous host cells) and maintained in the host cell or purified (partially or substantially) from the host cell and maintained in solution. In addition, polynucleotides or nucleic acids can be or can include a regulatory element such as a promoter, enhancer, ribosome binding site, internal ribosomal entry site (IRES), or a transcription terminator (e.g., polyadenylation signal). Further, polynucleotides or nucleic acids can comprise sequence elements encoding for regulatory amino acid sequences in the encoded protein product. Non-limiting examples of such regulatory amino acid sequences are intracellular localization signals in the (e.g., nuclear localization signals), self-processing cleavage sites (e.g., 2A peptides such as P2A, E2A, F2A, and T2A, or furin cleavage sites).

As used herein, the term “specifically hybridizes” refers to the ability of a molecule (e.g., a gRNA) to hybridize to one nucleic acid sequence (e.g., a target genomic region) with greater affinity than it hybridizes to another nucleic acid sequence. An oligonucleotide can specifically hybridizes to more than one target sequence.

As used herein, the term “modulate,” or “modulation” refers to a change of amount or quality of a function or activity when compared to the function or activity prior to modulation. Modulation includes either an increase (stimulation or induction) or a decrease (inhibition or reduction).

As used herein, the term “variant” refers to an alternative molecule (e.g., an alternative genomic sequence, transcript, or protein) that differs from the corresponding wild type molecule. As used herein, the term “wild type,” refers to a strain, a molecule (e.g., a nucleic acid (e.g., a gene or a transcript) or a protein) or characteristic, which prevails among individuals in natural conditions, and it is distinct from a variant of the strain, molecule (e.g., nucleic acid (e.g., gene or transcript) or protein), or characteristic. A variant may differ from the wild type molecule in any region of the molecule, for example, in any domain of a protein, in any regulatory region of a protein or nucleic acid molecule, or in any coding or non-coding region of a nucleic acid molecule, or any combination thereof. A variant may be present in any percentage of the population (e.g., <0.001%, or >10%). A variant may or may not cause phenotypic changes, and may or may not cause a disorder, disease, syndrome, or condition. A variant can be present in all the cells of an individual, or in some of the cells of the individual (e.g., in some cell lineages of the individual).

A variant genomic sequence or transcript can comprise one or more nucleotide differences with respect to the wild type sequence, such as indels (i.e., insertions and/or deletion), nucleotide expansions, transversions, translocations, inversions, chromosomal structure alterations, gene fusions, chromosome fusions, truncations, amplifications, duplications, chromosomal lesions, or any combination thereof. As a result, a variant nucleotide sequence can be altered at a single nucleotide, which may be added, deleted, or substituted, or at more than one nucleotide. Further the structure of a chromosome can be altered, with regions being flipped, deleted, duplicated, truncated, or translocated. The expression of a gene can also be amplified (or reduced) through increased (or decreased) copy number of a gene; this scenario is defined as “copy number variation.”

A variant genomic sequence can arise, for example, from unrepaired DNA damage, replication errors, or mobile genetic elements. A variant genomic sequence can be inherited from parents (i.e., may be present in the germline of one or both parents) or can be acquired over the life of an individual (i.e., appear at any time over the life of an individual in the somatic or germ line of the individual). It is also to be understood that “a copy of a gene that is not a wild type copy of the gene,” is herein intended to refer to a variant of the gene. It is understood, that a gene (e.g., the dystrophin gene) is to be intended as the whole genomic region, i.e., comprising coding regions, non-coding regions, regulatory regions, or any combination thereof.

It is to be understood that the term a “variant” further encompasses proteins encoded by variant genomic sequences or transcripts.

A variant transcript can also be encoded and/or produced from the same genomic region from which the wild type molecule is encoded and/or produced, but yet differ from the wild type molecule encoded and/or produced from the same genomic region as a results of differences introduced during the transcription process and/or during RNA processing (e.g., may be the product of alternative splicing).

Additionally, a variant protein can be encoded or produced from the same genomic region or transcript as the wild type protein, but yet differ from the wild type protein as a results of differences introduced during the translation process.

As used herein, the term “nucleotide” refers to monomeric units of nucleic acid polymers (e.g., deoxyribonucleic acid (DNA) and ribonucleic acid (RNA)). Naturally occurring nucleotides are composed of three subunit molecules: a naturally occurring nucleobase, a naturally occurring five-carbon sugar (ribose or deoxyribose), and a naturally occurring phosphate group consisting of one to three phosphates. A nucleotide can also be a modified nucleotide, which is a nucleotide comprising a modified nucleobase, a modified sugar moiety, a modified backbone, or any combination thereof.

As used herein, the term “nucleobases,” also known as “nitrogenous bases” or “bases,” refers to biological compounds that form nucleosides, which, in turn, are components of nucleotides. A nucleobase may be any naturally occurring, such as adenine, guanine, cytosine, thymine and uracil, or any synthetic or modified nucleobase that is capable of hydrogen bonding with a nucleobase present on a target pre-mRNA. Examples of modified nucleobases include, without limitation, hypoxanthine, xanthine, 7-methylguanine, 5,6-dihydrouracil, 5-methylcytosine, and 5-hydroxymethylcytosine.

A “nucleoside” comprises a nucleobase and a sugar moiety. Both the nucleobase and the sugar moiety can be naturally occurring or modified. Naturally occurring nucleotides includes deoxyribonucleotides and ribonucleotides. Modified nucleotides includes nucleotides with modified or substituted sugar groups, and/or modified nucleobases.

A “sugar moiety” may comprise ribose or deoxyribose, as present in naturally occurring nucleotides, or a modified sugar moiety or sugar analog, including a morpholine ring. Non-limiting examples of modified sugar moieties include 2′ substitutions such as 2′-O-methyl (2′-O-Me), 2′-O-methoxyethyl(2′MOE), 2′-O-aminoethyl, 2′F; N3′→P5′ phosphoramidate, 2′dimethylaminooxyethoxy, 2′dimethylaminoethoxyethoxy, 2′-guanidinium, 2′-O-guanidinium ethyl, carbamate modified sugars, and bicyclic modified sugars. A sugar moiety modification may include an extra bridge bond, such as in a locked nucleic acid (LNA). A sugar analog may contain a morpholine ring, such as phosphorodiamidate morpholino (PMO). A sugar moiety may comprise a ribofuranosyl or 2′deoxyribofuranosyl modification. A sugar moiety may comprise 2′4′-constrained 2′O-methyoxyethyl (cMOE) modifications. A sugar moiety may comprise cEt 2′,4′ constrained 2′-O ethyl BNA modifications. A sugar moiety may comprise tricycloDNA (tcDNA) modifications. A sugar moiety may comprise ethylene nucleic acid (ENA) modifications. A sugar moiety may comprise MCE modifications. Modifications are described in the literature, e.g., by Jarver, et al., 2014, “A Chemical View of Oligonucleotides for Exon Skipping and Related Drug Applications,” Nucleic Acid Therapeutics 24 (1): 37-47, incorporated by reference for this purpose herein.

As used herein, the term “backbone,” and “backbone structure,” refer to the connection between monomers of a nucleic acid. In naturally occurring oligonucleotides, the backbone comprises a 3′-5′ phosphodiester linkage connecting sugar moieties of the oligomer. The backbone structure may include, for example, phosphorothioate, phosphorodithioate, phosphoroselenoate, phosphorodiselenoate, phosphoroanilothioate, phosphoranilidate, phosphoramidate, and the like. See, e.g., LaPlanche et al. Nucleic Acids Res. 14:9081 (1986); Stec et al. J. Am. Chem. Soc. 106:6077 (1984), Stein et al. Nucleic Acids Res. 16:3209 (1988), Zon et al. Anti Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990). A backbone structure may not contain phosphorous but rather peptide bonds, for example in a peptide nucleic acid (PNA), or linking groups including carbamate, amides, and linear and cyclic hydrocarbon groups. A backbone modification can be a phosphothioate linkage, or a phosphoramidate linkage.

The term “vector,” as used herein, includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may comprise specific functional sequences needed for insertion and/or expression of the desired DNA fragments. A “vector” can be any vehicle for the cloning of and/or transfer of a nucleic acid into a host cell, such as a plasmid, phage, transposon, cosmid, chromosome, artificial chromosome, virus, virion, etc. The term “vector” includes both viral and nonviral vehicles for introducing the nucleic acid into a cell in vitro, ex vivo, or in vivo. In some aspects, insertion of a polynucleotide into a suitable vector can be accomplished by ligating the appropriate polynucleotide fragments into a chosen vector that may or not have complementary cohesive termini. Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes described in the literature include: genes providing resistance to neomycin, ampicillin, streptomycin, gentamycin, kanamycin, hygromycin, bialaphos herbicide, sulfonamide, and the like; and genes that are used as phenotypic markers, i.e., anthocyanin regulatory genes, isopentenyl transferase gene, and the like. Examples of reporters described in the literature include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

As used herein, the term “RNA” relates to a nucleic acid molecule that comprises ribonucleotide residues. In some aspects, the RNA contains all or a majority of ribonucleotide residues. As used herein, “ribonucleotide” refers to a nucleotide with a hydroxyl group at the 2′-position of a b-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered RNAs are considered analogs of naturally-occurring RNA. The term “mRNA,” as used herein, refers to a single stranded RNA that encodes the amino acid sequence of one or more peptide (e.g., oligopeptide, or polypeptide) or protein. The term “mRNA,” as used herein includes in vitro transcribed RNA (IVT RNA) or synthetic RNA. An mRNA molecule may also contain a 5′ untranslated region (5′-UTR), and/or a 3′ untranslated region (3′-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides.

The term “expression,” as used herein, refers to a process by which a gene produces a biochemical, for example, an RNA transcript or a protein. The process includes any manifestation of the functional presence of the gene within the cell including, without limitation, gene knock-in, as well as both transient expression and stable expression. It may include, without limitation, transcription of the gene into messenger RNA (mRNA), and the translation of such mRNA into protein(s). Expression of a gene produces a “gene product.” As used herein, a gene product can be either a nucleic acid, e.g., a messenger RNA, or a non-coding RNA, produced by transcription of a gene, or a protein which is translated from an mRNA transcript. Gene products described herein further include nucleic acids with post transcriptional modifications, e.g., mRNAs which are processed, for example, by capping, splicing, and/or polyadenylation, or peptides with post translational modifications, e.g., methylation, glycosylation, the addition of lipids, association with other protein subunits, proteolytic cleavage, and the like.

As used herein, the terms “protein,” “polypeptide,” and “peptide,” are used interchangeably, and are intended to encompass a singular “protein,” as well as plural “proteins,” and refer to a molecule composed of monomers (amino acids) linearly linked by amide bonds (also known as peptide bonds). The term “protein” refers to any chain or chains of two or more amino acids and does not refer to a specific length of the product. Thus, peptides, dipeptides, tripeptides, oligopeptides, polypeptide, protein, amino acid chain, or any other term used to refer to a chain or chains of two or more amino acids, are included within the definition of “protein,” and the term “protein” can be used instead of, or interchangeably with, any of these terms. The term “protein” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A protein can be derived from a natural biological source or produced by recombinant technology, but is not necessarily translated from a designated nucleic acid sequence. It can be generated in any manner, including by chemical synthesis.

Non-limiting examples of proteins include protein fragments, denatured/unstructured proteins, proteins having a primary, a secondary, a tertiary, or a quaternary or aggregated structure, etc.

A protein as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Proteins can have a defined three-dimensional structure, although they do not necessarily have such structure. Proteins with a defined three-dimensional structure are referred to as folded, and polypeptides that do not possess a defined three-dimensional structure, but rather can adopt a large number of different conformations, and are referred to as unfolded. Non-limiting examples of proteins include protein fragments, denatured/unstructured proteins, proteins having a primary, a secondary, a tertiary, or a quaternary or aggregated structure, etc.

As used herein, the term “coding sequence” or a sequence “encoding” refers to a particular molecule which is a nucleic acid that is transcribed (in the case of DNA) or translated (in the case of RNA) into protein, in vitro or in vivo, when operably linked to an appropriate regulatory sequence, such as a promoter. The boundaries of the coding sequence are determined by a start codon at the 5′ (amino) terminus and a translation stop codon at the 3′ (carboxy) terminus. Although a “stop codon” (e.g., TAG, TGA, or TAA) is not translated into an amino acid, it can be considered to be part of a coding region, but any flanking sequences, for example promoters, ribosome binding sites, transcriptional terminators, introns, and the like, are not part of a coding region. A coding sequence can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the coding sequence.

As used herein, the term “exon” refers to a coding section of a DNA molecule, or of an RNA molecule which is transcribed from a DNA molecule, that is translated into protein. Exons can be separated by intervening sections of DNA that do not code for proteins, known as “introns.” Therefore, the term “intron,” as used herein, refers to a segment of nucleic acid that is transcribed and is present in the “pre-mRNA” but excised by the splicing machinery and therefore not present in the mature mRNA transcript. Following transcription, new, immature strands of messenger RNA, called “pre-mRNA,” may contain both introns and exons. These pre-mRNA molecules go through a modification process in the nucleus called splicing during which the noncoding introns are cut out and only the coding exons remain in the “mature mRNA.” Splicing produces a mature messenger RNA molecule that is then translated into a protein. The term “first exon” refers to a coding sequence or sequence of nucleic acid that encodes a polypeptide or polypeptide region and the term “second exon” refers to a different second coding sequence or sequence of nucleic acid that encodes a second polypeptide region. Where the two exons are separated by an intervening intron in the pre-mRNA, the splicing machinery operates to remove the intervening intron and join the two exons in the mature mRNA.

The term “polyadenylation signal,” refers to a nucleic acid sequence present in the RNA transcript that allows for the transcript, when in the presence of the enzyme polyadenyl transferase, to be polyadenylated.

The term “promoter,” as used herein in, refers to a sequence sufficient to direct transcription, in a cell. A promoter is intended as a DNA region to which RNA polymerases bind and that directs the enzyme to transcribe an operably linked DNA sequence. A DNA sequence is operably linked to a promoter if the promoter is capable of directing transcription of that DNA sequence. Promoters for use in the invention include prokaryotic, eukaryotic (e.g., mammalian or yeast), and viral promoters, e.g., the CMV (mammalian cytomegalovirus) promoter, the CAG promoter (also known as CBA promoter; CMV early enhancer/chicken β actin promoter), the UbC (polyubiquitin C gene) promoter, or the CBh (an engineered CBA promoter in which the SV40 intron is replaced with a hybrid intron composed of a 5′ donor splice site from the chicken β-actin 5′ UTR and a 3′ acceptor splice site from MVM). A promoter can be a “constitutive” promoter that is a promoter that, when operably linked to a polynucleotide encoding a gene product, results in the production of a gene product in the cell under most or all conditions of the cell. A promoter can be a “regulatable promoter” that is a promoter whose activity is affected by a cis or trans acting factor (e.g., an inducible promoter, such as an external signal or agent). The term “inducible” promoter means that when the promoter is operably linked to a polynucleotide encoding a specified gene product, it results in the production of a gene in the cell basically only when the inducer corresponding to the promoter is present in the cell. A promoter can be a “ubiquitous promoter” that is a promoter that is active in a wide range of cells, tissues and cell cycles, or a “tissue-specific promoter” that is a promoter that has activity only or mostly in certain cell types, i.e., drives the expression of the operably linked nucleotide sequence only or mostly in certain cell types. A promoter can be a “bidirectional promoter” that is a promoter that is an intergenic region between two divergent genes located on complementary strands of the DNA, and drives their coordinated transcription in opposite directions.

As used herein, the term “regulatory sequence” refers to a nucleic acid sequence capable of regulating the expression of a nucleic acid sequence operably linked to said regulatory sequence, non-limiting examples of regulatory sequences are enhancers (a DNA sequence that increases the level of transcription of an operably linked gene), and silencers (a DNA sequence that decreases the level of transcription of an operably linked gene). The term “regulatory sequence” also refers to nucleic acid sequence in RNA transcripts capable of regulating, for example, the processing or the expression of said transcripts. Non-limiting examples of regulatory sequences that can be in RNA transcripts are nucleotide sequences that regulate localization or splicing of said RNA transcripts. The term “regulatory sequence” further refers to an amino acid sequence capable of regulating, for example, the localization (e.g., subcellular localization signals, such as nuclear localization signals), or the stability (e.g., degradation signals) of a protein.

As used herein the term “splicing” refers to the process by which introns are removed from primary transcripts (pre-mRNA) and exons are joined to form the mature mRNA. Introns are removed by the pre-mRNA by cleavage at conserved sequences called “splice sites,” or “splicing sites.” These sites are located at the 5′ and 3′ ends of introns. Most commonly, the RNA sequence that is removed begins with the dinucleotide GU at its 5′ end, and ends with AG at its 3′ end. These consensus sequences are known to be critical, because changing one of the conserved nucleotides may result in the inhibition of splicing. Another important sequence occurs at what is called the branch point, located anywhere from 18 to 40 nucleotides upstream from the 3′ end of an intron. The branch point always contains an adenine, but it is otherwise loosely conserved. A typical sequence is YNYYRAY, where Y indicates a pyrimidine, N denotes any nucleotide, R denotes any purine, and A denotes adenine. Rarely, splice site sequences are found that begin with the dinucleotide AU and end with AC, these are spliced through a similar mechanism.

Splicing occurs in several steps and is catalyzed by small nuclear ribonucleoproteins (snRNPs, commonly pronounced “snurps”). First, the pre-mRNA is cleaved at the 5′ end of the intron following the attachment of a snRNP called Ul to its complementary sequence within the intron. The cut end then attaches to the conserved branch point region downstream through pairing of guanine and adenine nucleotides from the 5′ end and the branch point, respectively, to form a looped structure known as a lariat. The bonding of the guanine and adenine bases takes place via a chemical reaction known as transesterification, in which a hydroxyl (OH) group on a carbon atom of the adenine attacks the bond of the guanine nucleotide at the splice site. The guanine residue is thus cleaved from the RNA strand and forms a new bond with the adenine.

Next, the snRNPs U2 and U4/U6, in some embodiments, contribute to positioning of the 5′ end and the branch point in proximity. With the participation of U5, the 3′ end of the intron is brought into proximity, cut, and joined to the 5′ end. This step occurs by transesterification; in this case, an OH group at the 3′ end of the exon attacks the phosphodiester bond at the 3′ splice site. The adjoining exons are covalently bound, and the resulting lariat is released with U2, U5, and U6 bound to it. In addition to consensus sequences at their splice sites, eukaryotic genes with long introns also contain exonic splicing enhancers (ESEs). These sequences, which help position the splicing apparatus, are found in the exons of genes and bind proteins that help recruit splicing machinery to the correct site. Most splicing occurs between exons on a single RNA transcript, but occasionally trans-splicing occurs, in which exons on different pre-mRNAs are ligated together.

The splicing process occurs in cellular machines called spliceosomes, in which the snRNPs are found along with additional proteins. The primary variety of spliceosome is one of the most plentiful structures in the cell, and recently, a secondary type of spliceosome has been identified that processes a minor category of introns. These introns are referred to as U12-type introns because they depend upon the action of a snRNP called U12 (the common introns described above are called U2-type introns). The role of U12-type introns is not yet defined, but their persistence throughout evolution and conservation between homologous genes of widely divergent species suggests an important functional basis.

As used herein, the term “alternative splicing” refers to a deviation from the constitutive splicing in which introns are removed and exon are in the order in which they appear in a gene. In alternative splicing certain exons are skipped resulting in various forms of mature mRNA from a single pre-RNA transcript. Weaker splicing signals at alternative splice sites, shorter exon length or higher sequence conservation surrounding orthologous alternative exons influence the exons that are ultimately included in the mature mRNA. Three possible mechanisms: exon shuffling, exonization of transposable elements and constitutively spliced exons, have been proposed for the origin of alternative splicing. Alternative splicing is the mechanism that accounts for the discrepancy between the number of protein-coding genes (~25,000) in humans and the >90,000 different proteins that are actually generated.

The terms “operatively linked,” “operatively inserted,” “operatively positioned,” “under control” means, with reference to two or more nucleic acid sequences, that the nucleic acid sequences are arranged in such a way that one of the to two or more nucleic acid sequences can mediate a function that is exerted upon at least one of the other two or more nucleic acid sequences. For example, a regulatory nucleic acid sequence (e.g., a promoter, an enhancer, or a silencer) can be “operatively linked,” to a coding nucleic acid sequence, that is the regulatory nucleic acid sequence is in the correct location and orientation in relation to the coding nucleic acid sequence to control expression of the coding nucleic acid sequence (e.g., via control of RNA polymerase initiation). Wherein a regulatory nucleic acid sequence (e.g., a promoter, an enhancer, or a silencer) is “operatively linked,” to a coding region, the coding region is “under transcriptional control” of the regulatory nucleic acid sequence (e.g., a promoter, an enhancer, or a silencer).

The term “operably linked” means that a nucleic acid sequence and a regulatory sequence(s) are arranged in such a way as to permit gene expression when the appropriate molecules (e.g., transcriptional activator proteins) are bound to the regulatory sequence(s). The term “operably inserted” means that a nucleic acid sequence of interest is positioned adjacent a regulatory nucleic acid sequence which directs transcription and translation of the nucleic acid sequence of interest (i.e., facilitates the production of, e.g., a polypeptide encoded by a DNA of interest).

As used herein, the term “recombinant DNA/RNA technology” refers to the manipulation of nucleic acid sequences outside of an organism. This technology comprises, but is not limited to, combining nucleic acid sequences (e.g., coding sequences, regulatory elements (e.g., promoters, enhancers, silencers, termination sequences), linkers (e.g., spacers, internal ribosome entry sites, cleavage sites)) derived from a variety of sources, inserting nucleic acid sequences from a variety of sources in appropriate vectors (e.g., delivery vectors, expression vectors, integrating vectors), modifying or altering nucleotide sequences (e.g., by mutagenesis, insertion of modified nucleotides, 5′-capping, polyadenylation), synthesizing artificial nucleotide sequence. A variety of techniques described in the literature (e.g., molecular cloning, polymerase chain reaction (PCR), digestion with restriction enzymes, in vitro ligation, mutagenesis, site-directed mutagenesis, prokaryotic and eukaryotic cell transformation or transduction, in vitro DNA/RNA synthesis, in vitro RNA-5′-capping, in vitro RNA-polyadenylation, complementary DNA (cDNA) synthesis, nucleic acid isolation, and the like) can be used to manipulate nucleic acid sequences outside an organism (see for example Green & Sambrook Molecular Cloning: A Laboratory Manual, volumes 1-3, 4th edition).

As used herein, the term “recombinant,” refers to any nucleic acid (e.g., DNA, or RNA), peptide (e.g., oligopeptide, polypeptide, or protein), cell, or organism, which is made by manipulation of nucleic acid sequences. For example, “recombinant DNA” molecules are DNA molecules derived from one organism and inserted in a host organism to produce new genetic combinations. For example, “recombinant RNA” molecule (e.g., recombinant mRNA molecules) are RNA molecules derived from one organism and inserted in a host organism to produce the expression of a desired genetic product in the host organism.

As used herein, the term “genetic locus,” refers to the physical site or location within a genome of a specific DNA sequence, for example a gene.

As used herein, the term “cell” or “cells” refers not only to the particular subject cell, but also to the progeny or to the potential progeny of such cell(s). The scope of the term as used herein also encompasses the progeny that may or may not in fact be identical to the parent cell because certain modifications may occur in succeeding generations due to either mutation or environmental influences.

As used herein, the term “DNA binding protein” refers to a protein that binds DNA in a sequence specific or non-specific manner. In some embodiments, the DNA binding protein is a protein which binds with significant affinity to a specific DNA sequence. In some embodiments, the DNA binding protein binds to a DNA sequence non-specifically. The term “DNA-binding fragment” refers to a fragment of a protein, e.g., a 7 kDa DNA-binding protein, a zinc finger protein, transcription activator-like effectors (TALE), or a transcription activator-like effector protein, and other transcriptional regulators, their homologues, orthologues, and mutants with preserved or enhanced basic functions of DNA-binding proteins.

As used herein, the term “administration” refers to the administration of a composition or substance to a subject or system. Administration to an animal subject (e.g., to a human) can be by any appropriate route. “Administering” refers to the physical introduction of a composition or substance, which may comprising a therapeutic agent, to a subject, using any of the various methods and delivery systems known to those skilled in the art. Examples of routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Administration can also be via a non-parenteral route, for example, orally. Other non-parenteral routes include a topical, epidermal or mucosal route of administration, for example, intranasally, vaginally, rectally, aborally, sublingually, or topically. Administering can also be performed, for example, once, a plurality of times, and/or over one or more extended periods.

As used herein, the terms “treat,” “treated,” and “treating” mean both therapeutic and prophylactic treatment or preventative measures wherein the object is to reverse, alleviate, ameliorate, lessen, inhibit, slow down progression, development, severity or recurrence of an undesired symptom, complication, condition, biochemical indicia of a disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. In some aspects, treatment includes eliciting a clinically significant response without excessive levels of side effects. In some aspects, treatment includes prolonging survival as compared to expected survival if not receiving treatment. As used herein, the term “amelioration” or “ameliorating” refers to a lessening of severity of at least one indicator of a condition or disease. As used herein, the term “preventing” or “prevention” refers to delaying or forestalling the onset, development or progression of a condition or disease for a period of time, including weeks, months, or years. As used herein, the term “prophylactic” (e.g., “prophylactic agent,” “prophylactic treatment,” “prophylactically effective amount”), refers to any complete or partial prevention of a disease or symptom thereof and/or can be therapeutic in terms of a partial or complete cure for a disease and/or adverse effect and/or symptom attributable to the disease.

As used herein, the term “gene therapy” refers to the administration into an individual's cells and/or tissues of an exogenous molecule (e.g., a nucleic acid sequence (e.g., a polynucleotide comprising a promoter operably linked to a nucleic acid encoding a gene product capable of interfering with the genomic sequence of the individual's cells and/or tissues)) to treat, reduce the symptoms of, or reduce the likelihood of a disease, disorder, syndrome, or condition. An exogenous molecule or sequence is understood to be molecule or sequence not normally occurring in the cell, tissue and/or individual to be treated. Both acquired and congenital diseases are amenable to gene therapy.

As used herein, the term “subject” refers to any organism to which a composition or a substance (e.g., a nucleotide molecule) can be administered, e.g., for experimental, diagnostic, prophylactic, and/or therapeutic purposes. Typical subjects include any animal (e.g., mammals such as mice, rats, rabbits, non-human primates, and humans). A subject can seek or be in need of treatment, require treatment, be receiving treatment, be receiving treatment in the future, or be a human or animal who is under care by a trained professional for a particular disease or condition.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary of Biochemistry and Molecular Biology, 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below and above are more fully defined by reference to the specification in its entirety.

Various aspects of the invention are described in further detail in the following subsections.

### 2. Cas-DNA Binding Protein (Cas-DNA-B) Fusion Protein

In some aspects, disclosed herein are engineered Cas enzymes having increase genome-editing efficiency. In some embodiment, the Cas enzyme is a dCas, or deficient Cas, which is a mutant form of Cas enzyme that has no or reduced endonuclease activity. Cas enzymes are part of CRISPR systems (“Clustered Regularly Interspaced Short Palindromic Repeats”). There are five main types of CRISPR systems that are distinguished by the repertoires of genes associated with CRISPR, the organization and sequence of Cas genes, and the structure of repeats within CRISPR matrices. These five types of systems have been divided into two classes: class I grouping together types I, III and IV which use a multimeric crRNA effector module, and class II grouping together types II, V and VI which use a monomeric crRNA effector module.

The class II and type II CRISPR systems are predominantly represented by the Cas9 enzymes (e.g., SpCas9, SaCas9, and NmeCas9), the class II and type V CRISPR systems are predominantly represented by Cas12 enzymes (e.g., Cas12f1, AsCas12f1, CasMINI, and SpCas12f1).

The inventors have demonstrated that it is possible to engineer the Cas proteins to increase genome-editing efficiency. They have in fact shown that fusion proteins comprising a Cas enzyme (e.g., a Cas12f1 enzyme) or its fragment, and a DNA-binding protein, or a DNA-binding fragment thereof, are capable of genome-editing with an efficiency higher than the same Cas enzyme (or its fragment) not fused to a DNA-binding protein or a DNA-binding fragment thereof.

In some embodiments, DNA-binding proteins are proteins that are capable of binding DNA via DNA-binding domains. In some embodiments, DNA-binding proteins can have a specific or general affinity for single- or double-stranded DNA. In some embodiments, DNA-binding proteins can interact with the major groove of DNA, because it exposes more functional groups that identify a base pair, or with the minor groove of DNA.

In some embodiments, DNA-binding proteins include transcription factors that modulate the process of transcription, various polymerases, nucleases which cleave DNA molecules, and histones which are involved in chromosome packaging and transcription in the cell nucleus. In some embodiments, DNA-binding proteins can comprise domains such as, but not limited to, zinc finger (ZF), helix-turn-helix, helix-loop-helix, leucine zipper, and transcription activator like effectors (TALE). In some embodiments, a fragment of DNA-binding proteins includes a domain within the transcription factors.

A zinc finger (ZN) is a protein structural motif that is characterized by the coordination of one or more zinc ions (Zn2+) in order to stabilize the fold. The vast majority ZN proteins (e.g., zinc finger nucleases and zinc finger transcription factors) typically function as interaction modules that bind DNA, RNA, proteins, or other small, useful molecules, and variations in structure serve primarily to alter the binding specificity of a particular protein.

ZN proteins binding properties depend on the amino acid sequence of the ZN finger domains and on the linker between fingers, as well as on the higher-order structures and the number of fingers. ZN proteins function, for example, in gene transcription, translation, mRNA trafficking, cytoskeleton organization, epithelial development, cell adhesion, protein folding, chromatin remodeling, and zinc sensing. Zinc-binding motifs are stable structures, and they rarely undergo conformational changes upon binding their target, and recognize specific DNA sequences through protein-DNA interactions.

Transcription activator-like effectors (TALEs) are proteins secreted by some proteobacteria. The TALE domain responsible for binding to DNA is known to have 1.5 to 33.5 short sequences that are repeated multiple times (i.e., tandem repeats). Each of these repeats is specific for a certain base pair of the DNA. These repeats also have repeat variable residues (RVD) that can detect specific DNA base pairs. TALEs recognize DNA sequences through a central repeat domain consisting of a variable number of ~34 amino acid repeats. There appears to be a one-to-one correspondence between the identity of two critical amino acids in each repeat and each DNA base in the target sequence.

“7 kDa DNA-binding” proteins are DNA-binding proteins derived from different sources. 7 kDa DNA-binding proteins can be found, for example, in archea (i.e., archeal 7 kDa DNA-binding proteins). An exemplary, but not limiting, “7 kDa DNA-binding” family, also known as the Sul7d family, is a family of DNA-binding proteins, found in archaeal (Kalichuk et al., Sci. Rep., 2016). Examples of 7 kDa DNA-binding proteins include, but are not limited to, Sac7d, Sac7e, Mse7, Mcu7, Aho7a, Aho7b, Aho7c, Sto7, Ssh7b, Sis7a, Sis7b, Ssh7a, and Sso7d. 7 kDa DNA-binding proteins were first isolated from Sulfolobus acidocaldarius, which produces five of them, named Sac7a, b, c, d, and e. Sac7d and Sac7e are encoded by distinct genes, while Sac7a and b are truncated versions of Sac7d. Highly similar homologs have been found in all Sulfolobus species, such as Sso7d from Sulfolobus solfataricus, and Ssh7a and Ssh7b (two proteins encoded by two distinct genes) from Sulfolobus shibatae. Sac7d and Sso7d are hyperthermostable (Tm=90.4° C. and 100.2° C., respectively) and are resistant from pH 0 up to at least pH 12. Sac7d and Sso7d both fold as an SH3-like domain capped by a C-terminal α-helix and that sharply kink the double DNA helix upon binding into the minor groove. Sac7d and Sso7d are general dsDNA binders (i.e., do not need specific nucleotide sequences to bind to a DNA molecule) with KD values varying in a salt dependent manner and with a slight preference for G/C rich sequences.

In some aspects, disclosed herein is Cas enzyme (or its fragment) engineered through fusion with a DNA binding protein or a DNA-binding fragment thereof having increase genome-editing efficiency. The Cas enzyme (or its fragment) engineered through fusion with a DNA binding protein, or a DNA-binding fragment thereof disclosed herein, is herein also referred to as Cas-DNA-B fusion protein.

In some aspects, the Cas enzyme (or its fragment) and the DNA binding protein or DNA-binding fragment thereof are linked via one or more chemical bonds, a linker, or a combination thereof.

In some aspects, the chemical bond is a covalent chemical bond. In some aspects, the Cas enzyme (or its fragment) and the DNA binding protein or DNA-binding fragment thereof are linked via one or more covalent bonds and/or one or more noncovalent bonds. For example, the Cas enzyme (or its fragment) and the DNA binding protein or DNA-binding fragment thereof can be linked by an amide bond (also known as a peptide bond) and/or by a ionic interaction, a hydrophobic interaction, a hydrogen bond, or any combination thereof. As an example, the C-terminus of the Cas enzyme (or its fragment) and can be linked by an amide bond to the N-terminus of the DNA binding protein or DNA-binding fragment thereof; the Cas enzyme (or its fragment) and the DNA binding protein or DNA-binding fragment thereof can be further linked by an ionic interaction between different amino acid residues on the Cas enzyme (or its fragment) and on the DNA binding protein or DNA-binding fragment thereof to form a complex structure.

In some aspects, the Cas enzyme (or its fragment) and the DNA binding protein or DNA-binding fragment thereof are linked via a linker. Linkers can be flexible linkers, rigid linker, or cleavable linkers. The linker can improve the folding or the stability of a fusion protein, increase expression, improve biological activity, enable targeting, or alter the chemical properties of a fusion protein. As an example, the C-terminus of the Cas enzyme (or its fragment) can be linked by an amide bond to the N-terminus of the DNA binding protein or DNA-binding fragment thereof; the Cas enzyme (or its fragment) and the DNA binding protein or DNA-binding fragment thereof can be further linked by an ionic interaction between different amino acid residues on the Cas enzyme (or its fragment) and on the DNA binding protein or DNA-binding fragment thereof to form a complex structure.

In some aspects, the Cas enzyme (or its fragment) and the DNA binding protein or DNA-binding fragment thereof are linked via one or more chemical bonds and via a linker. As an example, the C-terminus of the Cas enzyme (or its fragment) and can be linked by a linker to the N-terminus of the DNA binding protein or DNA-binding fragment thereof; the Cas enzyme (or its fragment) and the DNA binding protein or DNA-binding fragment thereof can be further linked by a ionic interaction between different amino acid residues to create a complex structure.

The linker used herein can be an amino acid chain, in some embodiment. Non-limiting examples of linkers are (G)ₙ, (G₄S)ₙ (SEQ ID NO: 125), (GSG)ₙ, (GGGGS)ₙ (SEQ ID NO: 58), (EAAK)ₙ (SEQ ID NO: 59), or (XP)ₙ, wherein n is any integer, e.g., any integer between 1 and 35, and X is any amino acid. For example, a linker can be (G) 6-8, (G₄S)₁₋₉, (GSG)₁₋₅, (GGGGS)₁₋₄, (EAAK)₁₋₃, or (XP), wherein X is A, L, or E, for example (AP)₇. In some aspects, n is an integer between 1 and 31. In some aspects, n is an integer that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35.

In some aspects, the Cas enzyme (or its fragment) and the DNA binding protein or DNA-binding fragment thereof can be linked via a (GSG)ₙ linker, wherein n is an integer between 1 and 31. In some aspects, the Cas enzyme (or its fragment) and the DNA binding protein or DNA-binding fragment thereof can be linked via a (GSG)ₙ linker, wherein n is an integer that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31. In some aspects, the Cas enzyme (or its fragment) and the DNA binding protein or DNA-binding fragment thereof are linked via a (GSG) 1 linker.

The Cas enzyme (or its fragment) and the DNA binding protein or DNA-binding fragment thereof can be linked in different conformations. For example, the DNA-binding protein or DNA-binding fragment thereof can be located upstream of the Cas enzyme (or its fragment) or downstream of the Cas enzyme.

In some aspects, the Cas enzymes are type II or type IV Cas enzymes. In some aspects, the Cas enzymes are Cas9 or Cas12 Cas enzymes. In some aspects, the Cas enzymes are SpCas9, SaCas9, NmeCas9, Cas12f1, AsCas12f1, CasMINI, or SpCas12f1.

In some aspects, the Cas enzymes are SpCas9 or SaCas9. In some aspects, the Cas enzymes (or their fragments) comprise an amino acid sequence at least 70%, at least 80%, at least 90%, at least 95%, or 100% identical to amino acids 41-1407 of SEQ ID NO: 25 (i.e., SEQ ID NO: 110). In some aspects, the Cas enzymes (or their fragments) comprise an amino acid sequence at least 70%, at least 80%, at least 90%, at least 95%, or 100% identical to amino acids 18-1069 of SEQ ID NO: 27 (i.e., SEQ ID NO: 111). In some aspects, the Cas enzymes (or their fragments) are encoded by a nucleotide sequence at least 70%, at least 80%, at least 90%, at least 95%, or 100% identical to nucleotides 121-4221 of SEQ ID NO: 24 (i.e., SEQ ID NO: 113). In some aspects, the Cas enzymes (or their fragments) are encoded by a nucleotide sequence at least 70%, at least 80%, at least 90%, at least 95%, or 100% identical to nucleotides 52-3207 of SEQ ID NO: 26 (i.e., SEQ ID NO: 114).

In some aspects, the Cas enzymes (or their fragments) comprise an amino acid sequence at least 70%, at least 80%, at least 90%, at least 95%, or 100% identical to an amino acid sequence of SEQ ID NO: 110 or 111. In some aspects, the Cas enzymes (or their fragments) are encoded by a nucleotide sequence at least 70%, at least 80%, at least 90%, at least 95%, or 100% identical to a nucleotide sequence of SEQ ID NO: 113 or 114.

In some aspects, the Cas enzymes (or their fragments) comprise an amino acid sequence at least 70%, at least 80%, at least 90%, at least 95%, or 100% identical to an amino acid sequence of SEQ ID NO: 25 or 27. In some aspects, the Cas enzymes (or their fragments) are encoded by a nucleotide sequence at least 70%, at least 80%, at least 90%, at least 95%, or 100% identical to a nucleotide sequence of SEQ ID NO: 24 or 26.

In some aspects, the Cas enzymes are Cas12f1 or SpCas12f1. In some aspects, the Cas enzymes (or their fragments) comprise an amino acid sequence at least 70%, at least 80%, at least 90%, at least 95%, or 100% identical to amino acids 17-545 of SEQ ID NO: 29 (i.e., SEQ ID NO: 112). In some aspects, the Cas enzymes (or their fragments) are encoded by a nucleotide sequence at least 70%, at least 80%, at least 90%, at least 95%, or 100% identical to nucleotides 49-1635 of SEQ ID NO: 28 (i.e., SEQ ID NO: 115).

In some aspects, the Cas enzymes (or their fragments) comprise an amino acid sequence at least 70%, at least 80%, at least 90%, at least 95%, or 100% identical to an amino acid sequence of SEQ ID NO: 112. In some aspects, the Cas enzyme (or their fragments) are encoded by a nucleotide sequence at least 70%, at least 80%, at least 90%, at least 95%, or 100% identical to a nucleotide sequence of SEQ ID NO: 115.

In some aspects, the Cas enzymes (or their fragments) comprise an amino acid sequence at least 70%, at least 80%, at least 90%, at least 95%, or 100% identical to an amino acid sequence of SEQ ID NO: 29. In some aspects, the Cas enzyme (or their fragments) are encoded by a nucleotide sequence at least 70%, at least 80%, at least 90%, at least 95%, or 100% identical to a nucleotide sequence of SEQ ID NO: 28.

In some aspects, the Cas enzymes (or their fragments) are fused to a DNA-binding protein, or to a DNA-binding fragment thereof. In some aspects, the DNA-binding protein, or DNA-binding fragment thereof, comprises a zinc finger (ZF), helix-turn-helix, helix-loop-helix, leucine zipper, or transcription activator like effectors (TALE) domain. In some aspects, the a DNA-binding protein, or a DNA-binding fragment thereof is a ZF protein. In some aspects, the a DNA-binding protein, or a DNA-binding fragment thereof is a TALE. In some aspects, the a DNA-binding protein, or a DNA-binding fragment thereof is a 7 kDa DNA-binding protein. In some aspects, the 7 kDa DNA-binding protein comprises Sac7d, Sac7e, Mse7, Mcu7, Aho7a, Aho7b, Aho7c, Sto7, Ssh7b, Sis7a, Sis7b, Ssh7a, or Sso7d. In some aspects, the 7 kDa DNA-binding protein is Sso7d. In some aspects, the 7 kDa DNA-binding protein comprises an amino acid sequence at least 70%, at least 80%, at least 90%, at least 95%, or 100% identical to an amino acid sequence of SEQ ID NO: 31 or 33. In some aspects, the 7 kDa DNA-binding protein is encoded by a nucleotide sequence at least 70%, at least 80%, at least 90%, at least 95%, or 100% identical to a nucleotide sequence of SEQ ID NO: 30 or 32.

In some aspects, the Cas-DNA-B fusion proteins disclosed herein comprises an amino acid sequence at least 70%, at least 80%, at least 90%, at least 95%, or 100% identical to an amino acid sequence of SEQ ID NO: 110, 111, or 112 and an amino acid sequence at least 70%, at least 80%, at least 90%, at least 95%, or 100% identical to an amino acid sequence of SEQ ID NO: 31 or 33.

In some aspects, the Cas-DNA-B fusion proteins disclosed herein comprise an amino acid sequence at least 70%, at least 80%, at least 90%, at least 95%, or 100% identical to an amino acid sequence of SEQ ID NO: 110, 111, or 112; an amino acid sequence at least 70%, at least 80%, at least 90%, at least 95%, or 100% identical to an amino acid sequence of SEQ ID NO: 31 or 33, and a linker selected from the group consisting of (G)ₙ, (G₄S)ₙ, (GSG)ₙ (SEQ ID NO: 125), (GGGGS)ₙ (SEQ ID NO: 58), (EAAK)ₙ (SEQ ID NO: 59), or (XP)ₙ, wherein n is an integer between 1 and 35 and X is any amino acid.

In some aspects, the Cas-DNA-B fusion proteins disclosed herein comprise an amino acid sequence at least 70%, at least 80%, at least 90%, at least 95%, or 100% identical to amino acids 17-612 of SEQ ID NO: 11 (i.e., SEQ ID NO: 117). In some aspects, the Cas-DNA-B fusion proteins disclosed herein comprise an amino acid sequence at least 70%, at least 80%, at least 90%, at least 95%, or 100% identical to amino acids 41-1474 of SEQ ID NO: 16 (i.e., SEQ ID NO: 119). In some aspects, the Cas-DNA-B fusion proteins disclosed herein comprise an amino acid sequence at least 70%, at least 80%, at least 90%, at least 95%, or 100% identical to amino acids 18-1136 of SEQ ID NO: 18 (i.e., SEQ ID NO: 121). In some aspects, the Cas-DNA-B fusion proteins disclosed herein comprise an amino acid sequence at least 70%, at least 80%, at least 90%, at least 95%, or 100% identical to amino acids 17-608 of SEQ ID NO: 20 (i.e., SEQ ID NO: 123). In some aspects, the Cas-DNA-B fusion proteins disclosed herein are encoded by a nucleotide sequence at least 70%, at least 80%, at least 90%, at least 95%, or 100% identical to nucleotides 49-1836 of SEQ ID NO: 10 (i.e., SEQ ID NO: 118). In some aspects, the Cas-DNA-B fusion proteins disclosed herein are encoded by a nucleotide sequence at least 70%, at least 80%, at least 90%, at least 95%, or 100% identical to nucleotides 121-4422 of SEQ ID NO: 15 (i.e., SEQ ID NO: 120). In some aspects, the Cas-DNA-B fusion proteins disclosed herein are encoded by a nucleotide sequence at least 70%, at least 80%, at least 90%, at least 95%, or 100% identical to nucleotides 52-3408 of SEQ ID NO: 17 (i.e., SEQ ID NO: 122). In some aspects, the Cas-DNA-B fusion proteins disclosed herein are encoded by a nucleotide sequence at least 70%, at least 80%, at least 90%, at least 95%, or 100% identical to nucleotides 49-1824 of SEQ ID NO: 19 (i.e., SEQ ID NO: 124).

In some aspects, the Cas-DNA-B fusion proteins disclosed herein comprise an amino acid sequence at least 70%, at least 80%, at least 90%, at least 95%, or 100% identical to an amino acid sequence of SEQ ID NO: 117, 119, 121, or 123. In some aspects, the Cas-DNA-B fusion proteins disclosed herein are encoded by a nucleotide sequence at least 70%, at least 80%, at least 90%, at least 95%, or 100% identical to a nucleotide sequence of SEQ ID NO: 118, 120, 122, or 124.

In some aspects, provided herein are expression cassettes encoding the Cas-DNA-B fusion proteins disclosed herein. In some aspects, the expression cassette further comprises additional sequence elements. Non-limiting examples of additional sequence elements that can be included in the expression cassettes encoding the Cas-DNA-B fusion proteins of the disclosure are: promoters (e.g., UbC (SEQ ID NO: 53) or CBh (SEQ ID NO: 54) promoters), enhancers, silencers, 3′-UTRs, 5′-UTR, introns, IRES, sequences encoding subcellular localization signals (e.g., NLSs), sequences encoding self-cleaving peptides (e.g., T2A (SEQ ID NO: 56)), molecular tags (e.g., HA tag (SEQ ID NO: 55)), sequences encoding selectable markers (e.g., sequences encoding products conferring antibiotic resistance (e.g., hygromycin-resistance conferring genes, or HygR (SEQ ID NO: 57)).

Nuclear localization signals (NLS) are generally short peptides that act as a signal fragment that mediates the transport of proteins from the cytoplasm into the nucleus. This NLS-dependent protein recognition, a process necessary for cargo proteins to pass the nuclear envelope through the nuclear pore complex, is facilitated by members of the importin superfamily. Non-limiting examples of NLS are:

(SEQ ID NO: 34)
PKLKRQ,
(SEQ ID NO: 35)
RPRK,
(SEQ ID NO: 36)
RRARRPRG,
(SEQ ID NO: 37)
GKRKLITSEEERSPAKRGRKS,
(SEQ ID NO: 38)
KGKKGRTQKEKKAARARSKGKN,
(SEQ ID NO: 39)
RKRCAAGVGGGPAGCPAPGSTPLKKPRR,
(SEQ ID NO: 40)
RKPVTAQERQREREEKRRRRQERAKEREKRRQERER,
(SEQ ID NO: 41)
RSGGNHRRNGRGGRGGYNRRNNGYHPY,
(SEQ ID NO: 42)
TLLLRETMNNLGVSDHAVLSRKTPQPY,
(SEQ ID NO: 43)
PGKMDKGEHRQERRDRPY,
(SEQ ID NO: 44)
GKKKKGKPGKRREQRKKKRRT,
(SEQ ID NO: 45)
SANKVTKNKSNSSPYLNKRKGKPGPDS,
(SEQ ID NO: 46)
VHSHKKKKIPTSPTFTTPKTLTLRRQPKYPRKSAPRRNKLDHY,
(SEQ ID NO: 47)
RKHKTNRKPR,
(SEQ ID NO: 48)
NRRAKAKR,
(SEQ ID NO: 49)
RNKKKK,
(SEQ ID NO: 50)
RKVIK,
(SEQ ID NO: 51)
PKKKRKV,
and
(SEQ ID NO: 52)
KRPAATKKAGQAKKKK.

In some aspects, the expression cassette further comprises one or more molecular tags that allow the detection or isolation of the amino acid sequence. Exemplary molecular tags are, but are not limited to, CBP, FLAG, GST, HA, HBH, MBP, Myc, polyHis, S-tag, SUMO, TAP, TRX, or V5.

In some aspects, the amino acid sequence encoded by the expression cassette comprises from the N-term to the C-term: a NLS, a Cas enzyme, a GSG linker, a DNA-binding protein or a DNA-binding fragment thereof, a molecular tag, a self-cleaving peptide, a selectable marker. In some aspects, the amino acid sequence encoded by the expression cassette comprises a self-cleaving peptide. In some aspects, the amino acid sequence encoded by the expression cassette undergoes self-cleavage upon translation. In some aspects, the amino acid sequence encoded by the expression cassette generates two distinct amino acid sequences upon self-cleavage, a first amino acid sequence comprising from the N-term to the C-term: a NLS, a Cas enzyme, a GSG linker, a DNA-binding protein or a DNA-binding fragment thereof, a molecular tag, and optionally portion of a self-cleaving peptide, and a second amino acid sequence comprising from the N-term to the C-term optionally portion of a self-cleaving peptide and, a selectable marker.

In some aspects, the expression cassettes comprise a nucleotide sequence at least 70%, at least 80%, at least 90%, at least 95%, or 100% identical to a nucleotide sequence of SEQ ID NO: 10, 15, 17 or 19.

In some aspects, the expression cassettes encode an amino acid sequence at least 70%, at least 80%, at least 90%, at least 95%, or 100% identical to an amino acid sequence of SEQ ID NO: 11, 16, 18, or 20.

In some aspects, the expression cassette further comprises a nucleotide sequence encoding at least one cell penetrating peptide. A cell penetrating peptide (CPP) is a peptide that can be linked, for example, to a fusion protein, to facilitate the intake and uptake of the fusion protein by the cells. The CPP can be linked to the fusion protein at any location (e.g., C-terminus, or N-terminus). The CPP can be linked to the fusion protein by one or more chemical bonds, or by a linker.

CPPs can have an amino acid composition that either contains a high relative abundance of positively charged amino acids (i.e., polycationic CPPs), or an alternating pattern of polar, charged amino acids and non-polar, hydrophobic amino acids (i.e., amphipathic CPPs). Alternatively, CPPs can be hydrophobic peptides, containing only apolar residues with low net charge or hydrophobic amino acid groups that are crucial for cellular uptake.

Non-limiting examples of CPPs are 2, 18A, α1H, α2H, A22p, Ac-18A-NH2, aCPP, AdVpVI(33-55), AIP6, all-d DsC18, αgliadin(31-43), Alyteserin-2a, ANG, ApoE(141-150), ApoE-derived, Arf(1-22), AT1002, AT1AR(304-318), Bac7, BGPC7-FHV, Bim, BP16, BP100, BPP13a, bPrPp(1-30), BR2, Buforin II, Buforin IIb, C6M1, C105Y, CADY, CAR, CA-Tat, CB5005 M, CDB3, CendR, cF<tR4, CGKRK, CIGB-300, CIGB-552, CLIP6, CooP, CpMTP, CPNT, CPP1, CPP33, CPP-C, CPPecp, C-peptide, CRGDK, Crotamine, cSN50, 65-2CTS, CWR8K, CyLoP-1, Cyt c(77-101), DAG, D-JNKI-1, DK17, DLP, DMBT1-derived, dNP2, DPV3, DPV1047, DRTTLTN, DS4.3, Dynorphin A, EA, EB1, EF, EHB, EPRNEEK, F3**, G₄R9L4, GALA, GeT, gH625, Gi3α(346-355), Glu-Lys, GV1001, GWH1, H2A derived, H6R6, H16, HA2(1-23), HAIYPRH, hBD3-3, HBP, hLF, Hph-1, HR9, Hst5, 11WL5W, 14WL5W, ID No. 2, IMT-P8, INF, INGR, isl-1, JB9, JB434, KAFAK, KALA, Kalata B1, (KFF) 3K, K-FGF, KH, KLA, KLAK, KLA-R7, KP, KST peptide, L1-6, L5a, L17E, lactoferrampin(265-284), lactoferricin(17-30), lactoferrin(19-40), LAH1, LALF(31-52), LB, L-CPP, LDP-NLS, LE10, LF chimera, linTT1, LK, LL37, LLIIL, LMWP, LP-12, LPAs, LTV, lycosin-I, Lyp1, M918, Maurocalcine, MAP, MAP12, MCOTI-I, MCoTI-II, MFK, Mgpe9, MitP, m(KLA)-iRGD, MMGP1, MPER fragment, MPG, MPG, MPG-8, mRVG, MT23, mtCPP1, MTM, MTD84, MTP, MTS, MTS1, Mut3DPT-C9h, Myr-ApoE, New modalities, NF1, NF51, NF55, NLS, NLS-StAx-h, NoLS, Novicidin, NPFSD, NYAD-1, Oct4-PTD, P007, P1, P2, P3, P7, P11, P11, P11LRR, P14LRR, p18, P21, P28, p28, p53 peptide MO6, PAF26, PAS, pCLIP6, pD-SP5, PenetraMax, Penetratin, Pep-1, pepM, pepR, Pept1, Peptide 599, Pep42, PepNeg, PepFect6, PepFect14, PG1, pHLIP, PHPs, PIP1, Pip5e, Pip6a, POD, PR9, PTD, PTD3, PTD4, Poly-Arg, pVEC, Pyrrhocoricin, R4K1, R6dGR, R8, R8-dGR, R9-H4A2, R6W3, R10W6, RA9, RALA, RDP, REDV, RF, CRGDfC, iRGD, RGE, RH9, RL9, RL16, RT53, RTP004, RV24, RVG, RVG-9R, RVG29, RW9, RW16, (RXR) 4, (rXr) 4, S155, S4 (13)-PV, SAP, SAP (E), all-D-SAP, SAPSp-lipo, SAR6EW, sC18, (sC18) 2, SMTP motif, SPACE, SRCRP2-11, STR-KV, SS-02, SS-20, SS-31, SynB1, T2, Tat (49-57), Tat (48-60), Tat (44-57), Tat (37-72), Tat analog, Tat-LK15, TCTP, TD-1, TD2.2, TH peptide, TM2, Transportan, TP10, TPk, Tpl, TPP, TT1, vAMP 059, vCU.S. Plant Pat. No. 769, vCPP 2319, VDAC (1-26), VP22, V peptide, VT5, W(RW)4, Xentry, X-pep, YKA, YTA2, YTA4, Z2, Z3, Z12, and ZXR-1 (see, for example, Ülo Langel, CPP, Cell-Penetrating Peptides, Springer Singapore (2019)). In some aspects, the Cas-DNA-B fusion proteins has a genome-editing efficiency that is about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, higher compared to the genome-editing efficiency of the same Cas enzyme (or its fragment) that is not in a Cas-DNA-B fusion protein.

In some aspects, the Cas-DNA-B fusion proteins comprises from the N-term to the C-term: a Cas enzyme, and a DNA-binding protein or a DNA-binding fragment thereof. In some aspects, the Cas-DNA-B fusion proteins comprises from the N-term to the C-term: a DNA-binding protein or a DNA-binding fragment thereof, and a Cas enzyme.

In some aspects, provided herein are vectors comprising the expression cassettes of the disclosure. In some aspects, the vectors disclosed herein are viral vectors or no viral vectors. In some aspects, the vectors described herein are adeno-associated viral (AAV) vectors, adenoviral vectors, lentiviral vectors, or retroviral vectors.

The expression cassettes and the vectors described herein can be generated by any of the nucleic acid engineering techniques disclosed in the published literature.

Further, the expression cassettes and the vectors described herein can be codon optimized for expression in the desired cell type (e.g., eukaryotic cells). Codon-optimization describes gene engineering approaches that use synonymous codon changes to increase protein production in a specific cell type (see, for example, Welch M, et al. You're one in a googol: optimizing genes for protein expression. Journal of the Royal Society, Interface/the Royal Society. 2009; 6 (Suppl 4): S467-476; Ward N J, et al. Codon optimization of human factor VIII cDNAs leads to high-level expression. Blood. 2011; 117:798-807).

The Cas-DNA-B fusion proteins disclosed herein are to be used in conjunction with appropriate guide RNAs (gRNAs). gRNAs are short synthetic RNAs composed of a scaffold sequence necessary for Cas-binding and a user-defined spacer, in the range of 17-25 nucleotides, that defines the genomic target to be modified. Thus, one can change the genomic target of the Cas protein by simply changing the target sequence present in the gRNA.

Thus, the methods of the disclosure involve introducing into a eukaryotic cell (a) at least one Cas-DNA-B fusion protein; and (b) at least one guide RNA or nucleic acid encoding at least one guide RNA.

For type II CRISPR systems, a guide RNA comprises (i) a CRISPR RNA (crRNA) that contains a guide sequence at the 5′ end that hybridizes with a target sequence and (ii) a transacting crRNA (tracrRNA) sequence that interacts with the Cas protein. The tracrRNA sequence is generally the same in guide RNAs designed to complex with a CRISPR protein from a particular bacterial species. For type V CRISPR systems, a guide RNA comprises (i) a CRISPR RNA (crRNA) that contains a guide sequence or protospacer at the 3′ end that hybridizes with a target sequence, and in some embodiments, (ii) a transacting crRNA (tracrRNA) sequence that interacts with the Cas protein. The crRNA guide sequence of each guide RNA is different (i.e., is sequence specific).

The crRNA guide sequence is designed to hybridize with a target sequence (i.e., protospacer) that is bordered by a protospacer adjacent motif (PAM) in a double-stranded sequence. PAM sequences for Cas9 proteins include 5′-NGG, 5′-NRN, 5′-NYN, 5′-NNGRRT, 5′-NNNRRT, 5′-NGGNG, 5′-NNAGAAW, and 5′-ACAY, PAM sequences for Cas12a include 5′-TTTN and TTTV (wherein N is defined as any nucleotide, W is defined as either A or T, R is defined as either A or G, and Y is defined as either C or T), PAM sequences for Cas12f1 include 5′ T-rich PAM sequences (e.g., 5′-NTTC, 5′-GTTT, 5′-GTTC, 5′-AATC, 5′-ACTC, 5′-GGTC, 5′-GCTC, 5′-TTRT, 5′-TTTN, 5′-TTTR, 5′-TTR, 5′-TTN, 5′-CTT, and 5′-TTC). Of note, some CRISPRs systems do not require a PAM sequence to be present in the target genome region. In some aspects, the complementarity between the crRNA guide sequence and the target sequence is at least 80%, at least 85%, at least 90%, at least 95%, or at least 99%. In some aspects, the complementarity is complete (i.e., 100%). In some aspects, the length of the crRNA guide sequence can range from about 15 nucleotides to about 25 nucleotides. For example, the crRNA guide sequence can be about 15, 16, 17, 18, 19, 20, 21, 22, 23, 24, or 25 nucleotides in length. In some aspects, the crRNA can be about 19, 20, 21, or 22 nucleotides in length.

The crRNA and tracrRNA comprise repeat sequences that form one or more one-stem loop structures, which can interact with the CRISPR protein. The length of each loop and stem can vary. For example, the one or more loops can range from about 3 to about 10 nucleotides in length, and the one or more stems can range from about 6 to about 20 base pairs in length. The one or more stems can comprise one or more bulges of 1 to about 10 nucleotides.

The crRNA can range in length from about 25 nucleotides to about 100 nucleotides. In various embodiments, the crRNA can range in length from about 25 to about 50 nucleotides, from about 50 to about 75 nucleotides, or from about 75 to about 100 nucleotides. The tracrRNA can range in length from about 50 nucleotides to about 300 nucleotides. In various embodiments, the tracrRNA can range in length from about 50 to about 90 nucleotides, from about 90 to about 110 nucleotides, from about 110 to about 130 nucleotides, from about 130 to about 150 nucleotides, from about 150 to about 170 nucleotides, from about 170 to about 200 nucleotides, from about 200 to about 250 nucleotides, or from about 250 to about 300 nucleotides.

The tracrRNA sequence in the guide RNA generally is based upon the coding sequence of wild type tracrRNA in the bacterial species of interest. In some aspects, the wild-type tracrRNA sequence (or the crRNA constant repeat region and the corresponding 5′ region of the tracrRNA that forms a duplex structure with the crRNA constant repeat region) can be modified to facilitate secondary structure formation, increase secondary structure stability, facilitate expression in eukaryotic cells, increase editing efficiency, and so forth. For example, one or more nucleotide changes can be introduced into the constant guide RNA sequence (see Example 8, below).

The crRNA sequence can be linked to the tracrRNA sequence in a single guide RNA molecule (i.e., a single guide RNA or sgRNA), or, the crRNA and the tracrRNA can be two separate RNA molecules. In some aspects, the guide RNA of type V CRISPR systems can comprise only crRNA.

In some aspects, the one or more stem-loop regions of the guide RNA can be modified to comprise one or more aptamer sequences (Konermann et al., Nature, 2015, 517 (7536): 583-588; Zalatan et al., Cell, 2015, 160 (1-2): 339-50). Examples of suitable RNA aptamer protein domains include MS2 coat protein (MCP), PP7 bacteriophage coat protein (PCP), Mu bacteriophage Com protein, lambda bacteriophage N22 protein, stem-loop binding protein (SLBP), Fragile X mental retardation syndrome-related protein 1 (FXR1), proteins derived from bacteriophage such as AP205, BZ13, f1, f2, fd, fr, ID2, JP34/GA, JP501, JP34, JP500, KU1, M11, M12, MX1, NL95, PP7, φCb5, φCb8r, φCb12r, φCb23r, Qβ, R17, SP-β, TW18, TW19, and VK, fragments thereof, or derivatives thereof. The length of the additional aptamer sequence can range from about 20 nucleotides to about 200 nucleotides.

The guide RNA can comprise standard ribonucleotides, modified ribonucleotides (e.g., pseudouridine), ribonucleotide isomers, and/or ribonucleotide analogs. In some embodiments, the guide RNA can further comprise at least one detectable label. The detectable label can be a fluorophore (e.g., FAM, TMR, Cy3, Cy5, Texas Red, Oregon Green, Alexa Fluors, Halo tags, or suitable fluorescent dye), a detection tag (e.g., biotin, digoxigenin, and the like), quantum dots, or gold particles. Those skilled in the art are familiar with gRNA design and construction, e.g., gRNA design tools are available on the internet or from commercial sources.

The guide RNA can be synthesized chemically, synthesized enzymatically, or a combination thereof. For example, the guide RNA can be synthesized using standard phosphoramidite-based solid-phase synthesis methods. Alternatively, the guide RNA can be synthesized in vitro by operably linking DNA encoding the guide RNA to a promoter control sequence that is recognized by a phage RNA polymerase. Examples of suitable phage promoter sequences include T7, T3, SP6 promoter sequences, or variations thereof. In aspects in which the guide RNA comprises two separate molecules (i.e., crRNA and tracrRNA), the crRNA can be chemically synthesized and the tracrRNA can be enzymatically synthesized.

Gene-editing, including CRISPR-mediated genome editing, in some embodiments, comprises the generation of a Cas-induced double-strand break that is repaired by non-homologous end joining (NHEJ) mechanisms or by homology directed repair (HDR). In some embodiments, gene editing comprises CRISPR/Cas-mediated single-base-pair editing systems that have been devised to bypass these limitations. Two classes of DNA base-editors (BEs) have been described thus far: cytosine base-editors (CBEs) and adenine base-editors (ABEs). These BEs can install all four transition mutations. In some embodiments, gene editing comprises Prime-editors (PEs) that are the latest addition to the CRISPR genome-engineering toolkit and represents a novel approach to expand the scope of donor-free precise DNA editing to not only all transition and transversion mutations, but small insertion and deletion mutations as well. In some embodiments, gene editing comprises CRISPR/dCas-mediated modulation of gene expression, either through the process of transcriptional repression called as CRISPR interference (CRISPRi) or the process of transcriptional activation called CRISPR activation (CRISPRa). In some embodiments, gene editing comprises epigenomic editing, which allows for site-specific epigenome modifications to modulate gene expression. Briefly, CRISPR-Cas-based epigenome editing utilizes a nuclease-deficient Cas (dCas) and a synthetic guide RNA to target specific DNA sequences. The fusion of dCas to an effector (e.g., VP64, NF-κB p65, Ten-Eleven Translocation (TET) protein, histone lysine demethylase (KDM), KRAB, or a DNA demethylase) can be used to regulate endogenous gene expression.

In an embodiment, the fusion protein that comprises a Cas enzyme (or its fragment) and a DNA-binding protein (or a fragment thereof) of this disclosure can be used in all modes of gene editing, e.g., genome editing, base editing, Prime editing, and epigenomic editing.

### 3. Methods of Treatment Comprising a Cas-DNA-B Fusion Proteins

In some aspects disclosed herein are methods of treating a subject suffering from a genetic disease disorder or condition.

Genetic diseases, disorders, syndromes or conditions can be a hereditary or nonhereditary and can be caused by a defect within one or more locations within the genome. The defects can be indels (i.e., insertions and/or deletion), nucleotide expansions, transversions, translocations, inversions, chromosomal structure alterations, gene fusions, chromosome fusions, truncations, amplifications, duplications, chromosomal lesions, or any combination thereof. As a result, locations within the genome can be altered at a single nucleotide, which may be added, deleted, or substituted, or at more than one nucleotide. Further, the structure of a chromosome can be altered, with regions being flipped, deleted, duplicated, truncated, or translocated. The expression of a gene can also be amplified (or reduced) through increased (or decreased) copy number of a gene (i.e., copy number variation).

Genetic diseases, disorders, syndromes or conditions can be autosomal dominant, autosomal recessive, X-linked dominant, X-linked recessive, Y-linked, mitochondrial, or multifactorial or polygenic.

Exemplary genetic diseases, disorders, syndromes or conditions, include, but are not limited to, achondroplasia, hereditary hemochromatosis, Down Syndrome, hereditary spherocytosis, Tay-Sachs Disease, Usher syndrome, hereditary fructose intolerance, hemophilia, muscular dystrophy (e.g., Duchenne muscular dystrophy or DMD), polygenic disorders, breast cancer, ovarian cancer, Parkinson's disease, Bardet-Biedl syndrome, Prader-Willi syndrome, diabetes, heart disease, arthritis, motor neuron disease, albinism, Cri-du-Chat syndrome, cystic fibrosis, fragile X syndrome, galactosemia, Huntington's disease, Jackson-Weiss syndrome, Klinefelter syndrome, Krabbe disease, Langer-Giedion syndrome, Lesch-Nyhan syndrome, Marfan syndrome, myotonic dystrophy, Nail-Patella syndrome, neurofibromatosis, Noonan syndrome, triple X syndrome, osteogenesis imperfecta, Patau syndrome, phenylketonuria, porphyria, retinoblastoma, Rett syndrome, sickle cell disease, Turner syndrome, Usher syndrome, Von Hippel-Lindau syndrome, Waardenburg syndrome, Wilson's disease, xeroderma pigmentosum, XXXX syndrome, or YY syndrome can be characterized by an impaired production of a protein, or by a defective splicing. A hereditary disease such as for example: achondroplasia, hereditary hemochromatosis, Down Syndrome, hereditary spherocytosis, Tay-Sachs Disease, Usher syndrome, hereditary fructose intolerance, hemophilia, muscular dystrophy (e.g., Duchenne muscular dystrophy or DMD), polygenic disorders, breast cancer, ovarian cancer, Parkinson's disease, Bardet-Biedl syndrome, Prader-Willi syndrome, diabetes, heart disease, arthritis, motor neuron disease, albinism, DM, Cri-du-Chat syndrome, cystic fibrosis, fragile X syndrome, galactosemia, Huntington's disease, Jackson-Weiss syndrome, Klinefelter syndrome, Krabbe disease, Langer-Giedion syndrome. Lesch-Nyhan syndrome, Marfan syndrome, myotonic dystrophy, Nail-Patella syndrome, neurofibromatosis, Noonan syndrome, triple X syndrome, osteogenesis imperfecta, Patau syndrome, phenylketonuria, porphyria, retinoblastoma, Rett syndrome, sickle cell disease, Turner syndrome, Usher syndrome, Von Hippel-Lindau syndrome, Waardenburg syndrome, Wilson's disease, xeroderma pigmentosum, XXXX syndrome, or YY syndrome.

Muscular dystrophy is a group of muscle diseases that can weaken the musculoskeletal system and can hamper locomotion. It can be characterized by the progressive skeletal muscle weakness, defects in muscle proteins, and the death of muscle cells and tissues. One common form of muscular dystrophy can be Duchenne muscular dystrophy (DMD). Additional forms of muscular dystrophy can include Becker, limb-girdle, congenital, facioscapulohumeral, myotonic, oculopharyngeal, distal, and Emery-Dreifuss muscular dystrophy.

Spinal muscular atrophy (SMA) is an autosomal recessive disorder that is one of the most common genetic causes of childhood mortality. The main characteristic of the disease can be a progressive loss of spinal cord motor neurons, resulting in skeletal muscle denervation with subsequent weakness, atrophy, and paralysis of voluntary muscles. The SMA locus can be mapped to a complex inverted repeat of ~500 kb on Chromosome 5q13 that contains several genes. The main cause of SMA can be homozygous loss of the telomeric copy of the survivor of motor neuron gene (SMN1) located within the inverted repeat. A duplicated gene within the centromeric copy of the inverted repeat (SMN2) can also be transcribed, but the SMN2 gene does not completely compensate for loss of SMN1 function.

Ataxia-telangiectasia (A-T) or Louis-Bar syndrome is a rare neurodegenerative inherited disease. The term “ataxia” refers to poor coordination and the term “telangiectasia” refers to small dilated blood vessels. Both terms characterize the hallmarks of this disease. A-T can impair the cerebellum and additional areas of the brain that can cause impaired movement and coordination. A-T can also weaken the immune system thereby increasing infection and can impair DNA repair, thereby increasing the risk of cancer. A-T can be associated with a defect in the gene ATM, which is responsible for managing cellular response to multiple form of stress.

X-linked agammaglobulinemia, also known as X-linked hypogammaglobulinemia. XLA, Bruton type agammaglobulinemia, Bruton syndrome, or Sex-linked agammaglobulinemia, is an X-linked genetic disorder that can affect the body's ability to fight infection. XLA patients lack mature B cells and as a result, lack the necessary antibodies to combat infection. Bruton's tyrosine kinase (BTK) can be associated with mediating B cell development and maturation and the BTK gene can be associated with XLA.

Myotonic dystrophy (DM) is a form of muscular dystrophy that affects muscles and many other organs in the body. The word “myotonic” is the adjectival form of the word “myotonia,” defined as an inability to relax muscles at will. The term “muscular dystrophy” means progressive muscle degeneration, with weakness and shrinkage of the muscle tissue. Myotonic dystrophy often is abbreviated as “DM” in reference to its Greek name, dystrophia myotonica. Another name used occasionally for this disorder is Steinert disease. DM is divided into two types. Type 1 DM (DM1), long known as Steinert disease, occurs when a gene on chromosome 19 called DMPK contains an abnormally expanded section located close to the regulation region of another gene, SIX5. Type 2 DM (DM2), recognized in 1994 as a milder version of DM1, is caused by an abnormally expanded section in a gene on chromosome 3 called ZNF9. DM2 was originally called PROMM, for proximal myotonic myopathy, a term that has remained in use but is somewhat less common than the term DM2. The expanded sections of DNA in these two genes appear to have many complex effects on various cellular processes. In both DM1 and DM2, the repeat expansion is transcribed into RNA but remains untranslated in protein.

Diabetes mellitus (commonly known as diabetes) is a group of metabolic diseases characterized by a high blood sugar level over a prolonged period. Symptoms of diabetes can include weight loss, polyuria or increased urination, polydipsia or increased thirst, and polyphagia or increased hunger. Diabetes can be classified into four categories: type 1, type 2, gestational diabetes, and other specific types of diabetes. Type 1 diabetes can be characterized by a loss of insulin-producing beta cells of the islets of Langerhans in the pancreas, leading to insulin deficiency. Type 2 diabetes can be characterized by insulin resistance, which can also be combined with a reduced insulin secretion. Gestational diabetes can resemble type 2 diabetes, and can involve combination of inadequate insulin secretion and responsiveness. Other specific types of diabetes can include prediabetes, latent autoimmune diabetes of adults (LADA) and congenital diabetes.

Cancer can be a solid tumor or a hematologic malignancy. A solid tumor can be a sarcoma or a carcinoma. Sarcoma can be a cancer of bone, cartilage, fat muscle, vascular or hematopoietic tissues. Exemplary sarcoma can include alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epithelioid cell differentiation, osteosarcoma, parosteal osteosarcoma, neoplasm with perivascular epithelioid cell differentiation, periosteal osteosarcoma, pleomorphic liposarcoma, pleomorphic rhabdomyo sarcoma, PNET/extraskeletal Ewing tumor, rhabdomyosarcoma, round cell liposarcoma, small cell osteosarcoma, solitary fibrous tumor, synovial sarcoma, telangiectatic osteosarcoma.

Carcinoma can be a cancer developed from epithelial cells. Exemplary carcinoma can include adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

Hematologic malignancy is a malignancy of the blood system and can include T-cell based and B-cell based malignancies. Exemplary hematologic malignancy can include myeloid leukaemia, myeloproliferative neoplasias, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, treatment-related T-cell lymphomas, chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

The fusion proteins, the expression cassettes, and the vectors disclosed herein may be administered by any route that results in a therapeutically effective outcome. These include, but are not limited to enteral, gastroenteral, epidural, oral, transdermal, epidural (peridural), intracerebral (into the cerebrum), intracerebroventricular (into the cerebral ventricles), intrastriatal (into the striatum), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In some aspects, compositions may be administered in a way that allows them to cross the blood-brain barrier, vascular barrier, or other epithelial barrier.

The fusion proteins, the expression cassettes, and the vectors disclosed herein may be administered to a subject in a single dose, or a multiple dose. In some aspects, the multiple dose comprises two, three, or four or more doses. In some aspects, the fusion proteins, the expression cassettes, and the vectors, or pharmaceutical formulation described herein are administered to the subject at regular intervals, for example, weekly, biweekly, monthly, every 2 months, every 3 months, every 4 months, every 5 months, every 6 months or yearly. In some aspects, the first set of doses (e.g., two, three, four, five, six, seven, eight or ten doses) are administered monthly, with further doses administered less frequently (e.g., every 3 months, every 6 months or yearly).

The fusion proteins, the expression cassettes, and the vectors disclosed herein may be included in any suitable pharmaceutical formulation. As used herein, the term “formulation,” or “pharmaceutical formulation” refers to an admixture comprising an effective amount of a therapeutically and/or prophylactic effective agent (e.g., the fusion proteins, the expression cassettes, and the vectors disclosed herein).

Provided herein are pharmaceutical formulation comprising a fusion protein, an expression cassette, or a vector disclosed herein and at least one pharmaceutically acceptable excipient (e.g., carrier, diluent, stabilizer, or any combination thereof). In some aspects, the composition comprises a pharmaceutically acceptable excipient, or pharmaceutically acceptable adjuvants, or any combination thereof.

Examples of pharmaceutically acceptable excipients are, but are not limited to, carriers, binders, diluents, lubricants, thickeners, surface active agents, preservatives, stabilizers, emulsifiers, buffers, flavoring agents, colorants, amino acids, stabilizers, bulking agents, surfactants, antimicrobials, preservatives, metal ions, chelators, cyclodextrin-based excipients, polyanions, polycations, salts, solubilizers, detergents, compatible solid or liquid fillers, encapsulating substances, or any combination thereof, which are suitable for administration to a subject. Specific examples of excipient include, without limitation, sterile water, Ringer, Ringer lactate, sterile sodium chloride solution, isotonic saline, polyalkylene glycols, hydrogenated naphthalenes and, biocompatible lactide polymers, lactide/glycolide copolymers or polyoxyethylene/polyoxy-propylene copolymers, or any combination thereof. Examples of pharmaceutically acceptable carriers are, but are not limited to, lipids, polymers, polysaccharides, peptides, proteins, lipidoids, and any combination thereof. In some aspects, the lipid is selected from the group consisting of: cationic lipids, non-cationic lipids, steroid lipids, ionizable lipids, PEG-conjugated lipids, and any combination thereof. In some aspects, the fusion proteins, the expression cassettes, and the vectors disclosed herein are complexed, or packaged in a liposome, a nanoliposome, a lipid nanoparticle, a lipoplex, a micell, a nanomicell, a nanoemulsion, an oil-in-water emulsions, a PEG-conjugated lipid nanoparticle, a polymeric nanoparticle, a lipid-polymer hybrid nanoparticle, a polysaccharidic nanocarrier, an RNA/DNA-peptide nanoparticle, an RNA/DNA-peptide nanocomplex, a biomimetic nanovesicle, a lipidoid-RNA/DNA complex, a virus-like particle, dendrimer nanoparticle, a nanogel, a metallic nanoparticle, a gold nanoparticle (AuPNs), a magnetic nanoparticle, a theranostic nanoparticle, or any combination thereof. In some aspects, the combination of the fusion proteins, the expression cassettes, and the vectors disclosed herein with one or more of the carriers described herein facilitates, enhances or enables administration of the fusion proteins, the expression cassettes, and the vectors disclosed herein to the subject. In some aspects, the combination of the fusion proteins, the expression cassettes, and the vectors disclosed herein with one or more of the carriers described herein facilitates, enhances or enables the delivery of the fusion proteins, the expression cassettes, and the vectors disclosed herein to a target cell.

Pharmaceutically acceptable excipients for therapeutic use are well known in the pharmaceutical art, and are described, for example, in Remington's Pharmaceutical Sciences, Mack Publishing Co. (A. R Gennaro edit. 1985). Pharmaceutical excipients can be selected with regard to the intended route of administration and standard pharmaceutical practice.

In some aspects, after administration to the subject of the fusion proteins, the expression cassettes, and the vectors disclosed herein, at least a portion of the fusion proteins, the expression cassettes, and the vectors disclosed herein are delivered to a target cell. In some aspects, the pharmaceutical formulation described herein are a dry power, granules, a solution, a suspension, an emulsion, or any combination thereof. In some aspects, the dry power is suitable for administration to a subject upon reconstitution in a pharmaceutically acceptable vehicle. In some aspects, the granules are suitable for administration to a subject upon reconstitution in a pharmaceutically acceptable vehicle. In some aspects, the solution is an aqueous solution, or an anhydrous solution.

### WORKING EXAMPLES

### Example 1: Generation and Molecular Cloning of Cas12f1-Sso7d C-Terminal Fusion

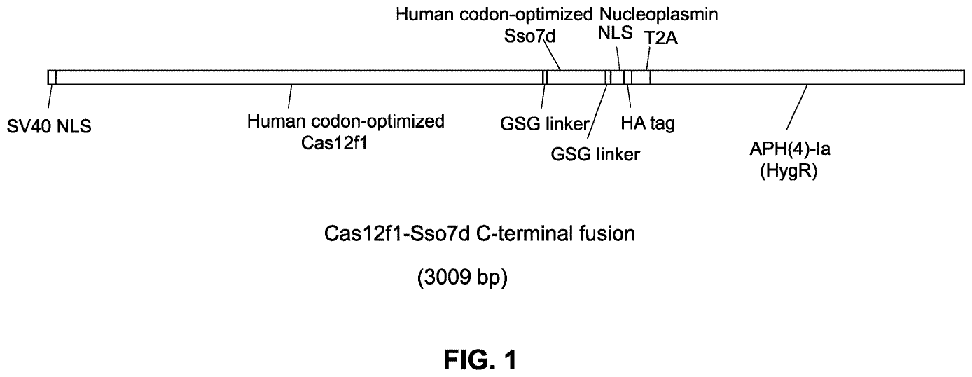

The Cas12f1-Sso7d C-terminal fusion construct was generated and cloned into the pLV-43 lentiviral expression vector, as described below. A first fragment (i.e., Fragment 1) comprising the sequence from the nucleoplasmin NLS through an Esp3I restriction site within the Hygromycin resistance sequence was amplified by PCR (NEBNext Ultra II Q5 Master Mix) using pLV-43 as the template and the set of primers of SEQ ID NOs: 1 and 2. Fragment 1 comprises: a sequence encoding the nucleoplasmin NLS, a sequence encoding an HA tag, a linker sequence, a sequence encoding a T2A self cleaving peptide, and a sequence corresponding to the first 91 nucleotides of a hygromycin gene. A second fragment (i.e., Fragment 2) comprising the GSG-Sso7d-GSG sequence was amplified by PCR using a nucleic acid molecule comprising the nucleotide sequence of SEQ ID NO: 3 as the template and the set of primers of SEQ ID NOs: 4 and 5. A third fragment (i.e., Fragment 3) comprising the region of the Cas12f1 coding sequence, encoding the C-terminal portion of the Cas12f1, was amplified by PCR using pLV-43 (SEQ ID NO: 9) as the template and the set of primers of SEQ ID NOs: 6 and 7. The pLV-43 lentiviral expression vector was linearized by restriction enzyme digestion with BamHI and Esp3I restriction enzymes. The high molecular weight fragment (i.e., Fragment 4) was isolated via agarose gel electrophoresis and purified via gel extraction (Takara). Fragments 1~4 were then assembled using the In-Fusion HD Cloning kit (Takara), using 25 fmol of Fragment 4 and a molar ratio of 10:10:3:1 for Fragments 1:2:3:4. Clones were then verified by Sanger sequencing (Azenta Life Sciences) using the primer of SEQ ID NO: 8. The resulting construct (pLV-EW8, SEQ ID NO: 12) comprises and expression cassette encoding a Cas12f1-Sso7d (FIG. 1, SEQ ID NO: 10 (DNA sequence) and SEQ ID NO: 11 (amino acid sequence)) under the control of a UbC promoter (i.e., UbC: Cas12f1-Sso7d). All sequences are human codon-optimized and expressed co-translationally, originating from a single polypeptide.

Similarly, the pLV-EW9 (SEQ ID NO: 13) construct comprising a CBh: Cas12f1 expression cassette, was engineered to obtain the pLV-EW11 (SEQ ID NO: 14) construct comprising a Cas12f1-Sso7d expression cassette under the control of a CBh promoter (i.e., CBh: Cas12f1-Sso7d).

Further, an expression cassette encoding a SpCas9-Sso7d fusion protein (SEQ ID NO: 15 (DNA sequence) and SEQ ID NO: 16 (amino acid sequence)), an expression cassette encoding a SaCas9-Sso7d fusion protein (SEQ ID NO: 17 (DNA sequence) and SEQ ID NO: 18 (amino acid sequence)), and an expression cassette encoding a Cas12f1-Aho7c (SEQ ID NO: 19 (DNA sequence) and SEQ ID NO: 20 (amino acid sequence)) were produced and cloned to obtained the corresponding vectors.

### Example 2: Cas12f1 Constructs Editing Efficiency Assay at the DMD Locus

Editing efficiency was assessed across 24 separate Cas12f1 targets (5′-TTTRN20-3′; PAM sequence underlined) identified within introns 50 and 51 of the DMD locus.

### Cell Culture and Transfection

Briefly, HEK293FT cells were seeded in a 96-well plate at 4×10⁴ cells/well in 150 ul DMEM supplemented with 10% FBS, 100U/ml Penicillin-Streptomycin, 1×MEM NEAA, 1×GlutaMAX. Cells were allowed to settle and adhere overnight. Cas12f1-expressing plasmids (pLV-43 (UbC: Cas12f1), pLV-EW8 (UbC: Cas12f1-Sso7d), pLV-EW9 (CBh: Cas12f1), or pLV-EW11 (CBh: Cas12f1-Sso7d)) and U6: gRNA PCR products (amplified using pLV-46 (SEQ ID NO: 21) as template with sets of primers comprising the nucleotide sequence of SEQ ID NO: 22 and 23, wherein *N₂₀ represents the reverse complement of the protospacer sequence for each reverse primer) were normalized to 200 ng/ul, and 150 ng of each plus 0.4 μ1 Lipofectamine 2000 were diluted in OptiMEM to a total volume of 25 μl. Transfection mixtures were vortexed and incubated at room temperature for 5 min, and the entire volume was transfected into each well. Cells were incubated at 37° C., 5% CO₂ and genomic DNA was harvested on the third day post-transfection. All transfections were performed in duplicate, including 18 non-targeting control wells which were transfected with 150 ng pL V-46 (representing an empty U6: gRNA cassette from which the U6: gRNA PCR products were generated).

### Illumina Library Preparation and Sequencing

The 150-250 bp region centered around each target site was amplified via 22 cycles of PCR (25 μl reaction volume) from 16 ng of genomic DNA input using primers with partial Illumina adapter sequences. A second round of PCR (9 cycles) was performed using 7.5 μl of the purified first PCR (~1 ng) and NEBNext Multiplex Oligos for Illumina to add full adapter sequences and unique i5/i7 indexes for each sample (with one sample representing one well from the assay). All PCRs were purified with the KingFisher Apex System using Ampure XP beads (0.9× volume relative to sample volume) and eluted in 40 μl 10 mM Tris-HCl, pH 8. The second round of PCRs were all normalized to 4 nM, and 5 μl of each were pooled to generate the library. The library was diluted to 12 μM and sequenced on a MiSeq using MiSeq Reagent Kit V2, 300 cycle PE cartridge and reagents.

### Indel Frequency Identification

Editing efficiency was assessed simultaneously for each replicate of a given target (including non-targeting controls) with the crispresso2 software (Clement et al., 2019).

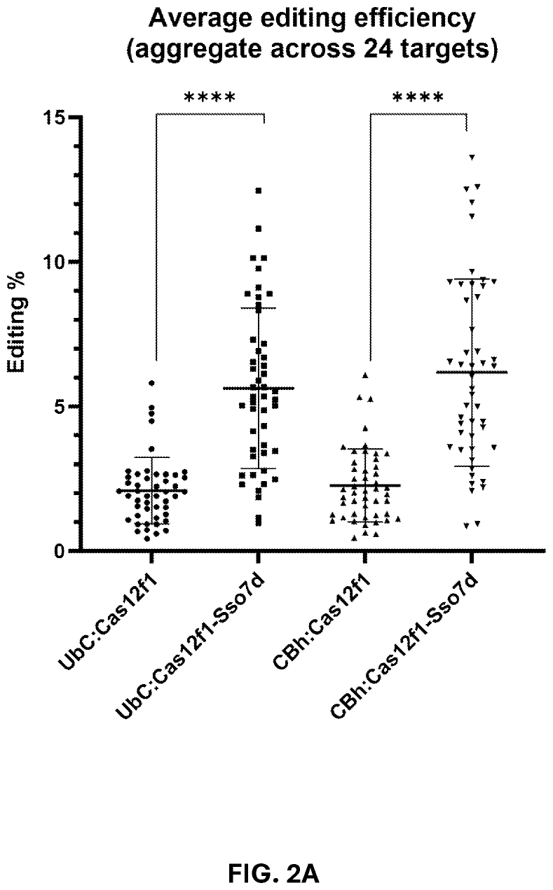

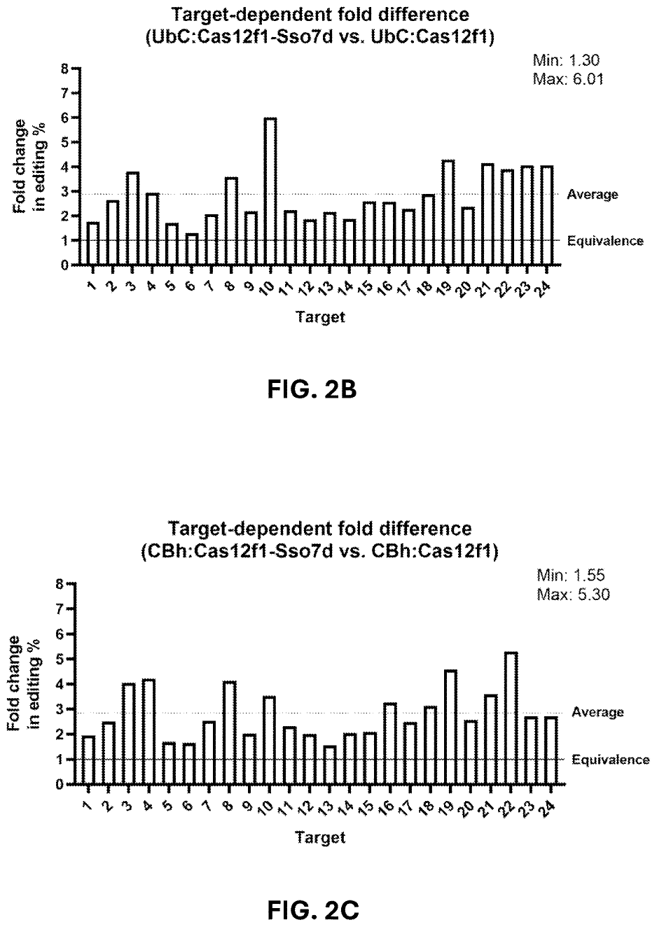

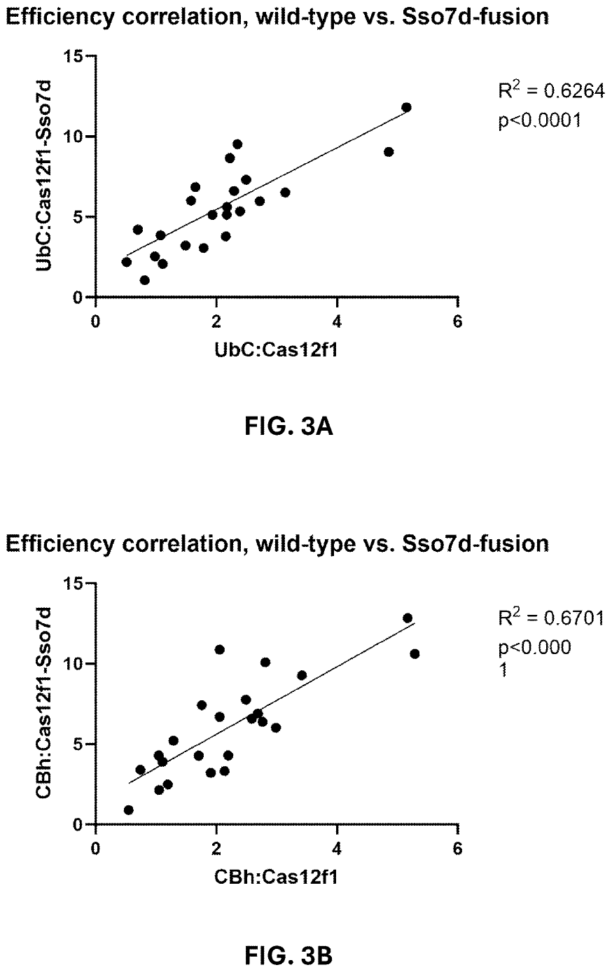

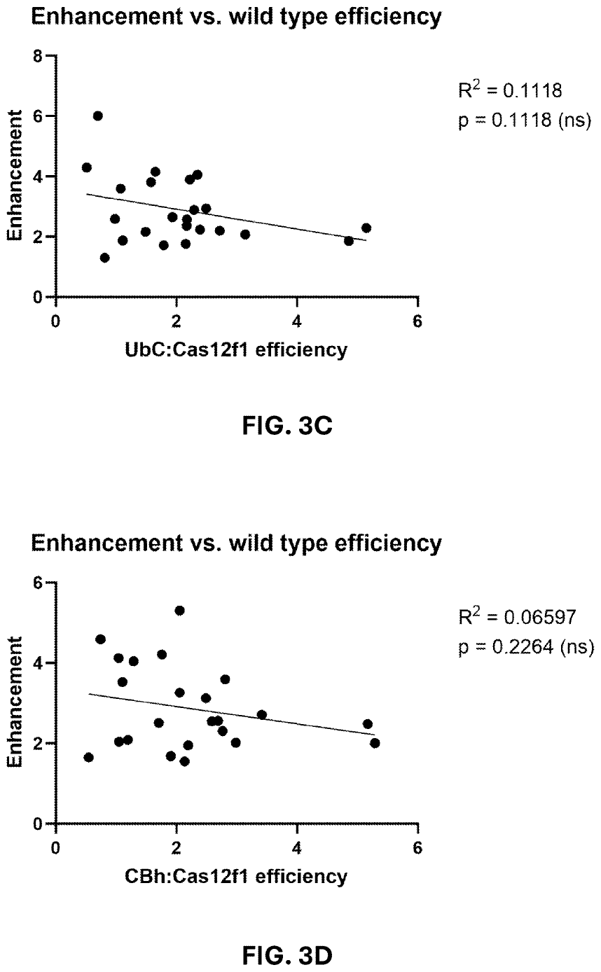

The Cas12f1-Sso7d fusion constructs yielded a significant increase in editing efficiency (~3× on average, p<0.0001) compared to the Cas12f1 without the Sso7d protein. Promoter choice did not appreciably affect editing efficiency in this assay (FIG. 2A). Notably, the Sso7d fusion resulted in an increase in editing efficiency for every target. The increase in efficiency ranged from 1.3× to 6×, depending on the target, both under the control of the UbC and under the control of the CBh promoters (FIG. 2B-C). The mean fold change when assessed in this manner was similar to the pooled mean at ~3×. The editing efficiencies exhibited a strong positive correlation, indicating that targets that are edited with high efficiency using wild type Cas12f1 are also likely to be edited with high efficiency by Cas12f1-Sso7d, both under the control of the UbC (FIG. 3A) and under the control of the CBh (FIG. 3B) promoters. The degree of editing efficiency enhancement provided by the Sso7d does not correlate strongly with ease of editing at a given target, assessed as fold change in editing vs. wild type editing efficiency, both under the control of the UbC (FIG. 3C) or of the CBh (FIG. 3D) promoters). This, coupled with the observation that the Sso7d fusion provided editing efficiency enhancements at all 24 targets, suggests that it is likely that the Sso7d fusion will provide a positive benefit (in terms of editing efficiency) at most targets, with large benefits being possible even at targets that already edit at high efficiency.

All further analyses were performed using the indel frequency values from crispresso2 analysis.

### Example 3: Cas12f1 Constructs-Mediated Large Deletion Assay

Cell culture, transfection, incubation, and gDNA isolation conditions were identical to those describe in Example 2, except that the transfection input was modified to include 100 ng Cas12f1-expressing plasmid, 100 ng U6: gRNA PCR product for target 1, and 100 ng U6: gRNA PCR product for target 2. Targets were situated such that a deletion event would remove DMD intron 50 splice acceptor, DMD exon 51, and DMD intron 51 splice donor sequences. 5% of each gDNA isolate (2 μl of a 40 μl elution) was assessed via amplification of a region spanning the potential deletion. PCRs were run to endpoint (35 cycles) with primers listed in Table 1. Each reaction was diluted 1:20 in water and 1 μl was analyzed using an Agilent TapeStation with High-Sensitivity D5000 screen tape and reagents. Deletion efficiency was assessed by dividing the deletion band concentration by the combined concentrations of the wild type and deletion bands, multiplied by 100 to express as a percent.

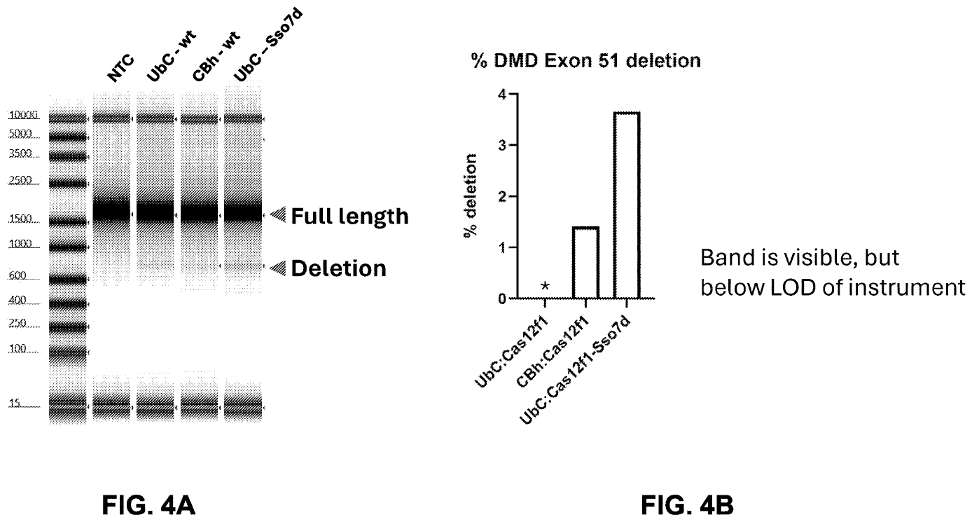

TapeStation analysis of deletion-spanning PCR products from unedited and edited genomic DNA showed that the expected deletion band was absent in the unedited sample but was visibly present in all edited samples (FIG. 4A-B). However, the band was too low in abundance to be automatically quantified by the analysis software for the UbC: Cas12f1 sample, and it was visibly denser for the UbC: Cas12f1-Sso7d sample compared to the UbC: Cas12f1 or CBh: Cas12f1 samples. The Cas12f1-Sso7d fusion yielded an enhancement in large deletion efficiency that was similar to the average enhancement observed in editing efficiency at individual targes (~3×). Given the similarities between the UbC and CBh promoters observed in previous assays, CBh: Cas12f1-Sso7d was not assessed in this assay.

### Example 4: Generation and Molecular Cloning of Cas12f1-Aho7c C-Terminal Fusion and Cas12f1 Constructs Editing Efficiency Assay at the DMD Locus

The lentiviral expression construct pLV-EW15, containing the C-terminal Aho7c fusion (SEQ ID NO: 116), was synthesized by Genscript. Editing efficiency was assessed for 2 separate Cas12f1 targets (5′-TTTRN20-3′; PAM sequence underlined) identified within introns 50 and 51 of the DMD locus.

### Cell Culture and Transfection

Briefly, HEK293FT cells were seeded in a 96-well plate at 4×10⁴ cells/well in 150 μl DMEM supplemented with 10% FBS, 100U/ml Penicillin-Streptomycin, 1×MEM NEAA, 1×GlutaMAX. Cells were allowed to settle and adhere overnight. Cas12f1-expressing plasmids (pLV-43, pLV-EW8, or pLV-EW15) and U6: gRNA PCR products (amplified using pLV-46 as template with primer EWc12f009 and primer set EWc12f-T.x) were normalized to 200 ng/ul, and 150 ng of each plus 0.4 μl Lipofectamine 2000 were diluted in OptiMEM to a total volume of 25 ul. Transfection mixtures were vortexed and incubated at room temperature for 5 min, and the entire volume was transfected into each well. Cells were incubated at 37° C., 5% CO₂ and genomic DNA was harvested on the third day post-transfection. All transfections were performed in duplicate, including 18 non-targeting control wells which were transfected with 150 ng pLV-46 (representing an empty U6: gRNA cassette from which the U6: gRNA PCR products were generated).

### Illumina Library Preparation and Sequencing

An ~150-250 bp region centered around each target site was amplified via 22 cycles of PCR (25 μl reaction volume) from 16 ng of genomic DNA input using primers with partial Illumina adapter sequences. A second round of PCR (9 cycles) was performed using 7.5 μl of the purified first PCR (~1 ng) and NEBNext Multiplex Oligos for Illumina to add full adapter sequences and unique i5/i7 indexes for each sample (with one sample representing one well from the assay). All PCRs were purified with the KingFisher Apex System using Ampure XP beads (0.9× volume relative to sample volume) and eluted in 40 μl 10 mM Tris-HCl, pH 8. The second round of PCRs were all normalized to 4 nM, and 5 μl of each were pooled to generate the library. The library was diluted to 12 μM and sequenced on a MiSeq using MiSeq Reagent Kit V2, 300 cycle PE cartridge and reagents.

### Indel Frequency Identification

Editing efficiency was assessed simultaneously for each replicate of a given target (including non-targeting controls) with the crispresso2 software (Clement et al., 2019) using the following parameters:
CRISPRessoBatch—batch_settings [input file list]—amplicon_seq [reference sequence]-g [protospacer sequence]-w 6-p 4-wc 6-bo [output path]

All further analyses were performed using the indel frequency values from crispresso2 analysis (Clement et al., 2019).

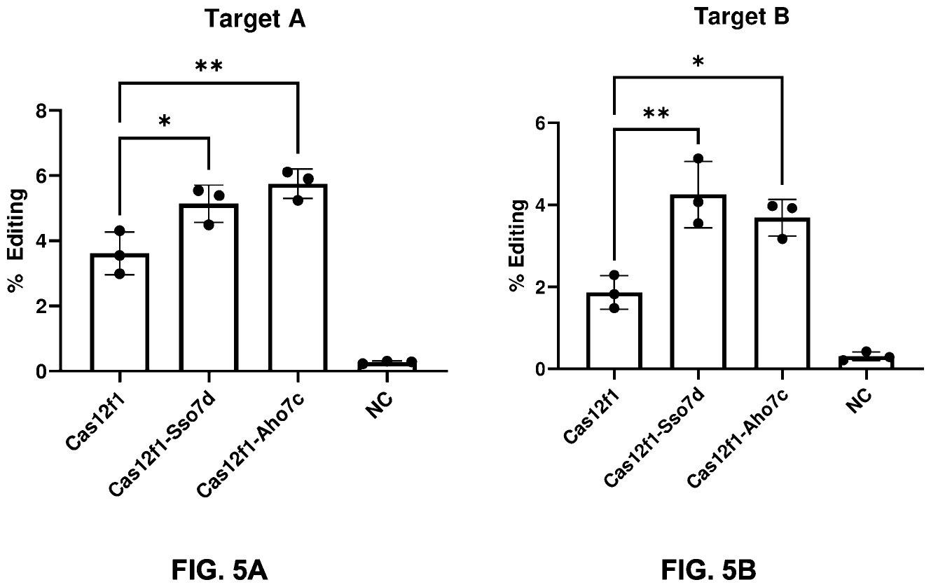

Average editing efficiency for both targets (here labeled “Target A” and “Target B”) for native Cas12f1, Cas12f1-Sso7d, and Cas12f1-Aho7c. In both cases, the Sso7d and Aho7c fusions yielded a significant increase in editing efficiency (*p<0.05; **p<0.01). No significant differences were observed between the Sso7d or Aho7c fusions (FIGS. 5A-B).

TABLE 1
Sequences

| SEQ ID | | |
|---|---|---|
| NO | Description | Sequence |
| 1 | Fragment | AAAAGGCCGGCGGCCACGAAAAAGGCCGGCCAGGCAAAAAAGAA |
| | 1 | AAAGTACCCATACGATGTTCCAGATTACG |
| | forward | |
| | primer | |
| 2 | Fragment | CCTCCGAGAGCTGCATCA |
| | 1 | |
| | reverse | |
| | primer | |
| 3 | Fragment | CTTCTTCTGCTTCTCGAGCATCTGCAACAATTCCTTTGGAGCGTCCT |
| | 2 | TTTCTGAAACGGCTCCTCTGCCAGTCTTTCCTCCTCCTTCATCGTAC |
| | template | GTAAAGCTGATCATCTTACCTACGCGCCACACTTTCTTGATTTTACT |
| | | GATGTCCACCTCTTTCTCTTCCCCTTTATACTTAAACTTCACTGTAG |
| | | CCATTCCGCTTC |
| 4 | Fragment | AGAGCACTAAGGAGGAGCCCGGAAGCGGAATGGCTACAGTG |
| | 2 | |
| | forward | |
| | primer | |
| 5 | Fragment | TTCGTGGCCGCCGGCCTTTTTCCGCTTCCCTTCTTCTGCTTCTCGAGC |
| | 2 | ATC |
| | reverse | |
| | primer | |
| 6 | Fragment | GCCAAAGATTGATAAGGGCGTG |
| | 3 | |
| | forward | |
| | primer | |
| 7 | Fragment | GGGCTCCTCCTTAGTGCT |
| | 3 | |
| | reverse | |
| | primer | |
| 8 | Sequencing | AGGTCTTGCAACGTGACAC |
| | primer | |
| 9 | pLV-43 | GTCGACGGATCGGGAGATCTCCCGATCCCCTATGGTGCACTCTCAG |
| | (comprising | TACAATCTGCTCTGATGCCGCATAGTTAAGCCAGTATCTGCTCCCT |
| | UbC: | GCTTGTGTGTTGGAGGTCGCTGAGTAGTGCGCGAGCAAAATTTAAG |
| | Cas12f1 | CTACAACAAGGCAAGGCTTGACCGACAATTGCATGAAGAATCTGCT |
| | expression | TAGGGTTAGGCGTTTTGCGCTGCTTCGCGATGTACGGGCCAGATAT |
| | cassette) | ACGCGTTGACATTGATTATTGACTAGTTATTAATAGTAATCAATTA |
| | | CGGGGTCATTAGTTCATAGCCCATATATGGAGTTCCGCGTTACATA |
| | | ACTTACGGTAAATGGCCCGCCTGGCTGACCGCCCAACGACCCCCGC |
| | | CCATTGACGTCAATAATGACGTATGTTCCCATAGTAACGCCAATAG |
| | | GGACTTTCCATTGACGTCAATGGGTGGAGTATTTACGGTAAACTGC |
| | | CCACTTGGCAGTACATCAAGTGTATCATATGCCAAGTACGCCCCCT |
| | | ATTGACGTCAATGACGGTAAATGGCCCGCCTGGCATTATGCCCAGT |
| | | ACATGACCTTATGGGACTTTCCTACTTGGCAGTACATCTACGTATT |
| | | AGTCATCGCTATTACCATGGTGATGCGGTTTTGGCAGTACATCAAT |
| | | GGGCGTGGATAGCGGTTTGACTCACGGGGATTTCCAAGTCTCCACC |
| | | CCATTGACGTCAATGGGAGTTTGTTTTGGCACCAAAATCAACGGGA |
| | | CTTTCCAAAATGTCGTAACAACTCCGCCCCATTGACGCAAATGGGC |
| | | GGTAGGCGTGTACGGTGGGAGGTCTATATAAGCAGCGCGTTTTGCC |
| | | TGTACTGGGTCTCTCTGGTTAGACCAGATCTGAGCCTGGGAGCTCT |
| | | CTGGCTAACTAGGGAACCCACTGCTTAAGCCTCAATAAAGCTTGCC |
| | | TTGAGTGCTTCAAGTAGTGTGTGCCCGTCTGTTGTGTGACTCTGGT |
| | | AACTAGAGATCCCTCAGACCCTTTTAGTCAGTGTGGAAAATCTCTA |
| | | GCAGTGGCGCCCGAACAGGGACTTGAAAGCGAAAGGGAAACCAGAG |
| | | GAGCTCTCTCGACGCAGGACTCGGCTTGCTGAAGCGCGCACGGCAA |
| | | GAGGCGAGGGGCGGCGACTGGTGAGTACGCCAAAAATTTTGACTAG |
| | | CGGAGGCTAGAAGGAGAGAGATGGGTGCGAGAGCGTCAGTATTAAG |
| | | CGGGGGAGAATTAGATCGCGATGGGAAAAAATTCGGTTAAGGCCAG |
| | | GGGGAAAGAAAAAATATAAATTAAAACATATAGTATGGGCAAGCAG |
| | | GGAGCTAGAACGATTCGCAGTTAATCCTGGCCTGTTAGAAACATCA |
| | | GAAGGCTGTAGACAAATACTGGGACAGCTACAACCATCCCTTCAGA |
| | | CAGGATCAGAAGAACTTAGATCATTATATAATACAGTAGCAACCCT |
| | | CTATTGTGTGCATCAAAGGATAGAGATAAAAGACACCAAGGAAGCT |
| | | TTAGACAAGATAGAGGAAGAGCAAAACAAAAGTAAGACCACCGCAC |
| | | AGCAAGCGGCCGCTGATCTTCAGACCTGGAGGAGGAGATATGAGGG |
| | | ACAATTGGAGAAGTGAATTATATAAATATAAAGTAGTAAAAATTGA |
| | | ACCATTAGGAGTAGCACCCACCAAGGCAAAGAGAAGAGTGGTGCAG |
| | | AGAGAAAAAAGAGCAGTGGGAATAGGAGCTTTGTTCCTTGGGTTCT |
| | | TGGGAGCAGCAGGAAGCACTATGGGCGCAGCGTCAATGACGCTGAC |
| | | GGTACAGGCCAGACAATTATTGTCTGGTATAGTGCAGCAGCAGAAC |
| | | AATTTGCTGAGGGCTATTGAGGCGCAACAGCATCTGTTGCAACTCA |
| | | CAGTCTGGGGCATCAAGCAGCTCCAGGCAAGAATCCTGGCTGTGGA |
| | | AAGATACCTAAAGGATCAACAGCTCCTGGGGATTTGGGGTTGCTCT |
| | | GGAAAACTCATTTGCACCACTGCTGTGCCTTGGAATGCTAGTTGGA |
| | | GTAATAAATCTCTGGAACAGATTTGGAATCACACGACCTGGATGGA |
| | | GTGGGACAGAGAAATTAACAATTACACAAGCTTAATACACTCCTTA |
| | | ATTGAAGAATCGCAAAACCAGCAAGAAAAGAATGAACAAGAATTAT |
| | | TGGAATTAGATAAATGGGCAAGTTTGTGGAATTGGTTTAACATAAC |
| | | AAATTGGCTGTGGTATATAAAATTATTCATAATGATAGTAGGAGGC |
| | | TTGGTAGGTTTAAGAATAGTTTTTGCTGTACTTTCTATAGTGAATA |
| | | GAGTTAGGCAGGGATATTCACCATTATCGTTTCAGACCCACCTCCC |
| | | AACCCCGAGGGGACCCGACAGGCCCGAAGGAATAGAAGAAGAAGGT |
| | | GGAGAGAGAGACAGAGACAGATCCATTCGATTAGTGAACGGATCGG |
| | | CACTGCGTGCGCCAATTCTGCAGACAAATGGCAGTATTCATCCACA |
| | | ATTTTAAAAGAAAAGGGGGGATTGGGGGGTACAGTGCAGGGGAAAG |
| | | AATAGTAGACATAATAGCAACAGACATACAAACTAAAGAATTACAA |
| | | AAACAAATTACAAAAATTCAAAATTTTCGGGTTTATTACAGGGACA |
| | | GCAGAGATCCAGTTTGGGCTAGCTTAATtaacctgcaggCCCGTGT |
| | | CGGCTCCAGATCTGGCCTCCGCGCCGGGTTTTGGCGCCTCCCGCGG |
| | | GCGCCCCCCTCCTCACGGCGAGCGCTGCCACGTCAGACGAAGGGCG |
| | | CAGCGAGCGTCCTGATCCTTCCGCCCGGACGCTCAGGACAGCGGCC |
| | | CGCTGCTCATAAGACTCGGCCTTAGAACCCCAGTATCAGCAGAAGG |
| | | ACATTTTAGGACGGGACTTGGGTGACTCTAGGGCACTGGTTTTCTT |
| | | TCCAGAGAGCGGAACAGGCGAGGAAAAGTAGTCCCTTCTCGGCGAT |
| | | TCTGCGGAGGGATCTCCGTGGGGCGGTGAACGCCGATGATTATATA |
| | | AGGACGCGCCGGGTGTGGCACAGCTAGTTCCGTCGCAGCCGGGATT |
| | | TGGGTCGCGGTTCTTGTTTGTGGATCGCTGTGATCGTCACTTGGTG |
| | | AGTAGCGGGCTGCTGGGCTGGCCGGGGCTTTCGTGGCCGCCGGGCC |
| | | GCTCGGTGGGACGGAAGCGTGTGGAGAGACCGCCAAGGGCTGTAGT |
| | | CTGGGTCCGCGAGCAAGGTTGCCCTGAACTGGGGGTTGGGGGGAGC |
| | | GCAGCAAAATGGCGGCTGTTCCCGAGTCTTGAATGGAAGACGCTTG |
| | | TGAGGCGGGCTGTGAGGTCGTTGAAACAAGGTGGGGGGCATGGTGG |
| | | GCGGCAAGAACCCAAGGTCTTGAGGCCTTCGCTAATGCGGGAAAGC |
| | | TCTTATTCGGGTGAGATGGGCTGGGGCACCATCTGGGGACCCTGAC |
| | | GTGAAGTTTGTCACTGACTGGAGAACTCGGTTTGTCGTCTGTTGCG |
| | | GGGGCGGCAGTTATGGCGGTGCCGTTGGGCAGTGCACCCGTACCTT |
| | | TGGGAGCGCGCGCCCTCGTCGTGTCGTGACGTCACCCGTTCTGTTG |
| | | GCTTATAATGCAGGGTGGGGCCACCTGCCGGTAGGTGTGCGGTAGG |
| | | CTTTTCTCCGTCGCAGGACGCAGGGTTCGGGCCTAGGGTAGGCTCT |
| | | CCTGAATCGACAGGCGCCGGACCTCTGGTGAGGGGAGGGATAAGTG |
| | | AGGCGTCAGTTTCTTTGGTCGGTTTTATGTACCTATCTTCTTAAGT |
| | | AGCTGAAGCTCCGGTTTTGAACTATGCGCTCGGGGTTGGCGAGTGT |
| | | GTTTTGTGAAGTTTTTTAGGCACCTTTTGAAATGTAATCATTTGGG |
| | | TCAATATGTAATTTTCAGTGTTAGACTAGTAAATTGTCCGCTAAAT |
| | | TCTGGCCGTTTTTGGCTTTTTTGTTAGACGAAGCTTGGGCTGCAGG |
| | | TCGACTCTAGGCCCaggCGGCCttgtacaaaaaagcACCGGTaggc |
| | | tgccaccATGCCAAAGAAGAAGCGGAAGGTCGGTATCCACGGAGTC |
| | | CCAGCAGCCATGGCCAAGAACACAATTACAAAGACACTGAAGCTGA |
| | | GGATCGTGAGACCATACAACAGCGCTGAGGTCGAGAAGATTGTGGC |
| | | TGATGAAAAGAACAACAGGGAAAAGATCGCCCTCGAGAAGAACAAG |
| | | GATAAGGTGAAGGAGGCCTGCTCTAAGCACCTGAAAGTGGCCGCCT |
| | | ACTGCACCACACAGGTGGAGAGGAACGCCTGTCTGTTTTGTAAAGC |
| | | TCGGAAGCTGGATGATAAGTTTTACCAGAAGCTGCGGGGCCAGTTC |
| | | CCCGATGCCGTCTTTTGGCAGGAGATTAGCGAGATCTTCAGACAGC |
| | | TGCAGAAGCAGGCCGCCGAGATCTACAACCAGAGCCTGATCGAGCT |
| | | CTACTACGAGATCTTCATCAAGGGCAAGGGCATTGCCAACGCCTCC |
| | | TCCGTGGAGCACTACCTGAGCGACGTGTGCTACACAAGAGCCGCCG |
| | | AGCTCTTTAAGAACGCCGCTATCGCTTCCGGGCTGAGGAGCAAGAT |
| | | TAAGAGTAACTTCCGGCTCAAGGAGCTGAAGAACATGAAGAGCGGC |
| | | CTGCCCACTACAAAGAGCGACAACTTCCCAATTCCACTGGTGAAGC |
| | | AGAAGGGGGGCCAGTACACAGGGTTCGAGATTTCCAACCACAACAG |
| | | CGACTTTATTATTAAGATCCCCTTTGGCAGGTGGCAGGTCAAGAAG |
| | | GAGATTGACAAGTACAGGCCCTGGGAGAAGTTTGATTTCGAGCAGG |
| | | TGCAGAAGAGCCCCAAGCCTATTTCCCTGCTGCTGTCCACACAGCG |
| | | GCGGAAGAGGAACAAGGGGTGGTCTAAGGATGAGGGGACCGAGGCC |
| | | GAGATTAAGAAAGTGATGAACGGCGACTACCAGACAAGCTACATCG |
| | | AGGTCAAGCGGGGCAGTAAGATTGGCGAGAAGAGCGCCTGGATGCT |
| | | GAACCTGAGCATTGACGTGCCAAAGATTGATAAGGGCGTGGATCCC |
| | | AGCATCATCGGAGGGATCGATGTGGGGGTCAAGAGCCCCCTCGTGT |
| | | GCGCCATCAACAACGCCTTCAGCAGGTACAGCATCTCCGATAACGA |
| | | CCTGTTCCACTTTAACAAGAAGATGTTCGCCCGGCGGAGGATTTTG |
| | | CTCAAGAAGAACCGGCACAAGCGGGCCGGACACGGGGCCAAGAACA |
| | | AGCTCAAGCCCATCACTATCCTGACCGAGAAGAGCGAGAGGTTCAG |
| | | GAAGAAGCTCATCGAGAGATGGGCCTGCGAGATCGCCGATTTCTTT |
| | | ATTAAGAACAAGGTCGGAACAGTGCAGATGGAGAACCTCGAGAGCA |
| | | TGAAGAGGAAGGAGGATTCCTACTTCAACATTCGGCTGAGGGGGTT |
| | | CTGGCCCTACGCTGAGATGCAGAACAAGATTGAGTTTAAGCTGAAG |
| | | CAGTACGGGATTGAGATCCGGAAGGTGGCCCCCAACAACACCAGCA |
| | | AGACCTGCAGCAAGTGCGGGCACCTCAACAACTACTTCAACTTCGA |
| | | GTACCGGAAGAAGAACAAGTTCCCACACTTCAAGTGCGAGAAGTGC |
| | | AACTTTAAGGAGAACGCCGATTACAACGCCGCCCTGAACATCAGCA |
| | | ACCCTAAGCTGAAGAGCACTAAGGAGGAGCCCAAAAGGCCGGCGGC |
| | | CACGAAAAAGGCCGGCCAGGCAAAAAAGAAAAAGtacccatacgat |
| | | gttccagattacgctggaagcggagagggcaggggaagtcttctaa |
| | | catgcggggacgtggaggaaaatcccggccccatgaaaaagcctga |
| | | actcaccgcgacgtctgtcgagaagtttctgatcgaaaagttcgac |
| | | agcgtctccgacctgatgcagctctcggagggcgaagaatctcgtg |
| | | ctttcagcttcgatgtaggagggcgtggatatgtcctgcgggtaaa |
| | | tagctgcgccgatggtttctacaaagatcgttatgtttatcggcac |
| | | tttgcatcggccgcgctcccgattccggaagtgcttgacattgggg |
| | | aatttagcgagagcctgacctattgcatctcccgccgtgcacaggg |
| | | tgtcacgttgcaagacctgcctgaaaccgaactgcccgctgttctg |
| | | cagccggtcgcggaggccatggatgcgatcgctgcggccgatctta |
| | | gccagacgagcgggttcggcccattcggaccgcaaggaatcggtca |
| | | atacactacatggcgtgatttcatatgcgcgattgctgatccccat |
| | | gtgtatcactggcaaactgtgatggacgacaccgtcagtgcgtccg |
| | | tcgcgcaggctctcgatgagctgatgctttgggccgaggactgccc |
| | | cgaagtccggcacctcgtgcacgcggatttcggctccaacaatgtc |
| | | ctgacggacaatggccgcataacagcggtcattgactggagcgagg |
| | | cgatgttcggggattcccaatacgaggtcgccaacatcttcttctg |
| | | gaggccgtggttggcttgtatggagcagcagacgcgctacttcgag |
| | | cggaggcatccggagcttgcaggatcgccgcggctccgggcgtata |
| | | tgctccgcattggtcttgaccaactctatcagagcttggttgacgg |
| | | caatttcgatgatgcagcttgggcgcagggtcgatgcgacgcaatc |
| | | gtccgatccggagccgggactgtcgggcgtacacaaatcgcccgca |
| | | gaagcgcggccgtctggaccgatggctgtgtagaagtactcgccga |
| | | tagtggaaaccgacgccccagcactcgtccgagggcaaaggaatag |
| | | acccgaattccagGGCCAggcCGGCCGATATCAAGCTTATCGATAA |
| | | TCAACCTCTGGATTACAAAATTTGTGAAAGATTGACTGGTATTCTT |
| | | AACTATGTTGCTCCTTTTACGCTATGTGGATACGCTGCTTTAATGC |
| | | CTTTGTATCATGCTATTGCTTCCCGTATGGCTTTCATTTTCTCCTC |
| | | CTTGTATAAATCCTGGTTGCTGTCTCTTTATGAGGAGTTGTGGCCC |
| | | GTTGTCAGGCAACGTGGCGTGGTGTGCACTGTGTTTGCTGACGCAA |
| | | CCCCCACTGGTTGGGGCATTGCCACCACCTGTCAGCTCCTTTCCGG |
| | | GACTTTCGCTTTCCCCCTCCCTATTGCCACGGCGGAACTCATCGCC |
| | | GCCTGCCTTGCCCGCTGCTGGACAGGGGCTCGGCTGTTGGGCACTG |
| | | ACAATTCCGTGGTGTTGTCGGGGAAATCATCGTCCTTTCCTTGGCT |
| | | GCTCGCCTGTGTTGCCACCTGGATTCTGCGCGGGACGTCCTTCTGC |
| | | TACGTCCCTTCGGCCCTCAATCCAGCGGACCTTCCTTCCCGCGGCC |
| | | TGCTGCCGGCTCTGCGGCCTCTTCCGCGTCTTCGCCTTCGCCCTCA |
| | | GACGAGTCGGATCTCCCTTTGGGCCGCCTCCCCGCATCGATACCGT |
| | | CGACCTCGAGACCTAGAAAAACATGGAGCAATCACAAGTAGCAATA |
| | | CAGCAGCTACCAATGCTGATTGTGCCTGGCTAGAAGCACAAGAGGA |
| | | GGAGGAGGTGGGTTTTCCAGTCACACCTCAGGTACCTTTAAGACCA |
| | | ATGACTTACAAGGCAGCTGTAGATCTTAGCCACTTTTTAAAAGAAA |
| | | AGGGGGGACTGGAAGGGCTAATTCACTCCCAACGAAGACAAGATAT |
| | | CCTTGATCTGTGGATCTACCACACACAAGGCTACTTCCCTGATTGG |
| | | CAGAACTACACACCAGGGCCAGGGATCAGATATCCACTGACCTTTG |
| | | GATGGTGCTACAAGCTAGTACCAGTTGAGCAAGAGAAGGTAGAAGA |
| | | AGCCAATGAAGGAGAGAACACCCGCTTGTTACACCCTGTGAGCCTG |
| | | CATGGGATGGATGACCCGGAGAGAGAAGTATTAGAGTGGAGGTTTG |
| | | ACAGCCGCCTAGCATTTCATCACATGGCCCGAGAGCTGCATCCGGA |
| | | CTGTACTGGGTCTCTCTGGTTAGACCAGATCTGAGCCTGGGAGCTC |
| | | TCTGGCTAACTAGGGAACCCACTGCTTAAGCCTCAATAAAGCTTGC |
| | | CTTGAGTGCTTCAAGTAGTGTGTGCCCGTCTGTTGTGTGACTCTGG |
| | | TAACTAGAGATCCCTCAGACCCTTTTAGTCAGTGTGGAAAATCTCT |
| | | AGCAGGGCCCGTTTAAACCCGCTGATCAGCCTCGACTGTGCCTTCT |
| | | AGTTGCCAGCCATCTGTTGTTTGCCCCTCCCCCGTGCCTTCCTTGA |
| | | CCCTGGAAGGTGCCACTCCCACTGTCCTTTCCTAATAAAATGAGGA |
| | | AATTGCATCGCATTGTCTGAGTAGGTGTCATTCTATTCTGGGGGGG |
| | | GGGTGGGGCAGGACAGCAAGGGGGAGGATTGGGAAGACAATAGCAG |
| | | GCATGCTGGGGATGCGGTGGGCTCTATGGCTTCTGAGGCGGAAAGT |
| | | CACCTAGATCCTTTTAAATTAAAAATGAAGTTTTAAATCAATCTAA |
| | | AGTATATATGAGTAAACTTGGTCTGACAGTTACCAATGCTTAATCA |
| | | GTGAGGCACCTATCTCAGCGATCTGTCTATTTCGTTCATCCATAGT |
| | | TGCCTGACTCCCCGTCGTGTAGATAACTACGATACGGGAGGGCTTA |
| | | CCATCTGGCCCCAGTGCTGCAATGATACCGCGAGACCCACGCTCAC |
| | | CGGCTCCAGATTTATCAGCAATAAACCAGCCAGCCGGAAGGGCCGA |
| | | GCGCAGAAGTGGTCCTGCAACTTTATCCGCCTCCATCCAGTCTATT |
| | | AATTGTTGCCGGGAAGCTAGAGTAAGTAGTTCGCCAGTTAATAGTT |
| | | TGCGCAACGTTGTTGCCATTGCTACAGGCATCGTGGTGTCACGCTC |
| | | GTCGTTTGGTATGGCTTCATTCAGCTCCGGTTCCCAACGATCAAGG |
| | | CGAGTTACATGATCCCCCATGTTGTGCAAAAAAGCGGTTAGCTCCT |
| | | TCGGTCCTCCGATCGTTGTCAGAAGTAAGTTGGCCGCAGTGTTATC |
| | | ACTCATGGTTATGGCAGCACTGCATAATTCTCTTACTGTCATGCCA |
| | | TCCGTAAGATGCTTTTCTGTGACTGGTGAGTACTCAACCAAGTCAT |
| | | TCTGAGAATAGTGTATGCGGCGACCGAGTTGCTCTTGCCCGGCGTC |
| | | AATACGGGATAATACCGCGCCACATAGCAGAACTTTAAAAGTGCTC |
| | | ATCATTGGAAAACGTTCTTCGGGGCGAAAACTCTCAAGGATCTTAC |
| | | CGCTGTTGAGATCCAGTTCGATGTAACCCACTCGTGCACCCAACTG |
| | | ATCTTCAGCATCTTTTACTTTCACCAGCGTTTCTGGGTGAGCAAAA |
| | | ACAGGAAGGCAAAATGCCGCAAAAAAGGGAATAAGGGCGACACGGA |
| | | AATGTTGAATACTCATACTCTTCCTTTTTCAATATTATTGAAGCAT |
| | | TTATCAGGGTTATTGTCTCATGAGCGGATACATATTTGAATGTATT |
| | | TAGAAAAATAAACAAATAGGGGTTCCGCGCACATTTCCCCGAAAAG |
| | | TGCCACCTGACAACCAGCTGGGGCTCTAGGGGGTATCCCCACGCGC |
| | | CCTGTAGCGGCGCATTAAGCGCGGCGGGTGTGGTGGTTACGCGCAG |
| | | CGTGACCGCTACACTTGCCAGCGCCCTAGCGCCCGCTCCTTTCGCT |
| | | TTCTTCCCTTCCTTTCTCGCCACGTTCGCCGGCTTTCCCCGTCAAG |
| | | CTCTAAATCGGGGGCTCCCTTTAGGGTTCCGATTTAGTGCTTTACG |
| | | GCACCTCGACCCCAAAAAACTTGATTAGGGTGATGGTTCACGTAGT |
| | | GGGCCATCGCCCTGATAGACGGTTTTTCGCCCTTTGACGTTGGAGT |
| | | CCACGTTCTTTAATAGTGGACTCTTGTTCCAAACTGGAACAACACT |
| | | CAACCCTATCTCGGTCTATTCTTTTGATTTATAAGGGATTTTGCCG |
| | | ATTTCGGCCTATTGGTTAAAAAATGAGCTGATTTAACAAAAATTTA |
| | | ACGCGAATTAATTCTGTGGAATGTGTGTCAGTTAGGGTGTGGAAAG |
| | | TCCCCAGGCTCCCCAGCAGGCAGAAGTATGCAAAGCATGCATCTCA |
| | | ATTAGTCAGCAACCAGGTGTGGAAAGTCCCCAGGCTCCCCAGCAGG |
| | | CAGAAGTATGCAAAGCATGCATCTCAATTAGTCAGCAACCATAGTC |
| | | CCGCCCCTAACTCCGCCCATCCCGCCCCTAACTCCGCCCAGTTCCG |
| | | CCCATTCTCCGCCCCATGGCTGACTAATTTTTTTTATTTATGCAGA |
| | | GGCCGAGGCCGCCTCTGCCTCTGAGCTATTCCAGAAGTAGTGAGGA |
| | | GGCTTTTTTGGAGGCCTAGGCTTTTGCAAAAAGCTCCCGGGAGCTT |
| | | GTATATCCATTTTCGGATCTGATCAGCACGTGTTGACAATTAATCA |
| | | TCGGCATAGTATATCGGCATAGTATAATACGACAAGGTGAGGAACT |
| | | AAACCATGGCCAAGTTGACCAGTGCCGTTCCGGTGCTCACCGCGCG |
| | | CGACGTCGCCGGAGCGGTCGAGTTCTGGACCGACCGGCTCGGGTTC |
| | | TCCCGGGACTTCGTGGAGGACGACTTCGCCGGTGTGGTCCGGGACG |
| | | ACGTGACCCTGTTCATCAGCGCGGTCCAGGACCAGGTGGTGCCGGA |
| | | CAACACCCTGGCCTGGGTGTGGGTGCGCGGCCTGGACGAGCTGTAC |
| | | GCCGAGTGGTCGGAGGTCGTGTCCACGAACTTCCGGGACGCCTCCG |
| | | GGCCGGCCATGACCGAGATCGGCGAGCAGCCGTGGGGGCGGGAGTT |
| | | CGCCCTGCGCGACCCGGCCGGCAACTGCGTGCACTTCGTGGCCGAG |
| | | GAGCAGGACTGACACGTGCTACGAGATTTCGATTCCACCGCCGCCT |
| | | TCTATGAAAGGTTGGGCTTCGGAATCGTTTTCCGGGACGCCGGCTG |
| | | GATGATCCTCCAGCGCGGGGATCTCATGCTGGAGTTCTTCGCCCAC |
| | | CCCAACTTGTTTATTGCAGCTTATAATGGTTACAAATAAAGCAATA |
| | | GCATCACAAATTTCACAAATAAAGCATTTTTTTCACTGCATTCTAG |
| | | TTGTGGTTTGTCCAAACTCATCAATGTATCTTATCATGTCTGTATA |
| | | CCGTCGACCTCTAGCTAGAGCTTGGCGTAATCATGGTCATAGCTGT |
| | | TTCCTGTGTGAAATTGTTATCCGCTCACAATTCCACACAACATACG |
| | | AGCCGGAAGCATAAAGTGTAAAGCCTGGGGTGCCTAATGAGTGAGC |
| | | TAACTCACATTAATTGCGTTGCGCTCACTGCCCGCTTTCCAGTCGG |
| | | GAAACCTGTCGTGCCAGCTGCATTAATGAATCGGCCAACGCGCGGG |
| | | GAGAGGCGGTTTGCGTATTGGGCGCTCTTCCGCTTCCTCGCTCACT |
| | | GACTCGCTGCGCTCGGTCGTTCGGCTGCGGCGAGCGGTATCAGCTC |
| | | ACTCAAAGGCGGTAATACGGTTATCCACAGAATCAGGGGATAACGC |
| | | AGGAAAGAACATGTGAGCAAAAGGCCAGCAAAAGGCCAGGAACCGT |
| | | AAAAAGGCCGCGTTGCTGGCGTTTTTCCATAGGCTCCGCCCCCCTG |
| | | ACGAGCATCACAAAAATCGACGCTCAAGTCAGAGGTGGCGAAACCC |
| | | GACAGGACTATAAAGATACCAGGCGTTTCCCCCTGGAAGCTCCCTC |
| | | GTGCGCTCTCCTGTTCCGACCCTGCCGCTTACCGGATACCTGTCCG |
| | | CCTTTCTCCCTTCGGGAAGCGTGGCGCTTTCTCATAGCTCACGCTG |
| | | TAGGTATCTCAGTTCGGTGTAGGTCGTTCGCTCCAAGCTGGGCTGT |
| | | GTGCACGAACCCCCCGTTCAGCCCGACCGCTGCGCCTTATCCGGTA |
| | | ACTATCGTCTTGAGTCCAACCCGGTAAGACACGACTTATCGCCACT |
| | | GGCAGCAGCCACTGGTAACAGGATTAGCAGAGCGAGGTATGTAGGC |
| | | GGTGCTACAGAGTTCTTGAAGTGGTGGCCTAACTACGGCTACACTA |
| | | GAAGAACAGTATTTGGTATCTGCGCTCTGCTGAAGCCAGTTACCTT |
| | | CGGAAAAAGAGTTGGTAGCTCTTGATCCGGCAAACAAACCACCGCT |
| | | GGTAGCGGTGGTTTTTTTGTTTGCAAGCAGCAGATTACGCGCAGAA |
| | | AAAAAGGATCTCAAGAAGATCCTTTGATCTTTTCTACGGGGTCTGA |
| | | CGCTCAGTGGAACGAAAACTCACGTTAAGGGATTTTGGTCATGAGA |
| | | TTATCAAAAAGGATCT |
| 10 | Cas12f | ATGCCAAAGAAGAAGCGGAAGGTCGGTATCCACGGAGTCCCAGCAG |
| | 1-Sso7d | CCATGGCCAAGAACACAATTACAAAGACACTGAAGCTGAGGATCGT |
| | Express | GAGACCATACAACAGCGCTGAGGTCGAGAAGATTGTGGCTGATGAA |
| | ion | AAGAACAACAGGGAAAAGATCGCCCTCGAGAAGAACAAGGATAAGG |
| | Cassette | TGAAGGAGGCCTGCTCTAAGCACCTGAAAGTGGCCGCCTACTGCAC |
| | (DNA) | CACACAGGTGGAGAGGAACGCCTGTCTGTTTTGTAAAGCTCGGAAG |
| | as | CTGGATGATAAGTTTTACCAGAAGCTGCGGGGCCAGTTCCCCGATG |
| | cloned | CCGTCTTTTGGCAGGAGATTAGCGAGATCTTCAGACAGCTGCAGAA |
| | in pLV- | GCAGGCCGCCGAGATCTACAACCAGAGCCTGATCGAGCTCTACTAC |
| | 43 to | GAGATCTTCATCAAGGGCAAGGGCATTGCCAACGCCTCCTCCGTGG |
| | obtain | AGCACTACCTGAGCGACGTGTGCTACACAAGAGCCGCCGAGCTCTT |
| | pLV- | TAAGAACGCCGCTATCGCTTCCGGGCTGAGGAGCAAGATTAAGAGT |
| | EW8 | AACTTCCGGCTCAAGGAGCTGAAGAACATGAAGAGCGGCCTGCCCA |
| | | CTACAAAGAGCGACAACTTCCCAATTCCACTGGTGAAGCAGAAGGG |
| | | GGGCCAGTACACAGGGTTCGAGATTTCCAACCACAACAGCGACTTT |
| | | ATTATTAAGATCCCCTTTGGCAGGTGGCAGGTCAAGAAGGAGATTG |
| | | ACAAGTACAGGCCCTGGGAGAAGTTTGATTTCGAGCAGGTGCAGAA |
| | | GAGCCCCAAGCCTATTTCCCTGCTGCTGTCCACACAGCGGCGGAAG |
| | | AGGAACAAGGGGTGGTCTAAGGATGAGGGGACCGAGGCCGAGATTA |
| | | AGAAAGTGATGAACGGCGACTACCAGACAAGCTACATCGAGGTCAA |
| | | GCGGGGCAGTAAGATTGGCGAGAAGAGCGCCTGGATGCTGAACCTG |
| | | AGCATTGACGTGCCAAAGATTGATAAGGGCGTGGATCCCAGCATCA |
| | | TCGGAGGGATCGATGTGGGGGTCAAGAGCCCCCTCGTGTGCGCCAT |
| | | CAACAACGCCTTCAGCAGGTACAGCATCTCCGATAACGACCTGTTC |
| | | CACTTTAACAAGAAGATGTTCGCCCGGCGGAGGATTTTGCTCAAGA |
| | | AGAACCGGCACAAGCGGGCCGGACACGGGGCCAAGAACAAGCTCAA |
| | | GCCCATCACTATCCTGACCGAGAAGAGCGAGAGGTTCAGGAAGAAG |
| | | CTCATCGAGAGATGGGCCTGCGAGATCGCCGATTTCTTTATTAAGA |
| | | ACAAGGTCGGAACAGTGCAGATGGAGAACCTCGAGAGCATGAAGAG |
| | | GAAGGAGGATTCCTACTTCAACATTCGGCTGAGGGGGTTCTGGCCC |
| | | TACGCTGAGATGCAGAACAAGATTGAGTTTAAGCTGAAGCAGTACG |
| | | GGATTGAGATCCGGAAGGTGGCCCCCAACAACACCAGCAAGACCTG |
| | | CAGCAAGTGCGGGCACCTCAACAACTACTTCAACTTCGAGTACCGG |
| | | AAGAAGAACAAGTTCCCACACTTCAAGTGCGAGAAGTGCAACTTTA |
| | | AGGAGAACGCCGATTACAACGCCGCCCTGAACATCAGCAACCCTAA |
| | | GCTGAAGAGCACTAAGGAGGAGCCCggatccggaATGGCTACAGTG |
| | | AAGTTTAAGTATAAAGGGGAAGAGAAAGAGGTGGACATCAGTAAAA |
| | | TCAAGAAAGTGTGGCGCGTAGGTAAGATGATCAGCTTTACGTACGA |
| | | TGAAGGAGGAGGAAAGACTGGCAGAGGAGCCGTTTCAGAAAAGGAC |
| | | GCTCCAAAGGAATTGTTGCAGATGCTCGAGAAGCAGAAGAAGggaa |
| | | gcggaAAAAGGCCGGCGGCCACGAAAAAGGCCGGCCAGGCAAAAAA |
| | | GAAAAAgtacccatacgatgttccagattacgctggaagcggagag |
| | | ggcaggggaagtcttctaacatgcggggacgtggaggaaaatcccg |
| | | gccccatgaaaaagcctgaactcaccgcgacgtctgtcgagaagtt |
| | | tctgatcgaaaagttcgacagcgtctccgacctgatgcagctctcg |
| | | gagggcgaagaatctcgtgctttcagcttcgatgtaggagggcgtg |
| | | gatatgtcctgcgggtaaatagctgcgccgatggtttctacaaaga |
| | | tcgttatgtttatcggcactttgcateggccgcgctcccgattccg |
| | | gaagtgcttgacattggggaatttagcgagagcctgacctattgca |
| | | tctcccgccgtgcacagggtgtcacgttgcaagacctgcctgaaac |
| | | cgaactgcccgctgttctgcagccggtcgcggaggccatggatgcg |
| | | atcgctgcggccgatcttagccagacgagcgggttcggcccattcg |
| | | gaccgcaaggaatcggtcaatacactacatggcgtgatttcatatg |
| | | cgcgattgctgatccccatgtgtatcactggcaaactgtgatggac |
| | | gacaccgtcagtgcgtccgtcgcgcaggctctcgatgagctgatgc |
| | | tttgggccgaggactgccccgaagtccggcacctcgtgcacgcgga |
| | | tttcggetccaacaatgtcctgacggacaatggccgcataacagcg |
| | | gtcattgactggagcgaggcgatgttcggggattcccaatacgagg |
| | | tcgccaacatcttcttctggaggccgtggttggcttgtatggagca |
| | | gcagacgcgctacttcgagcggaggcatccggagcttgcaggatcg |
| | | ccgcggctccgggcgtatatgctccgcattggtcttgaccaactct |
| | | atcagagcttggttgacggcaatttcgatgatgcagcttgggcgca |
| | | gggtcgatgcgacgcaatcgtccgatccggagccgggactgtcggg |
| | | cgtacacaaatcgcccgcagaagcgcggccgtctggaccgatggct |
| | | gtgtagaagtactcgccgatagtggaaaccgacgccccagcactcg |
| | | tccgagggcaaaggaatag |
| 11 | Cas12f | MPKKKRKVGIHGVPAAMAKNTITKTLKLRIVRPYNSAEVEKIVADE |
| | 1-Sso7d | KNNREKIALEKNKDKVKEACSKHLKVAAYCTTQVERNACLFCKARK |
| | Express | LDDKFYQKLRGQFPDAVFWQEISEIFRQLQKQAAEIYNQSLIELYY |
| | ion | EIFIKGKGIANASSVEHYLSDVCYTRAAELFKNAAIASGLRSKIKS |
| | Cassette | NFRLKELKNMKSGLPTTKSDNFPIPLVKQKGGQYTGFEISNHNSDF |
| | (amino | IIKIPFGRWQVKKEIDKYRPWEKFDFEQVQKSPKPISLLLSTQRRK |
| | acid) as | RNKGWSKDEGTEAEIKKVMNGDYQTSYIEVKRGSKIGEKSAWMLNL |
| | cloned | SIDVPKIDKGVDPSIIGGIDVGVKSPLVCAINNAFSRYSISDNDLF |
| | in pLV- | HFNKKMFARRRILLKKNRHKRAGHGAKNKLKPITILTEKSERFRKK |
| | 43 to | LIERWACEIADFFIKNKVGTVQMENLESMKRKEDSYFNIRLRGFWP |
| | obtain | YAEMQNKIEFKLKQYGIEIRKVAPNNTSKTCSKCGHLNNYFNFEYR |
| | pLV- | KKNKFPHFKCEKCNFKENADYNAALNISNPKLKSTKEEPGSGMATV |
| | EW8 | KFKYKGEEKEVDISKIKKVWRVGKMISFTYDEGGGKTGRGAVSEKD |
| | | APKELLQMLEKQKKGSGKRPAATKKAGQAKKKKYPYDVPDYAGSGE |
| | | GRGSLLTCGDVEENPGPMKKPELTATSVEKFLIEKFDSVSDLMQLS |
| | | EGEESRAFSFDVGGRGYVLRVNSCADGFYKDRYVYRHFASAALPIP |
| | | EVLDIGEFSESLTYCISRRAQGVTLQDLPETELPAVLQPVAEAMDA |
| | | IAAADLSQTSGFGPFGPQGIGQYTTWRDFICAIADPHVYHWQTVMD |
| | | DTVSASVAQALDELMLWAEDCPEVRHLVHADFGSNNVLTDNGRITA |
| | | VIDWSEAMFGDSQYEVANIFFWRPWLACMEQQTRYFERRHPELAGS |
| | | PRLRAYMLRIGLDQLYQSLVDGNFDDAAWAQGRCDAIVRSGAGTVG |
| | | RTQIARRSAAVWTDGCVEVLADSGNRRPSTRPRAKE* |
| 12 | pLV- | GCCGATATCAAGCTTATCGATAATCAACCTCTGGATTACAAAATTT |
| | EW8 = | GTGAAAGATTGACTGGTATTCTTAACTATGTTGCTCCTTTTACGCT |
| | pLV-43 | ATGTGGATACGCTGCTTTAATGCCTTTGTATCATGCTATTGCTTCC |
| | compri | CGTATGGCTTTCATTTTCTCCTCCTTGTATAAATCCTGGTTGCTGT |
| | sing | CTCTTTATGAGGAGTTGTGGCCCGTTGTCAGGCAACGTGGCGTGGT |
| | Cas12f | GTGCACTGTGTTTGCTGACGCAACCCCCACTGGTTGGGGCATTGCC |
| | 1-Sso7d | ACCACCTGTCAGCTCCTTTCCGGGACTTTCGCTTTCCCCCTCCCTA |
| | Expression | TTGCCACGGCGGAACTCATCGCCGCCTGCCTTGCCCGCTGCTGGAC |
| | Cassette | AGGGGCTCGGCTGTTGGGCACTGACAATTCCGTGGTGTTGTCGGGG |
| | | AAATCATCGTCCTTTCCTTGGCTGCTCGCCTGTGTTGCCACCTGGA |
| | | TTCTGCGCGGGACGTCCTTCTGCTACGTCCCTTCGGCCCTCAATCC |
| | | AGCGGACCTTCCTTCCCGCGGCCTGCTGCCGGCTCTGCGGCCTCTT |
| | | CCGCGTCTTCGCCTTCGCCCTCAGACGAGTCGGATCTCCCTTTGGG |
| | | CCGCCTCCCCGCATCGATACCGTCGACCTCGAGACCTAGAAAAACA |
| | | TGGAGCAATCACAAGTAGCAATACAGCAGCTACCAATGCTGATTGT |
| | | GCCTGGCTAGAAGCACAAGAGGAGGAGGAGGTGGGTTTTCCAGTCA |
| | | CACCTCAGGTACCTTTAAGACCAATGACTTACAAGGCAGCTGTAGA |
| | | TCTTAGCCACTTTTTAAAAGAAAAGGGGGGACTGGAAGGGCTAATT |
| | | CACTCCCAACGAAGACAAGATATCCTTGATCTGTGGATCTACCACA |
| | | CACAAGGCTACTTCCCTGATTGGCAGAACTACACACCAGGGCCAGG |
| | | GATCAGATATCCACTGACCTTTGGATGGTGCTACAAGCTAGTACCA |
| | | GTTGAGCAAGAGAAGGTAGAAGAAGCCAATGAAGGAGAGAACACCC |
| | | GCTTGTTACACCCTGTGAGCCTGCATGGGATGGATGACCCGGAGAG |
| | | AGAAGTATTAGAGTGGAGGTTTGACAGCCGCCTAGCATTTCATCAC |
| | | ATGGCCCGAGAGCTGCATCCGGACTGTACTGGGTCTCTCTGGTTAG |
| | | ACCAGATCTGAGCCTGGGAGCTCTCTGGCTAACTAGGGAACCCACT |
| | | GCTTAAGCCTCAATAAAGCTTGCCTTGAGTGCTTCAAGTAGTGTGT |
| | | GCCCGTCTGTTGTGTGACTCTGGTAACTAGAGATCCCTCAGACCCT |
| | | TTTAGTCAGTGTGGAAAATCTCTAGCAGGGCCCGTTTAAACCCGCT |
| | | GATCAGCCTCGACTGTGCCTTCTAGTTGCCAGCCATCTGTTGTTTG |
| | | CCCCTCCCCCGTGCCTTCCTTGACCCTGGAAGGTGCCACTCCCACT |
| | | GTCCTTTCCTAATAAAATGAGGAAATTGCATCGCATTGTCTGAGTA |
| | | GGTGTCATTCTATTCTGGGGGGTGGGGTGGGGCAGGACAGCAAGGG |
| | | GGAGGATTGGGAAGACAATAGCAGGCATGCTGGGGATGCGGTGGGC |
| | | TCTATGGCTTCTGAGGCGGAAAGAACCAGCTGGGGCTCTAGGGGGT |
| | | ATCCCCACGCGCCCTGTAGCGGCGCATTAAGCGCGGCGGGTGTGGT |
| | | GGTTACGCGCAGCGTGACCGCTACACTTGCCAGCGCCCTAGCGCCC |
| | | GCTCCTTTCGCTTTCTTCCCTTCCTTTCTCGCCACGTTCGCCGGCT |
| | | TTCCCCGTCAAGCTCTAAATCGGGGGCTCCCTTTAGGGTTCCGATT |
| | | TAGTGCTTTACGGCACCTCGACCCCAAAAAACTTGATTAGGGTGAT |
| | | GGTTCACGTAGTGGGCCATCGCCCTGATAGACGGTTTTTCGCCCTT |
| | | TGACGTTGGAGTCCACGTTCTTTAATAGTGGACTCTTGTTCCAAAC |
| | | TGGAACAACACTCAACCCTATCTCGGTCTATTCTTTTGATTTATAA |
| | | GGGATTTTGCCGATTTCGGCCTATTGGTTAAAAAATGAGCTGATTT |
| | | AACAAAAATTTAACGCGAATTAATTCTGTGGAATGTGTGTCAGTTA |
| | | GGGTGTGGAAAGTCCCCAGGCTCCCCAGCAGGCAGAAGTATGCAAA |
| | | GCATGCATCTCAATTAGTCAGCAACCAGGTGTGGAAAGTCCCCAGG |
| | | CTCCCCAGCAGGCAGAAGTATGCAAAGCATGCATCTCAATTAGTCA |
| | | GCAACCATAGTCCCGCCCCTAACTCCGCCCATCCCGCCCCTAACTC |
| | | CGCCCAGTTCCGCCCATTCTCCGCCCCATGGCTGACTAATTTTTTT |
| | | TATTTATGCAGAGGCCGAGGCCGCCTCTGCCTCTGAGCTATTCCAG |
| | | AAGTAGTGAGGAGGCTTTTTTGGAGGCCTAGGCTTTTGCAAAAAGC |
| | | TCCCGGGAGCTTGTATATCCATTTTCGGATCTGATCAGCACGTGTT |
| | | GACAATTAATCATCGGCATAGTATATCGGCATAGTATAATACGACA |
| | | AGGTGAGGAACTAAACCATGGCCAAGTTGACCAGTGCCGTTCCGGT |
| | | GCTCACCGCGCGCGACGTCGCCGGAGCGGTCGAGTTCTGGACCGAC |
| | | CGGCTCGGGTTCTCCCGGGACTTCGTGGAGGACGACTTCGCCGGTG |
| | | TGGTCCGGGACGACGTGACCCTGTTCATCAGCGCGGTCCAGGACCA |
| | | GGTGGTGCCGGACAACACCCTGGCCTGGGTGTGGGTGCGCGGCCTG |
| | | GACGAGCTGTACGCCGAGTGGTCGGAGGTCGTGTCCACGAACTTCC |
| | | GGGACGCCTCCGGGCCGGCCATGACCGAGATCGGCGAGCAGCCGTG |
| | | GGGGCGGGAGTTCGCCCTGCGCGACCCGGCCGGCAACTGCGTGCAC |
| | | TTCGTGGCCGAGGAGCAGGACTGACACGTGCTACGAGATTTCGATT |
| | | CCACCGCCGCCTTCTATGAAAGGTTGGGCTTCGGAATCGTTTTCCG |
| | | GGACGCCGGCTGGATGATCCTCCAGCGCGGGGATCTCATGCTGGAG |
| | | TTCTTCGCCCACCCCAACTTGTTTATTGCAGCTTATAATGGTTACA |
| | | AATAAAGCAATAGCATCACAAATTTCACAAATAAAGCATTTTTTTC |
| | | ACTGCATTCTAGTTGTGGTTTGTCCAAACTCATCAATGTATCTTAT |
| | | CATGTCTGTATACCGTCGACCTCTAGCTAGAGCTTGGCGTAATCAT |
| | | GGTCATAGCTGTTTCCTGTGTGAAATTGTTATCCGCTCACAATTCC |
| | | ACACAACATACGAGCCGGAAGCATAAAGTGTAAAGCCTGGGGTGCC |
| | | TAATGAGTGAGCTAACTCACATTAATTGCGTTGCGCTCACTGCCCG |
| | | CTTTCCAGTCGGGAAACCTGTCGTGCCAGCTGCATTAATGAATCGG |
| | | CCAACGCGCGGGGAGAGGCGGTTTGCGTATTGGGCGCTCTTCCGCT |
| | | TCCTCGCTCACTGACTCGCTGCGCTCGGTCGTTCGGCTGCGGCGAG |
| | | CGGTATCAGCTCACTCAAAGGCGGTAATACGGTTATCCACAGAATC |
| | | AGGGGATAACGCAGGAAAGAACATGTGAGCAAAAGGCCAGCAAAAG |
| | | GCCAGGAACCGTAAAAAGGCCGCGTTGCTGGCGTTTTTCCATAGGC |
| | | TCCGCCCCCCTGACGAGCATCACAAAAATCGACGCTCAAGTCAGAG |
| | | GTGGCGAAACCCGACAGGACTATAAAGATACCAGGCGTTTCCCCCT |
| | | GGAAGCTCCCTCGTGCGCTCTCCTGTTCCGACCCTGCCGCTTACCG |
| | | GATACCTGTCCGCCTTTCTCCCTTCGGGAAGCGTGGCGCTTTCTCA |
| | | TAGCTCACGCTGTAGGTATCTCAGTTCGGTGTAGGTCGTTCGCTCC |
| | | AAGCTGGGCTGTGTGCACGAACCCCCCGTTCAGCCCGACCGCTGCG |
| | | CCTTATCCGGTAACTATCGTCTTGAGTCCAACCCGGTAAGACACGA |
| | | CTTATCGCCACTGGCAGCAGCCACTGGTAACAGGATTAGCAGAGCG |
| | | AGGTATGTAGGCGGTGCTACAGAGTTCTTGAAGTGGTGGCCTAACT |
| | | ACGGCTACACTAGAAGAACAGTATTTGGTATCTGCGCTCTGCTGAA |
| | | GCCAGTTACCTTCGGAAAAAGAGTTGGTAGCTCTTGATCCGGCAAA |
| | | CAAACCACCGCTGGTAGCGGTGGTTTTTTTGTTTGCAAGCAGCAGA |
| | | TTACGCGCAGAAAAAAAGGATCTCAAGAAGATCCTTTGATCTTTTC |
| | | TACGGGGTCTGACGCTCAGTGGAACGAAAACTCACGTTAAGGGATT |
| | | TTGGTCATGAGATTATCAAAAAGGATCTTCACCTAGATCCTTTTAA |
| | | ATTAAAAATGAAGTTTTAAATCAATCTAAAGTATATATGAGTAAAC |
| | | TTGGTCTGACAGTTACCAATGCTTAATCAGTGAGGCACCTATCTCA |
| | | GCGATCTGTCTATTTCGTTCATCCATAGTTGCCTGACTCCCCGTCG |
| | | TGTAGATAACTACGATACGGGAGGGCTTACCATCTGGCCCCAGTGC |
| | | TGCAATGATACCGCGAGACCCACGCTCACCGGCTCCAGATTTATCA |
| | | GCAATAAACCAGCCAGCCGGAAGGGCCGAGCGCAGAAGTGGTCCTG |
| | | CAACTTTATCCGCCTCCATCCAGTCTATTAATTGTTGCCGGGAAGC |
| | | TAGAGTAAGTAGTTCGCCAGTTAATAGTTTGCGCAACGTTGTTGCC |
| | | ATTGCTACAGGCATCGTGGTGTCACGCTCGTCGTTTGGTATGGCTT |
| | | CATTCAGCTCCGGTTCCCAACGATCAAGGCGAGTTACATGATCCCC |
| | | CATGTTGTGCAAAAAAGCGGTTAGCTCCTTCGGTCCTCCGATCGTT |
| | | GTCAGAAGTAAGTTGGCCGCAGTGTTATCACTCATGGTTATGGCAG |
| | | CACTGCATAATTCTCTTACTGTCATGCCATCCGTAAGATGCTTTTC |
| | | TGTGACTGGTGAGTACTCAACCAAGTCATTCTGAGAATAGTGTATG |
| | | CGGCGACCGAGTTGCTCTTGCCCGGCGTCAATACGGGATAATACCG |
| | | CGCCACATAGCAGAACTTTAAAAGTGCTCATCATTGGAAAACGTTC |
| | | TTCGGGGCGAAAACTCTCAAGGATCTTACCGCTGTTGAGATCCAGT |
| | | TCGATGTAACCCACTCGTGCACCCAACTGATCTTCAGCATCTTTTA |
| | | CTTTCACCAGCGTTTCTGGGTGAGCAAAAACAGGAAGGCAAAATGC |
| | | CGCAAAAAAGGGAATAAGGGCGACACGGAAATGTTGAATACTCATA |
| | | CTCTTCCTTTTTCAATATTATTGAAGCATTTATCAGGGTTATTGTC |
| | | TCATGAGCGGATACATATTTGAATGTATTTAGAAAAATAAACAAAT |
| | | AGGGGTTCCGCGCACATTTCCCCGAAAAGTGCCACCTGACGTCGAC |
| | | GGATCGGGAGATCTCCCGATCCCCTATGGTGCACTCTCAGTACAAT |
| | | CTGCTCTGATGCCGCATAGTTAAGCCAGTATCTGCTCCCTGCTTGT |
| | | GTGTTGGAGGTCGCTGAGTAGTGCGCGAGCAAAATTTAAGCTACAA |
| | | CAAGGCAAGGCTTGACCGACAATTGCATGAAGAATCTGCTTAGGGT |
| | | TAGGCGTTTTGCGCTGCTTCGCGATGTACGGGCCAGATATACGCGT |
| | | TGACATTGATTATTGACTAGTTATTAATAGTAATCAATTACGGGGT |
| | | CATTAGTTCATAGCCCATATATGGAGTTCCGCGTTACATAACTTAC |
| | | GGTAAATGGCCCGCCTGGCTGACCGCCCAACGACCCCCGCCCATTG |
| | | ACGTCAATAATGACGTATGTTCCCATAGTAACGCCAATAGGGACTT |
| | | TCCATTGACGTCAATGGGTGGAGTATTTACGGTAAACTGCCCACTT |
| | | GGCAGTACATCAAGTGTATCATATGCCAAGTACGCCCCCTATTGAC |
| | | GTCAATGACGGTAAATGGCCCGCCTGGCATTATGCCCAGTACATGA |
| | | CCTTATGGGACTTTCCTACTTGGCAGTACATCTACGTATTAGTCAT |
| | | CGCTATTACCATGGTGATGCGGTTTTGGCAGTACATCAATGGGCGT |
| | | GGATAGCGGTTTGACTCACGGGGATTTCCAAGTCTCCACCCCATTG |
| | | ACGTCAATGGGAGTTTGTTTTGGCACCAAAATCAACGGGACTTTCC |
| | | AAAATGTCGTAACAACTCCGCCCCATTGACGCAAATGGGCGGTAGG |
| | | CGTGTACGGTGGGAGGTCTATATAAGCAGCGCGTTTTGCCTGTACT |
| | | GGGTCTCTCTGGTTAGACCAGATCTGAGCCTGGGAGCTCTCTGGCT |
| | | AACTAGGGAACCCACTGCTTAAGCCTCAATAAAGCTTGCCTTGAGT |
| | | GCTTCAAGTAGTGTGTGCCCGTCTGTTGTGTGACTCTGGTAACTAG |
| | | AGATCCCTCAGACCCTTTTAGTCAGTGTGGAAAATCTCTAGCAGTG |
| | | GCGCCCGAACAGGGACTTGAAAGCGAAAGGGAAACCAGAGGAGCTC |
| | | TCTCGACGCAGGACTCGGCTTGCTGAAGCGCGCACGGCAAGAGGCG |
| | | AGGGGCGGCGACTGGTGAGTACGCCAAAAATTTTGACTAGCGGAGG |
| | | CTAGAAGGAGAGAGATGGGTGCGAGAGCGTCAGTATTAAGCGGGGG |
| | | AGAATTAGATCGCGATGGGAAAAAATTCGGTTAAGGCCAGGGGGAA |
| | | AGAAAAAATATAAATTAAAACATATAGTATGGGCAAGCAGGGAGCT |
| | | AGAACGATTCGCAGTTAATCCTGGCCTGTTAGAAACATCAGAAGGC |
| | | TGTAGACAAATACTGGGACAGCTACAACCATCCCTTCAGACAGGAT |
| | | CAGAAGAACTTAGATCATTATATAATACAGTAGCAACCCTCTATTG |
| | | TGTGCATCAAAGGATAGAGATAAAAGACACCAAGGAAGCTTTAGAC |
| | | AAGATAGAGGAAGAGCAAAACAAAAGTAAGACCACCGCACAGCAAG |
| | | CGGCCGCTGATCTTCAGACCTGGAGGAGGAGATATGAGGGACAATT |
| | | GGAGAAGTGAATTATATAAATATAAAGTAGTAAAAATTGAACCATT |
| | | AGGAGTAGCACCCACCAAGGCAAAGAGAAGAGTGGTGCAGAGAGAA |
| | | AAAAGAGCAGTGGGAATAGGAGCTTTGTTCCTTGGGTTCTTGGGAG |
| | | CAGCAGGAAGCACTATGGGCGCAGCGTCAATGACGCTGACGGTACA |
| | | GGCCAGACAATTATTGTCTGGTATAGTGCAGCAGCAGAACAATTTG |
| | | CTGAGGGCTATTGAGGCGCAACAGCATCTGTTGCAACTCACAGTCT |
| | | GGGGCATCAAGCAGCTCCAGGCAAGAATCCTGGCTGTGGAAAGATA |
| | | CCTAAAGGATCAACAGCTCCTGGGGATTTGGGGTTGCTCTGGAAAA |
| | | CTCATTTGCACCACTGCTGTGCCTTGGAATGCTAGTTGGAGTAATA |
| | | AATCTCTGGAACAGATTTGGAATCACACGACCTGGATGGAGTGGGA |
| | | CAGAGAAATTAACAATTACACAAGCTTAATACACTCCTTAATTGAA |
| | | GAATCGCAAAACCAGCAAGAAAAGAATGAACAAGAATTATTGGAAT |
| | | TAGATAAATGGGCAAGTTTGTGGAATTGGTTTAACATAACAAATTG |
| | | GCTGTGGTATATAAAATTATTCATAATGATAGTAGGAGGCTTGGTA |
| | | GGTTTAAGAATAGTTTTTGCTGTACTTTCTATAGTGAATAGAGTTA |
| | | GGCAGGGATATTCACCATTATCGTTTCAGACCCACCTCCCAACCCC |
| | | GAGGGGACCCGACAGGCCCGAAGGAATAGAAGAAGAAGGTGGAGAG |
| | | AGAGACAGAGACAGATCCATTCGATTAGTGAACGGATCGGCACTGC |
| | | GTGCGCCAATTCTGCAGACAAATGGCAGTATTCATCCACAATTTTA |
| | | AAAGAAAAGGGGGGATTGGGGGGTACAGTGCAGGGGAAAGAATAGT |
| | | AGACATAATAGCAACAGACATACAAACTAAAGAATTACAAAAACAA |
| | | ATTACAAAAATTCAAAATTTTCGGGTTTATTACAGGGACAGCAGAG |
| | | ATCCAGTTTGGGCTAGCTTAATtaacctgcaggCCCGTGTCGGCTC |
| | | CAGATCTGGCCTCCGCGCCGGGTTTTGGCGCCTCCCGCGGGCGCCC |
| | | CCCTCCTCACGGCGAGCGCTGCCACGTCAGACGAAGGGCGCAGCGA |
| | | GCGTCCTGATCCTTCCGCCCGGACGCTCAGGACAGCGGCCCGCTGC |
| | | TCATAAGACTCGGCCTTAGAACCCCAGTATCAGCAGAAGGACATTT |
| | | TAGGACGGGACTTGGGTGACTCTAGGGCACTGGTTTTCTTTCCAGA |
| | | GAGCGGAACAGGCGAGGAAAAGTAGTCCCTTCTCGGCGATTCTGCG |
| | | GAGGGATCTCCGTGGGGCGGTGAACGCCGATGATTATATAAGGACG |
| | | CGCCGGGTGTGGCACAGCTAGTTCCGTCGCAGCCGGGATTTGGGTC |
| | | GCGGTTCTTGTTTGTGGATCGCTGTGATCGTCACTTGGTGAGTAGC |
| | | GGGCTGCTGGGCTGGCCGGGGCTTTCGTGGCCGCCGGGCCGCTCGG |
| | | TGGGACGGAAGCGTGTGGAGAGACCGCCAAGGGCTGTAGTCTGGGT |
| | | CCGCGAGCAAGGTTGCCCTGAACTGGGGGTTGGGGGGAGCGCAGCA |
| | | AAATGGCGGCTGTTCCCGAGTCTTGAATGGAAGACGCTTGTGAGGC |
| | | GGGCTGTGAGGTCGTTGAAACAAGGTGGGGGGCATGGTGGGCGGCA |
| | | AGAACCCAAGGTCTTGAGGCCTTCGCTAATGCGGGAAAGCTCTTAT |
| | | TCGGGTGAGATGGGCTGGGGCACCATCTGGGGACCCTGACGTGAAG |
| | | TTTGTCACTGACTGGAGAACTCGGTTTGTCGTCTGTTGCGGGGGCG |
| | | GCAGTTATGGCGGTGCCGTTGGGCAGTGCACCCGTACCTTTGGGAG |
| | | CGCGCGCCCTCGTCGTGTCGTGACGTCACCCGTTCTGTTGGCTTAT |
| | | AATGCAGGGTGGGGCCACCTGCCGGTAGGTGTGCGGTAGGCTTTTC |
| | | TCCGTCGCAGGACGCAGGGTTCGGGCCTAGGGTAGGCTCTCCTGAA |
| | | TCGACAGGCGCCGGACCTCTGGTGAGGGGAGGGATAAGTGAGGCGT |
| | | CAGTTTCTTTGGTCGGTTTTATGTACCTATCTTCTTAAGTAGCTGA |
| | | AGCTCCGGTTTTGAACTATGCGCTCGGGGTTGGCGAGTGTGTTTTG |
| | | TGAAGTTTTTTAGGCACCTTTTGAAATGTAATCATTTGGGTCAATA |
| | | TGTAATTTTCAGTGTTAGACTAGTAAATTGTCCGCTAAATTCTGGC |
| | | CGTTTTTGGCTTTTTTGTTAGACGAAGCTTGGGCTGCAGGTCGACT |
| | | CTAGGCCCaggCGGCCttgtacaaaaaagcACCGGTaggctgccac |
| | | cATGCCAAAGAAGAAGCGGAAGGTCGGTATCCACGGAGTCCCAGCA |
| | | GCCATGGCCAAGAACACAATTACAAAGACACTGAAGCTGAGGATCG |
| | | TGAGACCATACAACAGCGCTGAGGTCGAGAAGATTGTGGCTGATGA |
| | | AAAGAACAACAGGGAAAAGATCGCCCTCGAGAAGAACAAGGATAAG |
| | | GTGAAGGAGGCCTGCTCTAAGCACCTGAAAGTGGCCGCCTACTGCA |
| | | CCACACAGGTGGAGAGGAACGCCTGTCTGTTTTGTAAAGCTCGGAA |
| | | GCTGGATGATAAGTTTTACCAGAAGCTGCGGGGCCAGTTCCCCGAT |
| | | GCCGTCTTTTGGCAGGAGATTAGCGAGATCTTCAGACAGCTGCAGA |
| | | AGCAGGCCGCCGAGATCTACAACCAGAGCCTGATCGAGCTCTACTA |
| | | CGAGATCTTCATCAAGGGCAAGGGCATTGCCAACGCCTCCTCCGTG |
| | | GAGCACTACCTGAGCGACGTGTGCTACACAAGAGCCGCCGAGCTCT |
| | | TTAAGAACGCCGCTATCGCTTCCGGGCTGAGGAGCAAGATTAAGAG |
| | | TAACTTCCGGCTCAAGGAGCTGAAGAACATGAAGAGCGGCCTGCCC |
| | | ACTACAAAGAGCGACAACTTCCCAATTCCACTGGTGAAGCAGAAGG |
| | | GGGGCCAGTACACAGGGTTCGAGATTTCCAACCACAACAGCGACTT |
| | | TATTATTAAGATCCCCTTTGGCAGGTGGCAGGTCAAGAAGGAGATT |
| | | GACAAGTACAGGCCCTGGGAGAAGTTTGATTTCGAGCAGGTGCAGA |
| | | AGAGCCCCAAGCCTATTTCCCTGCTGCTGTCCACACAGCGGCGGAA |
| | | GAGGAACAAGGGGTGGTCTAAGGATGAGGGGACCGAGGCCGAGATT |
| | | AAGAAAGTGATGAACGGCGACTACCAGACAAGCTACATCGAGGTCA |
| | | AGCGGGGCAGTAAGATTGGCGAGAAGAGCGCCTGGATGCTGAACCT |
| | | GAGCATTGACGTGCCAAAGATTGATAAGGGCGTGGATCCCAGCATC |
| | | ATCGGAGGGATCGATGTGGGGGTCAAGAGCCCCCTCGTGTGCGCCA |
| | | TCAACAACGCCTTCAGCAGGTACAGCATCTCCGATAACGACCTGTT |
| | | CCACTTTAACAAGAAGATGTTCGCCCGGCGGAGGATTTTGCTCAAG |
| | | AAGAACCGGCACAAGCGGGCCGGACACGGGGCCAAGAACAAGCTCA |
| | | AGCCCATCACTATCCTGACCGAGAAGAGCGAGAGGTTCAGGAAGAA |
| | | GCTCATCGAGAGATGGGCCTGCGAGATCGCCGATTTCTTTATTAAG |
| | | AACAAGGTCGGAACAGTGCAGATGGAGAACCTCGAGAGCATGAAGA |
| | | GGAAGGAGGATTCCTACTTCAACATTCGGCTGAGGGGGTTCTGGCC |
| | | CTACGCTGAGATGCAGAACAAGATTGAGTTTAAGCTGAAGCAGTAC |
| | | GGGATTGAGATCCGGAAGGTGGCCCCCAACAACACCAGCAAGACCT |
| | | GCAGCAAGTGCGGGCACCTCAACAACTACTTCAACTTCGAGTACCG |
| | | GAAGAAGAACAAGTTCCCACACTTCAAGTGCGAGAAGTGCAACTTT |
| | | AAGGAGAACGCCGATTACAACGCCGCCCTGAACATCAGCAACCCTA |
| | | AGCTGAAGAGCACTAAGGAGGAGCCCggatccggaATGGCTACAGT |
| | | GAAGTTTAAGTATAAAGGGGAAGAGAAAGAGGTGGACATCAGTAAA |
| | | ATCAAGAAAGTGTGGCGCGTAGGTAAGATGATCAGCTTTACGTACG |
| | | ATGAAGGAGGAGGAAAGACTGGCAGAGGAGCCGTTTCAGAAAAGGA |
| | | CGCTCCAAAGGAATTGTTGCAGATGCTCGAGAAGCAGAAGAAGgga |
| | | agcggaAAAAGGCCGGCGGCCACGAAAAAGGCCGGCCAGGCAAAAA |
| | | AGAAAAAGtacccatacgatgttccagattacgctggaagcggaga |
| | | gggcaggggaagtcttctaacatgcggggacgtggaggaaaatccc |
| | | ggccccatgaaaaagcctgaactcaccgcgacgtctgtcgagaagt |
| | | ttctgatcgaaaagttcgacagcgtctccgacctgatgcagctctc |
| | | ggagggcgaagaatctcgtgctttcagcttcgatgtaggagggcgt |
| | | ggatatgtcctgcgggtaaatagctgcgccgatggtttctacaaag |
| | | atcgttatgtttatcggcactttgcatcggccgcgctcccgattcc |
| | | ggaagtgcttgacattggggaatttagcgagagcctgacctattgc |
| | | atctcccgccgtgcacagggtgtcacgttgcaagacctgcctgaaa |
| | | ccgaactgcccgctgttctgcagccggtcgcggaggccatggatgc |
| | | gatcgctgcggccgatcttagccagacgagegggttcggcccattc |
| | | ggaccgcaaggaatcggtcaatacactacatggcgtgatttcatat |
| | | gcgcgattgctgatccccatgtgtatcactggcaaactgtgatgga |
| | | cgacaccgtcagtgcgtccgtcgcgcaggctctcgatgagctgatg |
| | | ctttgggccgaggactgccccgaagtccggcacctcgtgcacgcgg |
| | | atttcggctccaacaatgtcctgacggacaatggccgcataacagc |
| | | ggtcattgactggagcgaggcgatgttcggggattcccaatacgag |
| | | gtcgccaacatcttcttctggaggccgtggttggcttgtatggagc |
| | | agcagacgcgctacttcgagcggaggcatccggagcttgcaggatc |
| | | gccgcggctccgggcgtatatgctccgcattggtcttgaccaactc |
| | | tatcagagcttggttgacggcaatttcgatgatgcagcttgggcgc |
| | | agggtcgatgcgacgcaatcgtccgatccggagccgggactgtcgg |
| | | gcgtacacaaatcgcccgcagaagcgcggccgtctggaccgatggc |
| | | tgtgtagaagtactcgccgatagtggaaaccgacgccccagcactc |
| | | gtccgagggcaaaggaatagacccgaattccagGGCCAggcCG |
| 13 | pLV- | gtcgacggatcgggagatctcccgatcccctatggtgcactctcag |
| | EW9 | tacaatctgctctgatgccgcatagttaagccagtatctgctccct |
| | (comprising | gcttgtgtgttggaggtcgctgagtagtgcgcgagcaaaatttaag |
| | CBh: | ctacaacaaggcaaggcttgaccgacaattgcatgaagaatctgct |
| | Cas12f1 | tagggttaggcgttttgcgctgcttcgcgatgtacgggccagatat |
| | expression | acgcgttgacattgattattgactagttattaatagtaatcaatta |
| | cassette) | cggggtcattagttcatagcccatatatggagttccgcgttacata |
| | | acttacggtaaatggcccgcctggctgaccgcccaacgacccccgc |
| | | ccattgacgtcaataatgacgtatgttcccatagtaacgccaatag |
| | | ggactttccattgacgtcaatgggtggagtatttacggtaaactgc |
| | | ccacttggcagtacatcaagtgtatcatatgccaagtacgccccct |
| | | attgacgtcaatgacggtaaatggcccgcctggcattatgcccagt |
| | | acatgaccttatgggactttcctacttggcagtacatctacgtatt |
| | | agtcatcgctattaccatggtgatgcggttttggcagtacatcaat |
| | | gggcgtggatagcggtttgactcacggggatttccaagtctccacc |
| | | ccattgacgtcaatgggagtttgttttggcaccaaaatcaacggga |
| | | ctttccaaaatgtcgtaacaactccgccccattgacgcaaatgggc |
| | | ggtaggcgtgtacggtgggaggtctatataagcagcgcgttttgcc |
| | | tgtactgggtctctctggttagaccagatctgagcctgggagctct |
| | | ctggctaactagggaacccactgcttaagcctcaataaagcttgcc |
| | | ttgagtgcttcaagtagtgtgtgcccgtctgttgtgtgactctggt |
| | | aactagagatccctcagacccttttagtcagtgtggaaaatctcta |
| | | gcagtggcgcccgaacagggacttgaaagcgaaagggaaaccagag |
| | | gagctctctcgacgcaggactcggcttgctgaagcgcgcacggcaa |
| | | gaggcgaggggcggcgactggtgagtacgccaaaaattttgactag |
| | | cggaggctagaaggagagagatgggtgcgagagcgtcagtattaag |
| | | cgggggagaattagatcgcgatgggaaaaaattcggttaaggccag |
| | | ggggaaagaaaaaatataaattaaaacatatagtatgggcaagcag |
| | | ggagctagaacgattcgcagttaatcctggcctgttagaaacatca |
| | | gaaggctgtagacaaatactgggacagctacaaccatcccttcaga |
| | | caggatcagaagaacttagatcattatataatacagtagcaaccct |
| | | ctattgtgtgcatcaaaggatagagataaaagacaccaaggaagct |
| | | ttagacaagatagaggaagagcaaaacaaaagtaagaccaccgcac |
| | | agcaagcggccgctgatcttcagacctggaggaggagatatgaggg |
| | | acaattggagaagtgaattatataaatataaagtagtaaaaattga |
| | | accattaggagtagcacccaccaaggcaaagagaagagtggtgcag |
| | | agagaaaaaagagcagtgggaataggagctttgttccttgggttct |
| | | tgggagcagcaggaagcactatgggcgcagcgtcaatgacgctgac |
| | | ggtacaggccagacaattattgtctggtatagtgcagcagcagaac |
| | | aatttgctgagggctattgaggcgcaacagcatctgttgcaactca |
| | | cagtctggggcatcaagcagctccaggcaagaatcctggctgtgga |
| | | aagatacctaaaggatcaacagctcctggggatttggggttgctct |
| | | ggaaaactcatttgcaccactgctgtgccttggaatgctagttgga |
| | | gtaataaatctctggaacagatttggaatcacacgacctggatgga |
| | | gtgggacagagaaattaacaattacacaagcttaatacactcctta |
| | | attgaagaatcgcaaaaccagcaagaaaagaatgaacaagaattat |
| | | tggaattagataaatgggcaagtttgtggaattggtttaacataac |
| | | aaattggctgtggtatataaaattattcataatgatagtaggaggc |
| | | ttggtaggtttaagaatagtttttgctgtactttctatagtgaata |
| | | gagttaggcagggatattcaccattatcgtttcagacccacctccc |
| | | aaccccgaggggacccgacaggcccgaaggaatagaagaagaaggt |
| | | ggagagagagacagagacagatccattcgattagtgaacggatcgg |
| | | cactgcgtgcgccaattctgcagacaaatggcagtattcatccaca |
| | | attttaaaagaaaaggggggattggggggtacagtgcaggggaaag |
| | | aatagtagacataatagcaacagacatacaaactaaagaattacaa |
| | | aaacaaattacaaaaattcaaaattttcgggtttattacagggaca |
| | | gcagagatccagtttgggctagcttaattaaccGGCCGCcgttaca |
| | | taacttacggtaaatggcccgcctggctgaccgcccaacgaccccc |
| | | gcccattgacgtcaatagtaacgccaatagggactttccattgacg |
| | | tcaatgggtggagtatttacggtaaactgcccacttggcagtacat |
| | | caagtgtatcatatgccaagtacgccccctattgacgtcaatgacg |
| | | gtaaatggcccgcctggcattgtgcccagtacatgaccttatggga |
| | | ctttcctacttggcagtacatctacgtattagtcatcgctattacc |
| | | atggtcgaggtgagccccacgttctgcttcactctccccatctccc |
| | | ccccctccccacccccaattttgtatttatttattttttaattatt |
| | | ttgtgcagcgatgggggcggggggggggggggggggggcgaggggg |
| | | gggcggggcgaggcggagaggtgcggcggcagccaatcagagcggc |
| | | gcgctccgaaagtttccttttatggcgaggcggcggcggcggcggc |
| | | cctataaaaagcgaagcgcgcggggggggagtcgctgcgcgctgcc |
| | | ttcgccccgtgccccgctcegccgccgcctegogccgcccgccccg |
| | | gctctgactgaccgcgttactcccacaggtgagcggggggacggcc |
| | | cttctcctccgggctgtaattagctgagcaagaggtaagggtttaa |
| | | gggatggttggttggtggggtattaatgtttaattacctggagcac |
| | | ctgcctgaaatcactttttttcaggttggtacgccggtaggctgcc |
| | | accatgccaaagaagaagcggaaggtcggtatccacggagtcccag |
| | | cagccatggccaagaacacaattacaaagacactgaagctgaggat |
| | | cgtgagaccatacaacagcgctgaggtcgagaagattgtggctgat |
| | | gaaaagaacaacagggaaaagatcgccctcgagaagaacaaggata |
| | | aggtgaaggaggcctgctctaagcacctgaaagtggccgcctactg |
| | | caccacacaggtggagaggaacgcctgtctgttttgtaaagctcgg |
| | | aagctggatgataagttttaccagaagctgcggggccagttccccg |
| | | atgccgtcttttggcaggagattagcgagatcttcagacagctgca |
| | | gaagcaggccgccgagatctacaaccagagcctgatcgagctctac |
| | | tacgagatcttcatcaagggcaagggcattgccaacgcctcctccg |
| | | tggagcactacctgagcgacgtgtgctacacaagagccgccgagct |
| | | ctttaagaacgccgctatcgcttccgggctgaggagcaagattaag |
| | | agtaacttccggctcaaggagctgaagaacatgaagagcggcctgc |
| | | ccactacaaagagcgacaacttcccaattccactggtgaagcagaa |
| | | ggggggccagtacacagggttcgagatttccaaccacaacagcgac |
| | | tttattattaagatcccctttggcaggtggcaggtcaagaaggaga |
| | | ttgacaagtacaggccctgggagaagtttgatttcgagcaggtgca |
| | | gaagagccccaagcctatttccctgctgctgtccacacagcggcgg |
| | | aagaggaacaaggggtggtctaaggatgaggggaccgaggccgaga |
| | | ttaagaaagtgatgaacggcgactaccagacaagctacatcgaggt |
| | | caagcggggcagtaagattggcgagaagagcgcctggatgctgaac |
| | | ctgagcattgacgtgccaaagattgataagggcgtggatcccagca |
| | | tcatcggagggatcgatgtgggggtcaagagccccctcgtgtgcgc |
| | | catcaacaacgccttcagcaggtacagcatctccgataacgacctg |
| | | ttccactttaacaagaagatgttcgcccggcggaggattttgctca |
| | | agaagaaccggcacaagcgggccggacacggggccaagaacaagct |
| | | caagcccatcactatcctgaccgagaagagcgagaggttcaggaag |
| | | aagctcatcgagagatgggcctgcgagatcgccgatttctttatta |
| | | agaacaaggtcggaacagtgcagatggagaacctcgagagcatgaa |
| | | gaggaaggaggattcctacttcaacattcggctgagggggttctgg |
| | | ccctacgctgagatgcagaacaagattgagtttaagctgaagcagt |
| | | acgggattgagatccggaaggtggcccccaacaacaccagcaagac |
| | | ctgcagcaagtgcgggcacctcaacaactacttcaacttcgagtac |
| | | cggaagaagaacaagttcccacacttcaagtgcgagaagtgcaact |
| | | ttaaggagaacgccgattacaacgccgccctgaacatcagcaaccc |
| | | taagctgaagagcactaaggaggagcccaaaaggccggcggccacg |
| | | aaaaaggccggccaggcaaaaaagaaaaagtacccatacgatgttc |
| | | cagattacgctggaagcggagagggcaggggaagtcttctaacatg |
| | | cggggacgtggaggaaaatcccggccccatgaaaaagcctgaactc |
| | | accgcgacgtctgtcgagaagtttctgatcgaaaagttcgacagcg |
| | | tctccgacctgatgcagctctcggagggcgaagaatctcgtgcttt |
| | | cagcttcgatgtaggagggcgtggatatgtcctgcgggtaaatagc |
| | | tgcgccgatggtttctacaaagatcgttatgtttatcggcactttg |
| | | catcggccgcgctcccgattccggaagtgcttgacattggggaatt |
| | | tagcgagagcctgacctattgcatctcccgccgtgcacagggtgtc |
| | | acgttgcaagacctgcctgaaaccgaactgcccgctgttctgcagc |
| | | cggtcgcggaggccatggatgcgatcgctgcggccgatcttagcca |
| | | gacgagcgggttcggcccatteggaccgcaaggaatcggtcaatac |
| | | actacatggcgtgatttcatatgcgcgattgctgatccccatgtgt |
| | | atcactggcaaactgtgatggacgacaccgtcagtgcgtccgtcgc |
| | | gcaggctctcgatgagctgatgctttgggccgaggactgccccgaa |
| | | gtccggcacctcgtgcacgcggatttcggctccaacaatgtcctga |
| | | cggacaatggccgcataacagcggtcattgactggagcgaggcgat |
| | | gttcggggattcccaatacgaggtcgccaacatcttcttctggagg |
| | | ccgtggttggcttgtatggagcagcagacgcgctacttcgagcgga |
| | | ggcatccggagcttgcaggatcgccgcggctccgggcgtatatgct |
| | | ccgcattggtcttgaccaactctatcagagcttggttgacggcaat |
| | | ttcgatgatgcagcttgggcgcagggtcgatgcgacgcaatcgtcc |
| | | gatccggagccgggactgtcgggcgtacacaaatcgcccgcagaag |
| | | cgcggccgtctggaccgatggctgtgtagaagtactcgccgatagt |
| | | ggaaaccgacgccccagcactcgtccgagggcaaaggaatagaccc |
| | | gaattccagggccaggccggccgatatcaagcttatcgataatcaa |
| | | cctctggattacaaaatttgtgaaagattgactggtattcttaact |
| | | atgttgctccttttacgctatgtggatacgctgctttaatgccttt |
| | | gtatcatgctattgcttcccgtatggctttcattttctcctccttg |
| | | tataaatcctggttgctgtctctttatgaggagttgtggcccgttg |
| | | tcaggcaacgtggcgtggtgtgcactgtgtttgctgacgcaacccc |
| | | cactggttggggcattgccaccacctgtcagctcctttccgggact |
| | | ttcgctttccccctccctattgccacggcggaactcatcgccgcct |
| | | gccttgcccgctgctggacaggggctcggctgttgggcactgacaa |
| | | ttccgtggtgttgtcggggaaatcatcgtcctttccttggctgctc |
| | | gcctgtgttgccacctggattctgcgcgggacgtccttctgctacg |
| | | tccctteggccctcaatccageggaccttccttcccgcggcctgct |
| | | gccggctctgcggcctcttccgcgtcttcgccttcgccctcagacg |
| | | agtoggatctccctttgggccgcctccccgcatcgataccgtcgac |
| | | ctcgagacctagaaaaacatggagcaatcacaagtagcaatacagc |
| | | agctaccaatgctgattgtgcctggctagaagcacaagaggaggag |
| | | gaggtgggttttccagtcacacctcaggtacctttaagaccaatga |
| | | cttacaaggcagctgtagatcttagccactttttaaaagaaaaggg |
| | | gggactggaagggctaattcactcccaacgaagacaagatatcctt |
| | | gatctgtggatctaccacacacaaggctacttccctgattggcaga |
| | | actacacaccagggccagggatcagatatccactgacctttggatg |
| | | gtgctacaagctagtaccagttgagcaagagaaggtagaagaagcc |
| | | aatgaaggagagaacacccgcttgttacaccctgtgagcctgcatg |
| | | ggatggatgacccggagagagaagtattagagtggaggtttgacag |
| | | ccgcctagcatttcatcacatggcccgagagctgcatccggactgt |
| | | actgggtctctctggttagaccagatctgagcctgggagctctctg |
| | | gctaactagggaacccactgcttaagcctcaataaagcttgccttg |
| | | agtgcttcaagtagtgtgtgcccgtctgttgtgtgactctggtaac |
| | | tagagatccctcagacccttttagtcagtgtggaaaatctctagca |
| | | gggcccgtttaaacccgctgatcagcctcgactgtgccttctagtt |
| | | gccagccatctgttgtttgcccctcccccgtgccttccttgaccct |
| | | ggaaggtgccactcccactgtcctttcctaataaaatgaggaaatt |
| | | gcatcgcattgtctgagtaggtgtcattctattctggggggggggt |
| | | ggggcaggacagcaagggggaggattgggaagacaatagcaggcat |
| | | gctggggatgcggtgggctctatggcttctgaggcggaaagaacca |
| | | gctggggctctagggggtatccccacgcgccctgtagcggcgcatt |
| | | aagcgcggcgggtgtggtggttacgcgcagcgtgaccgctacactt |
| | | gccagcgccctagegcccgctcctttcgctttcttcccttcctttc |
| | | tegccacgttcgccggctttccccgtcaagctctaaatcgggggct |
| | | ccctttagggttccgatttagtgctttacggcacctcgaccccaaa |
| | | aaacttgattagggtgatggttcacgtagtgggccatcgccctgat |
| | | agacggtttttcgccctttgacgttggagtccacgttctttaatag |
| | | tggactcttgttccaaactggaacaacactcaaccctatctcggtc |
| | | tattcttttgatttataagggattttgccgatttcggcctattggt |
| | | taaaaaatgagctgatttaacaaaaatttaacgcgaattaattctg |
| | | tggaatgtgtgtcagttagggtgtggaaagtccccaggctccccag |
| | | caggcagaagtatgcaaagcatgcatctcaattagtcagcaaccag |
| | | gtgtggaaagtccccaggctccccagcaggcagaagtatgcaaagc |
| | | atgcatctcaattagtcagcaaccatagtcccgcccctaactccgc |
| | | ccatcccgcccctaactccgcccagttccgcccattctccgcccca |
| | | tggctgactaattttttttatttatgcagaggccgaggccgcctct |
| | | gcctctgagctattccagaagtagtgaggaggcttttttggaggcc |
| | | taggcttttgcaaaaagctcccgggagcttgtatatccattttcgg |
| | | atctgatcagcacgtgttgacaattaatcatcggcatagtatatcg |
| | | gcatagtataatacgacaaggtgaggaactaaaccatggccaagtt |
| | | gaccagtgccgttccggtgctcaccgcgcgcgacgtcgccggagcg |
| | | gtcgagttctggaccgaccggctcgggttctcccgggacttcgtgg |
| | | aggacgacttcgccggtgtggtccgggacgacgtgaccctgttcat |
| | | cagcgcggtccaggaccaggtggtgccggacaacaccctggcctgg |
| | | gtgtgggtgcgcggcctggacgagctgtacgccgagtggtcggagg |
| | | tcgtgtccacgaacttccgggacgcctccgggccggccatgaccga |
| | | gatcggcgagcagccgtggggggggagttcgccctgcgcgacccgg |
| | | ccggcaactgcgtgcacttcgtggccgaggagcaggactgacacgt |
| | | gctacgagatttcgattccaccgccgccttctatgaaaggttgggc |
| | | ttcggaatcgttttccgggacgccggctggatgatcctccagcgcg |
| | | gggatctcatgctggagttcttcgcccaccccaacttgtttattgc |
| | | agcttataatggttacaaataaagcaatagcatcacaaatttcaca |
| | | aataaagcatttttttcactgcattctagttgtggtttgtccaaac |
| | | tcatcaatgtatottatcatgtctgtataccgtcgacctctagcta |
| | | gagcttggcgtaatcatggtcatagctgtttcctgtgtgaaattgt |
| | | tatccgctcacaattccacacaacatacgagccggaagcataaagt |
| | | gtaaagcctggggtgcctaatgagtgagctaactcacattaattgc |
| | | gttgcgctcactgcccgctttccagtcgggaaacctgtcgtgccag |
| | | ctgcattaatgaatcggccaacgcgcggggagaggcggtttgcgta |
| | | ttgggcgctcttccgcttcctcgctcactgactcgctgcgctcggt |
| | | cgttcggctgcggcgagcggtatcagctcactcaaaggcggtaata |
| | | cggttatccacagaatcaggggataacgcaggaaagaacatgtgag |
| | | caaaaggccagcaaaaggccaggaaccgtaaaaaggccgcgttgct |
| | | ggcgtttttccataggctccgcccccctgacgagcatcacaaaaat |
| | | cgacgctcaagtcagaggtggcgaaacccgacaggactataaagat |
| | | accaggcgtttccccctggaagctccctegtgcgctctcctgttcc |
| | | gaccctgccgcttaccggatacctgtccgcctttctcccttcggga |
| | | agcgtggcgctttctcatagctcacgctgtaggtatctcagttcgg |
| | | tgtaggtcgttcgctccaagctgggctgtgtgcacgaaccccccgt |
| | | tcagcccgaccgctgcgccttatccggtaactatcgtcttgagtcc |
| | | aacccggtaagacacgacttatcgccactggcagcagccactggta |
| | | acaggattagcagagcgaggtatgtaggcggtgctacagagttctt |
| | | gaagtggtggcctaactacggctacactagaagaacagtatttggt |
| | | atctgcgctctgctgaagccagttaccttcggaaaaagagttggta |
| | | gctcttgatccggcaaacaaaccaccgctggtagcggtggtttttt |
| | | tgtttgcaagcagcagattacgcgcagaaaaaaaggatctcaagaa |
| | | gatcctttgatcttttctacggggtctgacgctcagtggaacgaaa |
| | | actcacgttaagggattttggtcatgagattatcaaaaaggatctt |
| | | cacctagatccttttaaattaaaaatgaagttttaaatcaatctaa |
| | | agtatatatgagtaaacttggtctgacagttaccaatgcttaatca |
| | | gtgaggcacctatctcagcgatctgtctatttcgttcatccatagt |
| | | tgcctgactccccgtcgtgtagataactacgatacgggagggctta |
| | | ccatctggccccagtgctgcaatgataccgcgagacccacgctcac |
| | | cggctccagatttatcagcaataaaccagccagccggaagggccga |
| | | gcgcagaagtggtcctgcaactttatccgcctccatccagtctatt |
| | | aattgttgccgggaagctagagtaagtagttcgccagttaatagtt |
| | | tgcgcaacgttgttgccattgctacaggcatcgtggtgtcacgctc |
| | | gtcgtttggtatggcttcattcagctccggttcccaacgatcaagg |
| | | cgagttacatgatcccccatgttgtgcaaaaaagcggttagctcct |
| | | tcggtcctccgatcgttgtcagaagtaagttggccgcagtgttatc |
| | | actcatggttatggcagcactgcataattctcttactgtcatgcca |
| | | tccgtaagatgcttttctgtgactggtgagtactcaaccaagtcat |
| | | tctgagaatagtgtatgcggcgaccgagttgctcttgcccggcgtc |
| | | aatacgggataataccgcgccacatagcagaactttaaaagtgctc |
| | | atcattggaaaacgttcttcggggcgaaaactctcaaggatcttac |
| | | cgctgttgagatccagttcgatgtaacccactcgtgcacccaactg |
| | | atcttcagcatcttttactttcaccagcgtttctgggtgagcaaaa |
| | | acaggaaggcaaaatgccgcaaaaaagggaataagggcgacacgga |
| | | aatgttgaatactcatactcttcctttttcaatattattgaagcat |
| | | ttatcagggttattgtctcatgagcggatacatatttgaatgtatt |
| | | tagaaaaataaacaaataggggttccgcgcacatttccccgaaaag |
| | | tgccacctgac |
| 14 | pLV- | gtcgacggatcgggagatctcccgatcccctatggtgcactctcag |
| | EW11 | tacaatctgctctgatgccgcatagttaagccagtatctgctccct |
| | comprising | gcttgtgtgttggaggtcgctgagtagtgcgcgagcaaaatttaag |
| | CBh: | ctacaacaaggcaaggcttgaccgacaattgcatgaagaatctgct |
| | Cas12f1- | tagggttaggcgttttgcgctgcttcgcgatgtacgggccagatat |
| | Sso7d | acgcgttgacattgattattgactagttattaatagtaatcaatta |
| | expression | cggggtcattagttcatagcccatatatggagttccgcgttacata |
| | cassette | acttacggtaaatggcccgcctggctgaccgcccaacgacccccgc |
| | | ccattgacgtcaataatgacgtatgttcccatagtaacgccaatag |
| | | ggactttccattgacgtcaatgggtggagtatttacggtaaactgc |
| | | ccacttggcagtacatcaagtgtatcatatgccaagtacgccccct |
| | | attgacgtcaatgacggtaaatggcccgcctggcattatgcccagt |
| | | acatgaccttatgggactttcctacttggcagtacatctacgtatt |
| | | agtcatcgctattaccatggtgatgcggttttggcagtacatcaat |
| | | gggcgtggatagcggtttgactcacggggatttccaagtctccacc |
| | | ccattgacgtcaatgggagtttgttttggcaccaaaatcaacggga |
| | | ctttccaaaatgtcgtaacaactccgccccattgacgcaaatgggc |
| | | ggtaggcgtgtacggtgggaggtctatataagcagcgcgttttgcc |
| | | tgtactgggtctctctggttagaccagatctgagcctgggagctct |
| | | ctggctaactagggaacccactgcttaagcctcaataaagcttgcc |
| | | ttgagtgcttcaagtagtgtgtgcccgtctgttgtgtgactctggt |
| | | aactagagatccctcagacccttttagtcagtgtggaaaatctcta |
| | | gcagtggcgcccgaacagggacttgaaagcgaaagggaaaccagag |
| | | gagctctctcgacgcaggactcggcttgctgaagcgcgcacggcaa |
| | | gaggcgaggggcggcgactggtgagtacgccaaaaattttgactag |
| | | cggaggctagaaggagagagatgggtgcgagagcgtcagtattaag |
| | | cgggggagaattagatcgcgatgggaaaaaattcggttaaggccag |
| | | ggggaaagaaaaaatataaattaaaacatatagtatgggcaagcag |
| | | ggagctagaacgattcgcagttaatcctggcctgttagaaacatca |
| | | gaaggctgtagacaaatactgggacagctacaaccatcccttcaga |
| | | caggatcagaagaacttagatcattatataatacagtagcaaccct |
| | | ctattgtgtgcatcaaaggatagagataaaagacaccaaggaagct |
| | | ttagacaagatagaggaagagcaaaacaaaagtaagaccaccgcac |
| | | agcaagcggccgctgatcttcagacctggaggaggagatatgaggg |
| | | acaattggagaagtgaattatataaatataaagtagtaaaaattga |
| | | accattaggagtagcacccaccaaggcaaagagaagagtggtgcag |
| | | agagaaaaaagagcagtgggaataggagctttgttccttgggttct |
| | | tgggagcagcaggaagcactatgggcgcagcgtcaatgacgctgac |
| | | ggtacaggccagacaattattgtctggtatagtgcagcagcagaac |
| | | aatttgctgagggctattgaggcgcaacagcatctgttgcaactca |
| | | cagtctggggcatcaagcagctccaggcaagaatcctggctgtgga |
| | | aagatacctaaaggatcaacagctcctggggatttggggttgctct |
| | | ggaaaactcatttgcaccactgctgtgccttggaatgctagttgga |
| | | gtaataaatctctggaacagatttggaatcacacgacctggatgga |
| | | gtgggacagagaaattaacaattacacaagcttaatacactcctta |
| | | attgaagaatcgcaaaaccagcaagaaaagaatgaacaagaattat |
| | | tggaattagataaatgggcaagtttgtggaattggtttaacataac |
| | | aaattggctgtggtatataaaattattcataatgatagtaggaggc |
| | | ttggtaggtttaagaatagtttttgctgtactttctatagtgaata |
| | | gagttaggcagggatattcaccattatcgtttcagacccacctccc |
| | | aaccccgaggggacccgacaggcccgaaggaatagaagaagaaggt |
| | | ggagagagagacagagacagatccattcgattagtgaacggatcgg |
| | | cactgcgtgcgccaattctgcagacaaatggcagtattcatccaca |
| | | attttaaaagaaaaggggggattggggggtacagtgcaggggaaag |
| | | aatagtagacataatagcaacagacatacaaactaaagaattacaa |
| | | aaacaaattacaaaaattcaaaattttcgggtttattacagggaca |
| | | gcagagatccagtttgggctagcttaattaaccGGCCGCcgttaca |
| | | taacttacggtaaatggcccgcctggctgaccgcccaacgaccccc |
| | | gcccattgacgtcaatagtaacgccaatagggactttccattgacg |
| | | tcaatgggggagtatttacggtaaactgcccacttggcagtacatc |
| | | aagtgtatcatatgccaagtacgccccctattgacgtcaatgacgg |
| | | taaatggcccgcctggcattgtgcccagtacatgaccttatgggac |
| | | tttcctacttggcagtacatctacgtattagtcatcgctattacca |
| | | tggtcgaggtgagccccacgttctgcttcactctccccatctcccc |
| | | cccctccccacccccaattttgtatttatttattttttaattattt |
| | | tgtgcagcgatgggggcggggggggggggggggcggggcgaggggc |
| | | ggggcggggcgaggcggagaggtgcggcggcagccaatcagagcgg |
| | | cgcgctccgaaagtttccttttatggcgaggcggcggcggcggcgg |
| | | ccctataaaaagcgaagcgcgcggggggggagtcgctgcgcgctgc |
| | | cttcgccccgtgccccgetcegcegccgectegcgccgcccgcccc |
| | | ggctctgactgaccgcgttactcccacaggtgageggggggacggc |
| | | ccttctcctccgggctgtaattagctgagcaagaggtaagggttta |
| | | agggatggttggttggtggggtattaatgtttaattacctggagca |
| | | cctgcctgaaatcactttttttcaggttggtacgccggtaggctgc |
| | | caccatgccaaagaagaagcggaaggtcggtatccacggagtccca |
| | | gcagccatggccaagaacacaattacaaagacactgaagctgagga |
| | | tcgtgagaccatacaacagcgctgaggtcgagaagattgtggctga |
| | | tgaaaagaacaacagggaaaagatcgccctcgagaagaacaaggat |
| | | aaggtgaaggaggcctgctctaagcacctgaaagtggccgcctact |
| | | gcaccacacaggtggagaggaacgcctgtctgttttgtaaagctcg |
| | | gaagctggatgataagttttaccagaagctgcggggccagttcccc |
| | | gatgccgtcttttggcaggagattagcgagatcttcagacagctgc |
| | | agaagcaggccgccgagatctacaaccagagcctgatcgagctcta |
| | | ctacgagatcttcatcaagggcaagggcattgccaacgcctcctcc |
| | | gtggagcactacctgagcgacgtgtgctacacaagagccgccgagc |
| | | tctttaagaacgccgctatcgcttccgggctgaggagcaagattaa |
| | | gagtaacttccggctcaaggagctgaagaacatgaagagcggcctg |
| | | cccactacaaagagcgacaacttcccaattccactggtgaagcaga |
| | | aggggggccagtacacagggttcgagatttccaaccacaacagcga |
| | | ctttattattaagatcccctttggcaggtggcaggtcaagaaggag |
| | | attgacaagtacaggccctgggagaagtttgatttcgagcaggtgc |
| | | agaagagccccaagcctatttccctgctgctgtccacacagcggcg |
| | | gaagaggaacaaggggtggtctaaggatgaggggaccgaggccgag |
| | | attaagaaagtgatgaacggcgactaccagacaagctacatcgagg |
| | | tcaagcggggcagtaagattggcgagaagagcgcctggatgctgaa |
| | | cctgagcattgacgtgccaaagattgataagggcgtggatcccagc |
| | | atcatcggagggatcgatgtgggggtcaagagccccctcgtgtgcg |
| | | ccatcaacaacgccttcagcaggtacagcatctccgataacgacct |
| | | gttccactttaacaagaagatgttcgcccggcggaggattttgctc |
| | | aagaagaaccggcacaagcgggccggacacggggccaagaacaagc |
| | | tcaagcccatcactatcctgaccgagaagagcgagaggttcaggaa |
| | | gaagctcatcgagagatgggcctgcgagatcgccgatttctttatt |
| | | aagaacaaggtcggaacagtgcagatggagaacctcgagagcatga |
| | | agaggaaggaggattcctacttcaacattcggctgagggggttctg |
| | | gccctacgctgagatgcagaacaagattgagtttaagctgaagcag |
| | | tacgggattgagatccggaaggtggcccccaacaacaccagcaaga |
| | | cctgcagcaagtgcgggcacctcaacaactacttcaacttcgagta |
| | | ccggaagaagaacaagttcccacacttcaagtgcgagaagtgcaac |
| | | tttaaggagaacgccgattacaacgccgccctgaacatcagcaacc |
| | | ctaagctgaagagcactaaggaggagcccggatccggaATGGCTAC |
| | | AGTGAAGTTTAAGTATAAAGGGGAAGAGAAAGAGGTGGACATCAGT |
| | | AAAATCAAGAAAGTGTGGCGCGTAGGTAAGATGATCAGCTTTACGT |
| | | ACGATGAAGGAGGAGGAAAGACTGGCAGAGGAGCCGTTTCAGAAAA |
| | | GGACGCTCCAAAGGAATTGTTGCAGATGCTCGAGAAGCAGAAGAAG |
| | | ggaagcggaaaaaggccggcggccacgaaaaaggccggccaggcaa |
| | | aaaagaaaaagtacccatacgatgttccagattacgctggaagcgg |
| | | agagggcaggggaagtcttctaacatgcggggacgtggaggaaaat |
| | | cccggccccatgaaaaagcctgaactcaccgcgacgtctgtcgaga |
| | | agtttctgatcgaaaagttcgacagcgtctccgacctgatgcagct |
| | | ctcggagggcgaagaatctcgtgctttcagcttcgatgtaggaggg |
| | | cgtggatatgtcctgcgggtaaatagctgcgccgatggtttctaca |
| | | aagatcgttatgtttatcggcactttgcatcggccgcgctcccgat |
| | | tccggaagtgcttgacattggggaatttagcgagagcctgacctat |
| | | tgcatctcccgccgtgcacagggtgtcacgttgcaagacctgcctg |
| | | aaaccgaactgcccgctgttctgcagccggtcgcggaggccatgga |
| | | tgcgatcgctgcggccgatcttagccagacgagcgggttcggccca |
| | | ttcggaccgcaaggaatcggtcaatacactacatggcgtgatttca |
| | | tatgcgcgattgctgatccccatgtgtatcactggcaaactgtgat |
| | | ggacgacaccgtcagtgcgtccgtcgcgcaggctctcgatgagctg |
| | | atgctttgggccgaggactgccccgaagtccggcacctcgtgcacg |
| | | cggatttcggctccaacaatgtcctgacggacaatggccgcataac |
| | | agcggtcattgactggagcgaggcgatgttcggggattcccaatac |
| | | gaggtcgccaacatcttcttctggaggccgtggttggcttgtatgg |
| | | agcagcagacgcgctacttcgagcggaggcatccggagcttgcagg |
| | | atcgccgcggctccgggcgtatatgctccgcattggtcttgaccaa |
| | | ctctatcagagcttggttgacggcaatttcgatgatgcagcttggg |
| | | cgcagggtcgatgcgacgcaatcgtccgatccggagccgggactgt |
| | | cgggcgtacacaaatcgcccgcagaagcgcggccgtctggaccgat |
| | | ggctgtgtagaagtactcgccgatagtggaaaccgacgccccagca |
| | | ctcgtccgagggcaaaggaatagacccgaattccagggccaggccg |
| | | gccgatatcaagcttatcgataatcaacctctggattacaaaattt |
| | | gtgaaagattgactggtattcttaactatgttgctccttttacgct |
| | | atgtggatacgctgctttaatgcctttgtatcatgctattgcttcc |
| | | cgtatggctttcattttctcctccttgtataaatcctggttgctgt |
| | | ctctttatgaggagttgtggcccgttgtcaggcaacgtggcgtggt |
| | | gtgcactgtgtttgctgacgcaacccccactggttggggcattgcc |
| | | accacctgtcagctcctttccgggactttcgctttccccctcccta |
| | | ttgccacggcggaactcatcgccgcctgccttgcccgctgctggac |
| | | aggggctcggctgttgggcactgacaattccgtggtgttgtcgggg |
| | | aaatcatcgtcctttccttggctgctcgcctgtgttgccacctgga |
| | | ttctgcgcgggacgtccttctgctacgtcccttcggccctcaatcc |
| | | ageggaccttccttcccgcggcctgctgccggctctgcggcctett |
| | | ccgcgtcttcgccttcgccctcagacgagtcggatctccctttggg |
| | | ccgcctccccgcategataccgtcgacctcgagacctagaaaaaca |
| | | tggagcaatcacaagtagcaatacagcagctaccaatgctgattgt |
| | | gcctggctagaagcacaagaggaggaggaggtgggttttccagtca |
| | | cacctcaggtacctttaagaccaatgacttacaaggcagctgtaga |
| | | tcttagccactttttaaaagaaaaggggggactggaagggctaatt |
| | | cactcccaacgaagacaagatatccttgatctgtggatctaccaca |
| | | cacaaggctacttccctgattggcagaactacacaccagggccagg |
| | | gatcagatatccactgacctttggatggtgctacaagctagtacca |
| | | gttgagcaagagaaggtagaagaagccaatgaaggagagaacaccc |
| | | gcttgttacaccctgtgagcctgcatgggatggatgacccggagag |
| | | agaagtattagagtggaggtttgacagccgcctagcatttcatcac |
| | | atggcccgagagctgcatccggactgtactgggtctctctggttag |
| | | accagatctgagcctgggagctctctggctaactagggaacccact |
| | | gcttaagcctcaataaagcttgccttgagtgcttcaagtagtgtgt |
| | | gcccgtctgttgtgtgactctggtaactagagatccctcagaccct |
| | | tttagtcagtgtggaaaatctctagcagggcccgtttaaacccgct |
| | | gatcagcctcgactgtgccttctagttgccagccatctgttgtttg |
| | | cccctcccccgtgccttccttgaccctggaaggtgccactcccact |
| | | gtcctttcctaataaaatgaggaaattgcatcgcattgtctgagta |
| | | ggtgtcattctattctggggggtggggggggcaggacagcaagggg |
| | | gaggattgggaagacaatagcaggcatgctggggatgcggtgggct |
| | | ctatggcttctgaggcggaaagaaccagctggggctctagggggta |
| | | tccccacgcgccctgtagcggcgcattaagcgcggcgggtgtggtg |
| | | gttacgcgcagcgtgaccgctacacttgccagcgccctagcgcccg |
| | | ctcctttcgctttcttcccttcctttctcgccacgttcgccggctt |
| | | tccccgtcaagctctaaatcgggggctccctttagggttccgattt |
| | | agtgctttacggcacctegaccccaaaaaacttgattagggtgatg |
| | | gttcacgtagtgggccatcgccctgatagacggtttttcgcccttt |
| | | gacgttggagtccacgttctttaatagtggactcttgttccaaact |
| | | ggaacaacactcaaccctatctcggtctattcttttgatttataag |
| | | ggattttgccgatttcggcctattggttaaaaaatgagctgattta |
| | | acaaaaatttaacgcgaattaattctgtggaatgtgtgtcagttag |
| | | ggtgtggaaagtccccaggctccccagcaggcagaagtatgcaaag |
| | | catgcatctcaattagtcagcaaccaggtgtggaaagtccccaggc |
| | | tccccagcaggcagaagtatgcaaagcatgcatctcaattagtcag |
| | | caaccatagtcccgcccctaactccgcccatcccgcccctaactcc |
| | | gcccagttcegcccattctccgccccatggctgactaatttttttt |
| | | atttatgcagaggccgaggccgcctctgcctctgagctattccaga |
| | | agtagtgaggaggcttttttggaggcctaggcttttgcaaaaagct |
| | | cccgggagcttgtatatccattttoggatctgatcagcacgtgttg |
| | | acaattaatcatcggcatagtatatcggcatagtataatacgacaa |
| | | ggtgaggaactaaaccatggccaagttgaccagtgccgttccggtg |
| | | ctcaccgcgcgcgacgtcgccggagcggtcgagttctggaccgacc |
| | | ggctcgggttctcccgggacttcgtggaggacgacttcgccggtgt |
| | | ggtccgggacgacgtgaccctgttcatcagcgcggtccaggaccag |
| | | gtggtgccggacaacaccctggcctgggtgtgggtgcgcggcctgg |
| | | acgagctgtacgccgagtggtcggaggtcgtgtccacgaacttccg |
| | | ggacgcctccgggccggccatgaccgagatcggcgagcagccgtgg |
| | | gggcgggagttcgccctgcgcgacccggccggcaactgcgtgcact |
| | | tcgtggccgaggagcaggactgacacgtgctacgagatttcgattc |
| | | caccgccgccttctatgaaaggttgggcttcggaatcgttttccgg |
| | | gacgccggctggatgatcctccagcgcggggatctcatgctggagt |
| | | tcttcgcccaccccaacttgtttattgcagcttataatggttacaa |
| | | ataaagcaatagcatcacaaatttcacaaataaagcatttttttca |
| | | ctgcattctagttgtggtttgtccaaactcatcaatgtatcttatc |
| | | atgtctgtataccgtcgacctctagctagagcttggcgtaatcatg |
| | | gtcatagctgtttcctgtgtgaaattgttatccgctcacaattcca |
| | | cacaacatacgagccggaagcataaagtgtaaagcctggggtgcct |
| | | aatgagtgagctaactcacattaattgcgttgcgctcactgcccgc |
| | | tttccagtcgggaaacctgtcgtgccagctgcattaatgaatcggc |
| | | caacgcgcggggagaggcggtttgcgtattgggcgctcttccgctt |
| | | cctcgctcactgactcgctgcgctcggtcgttcggctgcggcgagc |
| | | ggtatcagctcactcaaaggcggtaatacggttatccacagaatca |
| | | ggggataacgcaggaaagaacatgtgagcaaaaggccagcaaaagg |
| | | ccaggaaccgtaaaaaggccgcgttgctggcgtttttccataggct |
| | | ccgcccccctgacgagcatcacaaaaatcgacgctcaagtcagagg |
| | | tggcgaaacccgacaggactataaagataccaggcgtttccccctg |
| | | gaagctccctcgtgcgctctcctgttccgaccctgccgcttaccgg |
| | | atacctgtccgcctttctcccttcgggaagcgtggcgctttctcat |
| | | agctcacgctgtaggtatctcagttcggtgtaggtcgttcgctcca |
| | | agctgggctgtgtgcacgaaccccccgttcagcccgaccgctgcgc |
| | | cttatccggtaactatcgtcttgagtccaacccggtaagacacgac |
| | | ttatcgccactggcagcagccactggtaacaggattagcagagcga |
| | | ggtatgtaggcggtgctacagagttcttgaagtggtggcctaacta |
| | | cggctacactagaagaacagtatttggtatctgcgctctgctgaag |
| | | ccagttaccttcggaaaaagagttggtagctcttgatccggcaaac |
| | | aaaccaccgctggtagcggtggtttttttgtttgcaagcagcagat |
| | | tacgcgcagaaaaaaaggatctcaagaagatcctttgatcttttct |
| | | acggggtctgacgctcagtggaacgaaaactcacgttaagggattt |
| | | tggtcatgagattatcaaaaaggatcttcacctagatccttttaaa |
| | | ttaaaaatgaagttttaaatcaatctaaagtatatatgagtaaact |
| | | tggtctgacagttaccaatgcttaatcagtgaggcacctatctcag |
| | | cgatctgtctatttcgttcatccatagttgcctgactccccgtcgt |
| | | gtagataactacgatacgggagggcttaccatctggccccagtgct |
| | | gcaatgataccgcgagacccacgctcaccggctccagatttatcag |
| | | caataaaccagccagccggaagggccgagcgcagaagtggtcctgc |
| | | aactttatccgcctccatccagtctattaattgttgccgggaagct |
| | | agagtaagtagttcgccagttaatagtttgcgcaacgttgttgcca |
| | | ttgctacaggcatcgtggtgtcacgctcgtcgtttggtatggcttc |
| | | attcagctccggttcccaacgatcaaggcgagttacatgatccccc |
| | | atgttgtgcaaaaaagcggttagctccttoggtcctccgatcgttg |
| | | tcagaagtaagttggccgcagtgttatcactcatggttatggcagc |
| | | actgcataattctcttactgtcatgccatccgtaagatgcttttct |
| | | gtgactggtgagtactcaaccaagtcattctgagaatagtgtatgc |
| | | ggcgaccgagttgctcttgcccggcgtcaatacgggataataccgc |
| | | gccacatagcagaactttaaaagtgctcatcattggaaaacgttct |
| | | tcggggcgaaaactctcaaggatcttaccgctgttgagatccagtt |
| | | cgatgtaacccactcgtgcacccaactgatcttcagcatcttttac |
| | | tttcaccagcgtttctgggtgagcaaaaacaggaaggcaaaatgcc |
| | | gcaaaaaagggaataagggcgacacggaaatgttgaatactcatac |
| | | tcttcctttttcaatattattgaagcatttatcagggttattgtct |
| | | catgagcggatacatatttgaatgtatttagaaaaataaacaaata |
| | | ggggttccgcgcacatttccccgaaaagtgccacctgac |
| 15 | SpCas9 | atggactataaggaccacgacggagactacaaggatcatgatattg |
| | -Sso7d | attacaaagacgatgacgataagatggccccaaagaagaagcggaa |
| | (DNA) | ggtcggtatccacggagtcccagcagccgacaagaagtacagcatc |
| | expression | ggcctggacatcggcaccaactctgtgggctgggccgtgatcaccg |
| | cassette | acgagtacaaggtgcccagcaagaaattcaaggtgctgggcaacac |
| | | cgaccggcacagcatcaagaagaacctgatcggagccctgctgttc |
| | | gacagcggcgaaacagccgaggccacccggctgaagagaaccgcca |
| | | gaagaagatacaccagacggaagaaccggatctgctatctgcaaga |
| | | gatcttcagcaacgagatggccaaggtggacgacagcttcttccac |
| | | agactggaagagtccttcctggtggaagaggataagaagcacgagc |
| | | ggcaccccatcttcggcaacatcgtggacgaggtggcctaccacga |
| | | gaagtaccccaccatctaccacctgagaaagaaactggtggacagc |
| | | accgacaaggccgacctgcggctgatctatctggccctggcccaca |
| | | tgatcaagttccggggccacttcctgatcgagggcgacctgaaccc |
| | | cgacaacagcgacgtggacaagctgttcatccagctggtgcagacc |
| | | tacaaccagctgttcgaggaaaaccccatcaacgccagcggcgtgg |
| | | acgccaaggccatcctgtctgccagactgagcaagagcagacggct |
| | | ggaaaatctgatcgcccagctgcccggcgagaagaagaatggcctg |
| | | ttcggaaacctgattgccctgagcctgggcctgacccccaacttca |
| | | agagcaacttcgacctggccgaggatgccaaactgcagctgagcaa |
| | | ggacacctacgacgacgacctggacaacctgctggcccagatcggc |
| | | gaccagtacgccgacctgtttctggccgccaagaacctgtccgacg |
| | | ccatcctgctgagcgacatcctgagagtgaacaccgagatcaccaa |
| | | ggcccccctgagcgcctctatgatcaagagatacgacgagcaccac |
| | | caggacctgaccctgctgaaagctctcgtgcggcagcagctgcctg |
| | | agaagtacaaagagattttcttcgaccagagcaagaacggctacgc |
| | | cggctacattgacggcggagccagccaggaagagttctacaagttc |
| | | atcaagcccatcctggaaaagatggacggcaccgaggaactgctcg |
| | | tgaagctgaacagagaggacctgctgcggaagcagcggaccttcga |
| | | caacggcagcatcccccaccagatccacctgggagagctgcacgcc |
| | | attctgcggcggcaggaagatttttacccattcctgaaggacaacc |
| | | gggaaaagatcgagaagatcctgaccttccgcatcccctactacgt |
| | | gggccctctggccaggggaaacagcagattcgcctggatgaccaga |
| | | aagagcgaggaaaccatcaccccctggaacttcgaggaagtggtgg |
| | | acaagggcgcttccgcccagagcttcatcgagcggatgaccaactt |
| | | cgataagaacctgcccaacgagaaggtgctgcccaagcacagcctg |
| | | ctgtacgagtacttcaccgtgtataacgagctgaccaaagtgaaat |
| | | acgtgaccgagggaatgagaaagcccgccttcctgagcggcgagca |
| | | gaaaaaggccatcgtggacctgctgttcaagaccaaccggaaagtg |
| | | accgtgaagcagctgaaagaggactacttcaagaaaatcgagtgct |
| | | tcgactccgtggaaatctccggcgtggaagatcggttcaacgcctc |
| | | cctgggcacataccacgatctgctgaaaattatcaaggacaaggac |
| | | ttcctggacaatgaggaaaacgaggacattctggaagatatcgtgc |
| | | tgaccctgacactgtttgaggacagagagatgatcgaggaacggct |
| | | gaaaacctatgcccacctgttcgacgacaaagtgatgaagcagctg |
| | | aagcggcggagatacaccggctggggcaggctgagccggaagctga |
| | | tcaacggcatccgggacaagcagtccggcaagacaatcctggattt |
| | | cctgaagtccgacggcttcgccaacagaaacttcatgcagctgatc |
| | | cacgacgacagcctgacctttaaagaggacatccagaaagcccagg |
| | | tgtccggccagggcgatagcctgcacgagcacattgccaatctggc |
| | | cggcagccccgccattaagaagggcatcctgcagacagtgaaggtg |
| | | gtggacgagctcgtgaaagtgatgggccggcacaagcccgagaaca |
| | | tcgtgatcgaaatggccagagagaaccagaccacccagaagggaca |
| | | gaagaacagccgcgagagaatgaagcggatcgaagagggcatcaaa |
| | | gagctgggcagccagatcctgaaagaacaccccgtggaaaacaccc |
| | | agctgcagaacgagaagctgtacctgtactacctgcagaatggggg |
| | | gatatgtacgtggaccaggaactggacatcaaccggctgtccgact |
| | | acgatgtggaccatatcgtgcctcagagctttctgaaggacgactc |
| | | catcgacaacaaggtgctgaccagaagcgacaagaaccggggcaag |
| | | agcgacaacgtgccctccgaagaggtcgtgaagaagatgaagaact |
| | | actggcggcagctgctgaacgccaagctgattacccagagaaagtt |
| | | cgacaatctgaccaaggccgagagaggcggcctgagcgaactggat |
| | | aaggccggcttcatcaagagacagctggtggaaacccggcagatca |
| | | caaagcacgtggcacagatcctggactcccggatgaacactaagta |
| | | cgacgagaatgacaagctgatccgggaagtgaaagtgatcaccctg |
| | | aagtccaagctggtgtccgatttccggaaggatttccagttttaca |
| | | aagtgcgcgagatcaacaactaccaccacgcccacgacgcctacct |
| | | gaacgccgtcgtgggaaccgccctgatcaaaaagtaccctaagctg |
| | | gaaagcgagttcgtgtacggcgactacaaggtgtacgacgtgcgga |
| | | agatgatcgccaagagcgagcaggaaatcggcaaggctaccgccaa |
| | | gtacttcttctacagcaacatcatgaactttttcaagaccgagatt |
| | | accctggccaacggcgagatccggaagcggcctctgatcgagacaa |
| | | acggcgaaaccggggagatcgtgtgggataagggccgggattttgc |
| | | caccgtgcggaaagtgctgagcatgccccaagtgaatatcgtgaaa |
| | | aagaccgaggtgcagacaggcggcttcagcaaagagtctatcctgc |
| | | ccaagaggaacagcgataagctgatcgccagaaagaaggactggga |
| | | ccctaagaagtacggcggcttcgacagccccaccgtggcctattct |
| | | gtgctggtggtggccaaagtggaaaagggcaagtccaagaaactga |
| | | agagtgtgaaagagctgctggggatcaccatcatggaaagaagcag |
| | | cttcgagaagaatcccatcgactttctggaagccaagggctacaaa |
| | | gaagtgaaaaaggacctgatcatcaagctgcctaagtactccctgt |
| | | tcgagctggaaaacggccggaagagaatgctggcctctgccggcga |
| | | actgcagaagggaaacgaactggccctgccctccaaatatgtgaac |
| | | ttcctgtacctggccagccactatgagaagctgaagggctcccccg |
| | | aggataatgagcagaaacagctgtttgtggaacagcacaagcacta |
| | | cctggacgagatcatcgagcagatcagcgagttctccaagagagtg |
| | | atcctggccgacgctaatctggacaaagtgctgtccgcctacaaca |
| | | agcaccgggataagcccatcagagagcaggccgagaatatcatcca |
| | | cctgtttaccctgaccaatctgggagcccctgccgccttcaagtac |
| | | tttgacaccaccatcgaccggaagaggtacaccagcaccaaagagg |
| | | tgctggacgccaccctgatccaccagagcatcaccggcctgtacga |
| | | gacacggatcgacctgtctcagctgggaggcgacggatccggaATG |
| | | GCTACAGTGAAGTTTAAGTATAAAGGGGAAGAGAAAGAGGTGGACA |
| | | TCAGTAAAATCAAGAAAGTGTGGCGCGTAGGTAAGATGATCAGCTT |
| | | TACGTACGATGAAGGAGGAGGAAAGACTGGCAGAGGAGCCGTTTCA |
| | | GAAAAGGACGCTCCAAAGGAATTGTTGCAGATGCTCGAGAAGCAGA |
| | | AGAAGggaagcggaaaaaggccggcggccacgaaaaaggccggcca |
| | | ggcaaaaaagaaaaagggaagcggagagggcaggggaagtcttcta |
| | | acatgcggggacgtggaggaaaatcccggccccatgaaaaagcctg |
| | | aactcaccgcgacgtctgtcgagaagtttctgatcgaaaagttcga |
| | | cagcgtctccgacctgatgcagctctcggagggcgaagaatctcgt |
| | | gctttcagcttcgatgtaggagggcgtggatatgtcctgcgggtaa |
| | | atagctgcgccgatggtttctacaaagatcgttatgtttatcggca |
| | | ctttgcatcggccgcgctcccgattccggaagtgcttgacattggg |
| | | gaatttagcgagagcctgacctattgcatctcccgccgtgcacagg |
| | | gtgtcacgttgcaagacctgcctgaaaccgaactgcccgctgttct |
| | | gcagccggtcgcggaggccatggatgcgatcgctgcggccgatctt |
| | | agccagacgagcgggttcggcccattcggaccgcaaggaatcggtc |
| | | aatacactacatggcgtgatttcatatgcgcgattgctgatcccca |
| | | tgtgtatcactggcaaactgtgatggacgacaccgtcagtgcgtcc |
| | | gtcgcgcaggctctcgatgagctgatgctttgggccgaggactgcc |
| | | ccgaagtccggcacctcgtgcacgcggatttcggctccaacaatgt |
| | | cctgacggacaatggccgcataacagcggtcattgactggagcgag |
| | | gcgatgttcggggattcccaatacgaggtcgccaacatcttcttct |
| | | ggaggccgtggttggcttgtatggagcagcagacgcgctacttcga |
| | | gcggaggcatccggagcttgcaggatcgccgcggctccgggcgtat |
| | | atgctccgcattggtcttgaccaactctatcagagcttggttgacg |
| | | gcaatttcgatgatgcagcttgggcgcagggtcgatgcgacgcaat |
| | | cgtccgatccggagccgggactgtcgggcgtacacaaatcgcccgc |
| | | agaagcgcggccgtctggaccgatggctgtgtagaagtactcgccg |
| | | atagtggaaaccgacgccccagcactcgtccgagggcaaaggaata |
| | | g |
| 16 | SpCas9 | MDYKDHDGDYKDHDIDYKDDDDKMAPKKKRKVGIHGVPAADKKYSI |
| | -Sso7d | GLDIGTNSVGWAVITDEYKVPSKKFKVLGNTDRHSIKKNLIGALLF |
| | (amino | DSGETAEATRLKRTARRRYTRRKNRICYLQEIFSNEMAKVDDSFFH |
| | acid) | RLEESFLVEEDKKHERHPIFGNIVDEVAYHEKYPTIYHLRKKLVDS |
| | expression | TDKADLRLIYLALAHMIKFRGHFLIEGDLNPDNSDVDKLFIQLVQT |
| | cassette | YNQLFEENPINASGVDAKAILSARLSKSRRLENLIAQLPGEKKNGL |
| | | FGNLIALSLGLTPNFKSNFDLAEDAKLQLSKDTYDDDLDNLLAQIG |
| | | DQYADLFLAAKNLSDAILLSDILRVNTEITKAPLSASMIKRYDEHH |
| | | QDLTLLKALVRQQLPEKYKEIFFDQSKNGYAGYIDGGASQEEFYKF |
| | | IKPILEKMDGTEELLVKLNREDLLRKQRTFDNGSIPHQIHLGELHA |
| | | ILRRQEDFYPFLKDNREKIEKILTFRIPYYVGPLARGNSRFAWMTR |
| | | KSEETITPWNFEEVVDKGASAQSFIERMTNFDKNLPNEKVLPKHSL |
| | | LYEYFTVYNELTKVKYVTEGMRKPAFLSGEQKKAIVDLLFKTNRKV |
| | | TVKQLKEDYFKKIECFDSVEISGVEDRFNASLGTYHDLLKIIKDKD |
| | | FLDNEENEDILEDIVLTLTLFEDREMIEERLKTYAHLFDDKVMKQL |
| | | KRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKV |
| | | VDELVKVMGRHKPENIVIEMARENQTTQKGQKNSRERMKRIEEGIK |
| | | ELGSQILKEHPVENTQLQNEKLYLYYLQNGRDMYVDQELDINRLSD |
| | | YDVDHIVPQSFLKDDSIDNKVLTRSDKNRGKSDNVPSEEVVKKMKN |
| | | YWRQLLNAKLITQRKFDNLTKAERGGLSELDKAGFIKRQLVETRQI |
| | | TKHVAQILDSRMNTKYDENDKLIREVKVITLKSKLVSDFRKDFQFY |
| | | KVREINNYHHAHDAYLNAVVGTALIKKYPKLESEFVYGDYKVYDVR |
| | | KMIAKSEQEIGKATAKYFFYSNIMNFFKTEITLANGEIRKRPLIET |
| | | NGETGEIVWDKGRDFATVRKVLSMPQVNIVKKTEVQTGGFSKESIL |
| | | PKRNSDKLIARKKDWDPKKYGGFDSPTVAYSVLVVAKVEKGKSKKL |
| | | KSVKELLGITIMERSSFEKNPIDFLEAKGYKEVKKDLIIKLPKYSL |
| | | FELENGRKRMLASAGELQKGNELALPSKYVNFLYLASHYEKLKGSP |
| | | EDNEQKQLFVEQHKHYLDEIIEQISEFSKRVILADANLDKVLSAYN |
| | | KHRDKPIREQAENIIHLFTLTNLGAPAAFKYFDTTIDRKRYTSTKE |
| | | VLDATLIHQSITGLYETRIDLSQLGGDGSGMATVKFKYKGEEKEVD |
| | | ISKIKKVWRVGKMISFTYDEGGGKTGRGAVSEKDAPKELLQMLEKQ |
| | | KKGSGKRPAATKKAGQAKKKKGSGEGRGSLLTCGDVEENPGPMKKP |
| | | ELTATSVEKFLIEKFDSVSDLMQLSEGEESRAFSFDVGGRGYVLRV |
| | | NSCADGFYKDRYVYRHFASAALPIPEVLDIGEFSESLTYCISRRAQ |
| | | GVTLQDLPETELPAVLQPVAEAMDAIAAADLSQTSGFGPFGPQGIG |
| | | QYTTWRDFICAIADPHVYHWQTVMDDTVSASVAQALDELMLWAEDC |
| | | PEVRHLVHADFGSNNVLTDNGRITAVIDWSEAMFGDSQYEVANIFF |
| | | WRPWLACMEQQTRYFERRHPELAGSPRLRAYMLRIGLDQLYQSLVD |
| | | GNFDDAAWAQGRCDAIVRSGAGTVGRTQIARRSAAVWTDGCVEVLA |
| | | DSGNRRPSTRPRAKE* |
| 17 | SaCas9 | atggccccaaagaagaagcggaaggtcggtatccacggagtcccag |
| | -Sso7d | cagccaagcggaactacatcctgggcctggacatcggcatcaccag |
| | (DNA) | cgtgggctacggcatcatcgactacgagacacgggacgtgatcgat |
| | expression | gccggcgtgcggctgttcaaagaggccaacgtggaaaacaacgagg |
| | cassette | gcaggcggagcaagagaggcgccagaaggctgaagcggcggaggcg |
| | | gcatagaatccagagagtgaagaagctgctgttcgactacaacctg |
| | | ctgaccgaccacagcgagctgagcggcatcaacccctacgaggcca |
| | | gagtgaagggcctgagccagaagctgagcgaggaagagttctctgc |
| | | cgccctgctgcacctggccaagagaagaggcgtgcacaacgtgaac |
| | | gaggtggaagaggacaccggcaacgagctgtccaccaaagagcaga |
| | | tcagccggaacagcaaggccctggaagagaaatacgtggccgaact |
| | | gcagctggaacggctgaagaaagacggcgaagtgcggggcagcatc |
| | | aacagattcaagaccagcgactacgtgaaagaagccaaacagctgc |
| | | tgaaggtgcagaaggcctaccaccagctggaccagagcttcatcga |
| | | cacctacatcgacctgctggaaacccggcggacctactatgaggga |
| | | cctggcgagggcagccccttcggctggaaggacatcaaagaatggt |
| | | acgagatgctgatgggccactgcacctacttccccgaggaactgcg |
| | | gagcgtgaagtacgcctacaacgccgacctgtacaacgccctgaac |
| | | gacctgaacaatctcgtgatcaccagggacgagaacgagaagctgg |
| | | aatattacgagaagttccagatcatcgagaacgtgttcaagcagaa |
| | | gaagaagcccaccctgaagcagatcgccaaagaaatcctcgtgaac |
| | | gaagaggatattaagggctacagagtgaccagcaccggcaagcccg |
| | | agttcaccaacctgaaggtgtaccacgacatcaaggacattaccgc |
| | | ccggaaagagattattgagaacgccgagctgctggatcagattgcc |
| | | aagatcctgaccatctaccagagcagcgaggacatccaggaagaac |
| | | tgaccaatctgaactccgagctgacccaggaagagatcgagcagat |
| | | ctctaatctgaagggctataccggcacccacaacctgagcctgaag |
| | | gccatcaacctgatcctggacgagctgtggcacaccaacgacaacc |
| | | agatcgctatcttcaaccggctgaagctggtgcccaagaaggtgga |
| | | cctgtcccagcagaaagagatccccaccaccctggtggacgacttc |
| | | atcctgagccccgtcgtgaagagaagcttcatccagagcatcaaag |
| | | tgatcaacgccatcatcaagaagtacggcctgcccaacgacatcat |
| | | tatcgagctggcccgcgagaagaactccaaggacgcccagaaaatg |
| | | atcaacgagatgcagaagcggaaccggcagaccaacgagcggatcg |
| | | aggaaatcatccggaccaccggcaaagagaacgccaagtacctgat |
| | | cgagaagatcaagctgcacgacatgcaggaaggcaagtgcctgtac |
| | | agcctggaagccatccctctggaagatctgctgaacaaccccttca |
| | | actatgaggtggaccacatcatccccagaagcgtgtccttcgacaa |
| | | cagcttcaacaacaaggtgctcgtgaagcaggaagaaaacagcaag |
| | | aagggcaaccggaccccattccagtacctgagcagcagcgacagca |
| | | agatcagctacgaaaccttcaagaagcacatcctgaatctggccaa |
| | | gggcaagggcagaatcagcaagaccaagaaagagtatctgctggaa |
| | | gaacgggacatcaacaggttctccgtgcagaaagacttcatcaacc |
| | | ggaacctggtggataccagatacgccaccagaggcctgatgaacct |
| | | gctgcggagctacttcagagtgaacaacctggacgtgaaagtgaag |
| | | tccatcaatggcggcttcaccagctttctgcggcggaagtggaagt |
| | | ttaagaaagagcggaacaaggggtacaagcaccacgccgaggacgc |
| | | cctgatcattgccaacgccgatttcatcttcaaagagtggaagaaa |
| | | ctggacaaggccaaaaaagtgatggaaaaccagatgttcgaggaaa |
| | | agcaggccgagagcatgcccgagatogaaaccgagcaggagtacaa |
| | | agagatcttcatcaccccccaccagatcaagcacattaaggacttc |
| | | aaggactacaagtacagccaccgggtggacaagaagcctaatagag |
| | | agctgattaacgacaccctgtactccacccggaaggacgacaaggg |
| | | caacaccctgatcgtgaacaatctgaacggcctgtacgacaaggac |
| | | aatgacaagctgaaaaagctgatcaacaagagccccgaaaagctgc |
| | | tgatgtaccaccacgacccccagacctaccagaaactgaagctgat |
| | | tatggaacagtacggcgacgagaagaatcccctgtacaagtactac |
| | | gaggaaaccgggaactacctgaccaagtactccaaaaaggacaacg |
| | | gccccgtgatcaagaagattaagtattacggcaacaaactgaacgc |
| | | ccatctggacatcaccgacgactaccccaacagcagaaacaaggtc |
| | | gtgaagctgtccctgaagccctacagattcgacgtgtacctggaca |
| | | atggcgtgtacaagttcgtgaccgtgaagaatctggatgtgatcaa |
| | | aaaagaaaactactacgaagtgaatagcaagtgctatgaggaagct |
| | | aagaagctgaagaagatcagcaaccaggccgagtttatcgcctcct |
| | | tctacaacaacgatctgatcaagatcaacggcgagctgtatagagt |
| | | gatcggcgtgaacaacgacctgctgaaccggatcgaagtgaacatg |
| | | atcgacatcacctaccgcgagtacctggaaaacatgaacgacaaga |
| | | ggccccccaggatcattaagacaatcgcctccaagacccagagcat |
| | | taagaagtacagcacagacattctgggcaacctgtatgaagtgaaa |
| | | tctaagaagcaccctcagatcatcaaaaagggcggatccggaATGG |
| | | CTACAGTGAAGTTTAAGTATAAAGGGGAAGAGAAAGAGGTGGACAT |
| | | CAGTAAAATCAAGAAAGTGTGGCGCGTAGGTAAGATGATCAGCTTT |
| | | ACGTACGATGAAGGAGGAGGAAAGACTGGCAGAGGAGCCGTTTCAG |
| | | AAAAGGACGCTCCAAAGGAATTGTTGCAGATGCTCGAGAAGCAGAA |
| | | GAAGggaagcggaaaaaggccggggccacgaaaaaggccggccagg |
| | | caaaaaagaaaaagtacccatacgatgttccagattacgctggaag |
| | | cggagagggcaggggaagtcttctaacatgcggggacgtggaggaa |
| | | aatcccggccccatgaaaaagcctgaactcaccgcgacgtctgtcg |
| | | agaagtttctgatcgaaaagttcgacagcgtctccgacctgatgca |
| | | gctctcggagggcgaagaatctcgtgctttcagcttcgatgtagga |
| | | gggcgtggatatgtcctgcgggtaaatagctgcgccgatggtttct |
| | | acaaagatcgttatgtttatcggcactttgcatcggccgcgctccc |
| | | gattccggaagtgcttgacattggggaatttagcgagagcctgacc |
| | | tattgcatctcccgccgtgcacagggtgtcacgttgcaagacctgc |
| | | ctgaaaccgaactgcccgctgttctgcagccggtcgcggaggccat |
| | | ggatgcgatcgctgcggccgatcttagccagacgagcgggttcggc |
| | | ccattcggaccgcaaggaatcggtcaatacactacatggcgtgatt |
| | | tcatatgcgcgattgctgatccccatgtgtatcactggcaaactgt |
| | | gatggacgacaccgtcagtgcgtccgtcgcgcaggctctcgatgag |
| | | ctgatgctttgggccgaggactgccccgaagtccggcacctcgtgc |
| | | acgcggatttcggctccaacaatgtcctgacggacaatggccgcat |
| | | aacagcggtcattgactggagcgaggcgatgttcggggattcccaa |
| | | tacgaggtcgccaacatcttcttctggaggccgtggttggcttgta |
| | | tggagcagcagacgcgctacttcgagcggaggcatccggagcttgc |
| | | aggatcgccgcggctccgggcgtatatgctccgcattggtcttgac |
| | | caactctatcagagcttggttgacggcaatttcgatgatgcagctt |
| | | gggcgcagggtcgatgcgacgcaatcgtccgatccggagccgggac |
| | | tgtcgggcgtacacaaatcgcccgcagaagcgcggccgtctggacc |
| | | gatggctgtgtagaagtactcgccgatagtggaaaccgacgcccca |
| | | gcactcgtccgagggcaaaggaatag |
| 18 | SaCas9 | MAPKKKRKVGIHGVPAAKRNYILGLDIGITSVGYGIIDYETRDVID |
| | -Sso7d | AGVRLFKEANVENNEGRRSKRGARRLKRRRRHRIQRVKKLLFDYNL |
| | (amino | LTDHSELSGINPYEARVKGLSQKLSEEEFSAALLHLAKRRGVHNVN |
| | acid) | EVEEDTGNELSTKEQISRNSKALEEKYVAELQLERLKKDGEVRGSI |
| | expression | NRFKTSDYVKEAKQLLKVQKAYHQLDQSFIDTYIDLLETRRTYYEG |
| | cassette | PGEGSPFGWKDIKEWYEMLMGHCTYFPEELRSVKYAYNADLYNALN |
| | | DLNNLVITRDENEKLEYYEKFQIIENVFKQKKKPTLKQIAKEILVN |
| | | EEDIKGYRVTSTGKPEFTNLKVYHDIKDITARKEIIENAELLDQIA |
| | | KILTIYQSSEDIQEELTNLNSELTQEEIEQISNLKGYTGTHNLSLK |
| | | AINLILDELWHTNDNQIAIFNRLKLVPKKVDLSQQKEIPTTLVDDF |
| | | ILSPVVKRSFIQSIKVINAIIKKYGLPNDIIIELAREKNSKDAQKM |
| | | INEMQKRNRQTNERIEEIIRTTGKENAKYLIEKIKLHDMQEGKCLY |
| | | SLEAIPLEDLLNNPFNYEVDHIIPRSVSFDNSFNNKVLVKQEENSK |
| | | KGNRTPFQYLSSSDSKISYETFKKHILNLAKGKGRISKTKKEYLLE |
| | | ERDINRFSVQKDFINRNLVDTRYATRGLMNLLRSYFRVNNLDVKVK |
| | | SINGGFTSFLRRKWKFKKERNKGYKHHAEDALIIANADFIFKEWKK |
| | | LDKAKKVMENQMFEEKQAESMPEIETEQEYKEIFITPHQIKHIKDF |
| | | KDYKYSHRVDKKPNRELINDTLYSTRKDDKGNTLIVNNLNGLYDKD |
| | | NDKLKKLINKSPEKLLMYHHDPQTYQKLKLIMEQYGDEKNPLYKYY |
| | | EETGNYLTKYSKKDNGPVIKKIKYYGNKLNAHLDITDDYPNSRNKV |
| | | VKLSLKPYRFDVYLDNGVYKFVTVKNLDVIKKENYYEVNSKCYEEA |
| | | KKLKKISNQAEFIASFYNNDLIKINGELYRVIGVNNDLLNRIEVNM |
| | | IDITYREYLENMNDKRPPRIIKTIASKTQSIKKYSTDILGNLYEVK |
| | | SKKHPQIIKKGGSGMATVKFKYKGEEKEVDISKIKKVWRVGKMISF |
| | | TYDEGGGKTGRGAVSEKDAPKELLQMLEKQKKGSGKRPAATKKAGQ |
| | | AKKKKYPYDVPDYAGSGEGRGSLLTCGDVEENPGPMKKPELTATSV |
| | | EKFLIEKFDSVSDLMQLSEGEESRAFSFDVGGRGYVLRVNSCADGF |
| | | YKDRYVYRHFASAALPIPEVLDIGEFSESLTYCISRRAQGVTLQDL |
| | | PETELPAVLQPVAEAMDAIAAADLSQTSGFGPFGPQGIGQYTTWRD |
| | | FICAIADPHVYHWQTVMDDTVSASVAQALDELMLWAEDCPEVRHLV |
| | | HADFGSNNVLTDNGRITAVIDWSEAMFGDSQYEVANIFFWRPWLAC |
| | | MEQQTRYFERRHPELAGSPRLRAYMLRIGLDQLYQSLVDGNFDDAA |
| | | WAQGRCDAIVRSGAGTVGRTQIARRSAAVWTDGCVEVLADSGNRRP |
| | | STRPRAKE* |
| 19 | Cas12f | ATGCCAAAGAAGAAGCGGAAGGTCGGTATCCACGGAGTCCCAGCAG |
| | 1- | CCATGGCCAAGAACACAATTACAAAGACACTGAAGCTGAGGATCGT |
| | Aho7c | GAGACCATACAACAGCGCTGAGGTCGAGAAGATTGTGGCTGATGAA |
| | (DNA) | AAGAACAACAGGGAAAAGATCGCCCTCGAGAAGAACAAGGATAAGG |
| | expression | TGAAGGAGGCCTGCTCTAAGCACCTGAAAGTGGCCGCCTACTGCAC |
| | cassette | CACACAGGTGGAGAGGAACGCCTGTCTGTTTTGTAAAGCTCGGAAG |
| | | CTGGATGATAAGTTTTACCAGAAGCTGCGGGGCCAGTTCCCCGATG |
| | | CCGTCTTTTGGCAGGAGATTAGCGAGATCTTCAGACAGCTGCAGAA |
| | | GCAGGCCGCCGAGATCTACAACCAGAGCCTGATCGAGCTCTACTAC |
| | | GAGATCTTCATCAAGGGCAAGGGCATTGCCAACGCCTCCTCCGTGG |
| | | AGCACTACCTGAGCGACGTGTGCTACACAAGAGCCGCCGAGCTCTT |
| | | TAAGAACGCCGCTATCGCTTCCGGGCTGAGGAGCAAGATTAAGAGT |
| | | AACTTCCGGCTCAAGGAGCTGAAGAACATGAAGAGCGGCCTGCCCA |
| | | CTACAAAGAGCGACAACTTCCCAATTCCACTGGTGAAGCAGAAGGG |
| | | GGGCCAGTACACAGGGTTCGAGATTTCCAACCACAACAGCGACTTT |
| | | ATTATTAAGATCCCCTTTGGCAGGTGGCAGGTCAAGAAGGAGATTG |
| | | ACAAGTACAGGCCCTGGGAGAAGTTTGATTTCGAGCAGGTGCAGAA |
| | | GAGCCCCAAGCCTATTTCCCTGCTGCTGTCCACACAGCGGCGGAAG |
| | | AGGAACAAGGGGTGGTCTAAGGATGAGGGGACCGAGGCCGAGATTA |
| | | AGAAAGTGATGAACGGCGACTACCAGACAAGCTACATCGAGGTCAA |
| | | GCGGGGCAGTAAGATTGGCGAGAAGAGCGCCTGGATGCTGAACCTG |
| | | AGCATTGACGTGCCAAAGATTGATAAGGGCGTGGATCCCAGCATCA |
| | | TCGGAGGGATCGATGTGGGGGTCAAGAGCCCCCTCGTGTGCGCCAT |
| | | CAACAACGCCTTCAGCAGGTACAGCATCTCCGATAACGACCTGTTC |
| | | CACTTTAACAAGAAGATGTTCGCCCGGCGGAGGATTTTGCTCAAGA |
| | | AGAACCGGCACAAGCGGGCCGGACACGGGGCCAAGAACAAGCTCAA |
| | | GCCCATCACTATCCTGACCGAGAAGAGCGAGAGGTTCAGGAAGAAG |
| | | CTCATCGAGAGATGGGCCTGCGAGATCGCCGATTTCTTTATTAAGA |
| | | ACAAGGTCGGAACAGTGCAGATGGAGAACCTCGAGAGCATGAAGAG |
| | | GAAGGAGGATTCCTACTTCAACATTCGGCTGAGGGGGTTCTGGCCC |
| | | TACGCTGAGATGCAGAACAAGATTGAGTTTAAGCTGAAGCAGTACG |
| | | GGATTGAGATCCGGAAGGTGGCCCCCAACAACACCAGCAAGACCTG |
| | | CAGCAAGTGCGGGCACCTCAACAACTACTTCAACTTCGAGTACCGG |
| | | AAGAAGAACAAGTTCCCACACTTCAAGTGCGAGAAGTGCAACTTTA |
| | | AGGAGAACGCCGATTACAACGCCGCCCTGAACATCAGCAACCCTAA |
| | | GCTGAAGAGCACTAAGGAGGAGCCCggatccggaATGGCGACTAAA |
| | | GTGAAGTTCAAATATAAGGGTGAAGAAAAGGAAGTAGATATATCCA |
| | | AGATCAAGAAGGTATGGCGGGTTGGAAAAATGATCAGCTTTACCTA |
| | | TGACGACAACGGAAAGACTGGTAGGGGTGCCGTATCTGAAAAAGAT |
| | | GCCCCCAAAGAATTGTTGGAAAAACTCAAGggaagcggaAAAAGGC |
| | | CGGCGGCCACGAAAAAGGCCGGCCAGGCAAAAAAGAAAAAgtaccc |
| | | atacgatgttccagattacgctggaagcggagagggcaggggaagt |
| | | cttctaacatgcggggacgtggaggaaaatcccggccccatgaaaa |
| | | agcctgaactcaccgcgacgtctgtcgagaagtttctgatcgaaaa |
| | | gttcgacagcgtctccgacctgatgcagctctcggagggcgaagaa |
| | | tctcgtgctttcagcttcgatgtaggagggcgtggatatgtcctgc |
| | | gggtaaatagctgcgccgatggtttctacaaagatcgttatgttta |
| | | tcggcactttgcatcggccgcgctcccgattccggaagtgcttgac |
| | | attggggaatttagcgagagcctgacctattgcatctcccgccgtg |
| | | cacagggtgtcacgttgcaagacctgcctgaaaccgaactgcccgc |
| | | tgttctgcagccggtcgcggaggccatggatgcgatcgctgcggcc |
| | | gatcttagccagacgagcgggttcggcccattcggaccgcaaggaa |
| | | tcggtcaatacactacatggcgtgatttcatatgcgcgattgctga |
| | | tccccatgtgtatcactggcaaactgtgatggacgacaccgtcagt |
| | | gcgtccgtcgcgcaggctctcgatgagctgatgctttgggccgagg |
| | | actgccccgaagtccggcacctcgtgcacgcggatttcggctccaa |
| | | caatgtcctgacggacaatggccgcataacagcggtcattgactgg |
| | | agcgaggcgatgttcggggattcccaatacgaggtegccaacatct |
| | | tcttctggaggccgtggttggcttgtatggagcagcagacgcgcta |
| | | cttcgagcggaggcatccggagcttgcaggatcgccgcggctccgg |
| | | gcgtatatgctccgcattggtcttgaccaactctatcagagcttgg |
| | | ttgacggcaatttcgatgatgcagcttgggcgcagggtcgatgcga |
| | | cgcaatcgtccgatccggagccgggactgtcgggcgtacacaaatc |
| | | gcccgcagaagcgcggccgtctggaccgatggctgtgtagaagtac |
| | | tcgccgatagtggaaaccgacgccccagcactcgtccgagggcaaa |
| | | ggaatag |
| 20 | Cas12f | MPKKKRKVGIHGVPAAMAKNTITKTLKLRIVRPYNSAEVEKIVADE |
| | 1- | KNNREKIALEKNKDKVKEACSKHLKVAAYCTTQVERNACLFCKARK |
| | Aho7c | LDDKFYQKLRGQFPDAVFWQEISEIFRQLQKQAAEIYNQSLIELYY |
| | (amino | EIFIKGKGIANASSVEHYLSDVCYTRAAELFKNAAIASGLRSKIKS |
| | acid) | NFRLKELKNMKSGLPTTKSDNFPIPLVKQKGGQYTGFEISNHNSDF |
| | express | IIKIPFGRWQVKKEIDKYRPWEKFDFEQVQKSPKPISLLLSTQRRK |
| | ion | RNKGWSKDEGTEAEIKKVMNGDYQTSYIEVKRGSKIGEKSAWMLNL |
| | cassette | SIDVPKIDKGVDPSIIGGIDVGVKSPLVCAINNAFSRYSISDNDLF |
| | | HFNKKMFARRRILLKKNRHKRAGHGAKNKLKPITILTEKSERFRKK |
| | | LIERWACEIADFFIKNKVGTVQMENLESMKRKEDSYFNIRLRGFWP |
| | | YAEMQNKIEFKLKQYGIEIRKVAPNNTSKTCSKCGHLNNYFNFEYR |
| | | KKNKFPHFKCEKCNFKENADYNAALNISNPKLKSTKEEPGSGMATK |
| | | VKFKYKGEEKEVDISKIKKVWRVGKMISFTYDDNGKTGRGAVSEKD |
| | | APKELLEKLKGSGKRPAATKKAGQAKKKKYPYDVPDYAGSGEGRGS |
| | | LLTCGDVEENPGPMKKPELTATSVEKFLIEKFDSVSDLMQLSEGEE |
| | | SRAFSFDVGGRGYVLRVNSCADGFYKDRYVYRHFASAALPIPEVLD |
| | | IGEFSESLTYCISRRAQGVTLQDLPETELPAVLQPVAEAMDAIAAA |
| | | DLSQTSGFGPFGPQGIGQYTTWRDFICAIADPHVYHWQTVMDDTVS |
| | | ASVAQALDELMLWAEDCPEVRHLVHADFGSNNVLTDNGRITAVIDW |
| | | SEAMFGDSQYEVANIFFWRPWLACMEQQTRYFERRHPELAGSPRLR |
| | | AYMLRIGLDQLYQSLVDGNFDDAAWAQGRCDAIVRSGAGTVGRTQI |
| | | ARRSAAVWTDGCVEVLADSGNRRPSTRPRAKE* |
| 21 | pLV-46 | GTCGACGGATCGGGAGATCTCCCGATCCCCTATGGTGCACTCTCAG |
| | comprising | TACAATCTGCTCTGATGCCGCATAGTTAAGCCAGTATCTGCTCCCT |
| | empty | GCTTGTGTGTTGGAGGTCGCTGAGTAGTGCGCGAGCAAAATTTAAG |
| | U6: | CTACAACAAGGCAAGGCTTGACCGACAATTGCATGAAGAATCTGCT |
| | gRNA | TAGGGTTAGGCGTTTTGCGCTGCTTCGCGATGTACGGGCCAGATAT |
| | cassette | ACGCGTTGACATTGATTATTGACTAGTTATTAATAGTAATCAATTA |
| | | CGGGGTCATTAGTTCATAGCCCATATATGGAGTTCCGCGTTACATA |
| | | ACTTACGGTAAATGGCCCGCCTGGCTGACCGCCCAACGACCCCCGC |
| | | CCATTGACGTCAATAATGACGTATGTTCCCATAGTAACGCCAATAG |
| | | GGACTTTCCATTGACGTCAATGGGTGGAGTATTTACGGTAAACTGC |
| | | CCACTTGGCAGTACATCAAGTGTATCATATGCCAAGTACGCCCCCT |
| | | ATTGACGTCAATGACGGTAAATGGCCCGCCTGGCATTATGCCCAGT |
| | | ACATGACCTTATGGGACTTTCCTACTTGGCAGTACATCTACGTATT |
| | | AGTCATCGCTATTACCATGGTGATGCGGTTTTGGCAGTACATCAAT |
| | | GGGCGTGGATAGCGGTTTGACTCACGGGGATTTCCAAGTCTCCACC |
| | | CCATTGACGTCAATGGGAGTTTGTTTTGGCACCAAAATCAACGGGA |
| | | CTTTCCAAAATGTCGTAACAACTCCGCCCCATTGACGCAAATGGGC |
| | | GGTAGGCGTGTACGGTGGGAGGTCTATATAAGCAGCGCGTTTTGCC |
| | | TGTACTGGGTCTCTCTGGTTAGACCAGATCTGAGCCTGGGAGCTCT |
| | | CTGGCTAACTAGGGAACCCACTGCTTAAGCCTCAATAAAGCTTGCC |
| | | TTGAGTGCTTCAAGTAGTGTGTGCCCGTCTGTTGTGTGACTCTGGT |
| | | AACTAGAGATCCCTCAGACCCTTTTAGTCAGTGTGGAAAATCTCTA |
| | | GCAGTGGCGCCCGAACAGGGACTTGAAAGCGAAAGGGAAACCAGAG |
| | | GAGCTCTCTCGACGCAGGACTCGGCTTGCTGAAGCGCGCACGGCAA |
| | | GAGGCGAGGGGCGGCGACTGGTGAGTACGCCAAAAATTTTGACTAG |
| | | CGGAGGCTAGAAGGAGAGAGATGGGTGCGAGAGCGTCAGTATTAAG |
| | | CGGGGGAGAATTAGATCGCGATGGGAAAAAATTCGGTTAAGGCCAG |
| | | GGGGAAAGAAAAAATATAAATTAAAACATATAGTATGGGCAAGCAG |
| | | GGAGCTAGAACGATTCGCAGTTAATCCTGGCCTGTTAGAAACATCA |
| | | GAAGGCTGTAGACAAATACTGGGACAGCTACAACCATCCCTTCAGA |
| | | CAGGATCAGAAGAACTTAGATCATTATATAATACAGTAGCAACCCT |
| | | CTATTGTGTGCATCAAAGGATAGAGATAAAAGACACCAAGGAAGCT |
| | | TTAGACAAGATAGAGGAAGAGCAAAACAAAAGTAAGACCACCGCAC |
| | | AGCAAGCGGCCGCTGATCTTCAGACCTGGAGGAGGAGATATGAGGG |
| | | ACAATTGGAGAAGTGAATTATATAAATATAAAGTAGTAAAAATTGA |
| | | ACCATTAGGAGTAGCACCCACCAAGGCAAAGAGAAGAGTGGTGCAG |
| | | AGAGAAAAAAGAGCAGTGGGAATAGGAGCTTTGTTCCTTGGGTTCT |
| | | TGGGAGCAGCAGGAAGCACTATGGGCGCAGCGTCAATGACGCTGAC |
| | | GGTACAGGCCAGACAATTATTGTCTGGTATAGTGCAGCAGCAGAAC |
| | | AATTTGCTGAGGGCTATTGAGGCGCAACAGCATCTGTTGCAACTCA |
| | | CAGTCTGGGGCATCAAGCAGCTCCAGGCAAGAATCCTGGCTGTGGA |
| | | AAGATACCTAAAGGATCAACAGCTCCTGGGGATTTGGGGTTGCTCT |
| | | GGAAAACTCATTTGCACCACTGCTGTGCCTTGGAATGCTAGTTGGA |
| | | GTAATAAATCTCTGGAACAGATTTGGAATCACACGACCTGGATGGA |
| | | GTGGGACAGAGAAATTAACAATTACACAAGCTTAATACACTCCTTA |
| | | ATTGAAGAATCGCAAAACCAGCAAGAAAAGAATGAACAAGAATTAT |
| | | TGGAATTAGATAAATGGGCAAGTTTGTGGAATTGGTTTAACATAAC |
| | | AAATTGGCTGTGGTATATAAAATTATTCATAATGATAGTAGGAGGC |
| | | TTGGTAGGTTTAAGAATAGTTTTTGCTGTACTTTCTATAGTGAATA |
| | | GAGTTAGGCAGGGATATTCACCATTATCGTTTCAGACCCACCTCCC |
| | | AACCCCGAGGGGACCCGACAGGCCCGAAGGAATAGAAGAAGAAGGT |
| | | GGAGAGAGAGACAGAGACAGATCCATTCGATTAGTGAACGGATCGG |
| | | CACTGCGTGCGCCAATTCTGCAGACAAATGGCAGTATTCATCCACA |
| | | ATTTTAAAAGAAAAGGGGGGATTGGGGGGTACAGTGCAGGGGAAAG |
| | | AATAGTAGACATAATAGCAACAGACATACAAACTAAAGAATTACAA |
| | | AAACAAATTACAAAAATTCAAAATTTTCGGGTTTATTACAGGGACA |
| | | GCAGAGATCCAGTTTGGGCTAGCTTAATTAAgtaaaacgacggcca |
| | | gtgagcgcgcgtaatacgactcactatagggcgaattgggtaccaa |
| | | gcttgcctatttcccatgattccttcatatttgcatatacgataca |
| | | aggctgttagagagataattggaattaatttgactgtaaacacaaa |
| | | gatattagtacaaaatacgtgacgtagaaagtaataatttcttggg |
| | | tagtttgcagttttaaaattatgttttaaaatggactatcatatgc |
| | | ttaccgtaacttgaaagtatttcgatttcttggctttatatatctt |
| | | gtggaaaggacgaaacaccgACCGCTTCACCGAGTGAAGGTGGGCT |
| | | GCTTGCATCAGCCTAATGTCGAGAAGTGCTTTCTTCGGAAAGTAAC |
| | | CCTCGAAACAAAgaaaGGAATGCAACggagacggacgtctcttttt |
| | | attttttgcggccgcccgcggtggagctccagcttttgttcccttt |
| | | agtgagggttaattgcgcgcttggcgtaatcatggtcatagctgtt |
| | | tcctgttaattaacctgcaggCCCGTGTCGGCTCCAGATCTGGCCT |
| | | CCGCGCCGGGTTTTGGCGCCTCCCGCGGGCGCCCCCCTCCTCACGG |
| | | CGAGCGCTGCCACGTCAGACGAAGGGCGCAGCGAGCGTCCTGATCC |
| | | TTCCGCCCGGACGCTCAGGACAGCGGCCCGCTGCTCATAAGACTCG |
| | | GCCTTAGAACCCCAGTATCAGCAGAAGGACATTTTAGGACGGGACT |
| | | TGGGTGACTCTAGGGCACTGGTTTTCTTTCCAGAGAGCGGAACAGG |
| | | CGAGGAAAAGTAGTCCCTTCTCGGCGATTCTGCGGAGGGATCTCCG |
| | | TGGGGCGGTGAACGCCGATGATTATATAAGGACGCGCCGGGTGTGG |
| | | CACAGCTAGTTCCGTCGCAGCCGGGATTTGGGTCGCGGTTCTTGTT |
| | | TGTGGATCGCTGTGATCGTCACTTGGTGAGTAGCGGGCTGCTGGGC |
| | | TGGCCGGGGCTTTCGTGGCCGCCGGGCCGCTCGGTGGGACGGAAGC |
| | | GTGTGGAGAGACCGCCAAGGGCTGTAGTCTGGGTCCGCGAGCAAGG |
| | | TTGCCCTGAACTGGGGGTTGGGGGGAGCGCAGCAAAATGGCGGCTG |
| | | TTCCCGAGTCTTGAATGGAAGACGCTTGTGAGGCGGGCTGTGAGGT |
| | | CGTTGAAACAAGGTGGGGGGCATGGTGGGCGGCAAGAACCCAAGGT |
| | | CTTGAGGCCTTCGCTAATGCGGGAAAGCTCTTATTCGGGTGAGATG |
| | | GGCTGGGGCACCATCTGGGGACCCTGACGTGAAGTTTGTCACTGAC |
| | | TGGAGAACTCGGTTTGTCGTCTGTTGCGGGGGCGGCAGTTATGGCG |
| | | GTGCCGTTGGGCAGTGCACCCGTACCTTTGGGAGCGCGCGCCCTCG |
| | | TCGTGTCGTGACGTCACCCGTTCTGTTGGCTTATAATGCAGGGTGG |
| | | GGCCACCTGCCGGTAGGTGTGCGGTAGGCTTTTCTCCGTCGCAGGA |
| | | CGCAGGGTTCGGGCCTAGGGTAGGCTCTCCTGAATCGACAGGCGCC |
| | | GGACCTCTGGTGAGGGGAGGGATAAGTGAGGCGTCAGTTTCTTTGG |
| | | TCGGTTTTATGTACCTATCTTCTTAAGTAGCTGAAGCTCCGGTTTT |
| | | GAACTATGCGCTCGGGGTTGGCGAGTGTGTTTTGTGAAGTTTTTTA |
| | | GGCACCTTTTGAAATGTAATCATTTGGGTCAATATGTAATTTTCAG |
| | | TGTTAGACTAGTAAATTGTCCGCTAAATTCTGGCCGTTTTTGGCTT |
| | | TTTTGTTAGACGAAGCTTGGGCTGCAGGTCGACTCTAGGCCCaggC |
| | | GGCCGGATCCCCGGGTACCGGTCGCCACCATGGATAGCACTGAGAA |
| | | CGTCATCAAGCCCTTCATGCGCTTCAAGGTGCACATGGAGGGCTCC |
| | | GTGAACGGCCACGAGTTCGAGATCGAGGGCGAGGGCGAGGGCAAGC |
| | | CcTACGAGGGCACCCAGACCGCCAAGCTGCAGGTGACCAAGGGCGG |
| | | CCCCCTGCCCTTCGCCTGGGACATCCTGTCCCCCCAGTTCCAGTAC |
| | | GGCTCCAAGGTGTACGTGAAGCACCCCGCCGACATCCCCGACTACA |
| | | AGAAGCTGTCCTTCCCCGAGGGCTTCAAGTGGGAGCGCGTGATGAA |
| | | CTTCGAGGACGGCGGCGTGGTGACCGTGACCCAGGACTCCTCTCTG |
| | | CAGGACGGCACCTTCATCTACCACGTGAAGTTCATCGGCGTGAACT |
| | | TCCCCTCCGACGGCCCCGTAATGCAGAAGAAGACTCTGGGCTGGGA |
| | | GCCCTCCACCGAGCGCCTGTACCCCCGCGACGGCGTGCTGAAGGGC |
| | | GAGATCCACAAGGCGCTGAAGCTGAAGGGCGGCGGCCACTACCTGG |
| | | TGGAGTTCAAGTCAATCTACATGGCCAAGAAGCCCGTGAAGCTGCC |
| | | CGGCTACTACTACGTGGACTCCAAGCTGGACATCACCTCCCACAAC |
| | | GAGGACTACACCGTGGTGGAGCAGTACGAGCGCGCCGAGGCCCGCC |
| | | ACCACCTGTTCCAGACTCACGGTATGGACGAATTGTACAAGCACGA |
| | | CGAATTGGAGGGCAGAGGAAGTCTTCTAACATGCGGTGACGTGGAG |
| | | GAGAATCCCGGCCCTATGACCGAGTACAAGCCCACGGTGCGCCTCG |
| | | CCACCCGCGACGACGTCCCCaGGGCCGTACGCACCCTCGCCGCCGC |
| | | GTTCGCCGACTACCCCGCCACGCGCCACACCGTCGATCCGGACCGC |
| | | CACATCGAGCGGGTCACCGAGCTGCAAGAACTCTTCCTCACGCGCG |
| | | TCGGGCTCGACATCGGCAAGGTGTGGGTCGCGGACGACGGCGCCGC |
| | | GGTGGCGGTCTGGACCACGCCGGAGAGCGTCGAAGCGGGGGCGGTG |
| | | TTCGCCGAGATCGGCCCGCGCATGGCCGAGTTGAGCGGTTCCCGGC |
| | | TGGCCGCGCAGCAACAGATGGAAGGCCTCCTGGCGCCGCACCGGCC |
| | | CAAGGAGCCCGCGTGGTTCCTGGCCACCGTCGGCGTGTCGCCCGAC |
| | | CACCAGGGCAAGGGTCTGGGCAGCGCCGTCGTGCTCCCCGGAGTGG |
| | | AGGCGGCCGAGCGCGCCGGGGTGCCCGCCTTCCTGGAGACCTCCGC |
| | | GCCCCGCAACCTCCCCTTCTACGAGCGGCTCGGCTTCACCGTCACC |
| | | GCCGACGTCGAGGTGCCCGAAGGACCGCGCACCTGGTGCATGACCC |
| | | GCAAGCCCGGTGCCTGATCTAGAATTCGGCCAggcCGGCCGATATC |
| | | AAGCTTATCGATAATCAACCTCTGGATTACAAAATTTGTGAAAGAT |
| | | TGACTGGTATTCTTAACTATGTTGCTCCTTTTACGCTATGTGGATA |
| | | CGCTGCTTTAATGCCTTTGTATCATGCTATTGCTTCCCGTATGGCT |
| | | TTCATTTTCTCCTCCTTGTATAAATCCTGGTTGCTGTCTCTTTATG |
| | | AGGAGTTGTGGCCCGTTGTCAGGCAACGTGGCGTGGTGTGCACTGT |
| | | GTTTGCTGACGCAACCCCCACTGGTTGGGGCATTGCCACCACCTGT |
| | | CAGCTCCTTTCCGGGACTTTCGCTTTCCCCCTCCCTATTGCCACGG |
| | | CGGAACTCATCGCCGCCTGCCTTGCCCGCTGCTGGACAGGGGCTCG |
| | | GCTGTTGGGCACTGACAATTCCGTGGTGTTGTCGGGGAAATCATCG |
| | | TCCTTTCCTTGGCTGCTCGCCTGTGTTGCCACCTGGATTCTGCGCG |
| | | GGACGTCCTTCTGCTACGTCCCTTCGGCCCTCAATCCAGCGGACCT |
| | | TCCTTCCCGCGGCCTGCTGCCGGCTCTGCGGCCTCTTCCGCGTCTT |
| | | CGCCTTCGCCCTCAGACGAGTCGGATCTCCCTTTGGGCCGCCTCCC |
| | | CGCATCGATACCGTCGACCTCGAGACCTAGAAAAACATGGAGCAAT |
| | | CACAAGTAGCAATACAGCAGCTACCAATGCTGATTGTGCCTGGCTA |
| | | GAAGCACAAGAGGAGGAGGAGGTGGGTTTTCCAGTCACACCTCAGG |
| | | TACCTTTAAGACCAATGACTTACAAGGCAGCTGTAGATCTTAGCCA |
| | | CTTTTTAAAAGAAAAGGGGGGACTGGAAGGGCTAATTCACTCCCAA |
| | | CGAAGACAAGATATCCTTGATCTGTGGATCTACCACACACAAGGCT |
| | | ACTTCCCTGATTGGCAGAACTACACACCAGGGCCAGGGATCAGATA |
| | | TCCACTGACCTTTGGATGGTGCTACAAGCTAGTACCAGTTGAGCAA |
| | | GAGAAGGTAGAAGAAGCCAATGAAGGAGAGAACACCCGCTTGTTAC |
| | | ACCCTGTGAGCCTGCATGGGATGGATGACCCGGAGAGAGAAGTATT |
| | | AGAGTGGAGGTTTGACAGCCGCCTAGCATTTCATCACATGGCCCGA |
| | | GAGCTGCATCCGGACTGTACTGGGTCTCTCTGGTTAGACCAGATCT |
| | | GAGCCTGGGAGCTCTCTGGCTAACTAGGGAACCCACTGCTTAAGCC |
| | | TCAATAAAGCTTGCCTTGAGTGCTTCAAGTAGTGTGTGCCCGTCTG |
| | | TTGTGTGACTCTGGTAACTAGAGATCCCTCAGACCCTTTTAGTCAG |
| | | TGTGGAAAATCTCTAGCAGGGCCCGTTTAAACCCGCTGATCAGCCT |
| | | CGACTGTGCCTTCTAGTTGCCAGCCATCTGTTGTTTGCCCCTCCCC |
| | | CGTGCCTTCCTTGACCCTGGAAGGTGCCACTCCCACTGTCCTTTCC |
| | | TAATAAAATGAGGAAATTGCATCGCATTGTCTGAGTAGGTGTCATT |
| | | CTATTCTGGGGGGTGGGGTGGGGCAGGACAGCAAGGGGGAGGATTG |
| | | GGAAGACAATAGCAGGCATGCTGGGGATGCGGTGGGCTCTATGGCT |
| | | TCTGAGGCGGAAAGAACCAGCTGGGGCTCTAGGGGGTATCCCCACG |
| | | CGCCCTGTAGCGGCGCATTAAGCGCGGCGGGTGTGGTGGTTACGCG |
| | | CAGCGTGACCGCTACACTTGCCAGCGCCCTAGCGCCCGCTCCTTTC |
| | | GCTTTCTTCCCTTCCTTTCTCGCCACGTTCGCCGGCTTTCCCCGTC |
| | | AAGCTCTAAATCGGGGGCTCCCTTTAGGGTTCCGATTTAGTGCTTT |
| | | ACGGCACCTCGACCCCAAAAAACTTGATTAGGGTGATGGTTCACGT |
| | | AGTGGGCCATCGCCCTGATAGACGGTTTTTCGCCCTTTGACGTTGG |
| | | AGTCCACGTTCTTTAATAGTGGACTCTTGTTCCAAACTGGAACAAC |
| | | ACTCAACCCTATCTCGGTCTATTCTTTTGATTTATAAGGGATTTTG |
| | | CCGATTTCGGCCTATTGGTTAAAAAATGAGCTGATTTAACAAAAAT |
| | | TTAACGCGAATTAATTCTGTGGAATGTGTGTCAGTTAGGGTGTGGA |
| | | AAGTCCCCAGGCTCCCCAGCAGGCAGAAGTATGCAAAGCATGCATC |
| | | TCAATTAGTCAGCAACCAGGTGTGGAAAGTCCCCAGGCTCCCCAGC |
| | | AGGCAGAAGTATGCAAAGCATGCATCTCAATTAGTCAGCAACCATA |
| | | GTCCCGCCCCTAACTCCGCCCATCCCGCCCCTAACTCCGCCCAGTT |
| | | CCGCCCATTCTCCGCCCCATGGCTGACTAATTTTTTTTATTTATGC |
| | | AGAGGCCGAGGCCGCCTCTGCCTCTGAGCTATTCCAGAAGTAGTGA |
| | | GGAGGCTTTTTTGGAGGCCTAGGCTTTTGCAAAAAGCTCCCGGGAG |
| | | CTTGTATATCCATTTTCGGATCTGATCAGCACGTGTTGACAATTAA |
| | | TCATCGGCATAGTATATCGGCATAGTATAATACGACAAGGTGAGGA |
| | | ACTAAACCATGGCCAAGTTGACCAGTGCCGTTCCGGTGCTCACCGC |
| | | GCGCGACGTCGCCGGAGCGGTCGAGTTCTGGACCGACCGGCTCGGG |
| | | TTCTCCCGGGACTTCGTGGAGGACGACTTCGCCGGTGTGGTCCGGG |
| | | ACGACGTGACCCTGTTCATCAGCGCGGTCCAGGACCAGGTGGTGCC |
| | | GGACAACACCCTGGCCTGGGTGTGGGTGCGCGGCCTGGACGAGCTG |
| | | TACGCCGAGTGGTCGGAGGTCGTGTCCACGAACTTCCGGGACGCCT |
| | | CCGGGCCGGCCATGACCGAGATCGGCGAGCAGCCGTGGGGGCGGGA |
| | | GTTCGCCCTGCGCGACCCGGCCGGCAACTGCGTGCACTTCGTGGCC |
| | | GAGGAGCAGGACTGACACGTGCTACGAGATTTCGATTCCACCGCCG |
| | | CCTTCTATGAAAGGTTGGGCTTCGGAATCGTTTTCCGGGACGCCGG |
| | | CTGGATGATCCTCCAGCGCGGGGATCTCATGCTGGAGTTCTTCGCC |
| | | CACCCCAACTTGTTTATTGCAGCTTATAATGGTTACAAATAAAGCA |
| | | ATAGCATCACAAATTTCACAAATAAAGCATTTTTTTCACTGCATTC |
| | | TAGTTGTGGTTTGTCCAAACTCATCAATGTATCTTATCATGTCTGT |
| | | ATACCGTCGACCTCTAGCTAGAGCTTGGCGTAATCATGGTCATAGC |
| | | TGTTTCCTGTGTGAAATTGTTATCCGCTCACAATTCCACACAACAT |
| | | ACGAGCCGGAAGCATAAAGTGTAAAGCCTGGGGTGCCTAATGAGTG |
| | | AGCTAACTCACATTAATTGCGTTGCGCTCACTGCCCGCTTTCCAGT |
| | | CGGGAAACCTGTCGTGCCAGCTGCATTAATGAATCGGCCAACGCGC |
| | | GGGGAGAGGCGGTTTGCGTATTGGGCGCTCTTCCGCTTCCTCGCTC |
| | | ACTGACTCGCTGCGCTCGGTCGTTCGGCTGCGGCGAGCGGTATCAG |
| | | CTCACTCAAAGGCGGTAATACGGTTATCCACAGAATCAGGGGATAA |
| | | CGCAGGAAAGAACATGTGAGCAAAAGGCCAGCAAAAGGCCAGGAAC |
| | | CGTAAAAAGGCCGCGTTGCTGGCGTTTTTCCATAGGCTCCGCCCCC |
| | | CTGACGAGCATCACAAAAATCGACGCTCAAGTCAGAGGTGGCGAAA |
| | | CCCGACAGGACTATAAAGATACCAGGCGTTTCCCCCTGGAAGCTCC |
| | | CTCGTGCGCTCTCCTGTTCCGACCCTGCCGCTTACCGGATACCTGT |
| | | CCGCCTTTCTCCCTTCGGGAAGCGTGGCGCTTTCTCATAGCTCACG |
| | | CTGTAGGTATCTCAGTTCGGTGTAGGTCGTTCGCTCCAAGCTGGGC |
| | | TGTGTGCACGAACCCCCCGTTCAGCCCGACCGCTGCGCCTTATCCG |
| | | GTAACTATCGTCTTGAGTCCAACCCGGTAAGACACGACTTATCGCC |
| | | ACTGGCAGCAGCCACTGGTAACAGGATTAGCAGAGCGAGGTATGTA |
| | | GGCGGTGCTACAGAGTTCTTGAAGTGGTGGCCTAACTACGGCTACA |
| | | CTAGAAGAACAGTATTTGGTATCTGCGCTCTGCTGAAGCCAGTTAC |
| | | CTTCGGAAAAAGAGTTGGTAGCTCTTGATCCGGCAAACAAACCACC |
| | | GCTGGTAGCGGTGGTTTTTTTGTTTGCAAGCAGCAGATTACGCGCA |
| | | GAAAAAAAGGATCTCAAGAAGATCCTTTGATCTTTTCTACGGGGTC |
| | | TGACGCTCAGTGGAACGAAAACTCACGTTAAGGGATTTTGGTCATG |
| | | AGATTATCAAAAAGGATCTTCACCTAGATCCTTTTAAATTAAAAAT |
| | | GAAGTTTTAAATCAATCTAAAGTATATATGAGTAAACTTGGTCTGA |
| | | CAGTTACCAATGCTTAATCAGTGAGGCACCTATCTCAGCGATCTGT |
| | | CTATTTCGTTCATCCATAGTTGCCTGACTCCCCGTCGTGTAGATAA |
| | | CTACGATACGGGAGGGCTTACCATCTGGCCCCAGTGCTGCAATGAT |
| | | ACCGCGAGACCCACGCTCACCGGCTCCAGATTTATCAGCAATAAAC |
| | | CAGCCAGCCGGAAGGGCCGAGCGCAGAAGTGGTCCTGCAACTTTAT |
| | | CCGCCTCCATCCAGTCTATTAATTGTTGCCGGGAAGCTAGAGTAAG |
| | | TAGTTCGCCAGTTAATAGTTTGCGCAACGTTGTTGCCATTGCTACA |
| | | GGCATCGTGGTGTCACGCTCGTCGTTTGGTATGGCTTCATTCAGCT |
| | | CCGGTTCCCAACGATCAAGGCGAGTTACATGATCCCCCATGTTGTG |
| | | CAAAAAAGCGGTTAGCTCCTTCGGTCCTCCGATCGTTGTCAGAAGT |
| | | AAGTTGGCCGCAGTGTTATCACTCATGGTTATGGCAGCACTGCATA |
| | | ATTCTCTTACTGTCATGCCATCCGTAAGATGCTTTTCTGTGACTGG |
| | | TGAGTACTCAACCAAGTCATTCTGAGAATAGTGTATGCGGCGACCG |
| | | AGTTGCTCTTGCCCGGCGTCAATACGGGATAATACCGCGCCACATA |
| | | GCAGAACTTTAAAAGTGCTCATCATTGGAAAACGTTCTTCGGGGCG |
| | | AAAACTCTCAAGGATCTTACCGCTGTTGAGATCCAGTTCGATGTAA |
| | | CCCACTCGTGCACCCAACTGATCTTCAGCATCTTTTACTTTCACCA |
| | | GCGTTTCTGGGTGAGCAAAAACAGGAAGGCAAAATGCCGCAAAAAA |
| | | GGGAATAAGGGCGACACGGAAATGTTGAATACTCATACTCTTCCTT |
| | | TTTCAATATTATTGAAGCATTTATCAGGGTTATTGTCTCATGAGCG |
| | | GATACATATTTGAATGTATTTAGAAAAATAAACAAATAGGGGTTCC |
| | | GCGCACATTTCCCCGAAAAGTGCCACCTGAC |
| 22 | U6: | GTAAAACGACGGCCAGTGA |
| | gRNA | |
| | forward | |
| | primer | |
| 23 | U6: | CGTCTCAAAAAATAAAAN20GTTGCATTCCTTTCTTTGTTTCG* |
| | gRNA | *N20 represents the reverse complement of the |
| | reverse | protospacer sequence |
| | primer | |
| | SpCas9 | |
| | (DNA) | |
| 24 | + NLSs | atggactataaggaccacgacggagactacaaggatcatgatattg |
| | + hygromycin | attacaaagacgatgacgataagatggccccaaagaagaagcggaa |
| | resistance | ggtcggtatccacggagtcccagcagccgacaagaagtacagcatc |
| | | ggcctggacatcggcaccaactctgtgggctgggccgtgatcaccg |
| | | acgagtacaaggtgcccagcaagaaattcaaggtgctgggcaacac |
| | | cgaccggcacagcatcaagaagaacctgatcggagccctgctgttc |
| | | gacagcggcgaaacagccgaggccacccggctgaagagaaccgcca |
| | | gaagaagatacaccagacggaagaaccggatctgctatctgcaaga |
| | | gatcttcagcaacgagatggccaaggtggacgacagcttcttccac |
| | | agactggaagagtccttcctggtggaagaggataagaagcacgagc |
| | | ggcaccccatcttcggcaacatcgtggacgaggtggcctaccacga |
| | | gaagtaccccaccatctaccacctgagaaagaaactggtggacagc |
| | | accgacaaggccgacctgcggctgatctatctggccctggcccaca |
| | | tgatcaagttccggggccacttcctgatcgagggcgacctgaaccc |
| | | cgacaacagcgacgtggacaagctgttcatccagctggtgcagacc |
| | | tacaaccagctgttcgaggaaaaccccatcaacgccagcggcgtgg |
| | | acgccaaggccatcctgtctgccagactgagcaagagcagacggct |
| | | ggaaaatctgatcgcccagctgcccggcgagaagaagaatggcctg |
| | | ttcggaaacctgattgccctgagcctgggcctgacccccaacttca |
| | | agagcaacttcgacctggccgaggatgccaaactgcagctgagcaa |
| | | ggacacctacgacgacgacctggacaacctgctggcccagateggc |
| | | gaccagtacgccgacctgtttctggccgccaagaacctgtccgacg |
| | | ccatcctgctgagcgacatcctgagagtgaacaccgagatcaccaa |
| | | ggcccccctgagcgcctctatgatcaagagatacgacgagcaccac |
| | | caggacctgaccctgctgaaagctctcgtgcggcagcagctgcctg |
| | | agaagtacaaagagattttcttcgaccagagcaagaacggctacgc |
| | | cggctacattgacggcggagccagccaggaagagttctacaagttc |
| | | atcaagcccatcctggaaaagatggacggcaccgaggaactgctcg |
| | | tgaagctgaacagagaggacctgctgcggaagcagcggaccttcga |
| | | caacggcagcatcccccaccagatccacctgggagagctgcacgcc |
| | | attctgcggcggcaggaagatttttacccattcctgaaggacaacc |
| | | gggaaaagategagaagatcctgaccttccgcatcccctactacgt |
| | | gggccctctggccaggggaaacagcagattcgcctggatgaccaga |
| | | aagagcgaggaaaccatcaccccctggaacttcgaggaagtggtgg |
| | | acaagggcgcttccgcccagagcttcatcgagcggatgaccaactt |
| | | cgataagaacctgcccaacgagaaggtgctgcccaagcacagcctg |
| | | ctgtacgagtacttcaccgtgtataacgagctgaccaaagtgaaat |
| | | acgtgaccgagggaatgagaaagcccgccttcctgagcggcgagca |
| | | gaaaaaggccatcgtggacctgctgttcaagaccaaccggaaagtg |
| | | accgtgaagcagctgaaagaggactacttcaagaaaatcgagtgct |
| | | tcgactccgtggaaatctccggcgtggaagatcggttcaacgcctc |
| | | cctgggcacataccacgatctgctgaaaattatcaaggacaaggac |
| | | ttcctggacaatgaggaaaacgaggacattctggaagatatcgtgc |
| | | tgaccctgacactgtttgaggacagagagatgatcgaggaacggct |
| | | gaaaacctatgcccacctgttcgacgacaaagtgatgaagcagctg |
| | | aagcggcggagatacaccggctggggcaggctgagccggaagctga |
| | | tcaacggcatccgggacaagcagtccggcaagacaatcctggattt |
| | | cctgaagtccgacggcttcgccaacagaaacttcatgcagctgatc |
| | | cacgacgacagcctgacctttaaagaggacatccagaaagcccagg |
| | | tgtccggccagggcgatagcctgcacgagcacattgccaatctggc |
| | | cggcagccccgccattaagaagggcatcctgcagacagtgaaggtg |
| | | gtggacgagctcgtgaaagtgatgggccggcacaagcccgagaaca |
| | | tcgtgatcgaaatggccagagagaaccagaccacccagaagggaca |
| | | gaagaacagccgcgagagaatgaagcggatcgaagagggcatcaaa |
| | | gagctgggcagccagatcctgaaagaacaccccgtggaaaacaccc |
| | | agctgcagaacgagaagctgtacctgtactacctgcagaatggggg |
| | | gatatgtacgtggaccaggaactggacatcaaccggctgtccgact |
| | | acgatgtggaccatatcgtgcctcagagctttctgaaggacgactc |
| | | catcgacaacaaggtgctgaccagaagcgacaagaaccggggcaag |
| | | agcgacaacgtgccctccgaagaggtcgtgaagaagatgaagaact |
| | | actggcggcagctgctgaacgccaagctgattacccagagaaagtt |
| | | cgacaatctgaccaaggccgagagaggcggcctgagcgaactggat |
| | | aaggccggcttcatcaagagacagctggtggaaacccggcagatca |
| | | caaagcacgtggcacagatcctggactcccggatgaacactaagta |
| | | cgacgagaatgacaagctgatccgggaagtgaaagtgatcaccctg |
| | | aagtccaagctggtgtccgatttccggaaggatttccagttttaca |
| | | aagtgcgcgagatcaacaactaccaccacgcccacgacgcctacct |
| | | gaacgccgtcgtgggaaccgccctgatcaaaaagtaccctaagctg |
| | | gaaagcgagttcgtgtacggcgactacaaggtgtacgacgtgcgga |
| | | agatgatcgccaagagcgagcaggaaatcggcaaggctaccgccaa |
| | | gtacttcttctacagcaacatcatgaactttttcaagaccgagatt |
| | | accctggccaacggcgagatccggaagcggcctctgatcgagacaa |
| | | acggcgaaaccggggagatcgtgtgggataagggccgggattttgc |
| | | caccgtgcggaaagtgctgagcatgccccaagtgaatatcgtgaaa |
| | | aagaccgaggtgcagacaggcggcttcagcaaagagtctatcctgc |
| | | ccaagaggaacagcgataagctgatcgccagaaagaaggactggga |
| | | ccctaagaagtacggcggcttcgacagccccaccgtggcctattct |
| | | gtgctggtggtggccaaagtggaaaagggcaagtccaagaaactga |
| | | agagtgtgaaagagctgctggggatcaccatcatggaaagaagcag |
| | | cttcgagaagaatcccatcgactttctggaagccaagggctacaaa |
| | | gaagtgaaaaaggacctgatcatcaagctgcctaagtactccctgt |
| | | tcgagctggaaaacggccggaagagaatgctggcctctgccggcga |
| | | actgcagaagggaaacgaactggccctgccctccaaatatgtgaac |
| | | ttcctgtacctggccagccactatgagaagctgaagggctcccccg |
| | | aggataatgagcagaaacagctgtttgtggaacagcacaagcacta |
| | | cctggacgagatcatcgagcagatcagcgagttctccaagagagtg |
| | | atcctggccgacgctaatctggacaaagtgctgtccgcctacaaca |
| | | agcaccgggataagcccatcagagagcaggccgagaatatcatcca |
| | | cctgtttaccctgaccaatctgggagcccctgccgccttcaagtac |
| | | tttgacaccaccatcgaccggaagaggtacaccagcaccaaagagg |
| | | tgctggacgccaccctgatccaccagagcatcaccggcctgtacga |
| | | gacacggatcgacctgtctcagctgggaggcgacaaaaggccggcg |
| | | gccacgaaaaaggccggccaggcaaaaaagaaaaagggaagcggag |
| | | agggcaggggaagtcttctaacatgcggggacgtggaggaaaatcc |
| | | cggccccatgaaaaagcctgaactcaccgcgacgtctgtcgagaag |
| | | tttctgatcgaaaagttcgacagcgtctccgacctgatgcagctct |
| | | cggagggcgaagaatctcgtgctttcagcttcgatgtaggagggcg |
| | | tggatatgtcctgcgggtaaatagctgcgccgatggtttctacaaa |
| | | gatcgttatgtttatcggcactttgcatcggccgcgctcccgattc |
| | | cggaagtgcttgacattggggaatttagcgagagcctgacctattg |
| | | catctcccgccgtgcacagggtgtcacgttgcaagacctgcctgaa |
| | | accgaactgcccgctgttctgcagccggtcgcggaggccatggatg |
| | | cgatcgctgcggccgatcttagccagacgagcgggttcggcccatt |
| | | cggaccgcaaggaatcggtcaatacactacatggcgtgatttcata |
| | | tgcgcgattgctgatccccatgtgtatcactggcaaactgtgatgg |
| | | acgacaccgtcagtgcgtccgtegcgcaggctctcgatgagctgat |
| | | gctttgggccgaggactgccccgaagtccggcacctcgtgcacgcg |
| | | gatttcggctccaacaatgtcctgacggacaatggccgcataacag |
| | | cggtcattgactggagcgaggcgatgttcggggattcccaatacga |
| | | ggtcgccaacatcttcttctggaggccgtggttggcttgtatggag |
| | | cagcagacgcgctacttcgagcggaggcatccggagcttgcaggat |
| | | cgccgcggctccgggcgtatatgctccgcattggtcttgaccaact |
| | | ctatcagagcttggttgacggcaatttcgatgatgcagcttgggcg |
| | | cagggtcgatgcgacgcaatcgtccgatccggagccgggactgtcg |
| | | ggcgtacacaaatcgcccgcagaagcgcggccgtctggaccgatgg |
| | | ctgtgtagaagtactcgccgatagtggaaaccgacgccccagcact |
| | | cgtccgagggcaaaggaatag |
| 25 | SpCas9 | MDYKDHDGDYKDHDIDYKDDDDKMAPKKKRKVGIHGVPAADKKYSI |
| | (amino | GLDIGTNSVGWAVITDEYKVPSKKFKVLGNTDRHSIKKNLIGALLF |
| | acid) + N | DSGETAEATRLKRTARRRYTRRKNRICYLQEIFSNEMAKVDDSFFH |
| | LSs + | RLEESFLVEEDKKHERHPIFGNIVDEVAYHEKYPTIYHLRKKLVDS |
| | hygromycin | TDKADLRLIYLALAHMIKFRGHFLIEGDLNPDNSDVDKLFIQLVQT |
| | resistance | YNQLFEENPINASGVDAKAILSARLSKSRRLENLIAQLPGEKKNGL |
| | | FGNLIALSLGLTPNFKSNFDLAEDAKLQLSKDTYDDDLDNLLAQIG |
| | | DQYADLFLAAKNLSDAILLSDILRVNTEITKAPLSASMIKRYDEHH |
| | | QDLTLLKALVRQQLPEKYKEIFFDQSKNGYAGYIDGGASQEEFYKF |
| | | IKPILEKMDGTEELLVKLNREDLLRKQRTFDNGSIPHQIHLGELHA |
| | | ILRRQEDFYPFLKDNREKIEKILTFRIPYYVGPLARGNSRFAWMTR |
| | | KSEETITPWNFEEVVDKGASAQSFIERMTNFDKNLPNEKVLPKHSL |
| | | LYEYFTVYNELTKVKYVTEGMRKPAFLSGEQKKAIVDLLFKTNRKV |
| | | TVKQLKEDYFKKIECFDSVEISGVEDRFNASLGTYHDLLKIIKDKD |
| | | FLDNEENEDILEDIVLTLTLFEDREMIEERLKTYAHLFDDKVMKQL |
| | | KRRRYTGWGRLSRKLINGIRDKQSGKTILDFLKSDGFANRNFMQLI |
| | | HDDSLTFKEDIQKAQVSGQGDSLHEHIANLAGSPAIKKGILQTVKV |
| | | VDELVKVMGRHKPENIVIEMARENQTTQKGQKNSRERMKRIEEGIK |
| | | ELGSQILKEHPVENTQLQNEKLYLYYLQNGRDMYVDQELDINRLSD |
| | | YDVDHIVPQSFLKDDSIDNKVLTRSDKNRGKSDNVPSEEVVKKMKN |
| | | YWRQLLNAKLITQRKFDNLTKAERGGLSELDKAGFIKRQLVETRQI |
| | | TKHVAQILDSRMNTKYDENDKLIREVKVITLKSKLVSDFRKDFQFY |
| | | KVREINNYHHAHDAYLNAVVGTALIKKYPKLESEFVYGDYKVYDVR |
| | | KMIAKSEQEIGKATAKYFFYSNIMNFFKTEITLANGEIRKRPLIET |
| | | NGETGEIVWDKGRDFATVRKVLSMPQVNIVKKTEVQTGGFSKESIL |
| | | PKRNSDKLIARKKDWDPKKYGGFDSPTVAYSVLVVAKVEKGKSKKL |
| | | KSVKELLGITIMERSSFEKNPIDFLEAKGYKEVKKDLIIKLPKYSL |
| | | FELENGRKRMLASAGELQKGNELALPSKYVNFLYLASHYEKLKGSP |
| | | EDNEQKQLFVEQHKHYLDEIIEQISEFSKRVILADANLDKVLSAYN |
| | | KHRDKPIREQAENIIHLFTLTNLGAPAAFKYFDTTIDRKRYTSTKE |
| | | VLDATLIHQSITGLYETRIDLSQLGGDKRPAATKKAGQAKKKKGSG |
| | | EGRGSLLTCGDVEENPGPMKKPELTATSVEKFLIEKFDSVSDLMQL |
| | | SEGEESRAFSFDVGGRGYVLRVNSCADGFYKDRYVYRHFASAALPI |
| | | PEVLDIGEFSESLTYCISRRAQGVTLQDLPETELPAVLQPVAEAMD |
| | | AIAAADLSQTSGFGPFGPQGIGQYTTWRDFICAIADPHVYHWQTVM |
| | | DDTVSASVAQALDELMLWAEDCPEVRHLVHADFGSNNVLTDNGRIT |
| | | AVIDWSEAMFGDSQYEVANIFFWRPWLACMEQQTRYFERRHPELAG |
| | | SPRLRAYMLRIGLDQLYQSLVDGNFDDAAWAQGRCDAIVRSGAGTV |
| | | GRTQIARRSAAVWTDGCVEVLADSGNRRPSTRPRAKE* |
| 26 | SaCas9 | atggccccaaagaagaagcggaaggtcggtatccacggagtcccag |
| | (DNA) | cagccaagcggaactacatcctgggcctggacatcggcatcaccag |
| | + NLSs | cgtgggctacggcatcatcgactacgagacacgggacgtgatcgat |
| | + hygromycin | gccggcgtgcggctgttcaaagaggccaacgtggaaaacaacgagg |
| | + resistance | gcaggcggagcaagagaggcgccagaaggctgaagcggcggaggcg |
| | + HA | gcatagaatccagagagtgaagaagctgctgttcgactacaacctg |
| | tag | ctgaccgaccacagcgagctgagcggcatcaacccctacgaggcca |
| | | gagtgaagggcctgagccagaagctgagcgaggaagagttctctgc |
| | | cgccctgctgcacctggccaagagaagaggcgtgcacaacgtgaac |
| | | gaggtggaagaggacaccggcaacgagctgtccaccaaagagcaga |
| | | tcagccggaacagcaaggccctggaagagaaatacgtggccgaact |
| | | gcagctggaacggctgaagaaagacggcgaagtgcggggcagcatc |
| | | aacagattcaagaccagcgactacgtgaaagaagccaaacagctgc |
| | | tgaaggtgcagaaggcctaccaccagctggaccagagcttcatcga |
| | | cacctacatcgacctgctggaaacccggcggacctactatgaggga |
| | | cctggcgagggcagccccttcggctggaaggacatcaaagaatggt |
| | | acgagatgctgatgggccactgcacctacttccccgaggaactgcg |
| | | gagcgtgaagtacgcctacaacgccgacctgtacaacgccctgaac |
| | | gacctgaacaatctcgtgatcaccagggacgagaacgagaagctgg |
| | | aatattacgagaagttccagatcatcgagaacgtgttcaagcagaa |
| | | gaagaagcccaccctgaagcagatcgccaaagaaatcctcgtgaac |
| | | gaagaggatattaagggctacagagtgaccagcaccggcaagcccg |
| | | agttcaccaacctgaaggtgtaccacgacatcaaggacattaccgc |
| | | ccggaaagagattattgagaacgccgagctgctggatcagattgcc |
| | | aagatcctgaccatctaccagagcagcgaggacatccaggaagaac |
| | | tgaccaatctgaactccgagctgacccaggaagagatcgagcagat |
| | | ctctaatctgaagggctataccggcacccacaacctgagcctgaag |
| | | gccatcaacctgatcctggacgagctgtggcacaccaacgacaacc |
| | | agatcgctatcttcaaccggctgaagctggtgcccaagaaggtgga |
| | | cctgtcccagcagaaagagatccccaccaccctggtggacgacttc |
| | | atcctgagccccgtcgtgaagagaagcttcatccagagcatcaaag |
| | | tgatcaacgccatcatcaagaagtacggcctgcccaacgacatcat |
| | | tatcgagctggcccgcgagaagaactccaaggacgcccagaaaatg |
| | | atcaacgagatgcagaagcggaaccggcagaccaacgagcggatcg |
| | | aggaaatcatccggaccaccggcaaagagaacgccaagtacctgat |
| | | cgagaagatcaagctgcacgacatgcaggaaggcaagtgcctgtac |
| | | agcctggaagccatccctctggaagatctgctgaacaaccccttca |
| | | actatgaggtggaccacatcatccccagaagcgtgtccttcgacaa |
| | | cagcttcaacaacaaggtgctcgtgaagcaggaagaaaacagcaag |
| | | aagggcaaccggaccccattccagtacctgagcagcagcgacagca |
| | | agatcagctacgaaaccttcaagaagcacatcctgaatctggccaa |
| | | gggcaagggcagaatcagcaagaccaagaaagagtatctgctggaa |
| | | gaacgggacatcaacaggttctccgtgcagaaagacttcatcaacc |
| | | ggaacctggtggataccagatacgccaccagaggcctgatgaacct |
| | | gctgcggagctacttcagagtgaacaacctggacgtgaaagtgaag |
| | | tccatcaatggcggcttcaccagctttctgcggcggaagtggaagt |
| | | ttaagaaagagcggaacaaggggtacaagcaccacgccgaggacgc |
| | | cctgatcattgccaacgccgatttcatcttcaaagagtggaagaaa |
| | | ctggacaaggccaaaaaagtgatggaaaaccagatgttcgaggaaa |
| | | agcaggccgagagcatgcccgagatcgaaaccgagcaggagtacaa |
| | | agagatcttcatcaccccccaccagatcaagcacattaaggacttc |
| | | aaggactacaagtacagccaccgggtggacaagaagcctaatagag |
| | | agctgattaacgacaccctgtactccacccggaaggacgacaaggg |
| | | caacaccctgatcgtgaacaatctgaacggcctgtacgacaaggac |
| | | aatgacaagctgaaaaagctgatcaacaagagccccgaaaagctgc |
| | | tgatgtaccaccacgacccccagacctaccagaaactgaagctgat |
| | | tatggaacagtacggcgacgagaagaatcccctgtacaagtactac |
| | | gaggaaaccgggaactacctgaccaagtactccaaaaaggacaacg |
| | | gccccgtgatcaagaagattaagtattacggcaacaaactgaacgc |
| | | ccatctggacatcaccgacgactaccccaacagcagaaacaaggtc |
| | | gtgaagctgtccctgaagccctacagattcgacgtgtacctggaca |
| | | atggcgtgtacaagttcgtgaccgtgaagaatctggatgtgatcaa |
| | | aaaagaaaactactacgaagtgaatagcaagtgctatgaggaagct |
| | | aagaagctgaagaagatcagcaaccaggccgagtttategcctcct |
| | | tctacaacaacgatctgatcaagatcaacggcgagctgtatagagt |
| | | gatcggcgtgaacaacgacctgctgaaccggatcgaagtgaacatg |
| | | atcgacatcacctaccgcgagtacctggaaaacatgaacgacaaga |
| | | ggccccccaggatcattaagacaatcgcctccaagacccagagcat |
| | | taagaagtacagcacagacattctgggcaacctgtatgaagtgaaa |
| | | tctaagaagcaccctcagatcatcaaaaagggcaaaaggccggcgg |
| | | ccacgaaaaaggccggccaggcaaaaaagaaaaagtacccatacga |
| | | tgttccagattacgctggaagcggagagggcaggggaagtcttcta |
| | | acatgcggggacgtggaggaaaatcccggccccatgaaaaagcctg |
| | | aactcaccgcgacgtctgtcgagaagtttctgatcgaaaagttcga |
| | | cagcgtctccgacctgatgcagctctcggagggcgaagaatctcgt |
| | | gctttcagcttcgatgtaggagggcgtggatatgtcctgcgggtaa |
| | | atagctgcgccgatggtttctacaaagatcgttatgtttatcggca |
| | | ctttgcatcggccgcgctcccgattccggaagtgcttgacattggg |
| | | gaatttagcgagagcctgacctattgcatctcccgccgtgcacagg |
| | | gtgtcacgttgcaagacctgcctgaaaccgaactgcccgctgttct |
| | | gcagccggtcgcggaggccatggatgcgatcgctgcggccgatctt |
| | | agccagacgagcgggttcggcccattcggaccgcaaggaatcggtc |
| | | aatacactacatggcgtgatttcatatgcgcgattgctgatcccca |
| | | tgtgtatcactggcaaactgtgatggacgacaccgtcagtgcgtcc |
| | | gtcgcgcaggctctcgatgagctgatgctttgggccgaggactgcc |
| | | ccgaagtccggcacctcgtgcacgcggatttcggctccaacaatgt |
| | | cctgacggacaatggccgcataacagcggtcattgactggagcgag |
| | | gcgatgttcggggattcccaatacgaggtcgccaacatcttcttct |
| | | ggaggccgtggttggcttgtatggagcagcagacgcgctacttcga |
| | | gcggaggcatccggagcttgcaggatcgccgcggctccgggcgtat |
| | | atgctccgcattggtcttgaccaactctatcagagcttggttgacg |
| | | gcaatttcgatgatgcagcttgggcgcagggtcgatgcgacgcaat |
| | | cgtccgatccggagccgggactgtcgggcgtacacaaatcgcccgc |
| | | agaagcgcggccgtctggaccgatggctgtgtagaagtactcgccg |
| | | atagtggaaaccgacgccccagcactcgtccgagggcaaaggaata |
| | | g |
| | SaCas9 | MAPKKKRKVGIHGVPAAKRNYILGLDIGITSVGYGIIDYETRDVID |
| 27 | (amino | AGVRLFKEANVENNEGRRSKRGARRLKRRRRHRIQRVKKLLFDYNL |
| | acid)+N | LTDHSELSGINPYEARVKGLSQKLSEEEFSAALLHLAKRRGVHNVN |
| | LSs | EVEEDTGNELSTKEQISRNSKALEEKYVAELQLERLKKDGEVRGSI |
| | hygrom | NRFKTSDYVKEAKQLLKVQKAYHQLDQSFIDTYIDLLETRRTYYEG |
| | ycin+re | PGEGSPFGWKDIKEWYEMLMGHCTYFPEELRSVKYAYNADLYNALN |
| | sistance | DLNNLVITRDENEKLEYYEKFQIIENVFKQKKKPTLKQIAKEILVN |
| | + HA | EEDIKGYRVTSTGKPEFTNLKVYHDIKDITARKEIIENAELLDQIA |
| | tag | KILTIYQSSEDIQEELTNLNSELTQEEIEQISNLKGYTGTHNLSLK |
| | | AINLILDELWHTNDNQIAIFNRLKLVPKKVDLSQQKEIPTTLVDDF |
| | | ILSPVVKRSFIQSIKVINAIIKKYGLPNDIIIELAREKNSKDAQKM |
| | | INEMQKRNRQTNERIEEIIRTTGKENAKYLIEKIKLHDMQEGKCLY |
| | | SLEAIPLEDLLNNPFNYEVDHIIPRSVSFDNSFNNKVLVKQEENSK |
| | | KGNRTPFQYLSSSDSKISYETFKKHILNLAKGKGRISKTKKEYLLE |
| | | ERDINRFSVQKDFINRNLVDTRYATRGLMNLLRSYFRVNNLDVKVK |
| | | SINGGFTSFLRRKWKFKKERNKGYKHHAEDALIIANADFIFKEWKK |
| | | LDKAKKVMENQMFEEKQAESMPEIETEQEYKEIFITPHQIKHIKDF |
| | | KDYKYSHRVDKKPNRELINDTLYSTRKDDKGNTLIVNNLNGLYDKD |
| | | NDKLKKLINKSPEKLLMYHHDPQTYQKLKLIMEQYGDEKNPLYKYY |
| | | EETGNYLTKYSKKDNGPVIKKIKYYGNKLNAHLDITDDYPNSRNKV |
| | | VKLSLKPYRFDVYLDNGVYKFVTVKNLDVIKKENYYEVNSKCYEEA |
| | | KKLKKISNQAEFIASFYNNDLIKINGELYRVIGVNNDLLNRIEVNM |
| | | IDITYREYLENMNDKRPPRIIKTIASKTQSIKKYSTDILGNLYEVK |
| | | SKKHPQIIKKGKRPAATKKAGQAKKKKYPYDVPDYAGSGEGRGSLL |
| | | TCGDVEENPGPMKKPELTATSVEKFLIEKFDSVSDLMQLSEGEESR |
| | | AFSFDVGGRGYVLRVNSCADGFYKDRYVYRHFASAALPIPEVLDIG |
| | | EFSESLTYCISRRAQGVTLQDLPETELPAVLQPVAEAMDAIAAADL |
| | | SQTSGFGPFGPQGIGQYTTWRDFICAIADPHVYHWQTVMDDTVSAS |
| | | VAQALDELMLWAEDCPEVRHLVHADFGSNNVLTDNGRITAVIDWSE |
| | | AMFGDSQYEVANIFFWRPWLACMEQQTRYFERRHPELAGSPRLRAY |
| | | MLRIGLDQLYQSLVDGNFDDAAWAQGRCDAIVRSGAGTVGRTQIAR |
| | | RSAAVWTDGCVEVLADSGNRRPSTRPRAKE* |
| 28 | Cas12f | ATGCCAAAGAAGAAGCGGAAGGTCGGTATCCACGGAGTCCCAGCAG |
| | 1 | CCATGGCCAAGAACACAATTACAAAGACACTGAAGCTGAGGATCGT |
| | (DNA) | GAGACCATACAACAGCGCTGAGGTCGAGAAGATTGTGGCTGATGAA |
| | | AAGAACAACAGGGAAAAGATCGCCCTCGAGAAGAACAAGGATAAGG |
| | | TGAAGGAGGCCTGCTCTAAGCACCTGAAAGTGGCCGCCTACTGCAC |
| | | CACACAGGTGGAGAGGAACGCCTGTCTGTTTTGTAAAGCTCGGAAG |
| | | CTGGATGATAAGTTTTACCAGAAGCTGCGGGGCCAGTTCCCCGATG |
| | | CCGTCTTTTGGCAGGAGATTAGCGAGATCTTCAGACAGCTGCAGAA |
| | | GCAGGCCGCCGAGATCTACAACCAGAGCCTGATCGAGCTCTACTAC |
| | | GAGATCTTCATCAAGGGCAAGGGCATTGCCAACGCCTCCTCCGTGG |
| | | AGCACTACCTGAGCGACGTGTGCTACACAAGAGCCGCCGAGCTCTT |
| | | TAAGAACGCCGCTATCGCTTCCGGGCTGAGGAGCAAGATTAAGAGT |
| | | AACTTCCGGCTCAAGGAGCTGAAGAACATGAAGAGCGGCCTGCCCA |
| | | CTACAAAGAGCGACAACTTCCCAATTCCACTGGTGAAGCAGAAGGG |
| | | GGGCCAGTACACAGGGTTCGAGATTTCCAACCACAACAGCGACTTT |
| | | ATTATTAAGATCCCCTTTGGCAGGTGGCAGGTCAAGAAGGAGATTG |
| | | ACAAGTACAGGCCCTGGGAGAAGTTTGATTTCGAGCAGGTGCAGAA |
| | | GAGCCCCAAGCCTATTTCCCTGCTGCTGTCCACACAGCGGCGGAAG |
| | | AGGAACAAGGGGTGGTCTAAGGATGAGGGGACCGAGGCCGAGATTA |
| | | AGAAAGTGATGAACGGCGACTACCAGACAAGCTACATCGAGGTCAA |
| | | GCGGGGCAGTAAGATTGGCGAGAAGAGCGCCTGGATGCTGAACCTG |
| | | AGCATTGACGTGCCAAAGATTGATAAGGGCGTGGATCCCAGCATCA |
| | | TCGGAGGGATCGATGTGGGGGTCAAGAGCCCCCTCGTGTGCGCCAT |
| | | CAACAACGCCTTCAGCAGGTACAGCATCTCCGATAACGACCTGTTC |
| | | CACTTTAACAAGAAGATGTTCGCCCGGCGGAGGATTTTGCTCAAGA |
| | | AGAACCGGCACAAGCGGGCCGGACACGGGGCCAAGAACAAGCTCAA |
| | | GCCCATCACTATCCTGACCGAGAAGAGCGAGAGGTTCAGGAAGAAG |
| | | CTCATCGAGAGATGGGCCTGCGAGATCGCCGATTTCTTTATTAAGA |
| | | ACAAGGTCGGAACAGTGCAGATGGAGAACCTCGAGAGCATGAAGAG |
| | | GAAGGAGGATTCCTACTTCAACATTCGGCTGAGGGGGTTCTGGCCC |
| | | TACGCTGAGATGCAGAACAAGATTGAGTTTAAGCTGAAGCAGTACG |
| | | GGATTGAGATCCGGAAGGTGGCCCCCAACAACACCAGCAAGACCTG |
| | | CAGCAAGTGCGGGCACCTCAACAACTACTTCAACTTCGAGTACCGG |
| | | AAGAAGAACAAGTTCCCACACTTCAAGTGCGAGAAGTGCAACTTTA |
| | | AGGAGAACGCCGATTACAACGCCGCCCTGAACATCAGCAACCCTAA |
| | | GCTGAAGAGCACTAAGGAGGAGCCCAAAAGGCCGGCGGCCACGAAA |
| | | AAGGCCGGCCAGGCAAAAAAGAAAAAGtacccatacgatgttccag |
| | | attacgctggaagcggagagggcaggggaagtcttctaacatgcgg |
| | | ggacgtggaggaaaatcccggccccatgaaaaagcctgaactcacc |
| | | gcgacgtctgtcgagaagtttctgatcgaaaagttcgacagcgtct |
| | | ccgacctgatgcagctctcggagggcgaagaatctcgtgctttcag |
| | | cttcgatgtaggagggcgtggatatgtcctgcgggtaaatagctgc |
| | | gccgatggtttctacaaagatcgttatgtttatcggcactttgcat |
| | | cggccgcgctcccgattccggaagtgcttgacattggggaatttag |
| | | cgagagcctgacctattgcatctcccgccgtgcacagggtgtcacg |
| | | ttgcaagacctgcctgaaaccgaactgcccgctgttctgcagccgg |
| | | tcgcggaggccatggatgcgatcgctgcggccgatcttagccagac |
| | | gagcgggttcggcccattcggaccgcaaggaatcggtcaatacact |
| | | acatggcgtgatttcatatgcgcgattgctgatccccatgtgtatc |
| | | actggcaaactgtgatggacgacaccgtcagtgcgtccgtcgcgca |
| | | ggctctcgatgagctgatgctttgggccgaggactgccccgaagtc |
| | | cggcacctcgtgcacgcggatttcggctccaacaatgtcctgacgg |
| | | acaatggccgcataacagcggtcattgactggagcgaggcgatgtt |
| | | cggggattcccaatacgaggtcgccaacatcttcttctggaggccg |
| | | tggttggcttgtatggagcagcagacgcgctacttcgagcggaggc |
| | | atccggagcttgcaggatcgccgcggctccgggcgtatatgctccg |
| | | cattggtcttgaccaactctatcagagcttggttgacggcaatttc |
| | | gatgatgcagcttgggcgcagggtcgatgcgacgcaatcgtccgat |
| | | ccggagccgggactgtcgggcgtacacaaatcgcccgcagaagcgc |
| | | ggccgtctggaccgatggctgtgtagaagtactcgccgatagtgga |
| | | aaccgacgccccagcactcgtccgagggcaaaggaatag |
| 29 | Cas12f | MPKKKRKVGIHGVPAAMAKNTITKTLKLRIVRPYNSAEVEKIVADE |
| | 1 | KNNREKIALEKNKDKVKEACSKHLKVAAYCTTQVERNACLFCKARK |
| | (amino | LDDKFYQKLRGQFPDAVFWQEISEIFRQLQKQAAEIYNQSLIELYY |
| | acid)+ | EIFIKGKGIANASSVEHYLSDVCYTRAAELFKNAAIASGLRSKIKS |
| | NLSs | NFRLKELKNMKSGLPTTKSDNFPIPLVKQKGGQYTGFEISNHNSDF |
| | hygromycin | IIKIPFGRWQVKKEIDKYRPWEKFDFEQVQKSPKPISLLLSTQRRK |
| | resistance + | RNKGWSKDEGTEAEIKKVMNGDYQTSYIEVKRGSKIGEKSAWMLNL |
| | HA | SIDVPKIDKGVDPSIIGGIDVGVKSPLVCAINNAFSRYSISDNDLF |
| | tag | HFNKKMFARRRILLKKNRHKRAGHGAKNKLKPITILTEKSERFRKK |
| | | LIERWACEIADFFIKNKVGTVQMENLESMKRKEDSYFNIRLRGFWP |
| | | YAEMQNKIEFKLKQYGIEIRKVAPNNTSKTCSKCGHLNNYFNFEYR |
| | | KKNKFPHFKCEKCNFKENADYNAALNISNPKLKSTKEEPKRPAATK |
| | | KAGQAKKKKYPYDVPDYAGSGEGRGSLLTCGDVEENPGPMKKPELT |
| | | ATSVEKFLIEKFDSVSDLMQLSEGEESRAFSFDVGGRGYVLRVNSC |
| | | ADGFYKDRYVYRHFASAALPIPEVLDIGEFSESLTYCISRRAQGVT |
| | | LQDLPETELPAVLQPVAEAMDAIAAADLSQTSGFGPFGPQGIGQYT |
| | | TWRDFICAIADPHVYHWQTVMDDTVSASVAQALDELMLWAEDCPEV |
| | | RHLVHADFGSNNVLTDNGRITAVIDWSEAMFGDSQYEVANIFFWRP |
| | | WLACMEQQTRYFERRHPELAGSPRLRAYMLRIGLDQLYQSLVDGNF |
| | | DDAAWAQGRCDAIVRSGAGTVGRTQIARRSAAVWTDGCVEVLADSG |
| | | NRRPSTRPRAKE* |
| 30 | Sso7d | ATGGCTACAGTGAAGTTTAAGTATAAAGGGGAAGAGAAAGAGGTGG |
| | (DNA) | ACATCAGTAAAATCAAGAAAGTGTGGCGCGTAGGTAAGATGATCAG |
| | | CTTTACGTACGATGAAGGAGGAGGAAAGACTGGCAGAGGAGCCGTT |
| | | TCAGAAAAGGACGCTCCAAAGGAATTGTTGCAGATGCTCGAGAAGC |
| | | AGAAGAAG |
| 31 | Sso7d | MATVKFKYKGEEKEVDISKIKKVWRVGKMISFTYDEGGGKTGRGAV |
| | (amino | SEKDAPKELLQMLEKQKK |
| | acid) | |
| 32 | Aho7c | ATGGCGACTAAAGTGAAGTTCAAATATAAGGGTGAAGAAAAGGAAG |
| | (DNA) | TAGATATATCCAAGATCAAGAAGGTATGGCGGGTTGGAAAAATGAT |
| | | CAGCTTTACCTATGACGACAACGGAAAGACTGGTAGGGGTGCCGTA |
| | | TCTGAAAAAGATGCCCCCAAAGAATTGTTGGAAAAACTCAAG |
| 33 | Aho7c | MATKVKFKYKGEEKEVDISKIKKVWRVGKMISFTYDDNGKTGRGAV |
| | (amino | SEKDAPKELLEKLK |
| | acid) | |
| 34 | NLS1 | PKLKRQ |
| 35 | NLS2 | RPRK |
| 36 | NLS3 | RRARRPRG |
| 37 | NLS4 | GKRKLITSEEERSPAKRGRKS |
| 38 | NLS5 | KGKKGRTQKEKKAARARSKGKN |
| 39 | NLS6 | RKRCAAGVGGGPAGCPAPGSTPLKKPRR |
| 40 | NLS7 | RKPVTAQERQREREEKRRRRQERAKEREKRRQERER |
| 41 | NLS8 | RSGGNHRRNGRGGRGGYNRRNNGYHPY |
| 42 | NLS9 | TLLLRETMNNLGVSDHAVLSRKTPQPY |
| 43 | NLS10 | PGKMDKGEHRQERRDRPY |
| 44 | NLS11 | GKKKKGKPGKRREQRKKKRRT |
| 45 | NLS12 | SANKVTKNKSNSSPYLNKRKGKPGPDS |
| 46 | NLS13 | VHSHKKKKIPTSPTFTTPKTLTLRRQPKYPRKSAPRRNKLDHY |
| 47 | NLS14 | RKHKTNRKPR |
| 48 | NLS15 | NRRAKAKR |
| 49 | NLS16 | RNKKKK |
| 50 | NLS17 | RKVIK |
| 51 | NLS18 | PKKKRKV |
| 52 | NLS19 | KRPAATKKAGQAKKKK |
| 53 | UbC | GGCCTCCGCGCCGGGTTTTGGCGCCTCCCGCGGGCGCCCCCCTCCT |
| | promoter | CACGGCGAGCGCTGCCACGTCAGACGAAGGGCGCAGCGAGCGTCCT |
| | | GATCCTTCCGCCCGGACGCTCAGGACAGCGGCCCGCTGCTCATAAG |
| | | ACTCGGCCTTAGAACCCCAGTATCAGCAGAAGGACATTTTAGGACG |
| | | GGACTTGGGTGACTCTAGGGCACTGGTTTTCTTTCCAGAGAGCGGA |
| | | ACAGGCGAGGAAAAGTAGTCCCTTCTCGGCGATTCTGCGGAGGGAT |
| | | CTCCGTGGGGCGGTGAACGCCGATGATTATATAAGGACGCGCCGGG |
| | | TGTGGCACAGCTAGTTCCGTCGCAGCCGGGATTTGGGTCGCGGTTC |
| | | TTGTTTGTGGATCGCTGTGATCGTCACTTGGTGAGTAGCGGGCTGC |
| | | TGGGCTGGCCGGGGCTTTCGTGGCCGCCGGGCCGCTCGGTGGGACG |
| | | GAAGCGTGTGGAGAGACCGCCAAGGGCTGTAGTCTGGGTCCGCGAG |
| | | CAAGGTTGCCCTGAACTGGGGGTTGGGGGGAGCGCAGCAAAATGGC |
| | | GGCTGTTCCCGAGTCTTGAATGGAAGACGCTTGTGAGGCGGGCTGT |
| | | GAGGTCGTTGAAACAAGGTGGGGGGCATGGTGGGCGGCAAGAACCC |
| | | AAGGTCTTGAGGCCTTCGCTAATGCGGGAAAGCTCTTATTCGGGTG |
| | | AGATGGGCTGGGGCACCATCTGGGGACCCTGACGTGAAGTTTGTCA |
| | | CTGACTGGAGAACTCGGTTTGTCGTCTGTTGCGGGGGCGGCAGTTA |
| | | TGGCGGTGCCGTTGGGCAGTGCACCCGTACCTTTGGGAGCGCGCGC |
| | | CCTCGTCGTGTCGTGACGTCACCCGTTCTGTTGGCTTATAATGCAG |
| | | GGTGGGGCCACCTGCCGGTAGGTGTGCGGTAGGCTTTTCTCCGTCG |
| | | CAGGACGCAGGGTTCGGGCCTAGGGTAGGCTCTCCTGAATCGACAG |
| | | GCGCCGGACCTCTGGTGAGGGGAGGGATAAGTGAGGCGTCAGTTTC |
| | | TTTGGTCGGTTTTATGTACCTATCTTCTTAAGTAGCTGAAGCTCCG |
| | | GTTTTGAACTATGCGCTCGGGGTTGGCGAGTGTGTTTTGTGAAGTT |
| | | TTTTAGGCACCTTTTGAAATGTAATCATTTGGGTCAATATGTAATT |
| | | TTCAGTGTTAGACTAGTAAATTGTCCGCTAAATTCTGGCCGTTTTT |
| | | GGCTTTTTTGTTAGAC |
| 54 | CBh | Ccgttacataacttacggtaaatggcccgcctggctgaccgcccaa |
| | promoter | cgacccccgcccattgacgtcaatagtaacgccaatagggactttc |
| | | cattgacgtcaatgggtggagtatttacggtaaactgcccacttgg |
| | | cagtacatcaagtgtatcatatgccaagtacgccccctattgacgt |
| | | caatgacggtaaatggcccgcctggcattgtgcccagtacatgacc |
| | | ttatgggactttcctacttggcagtacatctacgtattagtcatcg |
| | | ctattaccatggtcgaggtgagccccacgttctgcttcactctccc |
| | | catctcccccccctccccacccccaattttgtatttatttattttt |
| | | taattattttgtgcagcgatgggggcggggggggggggggggcggg |
| | | gcgaggggcggggggggcgaggcggagaggtgcggcggcagccaat |
| | | cagagcggcgcgctccgaaagtttccttttatggcgaggggggggg |
| | | gggccctataaaaagcgaagcgcgcggggggggagtcgctgcgcgc |
| | | tgccttcgccccgtgccccgctcegccgccgcctcgcgccgcccgc |
| | | cccggctctgactgaccgcgttactcccacaggtgageggggggac |
| | | ggcccttctcctccgggctgtaattagctgagcaagaggtaagggt |
| | | ttaagggatggttggttggtggggtattaatgtttaattacctgga |
| | | gcacctgcctgaaatcactttttttcaggttgg |
| 55 | HA tag | YPYDVPDYA |
| 56 | T2A | EGRGSLLTCGDVEENPGP |
| | peptide | |
| 57 | HygR | MKKPELTATSVEKFLIEKFDSVSDLMQLSEGEESRAFSFDVGGRGY |
| | | VLRVNSCADGFYKDRYVYRHFASAALPIPEVLDIGEFSESLTYCIS |
| | | RRAQGVTLQDLPETELPAVLQPVAEAMDAIAAADLSQTSGFGPFGP |
| | | QGIGQYTTWRDFICAIADPHVYHWQTVMDDTVSASVAQALDELMLW |
| | | AEDCPEVRHLVHADFGSNNVLTDNGRITAVIDWSEAMFGDSQYEVA |
| | | NIFFWRPWLACMEQQTRYFERRHPELAGSPRLRAYMLRIGLDQLYQ |
| | | SLVDGNFDDAAWAQGRCDAIVRSGAGTVGRTQIARRSAAVWTDGCV |
| | | EVLADSGNRRPSTRPRAKE* |
| 58 | Linker | (GGGGS)ₙ** |
| | 1 | **wherein n is any integer, such as an integer |
| | | in the rage of 1-35 |
| 59 | Linker | (EAAK)ₙ*** |
| | 2 | ***wherein n is any integer, such as an integer |
| | | in the rage of 1-35 |
| 60 | T01 | ACACTCTTTCCCTACACGACGCTCTTCCGATCTCCGCTTTTATAAGG |
| | NGS | GCACTGAT |
| | forward | |
| | primer | |
| 61 | T02 | ACACTCTTTCCCTACACGACGCTCTTCCGATCTCTTTGTGGAACTCG |
| | NGS | CAA |
| | forward | |
| | primer | |
| 62 | T03 | ACACTCTTTCCCTACACGACGCTCTTCCGATCTCAGCTTGTGGATAA |
| | NGS | GAAAGGATT |
| | forward | |
| | primer | |
| 63 | T04 | ACACTCTTTCCCTACACGACGCTCTTCCGATCTGTTGGACACTGTAG |
| | NGS | AGTAAGTCAG |
| | forward | |
| | primer | |
| 64 | T05 | ACACTCTTTCCCTACACGACGCTCTTCCGATCTGGGAATAATCGAA |
| | NGS | ATGAAAGCAGGT |
| | forward | |
| | primer | |
| 65 | T06 | ACACTCTTTCCCTACACGACGCTCTTCCGATCTCAAAATCTGTGGGC |
| | NGS | TCCTG |
| | forward | |
| | primer | |
| 66 | T07 | ACACTCTTTCCCTACACGACGCTCTTCCGATCTCCGCTTTTATAAGG |
| | NGS | GCACTGAT |
| | forward | |
| | primer | |
| 67 | T08 | ACACTCTTTCCCTACACGACGCTCTTCCGATCTGATATAAGTTCCCA |
| | NGS | CTTTGAGCT |
| | forward | |
| | primer | |
| 68 | T09 | ACACTCTTTCCCTACACGACGCTCTTCCGATCTCGCTATGGAACTAT |
| | NGS | GCTAGC |
| | forward | |
| | primer | |
| 69 | T10 | ACACTCTTTCCCTACACGACGCTCTTCCGATCTCAATTACAGTGAAA |
| | NGS | CATGTTTCTCTCT |
| | forward | |
| | primer | |
| 70 | T11 | ACACTCTTTCCCTACACGACGCTCTTCCGATCTAGTATCTCACAATC |
| | NGS | TAGAAAGCACA |
| | forward | |
| | primer | |
| 71 | T12 | ACACTCTTTCCCTACACGACGCTCTTCCGATCTTTGACATGTGGACT |
| | NGS | GAAACC |
| | forward | |
| | primer | |
| 72 | T13 | ACACTCTTTCCCTACACGACGCTCTTCCGATCTCAGTCTCTGGCATT |
| | NGS | CCTTTATA |
| | forward | |
| | primer | |
| 73 | T14 | ACACTCTTTCCCTACACGACGCTCTTCCGATCTACAGTGAACATATG |
| | NGS | TGCTAACAAA |
| | forward | |
| | primer | |
| 74 | T15 | ACACTCTTTCCCTACACGACGCTCTTCCGATCTAGGGATTTATCTGG |
| | NGS | GAAAACA |
| | forward | |
| | primer | |
| 75 | T16 | ACACTCTTTCCCTACACGACGCTCTTCCGATCTGGTGCTTTGGAATT |
| | NGS | ATAATCCTCA |
| | forward | |
| | primer | |
| 76 | T17 | ACACTCTTTCCCTACACGACGCTCTTCCGATCTTATCACAGTCAACA |
| | NGS | CTAGAGGC |
| | forward | |
| | primer | |
| 77 | T18 | ACACTCTTTCCCTACACGACGCTCTTCCGATCTGAGAATTCATCATG |
| | NGS | GATTCCACTAG |
| | forward | |
| | primer | |
| 78 | T19 | ACACTCTTTCCCTACACGACGCTCTTCCGATCTGCCTTAAAGTCTTC |
| | NGS | TCCTCTTTCA |
| | forward | |
| | primer | |
| 79 | T20 | ACACTCTTTCCCTACACGACGCTCTTCCGATCTTGACTCCCTGAGGA |
| | NGS | AATCA |
| | forward | |
| | primer | |
| 80 | T21 | ACACTCTTTCCCTACACGACGCTCTTCCGATCTCCATATGCCAAAAG |
| | NGS | AGTCTATCA |
| | forward | |
| | primer | |
| 81 | T22 | ACACTCTTTCCCTACACGACGCTCTTCCGATCTTGCAGAATGGATGT |
| | NGS | TGTGTTAG |
| | forward | |
| | primer | |
| 82 | T23 | ACACTCTTTCCCTACACGACGCTCTTCCGATCTGCCAGAACACACA |
| | NGS | AAACATT |
| | forward | |
| | primer | |
| 83 | T24 | ACACTCTTTCCCTACACGACGCTCTTCCGATCTACGTAATAGTCTAC |
| | NGS | AGACCACATAT |
| | forward | |
| | primer | |
| 84 | T01 | GTGACTGGAGTTCAGACGTGTGCTCTTCCGATCTTCAAGTCATGCA |
| | NGS | CTGGATGTG |
| | reverse | |
| | primer | |
| 85 | T02 | GTGACTGGAGTTCAGACGTGTGCTCTTCCGATCTGATGGCAGAGCT |
| | NGS | AACAG |
| | reverse | |
| | primer | |
| 86 | T03 | GTGACTGGAGTTCAGACGTGTGCTCTTCCGATCTGATGAGACCTCT |
| | NGS | GAGAAGAAAAG |
| | reverse | |
| | primer | |
| 87 | T04 | GTGACTGGAGTTCAGACGTGTGCTCTTCCGATCTCCTTGCTTACTGC |
| | NGS | TTATTGCTAGTA |
| | reverse | |
| | primer | |
| 88 | T05 | GTGACTGGAGTTCAGACGTGTGCTCTTCCGATCTGGGTGCTTTAAC |
| | NGS | AACATAAGTCTGAT |
| | reverse | |
| | primer | |
| 89 | T06 | GTGACTGGAGTTCAGACGTGTGCTCTTCCGATCTGGAGTCACACGC |
| | NGS | CATTTTC |
| | reverse | |
| | primer | |
| 90 | T07 | GTGACTGGAGTTCAGACGTGTGCTCTTCCGATCTTCAAGTCATGCA |
| | NGS | CTGGATGTG |
| | reverse | |
| | primer | |
| 91 | T08 | GTGACTGGAGTTCAGACGTGTGCTCTTCCGATCTGAAATAATCCCA |
| | NGS | TTGTACGTTGA |
| | reverse | |
| | primer | |
| 92 | T09 | GTGACTGGAGTTCAGACGTGTGCTCTTCCGATCTCTTTTGCTAGACT |
| | NGS | CAGCTGTG |
| | reverse | |
| | primer | |
| 93 | T10 | GTGACTGGAGTTCAGACGTGTGCTCTTCCGATCTGCAGTTATCCTTG |
| | NGS | GTGAAAAATGATA |
| | reverse | |
| | primer | |
| 94 | T11 | GTGACTGGAGTTCAGACGTGTGCTCTTCCGATCTCAATTCAAATGG |
| | NGS | TCTTTTGGTTACAG |
| | reverse | |
| | primer | |
| | T12 | GTGACTGGAGTTCAGACGTGTGCTCTTCCGATCTGCAGATTGTTTCC |
| 95 | NGS | ATTGCATTC |
| | reverse | |
| | primer | |
| 96 | T13 | GTGACTGGAGTTCAGACGTGTGCTCTTCCGATCTTAGTCTGCATTGC |
| | NGS | TCTATGG |
| | reverse | |
| | primer | |
| 97 | T14 | GTGACTGGAGTTCAGACGTGTGCTCTTCCGATCTCCCTAGTTTTAGT |
| | NGS | CTAAACCAGT |
| | reverse | |
| | primer | |
| 98 | T15 | GTGACTGGAGTTCAGACGTGTGCTCTTCCGATCTAACCAGGCACTG |
| | NGS | TTAAAAGAT |
| | reverse | |
| | primer | |
| 99 | T16 | GTGACTGGAGTTCAGACGTGTGCTCTTCCGATCTCACAACGTGTAG |
| | NGS | ACCTGAAATAAA |
| | reverse | |
| | primer | |
| 100 | T17 | GTGACTGGAGTTCAGACGTGTGCTCTTCCGATCTGGCACCATTCCA |
| | NGS | AAGAATACA |
| | reverse | |
| | primer | |
| 101 | T18 | GTGACTGGAGTTCAGACGTGTGCTCTTCCGATCTGTAATTTTGTCTA |
| | NGS | AAAGCCTAAAATG |
| | reverse | |
| | primer | |
| 102 | T19 | GTGACTGGAGTTCAGACGTGTGCTCTTCCGATCTTACATTTGATTGG |
| | NGS | CATTTCCTCC |
| | reverse | |
| | primer | |
| 103 | T20 | GTGACTGGAGTTCAGACGTGTGCTCTTCCGATCTTGAATGAACAAA |
| | NGS | GCTGAGCT |
| | reverse | |
| | primer | |
| 104 | T21 | GTGACTGGAGTTCAGACGTGTGCTCTTCCGATCTCTCAGTATCTCCA |
| | NGS | TTTCACAGTG |
| | reverse | |
| | primer | |
| 105 | T22 | GTGACTGGAGTTCAGACGTGTGCTCTTCCGATCTGTTTGATGAACAT |
| | NGS | TTTGAGCCCT |
| | reverse | |
| | primer | |
| 106 | T23 | GTGACTGGAGTTCAGACGTGTGCTCTTCCGATCTCAGAGTGGTGTTT |
| | NGS | CCTAGG |
| | reverse | |
| | primer | |
| 107 | T24 | GTGACTGGAGTTCAGACGTGTGCTCTTCCGATCTTAAAGCTACTCCT |
| | NGS | TGTCCTGAAATTC |
| | reverse | |
| | primer | |
| 108 | DMD | ACAGAGCTAAGACAAGAACG |
| | AExon | |
| | 51 | |
| | forward | |
| 109 | DMD | GGCTTGAAAGCTGTTACAAG |
| 110 | AExon | MDKKYSIGLDIGTNSVGWAVITDEYKVPSKKFKVLGNTDRHSIKKN |
| | 51 | LIGALLFDSGETAEATRLKRTARRRYTRRKNRICYLQEIFSNEMAK |
| | reverse | VDDSFFHRLEESFLVEEDKKHERHPIFGNIVDEVAYHEKYPTIYHL |
| | SpCas9 | RKKLVDSTDKADLRLIYLALAHMIKFRGHFLIEGDLNPDNSDVDKL |
| | (amino | FIQLVQTYNQLFEENPINASGVDAKAILSARLSKSRRLENLIAQLP |
| | acid) | GEKKNGLFGNLIALSLGLTPNFKSNFDLAEDAKLQLSKDTYDDDLD |
| | | NLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQI |
| | | HLGELHAILRRQEDFYPFLKDNREKIEKILTFRIPYYVGPLARGNS |
| | | RFAWMTRKSEETITPWNFEEVVDKGASAQSFIERMTNFDKNLPNEK |
| | | VLPKHSLLYEYFTVYNELTKVKYVTEGMRKPAFLSGEQKKAIVDLL |
| | | FKTNRKVTVKQLKEDYFKKIECFDSVEISGVEDRFNASLGTYHDLL |
| | | KIIKDKDFLDNEENEDILEDIVLTLTLFEDREMIEERLKTYAHLFD |
| | | DKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKG |
| | | ILQTVKVVDELVKVMGRHKPENIVIEMARENQTTQKGQKNSRERMK |
| | | RIEEGIKELGSQILKEHPVENTQLQNEKLYLYYLQNGRDMYVDQEL |
| | | DINRLSDYDVDHIVPQSFLKDDSIDNKVLTRSDKNRGKSDNVPSEE |
| | | VVKKMKNYWRQLLNAKLITQRKFDNLTKAERGGLSELDKAGFIKRQ |
| | | LVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIR |
| | | KRPLIETNGETGEIVWDKGRDFATVRKVLSMPQVNIVKKTEVQTGG |
| | | FSKESILPKRNSDKLIARKKDWDPKKYGGFDSPTVAYSVLVVAKVE |
| | | KGKSKKLKSVKELLGITIMERSSFEKNPIDFLEAKGYKEVKKDLII |
| | | KLPKYSLFELENGRKRMLASAGELQKGNELALPSKYVNFLYLASHY |
| | | EKLKGSPEDNEQKQLFVEQHKHYLDEIIEQISEFSKRVILADANLD |
| | | KVLSAYNKHRDKPIREQAENIIHLFTLTNLGAPAAFKYFDTTIDRK |
| | | RYTSTKEVLDATLIHQSITGLYETRIDLSQLGGD |
| 111 | SaCas9 | MKRNYILGLDIGITSVGYGIIDYETRDVIDAGVRLFKEANVENNEG |
| | (amino | RRSKRGARRLKRRRRHRIQRVKKLLFDYNLLTDHSELSGINPYEAR |
| | acid) | VKGLSQKLSEEEFSAALLHLAKRRGVHNVNEVEEDTGNELSTKEQI |
| | | SRNSKALEEKYVAELQLERLKKDGEVRGSINRFKTSDYVKEAKQLL |
| | | KVQKAYHQLDQSFIDTYIDLLETRRTYYEGPGEGSPFGWKDIKEWY |
| | | EMLMGHCTYFPEELRSVKYAYNADLYNALNDLNNLVITRDENEKLE |
| | | YYEKFQIIENVFKQKKKPTLKQIAKEILVNEEDIKGYRVTSTGKPE |
| | | FTNLKVYHDIKDITARKEIIENAELLDQIAKILTIYQSSEDIQEEL |
| | | TNLNSELTQEEIEQISNLKGYTGTHNLSLKAINLILDELWHTNDNQ |
| | | IAIFNRLKLVPKKVDLSQQKEIPTTLVDDFILSPVVKRSFIQSIKV |
| | | INAIIKKYGLPNDIIIELAREKNSKDAQKMINEMQKRNRQTNERIE |
| | | EIIRTTGKENAKYLIEKIKLHDMQEGKCLYSLEAIPLEDLLNNPFN |
| | | YEVDHIIPRSVSFDNSFNNKVLVKQEENSKKGNRTPFQYLSSSDSK |
| | | ISYETFKKHILNLAKGKGRISKTKKEYLLEERDINRFSVQKDFINR |
| | | NLVDTRYATRGLMNLLRSYFRVNNLDVKVKSINGGFTSFLRRKWKF |
| | | KKERNKGYKHHAEDALIIANADFIFKEWKKLDKAKKVMENQMFEEK |
| | | QAESMPEIETEQEYKEIFITPHQIKHIKDFKDYKYSHRVDKKPNRE |
| | | LINDTLYSTRKDDKGNTLIVNNLNGLYDKDNDKLKKLINKSPEKLL |
| | | MYHHDPQTYQKLKLIMEQYGDEKNPLYKYYEETGNYLTKYSKKDNG |
| | | PVIKKIKYYGNKLNAHLDITDDYPNSRNKVVKLSLKPYRFDVYLDN |
| | | GVYKFVTVKNLDVIKKENYYEVNSKCYEEAKKLKKISNQAEFIASF |
| | | YNNDLIKINGELYRVIGVNNDLLNRIEVNMIDITYREYLENMNDKR |
| | | PPRIIKTIASKTQSIKKYSTDILGNLYEVKSKKHPQIIKKG |
| 112 | Cas12f | MAKNTITKTLKLRIVRPYNSAEVEKIVADEKNNREKIALEKNKDKV |
| | 1 | KEACSKHLKVAAYCTTQVERNACLFCKARKLDDKFYQKLRGQFPDA |
| | (amino | VFWQEISEIFRQLQKQAAEIYNQSLIELYYEIFIKGKGIANASSVE |
| | acid) | HYLSDVCYTRAAELFKNAAIASGLRSKIKSNFRLKELKNMKSGLPT |
| | | TKSDNFPIPLVKQKGGQYTGFEISNHNSDFIIKIPFGRWQVKKEID |
| | | KYRPWEKFDFEQVQKSPKPISLLLSTQRRKRNKGWSKDEGTEAEIK |
| | | KVMNGDYQTSYIEVKRGSKIGEKSAWMLNLSIDVPKIDKGVDPSII |
| | | GGIDVGVKSPLVCAINNAFSRYSISDNDLFHFNKKMFARRRILLKK |
| | | NRHKRAGHGAKNKLKPITILTEKSERFRKKLIERWACEIADFFIKN |
| | | KVGTVQMENLESMKRKEDSYFNIRLRGFWPYAEMQNKIEFKLKQYG |
| | | IEIRKVAPNNTSKTCSKCGHLNNYFNFEYRKKNKFPHFKCEKCNFK |
| | | ENADYNAALNISNPKLKSTKEEP |
| 113 | SpCas9 | atggacaagaagtacagcatcggcctggacatcggcaccaactctg |
| | (DNA) | tgggctgggccgtgatcaccgacgagtacaaggtgcccagcaagaa |
| | | attcaaggtgctgggcaacaccgaccggcacagcatcaagaagaac |
| | | ctgatcggagccctgctgttcgacagcggcgaaacagccgaggcca |
| | | cccggctgaagagaaccgccagaagaagatacaccagacggaagaa |
| | | ccggatctgctatctgcaagagatcttcagcaacgagatggccaag |
| | | gtggacgacagcttcttccacagactggaagagtccttcctggtgg |
| | | aagaggataagaagcacgagcggcaccccatcttcggcaacatcgt |
| | | ggacgaggtggcctaccacgagaagtaccccaccatctaccacctg |
| | | agaaagaaactggtggacagcaccgacaaggccgacctgcggctga |
| | | tctatctggccctggcccacatgatcaagttccggggccacttcct |
| | | gatcgagggcgacctgaaccccgacaacagcgacgtggacaagctg |
| | | ttcatccagctggtgcagacctacaaccagctgttcgaggaaaacc |
| | | ccatcaacgccagcggcgtggacgccaaggccatcctgtctgccag |
| | | actgagcaagagcagacggctggaaaatctgatcgcccagctgccc |
| | | ggcgagaagaagaatggcctgttcggaaacctgattgccctgagcc |
| | | tgggcctgacccccaacttcaagagcaacttcgacctggccgagga |
| | | tgccaaactgcagctgagcaaggacacctacgacgacgacctggac |
| | | aacctgctggcccagatcggcgaccagtacgccgacctgtttctgg |
| | | ccgccaagaacctgtccgacgccatcctgctgagcgacatcctgag |
| | | agtgaacaccgagatcaccaaggcccccctgagcgcctctatgatc |
| | | aagagatacgacgagcaccaccaggacctgaccctgctgaaagctc |
| | | tcgtgcggcagcagctgcctgagaagtacaaagagattttcttcga |
| | | ccagagcaagaacggctacgccggctacattgacggcggagccagc |
| | | caggaagagttctacaagttcatcaagcccatcctggaaaagatgg |
| | | acggcaccgaggaactgctcgtgaagctgaacagagaggacctgct |
| | | gcggaagcagcggaccttcgacaacggcagcatcccccaccagatc |
| | | cacctgggagagctgcacgccattctgcggcggcaggaagattttt |
| | | acccattcctgaaggacaaccgggaaaagatcgagaagatcctgac |
| | | cttccgcatcccctactacgtgggccctctggccaggggaaacagc |
| | | agattcgcctggatgaccagaaagagcgaggaaaccatcaccccct |
| | | ggaacttcgaggaagtggtggacaagggcgcttccgcccagagctt |
| | | catcgagcggatgaccaacttcgataagaacctgcccaacgagaag |
| | | gtgctgcccaagcacagcctgctgtacgagtacttcaccgtgtata |
| | | acgagctgaccaaagtgaaatacgtgaccgagggaatgagaaagcc |
| | | cgccttcctgagcggcgagcagaaaaaggccatcgtggacctgctg |
| | | ttcaagaccaaccggaaagtgaccgtgaagcagctgaaagaggact |
| | | acttcaagaaaatcgagtgcttcgactccgtggaaatctccggcgt |
| | | ggaagatcggttcaacgcctccctgggcacataccacgatctgctg |
| | | aaaattatcaaggacaaggacttcctggacaatgaggaaaacgagg |
| | | acattctggaagatatcgtgctgaccctgacactgtttgaggacag |
| | | agagatgatcgaggaacggctgaaaacctatgcccacctgttcgac |
| | | gacaaagtgatgaagcagctgaagcggcggagatacaccggctggg |
| | | gcaggctgagccggaagctgatcaacggcatccgggacaagcagtc |
| | | cggcaagacaatcctggatttcctgaagtccgacggcttcgccaac |
| | | agaaacttcatgcagctgatccacgacgacagcctgacctttaaag |
| | | aggacatccagaaagcccaggtgtccggccagggcgatagcctgca |
| | | cgagcacattgccaatctggccggcagccccgccattaagaagggc |
| | | atcctgcagacagtgaaggtggtggacgagctcgtgaaagtgatgg |
| | | gccggcacaagcccgagaacatcgtgatcgaaatggccagagagaa |
| | | ccagaccacccagaagggacagaagaacagccgcgagagaatgaag |
| | | cggatcgaagagggcatcaaagagctgggcagccagatcctgaaag |
| | | aacaccccgtggaaaacacccagctgcagaacgagaagctgtacct |
| | | gtactacctgcagaatgggcgggatatgtacgtggaccaggaactg |
| | | gacatcaaccggctgtccgactacgatgtggaccatatcgtgcctc |
| | | agagctttctgaaggacgactccatcgacaacaaggtgctgaccag |
| | | aagcgacaagaaccggggcaagagcgacaacgtgccctccgaagag |
| | | gtcgtgaagaagatgaagaactactggcggcagctgctgaacgcca |
| | | agctgattacccagagaaagttcgacaatctgaccaaggccgagag |
| | | aggcggcctgagcgaactggataaggccggcttcatcaagagacag |
| | | ctggtggaaacccggcagatcacaaagcacgtggcacagatcctgg |
| | | actcccggatgaacactaagtacgacgagaatgacaagctgatccg |
| | | ggaagtgaaagtgatcaccctgaagtccaagctggtgtccgatttc |
| | | cggaaggatttccagttttacaaagtgcgcgagatcaacaactacc |
| | | accacgcccacgacgcctacctgaacgccgtcgtgggaaccgccct |
| | | gatcaaaaagtaccctaagctggaaagcgagttcgtgtacggcgac |
| | | tacaaggtgtacgacgtgcggaagatgatcgccaagagcgagcagg |
| | | aaatcggcaaggctaccgccaagtacttcttctacagcaacatcat |
| | | gaactttttcaagaccgagattaccctggccaacggcgagatccgg |
| | | aageggcctctgatcgagacaaacggcgaaaccggggagatcgtgt |
| | | gggataagggccgggattttgccaccgtgcggaaagtgctgagcat |
| | | gccccaagtgaatatcgtgaaaaagaccgaggtgcagacaggcggc |
| | | ttcagcaaagagtctatcctgcccaagaggaacagcgataagctga |
| | | tcgccagaaagaaggactgggaccctaagaagtacggcggcttcga |
| | | cagccccaccgtggcctattctgtgctggtggtggccaaagtggaa |
| | | aagggcaagtccaagaaactgaagagtgtgaaagagctgctgggga |
| | | tcaccatcatggaaagaagcagcttcgagaagaatcccatcgactt |
| | | tctggaagccaagggctacaaagaagtgaaaaaggacctgatcatc |
| | | aagctgcctaagtactccctgttcgagctggaaaacggccggaaga |
| | | gaatgctggcctctgccggcgaactgcagaagggaaacgaactggc |
| | | cctgccctccaaatatgtgaacttcctgtacctggccagccactat |
| | | gagaagctgaagggctcccccgaggataatgagcagaaacagctgt |
| | | ttgtggaacagcacaagcactacctggacgagatcatcgagcagat |
| | | cagcgagttctccaagagagtgatcctggccgacgctaatctggac |
| | | aaagtgctgtccgcctacaacaagcaccgggataagcccatcagag |
| | | agcaggccgagaatatcatccacctgtttaccctgaccaatctggg |
| | | agcccctgccgccttcaagtactttgacaccaccatcgaccggaag |
| | | aggtacaccagcaccaaagaggtgctggacgccaccctgatccacc |
| | | agagcatcaccggcctgtacgagacacggatcgacctgtctcagct |
| | | gggaggcgac |
| 114 | SaCas9 | atgaagcggaactacatcctgggcctggacatcggcatcaccagcg |
| | (DNA) | tgggctacggcatcatcgactacgagacacgggacgtgatcgatgc |
| | | cggcgtgcggctgttcaaagaggccaacgtggaaaacaacgagggc |
| | | aggcggagcaagagaggcgccagaaggctgaagcggcggaggcggc |
| | | atagaatccagagagtgaagaagctgctgttcgactacaacctgct |
| | | gaccgaccacagcgagctgagcggcatcaacccctacgaggccaga |
| | | gtgaagggcctgagccagaagctgagcgaggaagagttctctgccg |
| | | ccctgctgcacctggccaagagaagaggcgtgcacaacgtgaacga |
| | | ggtggaagaggacaccggcaacgagctgtccaccaaagagcagatc |
| | | agccggaacagcaaggccctggaagagaaatacgtggccgaactgc |
| | | agctggaacggctgaagaaagacggcgaagtgcggggcagcatcaa |
| | | cagattcaagaccagcgactacgtgaaagaagccaaacagctgctg |
| | | aaggtgcagaaggcctaccaccagctggaccagagcttcatcgaca |
| | | cctacatcgacctgctggaaacccggcggacctactatgagggacc |
| | | tggcgagggcagccccttcggctggaaggacatcaaagaatggtac |
| | | gagatgctgatgggccactgcacctacttccccgaggaactgcgga |
| | | gcgtgaagtacgcctacaacgccgacctgtacaacgccctgaacga |
| | | cctgaacaatctcgtgatcaccagggacgagaacgagaagctggaa |
| | | tattacgagaagttccagatcatcgagaacgtgttcaagcagaaga |
| | | agaagcccaccctgaagcagatcgccaaagaaatcctcgtgaacga |
| | | agaggatattaagggctacagagtgaccagcaccggcaagcccgag |
| | | ttcaccaacctgaaggtgtaccacgacatcaaggacattaccgccc |
| | | ggaaagagattattgagaacgccgagctgctggatcagattgccaa |
| | | gatcctgaccatctaccagagcagcgaggacatccaggaagaactg |
| | | accaatctgaactccgagctgacccaggaagagatcgagcagatct |
| | | ctaatctgaagggctataccggcacccacaacctgagcctgaaggc |
| | | catcaacctgatcctggacgagctgtggcacaccaacgacaaccag |
| | | atcgctatcttcaaccggctgaagctggtgcccaagaaggtggacc |
| | | tgtcccagcagaaagagatccccaccaccctggtggacgacttcat |
| | | cctgagccccgtcgtgaagagaagcttcatccagagcatcaaagtg |
| | | atcaacgccatcatcaagaagtacggcctgcccaacgacatcatta |
| | | tcgagctggcccgcgagaagaactccaaggacgcccagaaaatgat |
| | | caacgagatgcagaagcggaaccggcagaccaacgagcggatcgag |
| | | gaaatcatccggaccaccggcaaagagaacgccaagtacctgatcg |
| | | agaagatcaagctgcacgacatgcaggaaggcaagtgcctgtacag |
| | | cctggaagccatccctctggaagatctgctgaacaaccccttcaac |
| | | tatgaggtggaccacatcatccccagaagcgtgtccttcgacaaca |
| | | gcttcaacaacaaggtgctcgtgaagcaggaagaaaacagcaagaa |
| | | gggcaaccggaccccattccagtacctgagcagcagcgacagcaag |
| | | atcagctacgaaaccttcaagaagcacatcctgaatctggccaagg |
| | | gcaagggcagaatcagcaagaccaagaaagagtatctgctggaaga |
| | | acgggacatcaacaggttctccgtgcagaaagacttcatcaaccgg |
| | | aacctggtggataccagatacgccaccagaggcctgatgaacctgc |
| | | tgcggagctacttcagagtgaacaacctggacgtgaaagtgaagtc |
| | | catcaatggcggcttcaccagctttctgcggcggaagtggaagttt |
| | | aagaaagagcggaacaaggggtacaagcaccacgccgaggacgccc |
| | | tgatcattgccaacgccgatttcatcttcaaagagtggaagaaact |
| | | ggacaaggccaaaaaagtgatggaaaaccagatgttcgaggaaaag |
| | | caggccgagagcatgcccgagategaaaccgagcaggagtacaaag |
| | | agatcttcatcaccccccaccagatcaagcacattaaggacttcaa |
| | | ggactacaagtacagccaccgggtggacaagaagcctaatagagag |
| | | ctgattaacgacaccctgtactccacccggaaggacgacaagggca |
| | | acaccctgatcgtgaacaatctgaacggcctgtacgacaaggacaa |
| | | tgacaagctgaaaaagctgatcaacaagagccccgaaaagctgctg |
| | | atgtaccaccacgacccccagacctaccagaaactgaagctgatta |
| | | tggaacagtacggcgacgagaagaatcccctgtacaagtactacga |
| | | ggaaaccgggaactacctgaccaagtactccaaaaaggacaacggc |
| | | cccgtgatcaagaagattaagtattacggcaacaaactgaacgccc |
| | | atctggacatcaccgacgactaccccaacagcagaaacaaggtcgt |
| | | gaagctgtccctgaagccctacagattcgacgtgtacctggacaat |
| | | ggcgtgtacaagttcgtgaccgtgaagaatctggatgtgatcaaaa |
| | | aagaaaactactacgaagtgaatagcaagtgctatgaggaagctaa |
| | | gaagctgaagaagatcagcaaccaggccgagtttatcgcctccttc |
| | | tacaacaacgatctgatcaagatcaacggcgagctgtatagagtga |
| | | tcggcgtgaacaacgacctgctgaaccggatcgaagtgaacatgat |
| | | cgacatcacctaccgcgagtacctggaaaacatgaacgacaagagg |
| | | ccccccaggatcattaagacaatcgcctccaagacccagagcatta |
| | | agaagtacagcacagacattctgggcaacctgtatgaagtgaaatc |
| | | taagaagcaccctcagatcatcaaaaagggc |
| 115 | Cas12f | ATGGCCAAGAACACAATTACAAAGACACTGAAGCTGAGGATCGTGA |
| | 1 | GACCATACAACAGCGCTGAGGTCGAGAAGATTGTGGCTGATGAAAA |
| | (DNA) | GAACAACAGGGAAAAGATCGCCCTCGAGAAGAACAAGGATAAGGTG |
| | | AAGGAGGCCTGCTCTAAGCACCTGAAAGTGGCCGCCTACTGCACCA |
| | | CACAGGTGGAGAGGAACGCCTGTCTGTTTTGTAAAGCTCGGAAGCT |
| | | GGATGATAAGTTTTACCAGAAGCTGCGGGGCCAGTTCCCCGATGCC |
| | | GTCTTTTGGCAGGAGATTAGCGAGATCTTCAGACAGCTGCAGAAGC |
| | | AGGCCGCCGAGATCTACAACCAGAGCCTGATCGAGCTCTACTACGA |
| | | GATCTTCATCAAGGGCAAGGGCATTGCCAACGCCTCCTCCGTGGAG |
| | | CACTACCTGAGCGACGTGTGCTACACAAGAGCCGCCGAGCTCTTTA |
| | | AGAACGCCGCTATCGCTTCCGGGCTGAGGAGCAAGATTAAGAGTAA |
| | | CTTCCGGCTCAAGGAGCTGAAGAACATGAAGAGCGGCCTGCCCACT |
| | | ACAAAGAGCGACAACTTCCCAATTCCACTGGTGAAGCAGAAGGGGG |
| | | GCCAGTACACAGGGTTCGAGATTTCCAACCACAACAGCGACTTTAT |
| | | TATTAAGATCCCCTTTGGCAGGTGGCAGGTCAAGAAGGAGATTGAC |
| | | AAGTACAGGCCCTGGGAGAAGTTTGATTTCGAGCAGGTGCAGAAGA |
| | | GCCCCAAGCCTATTTCCCTGCTGCTGTCCACACAGCGGCGGAAGAG |
| | | GAACAAGGGGTGGTCTAAGGATGAGGGGACCGAGGCCGAGATTAAG |
| | | AAAGTGATGAACGGCGACTACCAGACAAGCTACATCGAGGTCAAGC |
| | | GGGGCAGTAAGATTGGCGAGAAGAGCGCCTGGATGCTGAACCTGAG |
| | | CATTGACGTGCCAAAGATTGATAAGGGCGTGGATCCCAGCATCATC |
| | | GGAGGGATCGATGTGGGGGTCAAGAGCCCCCTCGTGTGCGCCATCA |
| | | ACAACGCCTTCAGCAGGTACAGCATCTCCGATAACGACCTGTTCCA |
| | | CTTTAACAAGAAGATGTTCGCCCGGCGGAGGATTTTGCTCAAGAAG |
| | | AACCGGCACAAGCGGGCCGGACACGGGGCCAAGAACAAGCTCAAGC |
| | | CCATCACTATCCTGACCGAGAAGAGCGAGAGGTTCAGGAAGAAGCT |
| | | CATCGAGAGATGGGCCTGCGAGATCGCCGATTTCTTTATTAAGAAC |
| | | AAGGTCGGAACAGTGCAGATGGAGAACCTCGAGAGCATGAAGAGGA |
| | | AGGAGGATTCCTACTTCAACATTCGGCTGAGGGGGTTCTGGCCCTA |
| | | CGCTGAGATGCAGAACAAGATTGAGTTTAAGCTGAAGCAGTACGGG |
| | | ATTGAGATCCGGAAGGTGGCCCCCAACAACACCAGCAAGACCTGCA |
| | | GCAAGTGCGGGCACCTCAACAACTACTTCAACTTCGAGTACCGGAA |
| | | GAAGAACAAGTTCCCACACTTCAAGTGCGAGAAGTGCAACTTTAAG |
| | | GAGAACGCCGATTACAACGCCGCCCTGAACATCAGCAACCCTAAGC |
| | | TGAAGAGCACTAAGGAGGAGCCC |
| 116 | pLV- | GTCGACGGATCGGGAGATCTCCCGATCCCCTATGGTGCACTCTCAG |
| | EW15 | TACAATCTGCTCTGATGCCGCATAGTTAAGCCAGTATCTGCTCCCT |
| | (contains | GCTTGTGTGTTGGAGGTCGCTGAGTAGTGCGCGAGCAAAATTTAAG |
| | UbC: | CTACAACAAGGCAAGGCTTGACCGACAATTGCATGAAGAATCTGCT |
| | Cas12f1- | TAGGGTTAGGCGTTTTGCGCTGCTTCGCGATGTACGGGCCAGATAT |
| | Aho7c | ACGCGTTGACATTGATTATTGACTAGTTATTAATAGTAATCAATTA |
| | expression | CGGGGTCATTAGTTCATAGCCCATATATGGAGTTCCGCGTTACATA |
| | cassette) | ACTTACGGTAAATGGCCCGCCTGGCTGACCGCCCAACGACCCCCGC |
| | | CCATTGACGTCAATAATGACGTATGTTCCCATAGTAACGCCAATAG |
| | | GGACTTTCCATTGACGTCAATGGGTGGAGTATTTACGGTAAACTGC |
| | | CCACTTGGCAGTACATCAAGTGTATCATATGCCAAGTACGCCCCCT |
| | | ATTGACGTCAATGACGGTAAATGGCCCGCCTGGCATTATGCCCAGT |
| | | ACATGACCTTATGGGACTTTCCTACTTGGCAGTACATCTACGTATT |
| | | AGTCATCGCTATTACCATGGTGATGCGGTTTTGGCAGTACATCAAT |
| | | GGGCGTGGATAGCGGTTTGACTCACGGGGATTTCCAAGTCTCCACC |
| | | CCATTGACGTCAATGGGAGTTTGTTTTGGCACCAAAATCAACGGGA |
| | | CTTTCCAAAATGTCGTAACAACTCCGCCCCATTGACGCAAATGGGC |
| | | GGTAGGCGTGTACGGTGGGAGGTCTATATAAGCAGCGCGTTTTGCC |
| | | TGTACTGGGTCTCTCTGGTTAGACCAGATCTGAGCCTGGGAGCTCT |
| | | CTGGCTAACTAGGGAACCCACTGCTTAAGCCTCAATAAAGCTTGCC |
| | | TTGAGTGCTTCAAGTAGTGTGTGCCCGTCTGTTGTGTGACTCTGGT |
| | | AACTAGAGATCCCTCAGACCCTTTTAGTCAGTGTGGAAAATCTCTA |
| | | GCAGTGGCGCCCGAACAGGGACTTGAAAGCGAAAGGGAAACCAGAG |
| | | GAGCTCTCTCGACGCAGGACTCGGCTTGCTGAAGCGCGCACGGCAA |
| | | GAGGCGAGGGGCGGCGACTGGTGAGTACGCCAAAAATTTTGACTAG |
| | | CGGAGGCTAGAAGGAGAGAGATGGGTGCGAGAGCGTCAGTATTAAG |
| | | CGGGGGAGAATTAGATCGCGATGGGAAAAAATTCGGTTAAGGCCAG |
| | | GGGGAAAGAAAAAATATAAATTAAAACATATAGTATGGGCAAGCAG |
| | | GGAGCTAGAACGATTCGCAGTTAATCCTGGCCTGTTAGAAACATCA |
| | | GAAGGCTGTAGACAAATACTGGGACAGCTACAACCATCCCTTCAGA |
| | | CAGGATCAGAAGAACTTAGATCATTATATAATACAGTAGCAACCCT |
| | | CTATTGTGTGCATCAAAGGATAGAGATAAAAGACACCAAGGAAGCT |
| | | TTAGACAAGATAGAGGAAGAGCAAAACAAAAGTAAGACCACCGCAC |
| | | AGCAAGCGGCCGCTGATCTTCAGACCTGGAGGAGGAGATATGAGGG |
| | | ACAATTGGAGAAGTGAATTATATAAATATAAAGTAGTAAAAATTGA |
| | | ACCATTAGGAGTAGCACCCACCAAGGCAAAGAGAAGAGTGGTGCAG |
| | | AGAGAAAAAAGAGCAGTGGGAATAGGAGCTTTGTTCCTTGGGTTCT |
| | | TGGGAGCAGCAGGAAGCACTATGGGCGCAGCGTCAATGACGCTGAC |
| | | GGTACAGGCCAGACAATTATTGTCTGGTATAGTGCAGCAGCAGAAC |
| | | AATTTGCTGAGGGCTATTGAGGCGCAACAGCATCTGTTGCAACTCA |
| | | CAGTCTGGGGCATCAAGCAGCTCCAGGCAAGAATCCTGGCTGTGGA |
| | | AAGATACCTAAAGGATCAACAGCTCCTGGGGATTTGGGGTTGCTCT |
| | | GGAAAACTCATTTGCACCACTGCTGTGCCTTGGAATGCTAGTTGGA |
| | | GTAATAAATCTCTGGAACAGATTTGGAATCACACGACCTGGATGGA |
| | | GTGGGACAGAGAAATTAACAATTACACAAGCTTAATACACTCCTTA |
| | | ATTGAAGAATCGCAAAACCAGCAAGAAAAGAATGAACAAGAATTAT |
| | | TGGAATTAGATAAATGGGCAAGTTTGTGGAATTGGTTTAACATAAC |
| | | AAATTGGCTGTGGTATATAAAATTATTCATAATGATAGTAGGAGGC |
| | | TTGGTAGGTTTAAGAATAGTTTTTGCTGTACTTTCTATAGTGAATA |
| | | GAGTTAGGCAGGGATATTCACCATTATCGTTTCAGACCCACCTCCC |
| | | AACCCCGAGGGGACCCGACAGGCCCGAAGGAATAGAAGAAGAAGGT |
| | | GGAGAGAGAGACAGAGACAGATCCATTCGATTAGTGAACGGATCGG |
| | | CACTGCGTGCGCCAATTCTGCAGACAAATGGCAGTATTCATCCACA |
| | | ATTTTAAAAGAAAAGGGGGGATTGGGGGGTACAGTGCAGGGGAAAG |
| | | AATAGTAGACATAATAGCAACAGACATACAAACTAAAGAATTACAA |
| | | AAACAAATTACAAAAATTCAAAATTTTCGGGTTTATTACAGGGACA |
| | | GCAGAGATCCAGTTTGGGCTAGCTTAATTAACCTGCAGGCCCGTGT |
| | | CGGCTCCAGATCTGGCCTCCGCGCCGGGTTTTGGCGCCTCCCGCGG |
| | | GCGCCCCCCTCCTCACGGCGAGCGCTGCCACGTCAGACGAAGGGCG |
| | | CAGCGAGCGTCCTGATCCTTCCGCCCGGACGCTCAGGACAGCGGCC |
| | | CGCTGCTCATAAGACTCGGCCTTAGAACCCCAGTATCAGCAGAAGG |
| | | ACATTTTAGGACGGGACTTGGGTGACTCTAGGGCACTGGTTTTCTT |
| | | TCCAGAGAGCGGAACAGGCGAGGAAAAGTAGTCCCTTCTCGGCGAT |
| | | TCTGCGGAGGGATCTCCGTGGGGCGGTGAACGCCGATGATTATATA |
| | | AGGACGCGCCGGGTGTGGCACAGCTAGTTCCGTCGCAGCCGGGATT |
| | | TGGGTCGCGGTTCTTGTTTGTGGATCGCTGTGATCGTCACTTGGTG |
| | | AGTAGCGGGCTGCTGGGCTGGCCGGGGCTTTCGTGGCCGCCGGGCC |
| | | GCTCGGTGGGACGGAAGCGTGTGGAGAGACCGCCAAGGGCTGTAGT |
| | | CTGGGTCCGCGAGCAAGGTTGCCCTGAACTGGGGGTTGGGGGGAGC |
| | | GCAGCAAAATGGCGGCTGTTCCCGAGTCTTGAATGGAAGACGCTTG |
| | | TGAGGCGGGCTGTGAGGTCGTTGAAACAAGGTGGGGGGCATGGTGG |
| | | GCGGCAAGAACCCAAGGTCTTGAGGCCTTCGCTAATGCGGGAAAGC |
| | | TCTTATTCGGGTGAGATGGGCTGGGGCACCATCTGGGGACCCTGAC |
| | | GTGAAGTTTGTCACTGACTGGAGAACTCGGTTTGTCGTCTGTTGCG |
| | | GGGGCGGCAGTTATGGCGGTGCCGTTGGGCAGTGCACCCGTACCTT |
| | | TGGGAGCGCGCGCCCTCGTCGTGTCGTGACGTCACCCGTTCTGTTG |
| | | GCTTATAATGCAGGGTGGGGCCACCTGCCGGTAGGTGTGCGGTAGG |
| | | CTTTTCTCCGTCGCAGGACGCAGGGTTCGGGCCTAGGGTAGGCTCT |
| | | CCTGAATCGACAGGCGCCGGACCTCTGGTGAGGGGAGGGATAAGTG |
| | | AGGCGTCAGTTTCTTTGGTCGGTTTTATGTACCTATCTTCTTAAGT |
| | | AGCTGAAGCTCCGGTTTTGAACTATGCGCTCGGGGTTGGCGAGTGT |
| | | GTTTTGTGAAGTTTTTTAGGCACCTTTTGAAATGTAATCATTTGGG |
| | | TCAATATGTAATTTTCAGTGTTAGACTAGTAAATTGTCCGCTAAAT |
| | | TCTGGCCGTTTTTGGCTTTTTTGTTAGACGAAGCTTGGGCTGCAGG |
| | | TCGACTCTAGGCCCAGGCGGCCTTGTACAAAAAAGCACCGGTAGGC |
| | | TGCCACCATGCCAAAGAAGAAGCGGAAGGTCGGTATCCACGGAGTC |
| | | CCAGCAGCCATGGCCAAGAACACAATTACAAAGACACTGAAGCTGA |
| | | GGATCGTGAGACCATACAACAGCGCTGAGGTCGAGAAGATTGTGGC |
| | | TGATGAAAAGAACAACAGGGAAAAGATCGCCCTCGAGAAGAACAAG |
| | | GATAAGGTGAAGGAGGCCTGCTCTAAGCACCTGAAAGTGGCCGCCT |
| | | ACTGCACCACACAGGTGGAGAGGAACGCCTGTCTGTTTTGTAAAGC |
| | | TCGGAAGCTGGATGATAAGTTTTACCAGAAGCTGCGGGGCCAGTTC |
| | | CCCGATGCCGTCTTTTGGCAGGAGATTAGCGAGATCTTCAGACAGC |
| | | TGCAGAAGCAGGCCGCCGAGATCTACAACCAGAGCCTGATCGAGCT |
| | | CTACTACGAGATCTTCATCAAGGGCAAGGGCATTGCCAACGCCTCC |
| | | TCCGTGGAGCACTACCTGAGCGACGTGTGCTACACAAGAGCCGCCG |
| | | AGCTCTTTAAGAACGCCGCTATCGCTTCCGGGCTGAGGAGCAAGAT |
| | | TAAGAGTAACTTCCGGCTCAAGGAGCTGAAGAACATGAAGAGCGGC |
| | | CTGCCCACTACAAAGAGCGACAACTTCCCAATTCCACTGGTGAAGC |
| | | AGAAGGGGGGCCAGTACACAGGGTTCGAGATTTCCAACCACAACAG |
| | | CGACTTTATTATTAAGATCCCCTTTGGCAGGTGGCAGGTCAAGAAG |
| | | GAGATTGACAAGTACAGGCCCTGGGAGAAGTTTGATTTCGAGCAGG |
| | | TGCAGAAGAGCCCCAAGCCTATTTCCCTGCTGCTGTCCACACAGCG |
| | | GCGGAAGAGGAACAAGGGGTGGTCTAAGGATGAGGGGACCGAGGCC |
| | | GAGATTAAGAAAGTGATGAACGGCGACTACCAGACAAGCTACATCG |
| | | AGGTCAAGCGGGGCAGTAAGATTGGCGAGAAGAGCGCCTGGATGCT |
| | | GAACCTGAGCATTGACGTGCCAAAGATTGATAAGGGCGTGGATCCC |
| | | AGCATCATCGGAGGGATCGATGTGGGGGTCAAGAGCCCCCTCGTGT |
| | | GCGCCATCAACAACGCCTTCAGCAGGTACAGCATCTCCGATAACGA |
| | | CCTGTTCCACTTTAACAAGAAGATGTTCGCCCGGCGGAGGATTTTG |
| | | CTCAAGAAGAACCGGCACAAGCGGGCCGGACACGGGGCCAAGAACA |
| | | AGCTCAAGCCCATCACTATCCTGACCGAGAAGAGCGAGAGGTTCAG |
| | | GAAGAAGCTCATCGAGAGATGGGCCTGCGAGATCGCCGATTTCTTT |
| | | ATTAAGAACAAGGTCGGAACAGTGCAGATGGAGAACCTCGAGAGCA |
| | | TGAAGAGGAAGGAGGATTCCTACTTCAACATTCGGCTGAGGGGGTT |
| | | CTGGCCCTACGCTGAGATGCAGAACAAGATTGAGTTTAAGCTGAAG |
| | | CAGTACGGGATTGAGATCCGGAAGGTGGCCCCCAACAACACCAGCA |
| | | AGACCTGCAGCAAGTGCGGGCACCTCAACAACTACTTCAACTTCGA |
| | | GTACCGGAAGAAGAACAAGTTCCCACACTTCAAGTGCGAGAAGTGC |
| | | AACTTTAAGGAGAACGCCGATTACAACGCCGCCCTGAACATCAGCA |
| | | ACCCTAAGCTGAAGAGCACTAAGGAGGAGCCCGGATCCGGAATGGC |
| | | GACTAAAGTGAAGTTCAAATATAAGGGTGAAGAAAAGGAAGTAGAT |
| | | ATATCCAAGATCAAGAAGGTATGGCGGGTTGGAAAAATGATCAGCT |
| | | TTACCTATGACGACAACGGAAAGACTGGTAGGGGTGCCGTATCTGA |
| | | AAAAGATGCCCCCAAAGAATTGTTGGAAAAACTCAAGGGAAGCGGA |
| | | AAAAGGCCGGCGGCCACGAAAAAGGCCGGCCAGGCAAAAAAGAAAA |
| | | AGTACCCATACGATGTTCCAGATTACGCTGGAAGCGGAGAGGGCAG |
| | | GGGAAGTCTTCTAACATGCGGGGACGTGGAGGAAAATCCCGGCCCC |
| | | ATGAAAAAGCCTGAACTCACCGCGACGTCTGTCGAGAAGTTTCTGA |
| | | TCGAAAAGTTCGACAGCGTCTCCGACCTGATGCAGCTCTCGGAGGG |
| | | CGAAGAATCTCGTGCTTTCAGCTTCGATGTAGGAGGGCGTGGATAT |
| | | GTCCTGCGGGTAAATAGCTGCGCCGATGGTTTCTACAAAGATCGTT |
| | | ATGTTTATCGGCACTTTGCATCGGCCGCGCTCCCGATTCCGGAAGT |
| | | GCTTGACATTGGGGAATTTAGCGAGAGCCTGACCTATTGCATCTCC |
| | | CGCCGTGCACAGGGTGTCACGTTGCAAGACCTGCCTGAAACCGAAC |
| | | TGCCCGCTGTTCTGCAGCCGGTCGCGGAGGCCATGGATGCGATCGC |
| | | TGCGGCCGATCTTAGCCAGACGAGCGGGTTCGGCCCATTCGGACCG |
| | | CAAGGAATCGGTCAATACACTACATGGCGTGATTTCATATGCGCGA |
| | | TTGCTGATCCCCATGTGTATCACTGGCAAACTGTGATGGACGACAC |
| | | CGTCAGTGCGTCCGTCGCGCAGGCTCTCGATGAGCTGATGCTTTGG |
| | | GCCGAGGACTGCCCCGAAGTCCGGCACCTCGTGCACGCGGATTTCG |
| | | GCTCCAACAATGTCCTGACGGACAATGGCCGCATAACAGCGGTCAT |
| | | TGACTGGAGCGAGGCGATGTTCGGGGATTCCCAATACGAGGTCGCC |
| | | AACATCTTCTTCTGGAGGCCGTGGTTGGCTTGTATGGAGCAGCAGA |
| | | CGCGCTACTTCGAGCGGAGGCATCCGGAGCTTGCAGGATCGCCGCG |
| | | GCTCCGGGCGTATATGCTCCGCATTGGTCTTGACCAACTCTATCAG |
| | | AGCTTGGTTGACGGCAATTTCGATGATGCAGCTTGGGCGCAGGGTC |
| | | GATGCGACGCAATCGTCCGATCCGGAGCCGGGACTGTCGGGCGTAC |
| | | ACAAATCGCCCGCAGAAGCGCGGCCGTCTGGACCGATGGCTGTGTA |
| | | GAAGTACTCGCCGATAGTGGAAACCGACGCCCCAGCACTCGTCCGA |
| | | GGGCAAAGGAATAGACCCGAATTCCAGGGCCAGGCCGGCCGATATC |
| | | AAGCTTATCGATAATCAACCTCTGGATTACAAAATTTGTGAAAGAT |
| | | TGACTGGTATTCTTAACTATGTTGCTCCTTTTACGCTATGTGGATA |
| | | CGCTGCTTTAATGCCTTTGTATCATGCTATTGCTTCCCGTATGGCT |
| | | TTCATTTTCTCCTCCTTGTATAAATCCTGGTTGCTGTCTCTTTATG |
| | | AGGAGTTGTGGCCCGTTGTCAGGCAACGTGGCGTGGTGTGCACTGT |
| | | GTTTGCTGACGCAACCCCCACTGGTTGGGGCATTGCCACCACCTGT |
| | | CAGCTCCTTTCCGGGACTTTCGCTTTCCCCCTCCCTATTGCCACGG |
| | | CGGAACTCATCGCCGCCTGCCTTGCCCGCTGCTGGACAGGGGCTCG |
| | | GCTGTTGGGCACTGACAATTCCGTGGTGTTGTCGGGGAAATCATCG |
| | | TCCTTTCCTTGGCTGCTCGCCTGTGTTGCCACCTGGATTCTGCGCG |
| | | GGACGTCCTTCTGCTACGTCCCTTCGGCCCTCAATCCAGCGGACCT |
| | | TCCTTCCCGCGGCCTGCTGCCGGCTCTGCGGCCTCTTCCGCGTCTT |
| | | CGCCTTCGCCCTCAGACGAGTCGGATCTCCCTTTGGGCCGCCTCCC |
| | | CGCATCGATACCGTCGACCTCGAGACCTAGAAAAACATGGAGCAAT |
| | | CACAAGTAGCAATACAGCAGCTACCAATGCTGATTGTGCCTGGCTA |
| | | GAAGCACAAGAGGAGGAGGAGGTGGGTTTTCCAGTCACACCTCAGG |
| | | TACCTTTAAGACCAATGACTTACAAGGCAGCTGTAGATCTTAGCCA |
| | | CTTTTTAAAAGAAAAGGGGGGACTGGAAGGGCTAATTCACTCCCAA |
| | | CGAAGACAAGATATCCTTGATCTGTGGATCTACCACACACAAGGCT |
| | | ACTTCCCTGATTGGCAGAACTACACACCAGGGCCAGGGATCAGATA |
| | | TCCACTGACCTTTGGATGGTGCTACAAGCTAGTACCAGTTGAGCAA |
| | | GAGAAGGTAGAAGAAGCCAATGAAGGAGAGAACACCCGCTTGTTAC |
| | | ACCCTGTGAGCCTGCATGGGATGGATGACCCGGAGAGAGAAGTATT |
| | | AGAGTGGAGGTTTGACAGCCGCCTAGCATTTCATCACATGGCCCGA |
| | | GAGCTGCATCCGGACTGTACTGGGTCTCTCTGGTTAGACCAGATCT |
| | | GAGCCTGGGAGCTCTCTGGCTAACTAGGGAACCCACTGCTTAAGCC |
| | | TCAATAAAGCTTGCCTTGAGTGCTTCAAGTAGTGTGTGCCCGTCTG |
| | | TTGTGTGACTCTGGTAACTAGAGATCCCTCAGACCCTTTTAGTCAG |
| | | TGTGGAAAATCTCTAGCAGGGCCCGTTTAAACCCGCTGATCAGCCT |
| | | CGACTGTGCCTTCTAGTTGCCAGCCATCTGTTGTTTGCCCCTCCCC |
| | | CGTGCCTTCCTTGACCCTGGAAGGTGCCACTCCCACTGTCCTTTCC |
| | | TAATAAAATGAGGAAATTGCATCGCATTGTCTGAGTAGGTGTCATT |
| | | CTATTCTGGGGGGTGGGGTGGGGCAGGACAGCAAGGGGGAGGATTG |
| | | GGAAGACAATAGCAGGCATGCTGGGGATGCGGTGGGCTCTATGGCT |
| | | TCTGAGGCGGAAAGAACCAGCTGGGGCTCTAGGGGGTATCCCCACG |
| | | CGCCCTGTAGCGGCGCATTAAGCGCGGCGGGTGTGGTGGTTACGCG |
| | | CAGCGTGACCGCTACACTTGCCAGCGCCCTAGCGCCCGCTCCTTTC |
| | | GCTTTCTTCCCTTCCTTTCTCGCCACGTTCGCCGGCTTTCCCCGTC |
| | | AAGCTCTAAATCGGGGGCTCCCTTTAGGGTTCCGATTTAGTGCTTT |
| | | ACGGCACCTCGACCCCAAAAAACTTGATTAGGGTGATGGTTCACGT |
| | | AGTGGGCCATCGCCCTGATAGACGGTTTTTCGCCCTTTGACGTTGG |
| | | AGTCCACGTTCTTTAATAGTGGACTCTTGTTCCAAACTGGAACAAC |
| | | ACTCAACCCTATCTCGGTCTATTCTTTTGATTTATAAGGGATTTTG |
| | | CCGATTTCGGCCTATTGGTTAAAAAATGAGCTGATTTAACAAAAAT |
| | | TTAACGCGAATTAATTCTGTGGAATGTGTGTCAGTTAGGGTGTGGA |
| | | AAGTCCCCAGGCTCCCCAGCAGGCAGAAGTATGCAAAGCATGCATC |
| | | TCAATTAGTCAGCAACCAGGTGTGGAAAGTCCCCAGGCTCCCCAGC |
| | | AGGCAGAAGTATGCAAAGCATGCATCTCAATTAGTCAGCAACCATA |
| | | GTCCCGCCCCTAACTCCGCCCATCCCGCCCCTAACTCCGCCCAGTT |
| | | CCGCCCATTCTCCGCCCCATGGCTGACTAATTTTTTTTATTTATGC |
| | | AGAGGCCGAGGCCGCCTCTGCCTCTGAGCTATTCCAGAAGTAGTGA |
| | | GGAGGCTTTTTTGGAGGCCTAGGCTTTTGCAAAAAGCTCCCGGGAG |
| | | CTTGTATATCCATTTTCGGATCTGATCAGCACGTGTTGACAATTAA |
| | | TCATCGGCATAGTATATCGGCATAGTATAATACGACAAGGTGAGGA |
| | | ACTAAACCATGGCCAAGTTGACCAGTGCCGTTCCGGTGCTCACCGC |
| | | GCGCGACGTCGCCGGAGCGGTCGAGTTCTGGACCGACCGGCTCGGG |
| | | TTCTCCCGGGACTTCGTGGAGGACGACTTCGCCGGTGTGGTCCGGG |
| | | ACGACGTGACCCTGTTCATCAGCGCGGTCCAGGACCAGGTGGTGCC |
| | | GGACAACACCCTGGCCTGGGTGTGGGTGCGCGGCCTGGACGAGCTG |
| | | TACGCCGAGTGGTCGGAGGTCGTGTCCACGAACTTCCGGGACGCCT |
| | | CCGGGCCGGCCATGACCGAGATCGGCGAGCAGCCGTGGGGGCGGGA |
| | | GTTCGCCCTGCGCGACCCGGCCGGCAACTGCGTGCACTTCGTGGCC |
| | | GAGGAGCAGGACTGACACGTGCTACGAGATTTCGATTCCACCGCCG |
| | | CCTTCTATGAAAGGTTGGGCTTCGGAATCGTTTTCCGGGACGCCGG |
| | | CTGGATGATCCTCCAGCGCGGGGATCTCATGCTGGAGTTCTTCGCC |
| | | CACCCCAACTTGTTTATTGCAGCTTATAATGGTTACAAATAAAGCA |
| | | ATAGCATCACAAATTTCACAAATAAAGCATTTTTTTCACTGCATTC |
| | | TAGTTGTGGTTTGTCCAAACTCATCAATGTATCTTATCATGTCTGT |
| | | ATACCGTCGACCTCTAGCTAGAGCTTGGCGTAATCATGGTCATAGC |
| | | TGTTTCCTGTGTGAAATTGTTATCCGCTCACAATTCCACACAACAT |
| | | ACGAGCCGGAAGCATAAAGTGTAAAGCCTGGGGTGCCTAATGAGTG |
| | | AGCTAACTCACATTAATTGCGTTGCGCTCACTGCCCGCTTTCCAGT |
| | | CGGGAAACCTGTCGTGCCAGCTGCATTAATGAATCGGCCAACGCGC |
| | | GGGGAGAGGCGGTTTGCGTATTGGGCGCTCTTCCGCTTCCTCGCTC |
| | | ACTGACTCGCTGCGCTCGGTCGTTCGGCTGCGGCGAGCGGTATCAG |
| | | CTCACTCAAAGGCGGTAATACGGTTATCCACAGAATCAGGGGATAA |
| | | CGCAGGAAAGAACATGTGAGCAAAAGGCCAGCAAAAGGCCAGGAAC |
| | | CGTAAAAAGGCCGCGTTGCTGGCGTTTTTCCATAGGCTCCGCCCCC |
| | | CTGACGAGCATCACAAAAATCGACGCTCAAGTCAGAGGTGGCGAAA |
| | | CCCGACAGGACTATAAAGATACCAGGCGTTTCCCCCTGGAAGCTCC |
| | | CTCGTGCGCTCTCCTGTTCCGACCCTGCCGCTTACCGGATACCTGT |
| | | CCGCCTTTCTCCCTTCGGGAAGCGTGGCGCTTTCTCATAGCTCACG |
| | | CTGTAGGTATCTCAGTTCGGTGTAGGTCGTTCGCTCCAAGCTGGGC |
| | | TGTGTGCACGAACCCCCCGTTCAGCCCGACCGCTGCGCCTTATCCG |
| | | GTAACTATCGTCTTGAGTCCAACCCGGTAAGACACGACTTATCGCC |
| | | ACTGGCAGCAGCCACTGGTAACAGGATTAGCAGAGCGAGGTATGTA |
| | | GGCGGTGCTACAGAGTTCTTGAAGTGGTGGCCTAACTACGGCTACA |
| | | CTAGAAGAACAGTATTTGGTATCTGCGCTCTGCTGAAGCCAGTTAC |
| | | CTTCGGAAAAAGAGTTGGTAGCTCTTGATCCGGCAAACAAACCACC |
| | | GCTGGTAGCGGTGGTTTTTTTGTTTGCAAGCAGCAGATTACGCGCA |
| | | GAAAAAAAGGATCTCAAGAAGATCCTTTGATCTTTTCTACGGGGTC |
| | | TGACGCTCAGTGGAACGAAAACTCACGTTAAGGGATTTTGGTCATG |
| | | AGATTATCAAAAAGGATCTTCACCTAGATCCTTTTAAATTAAAAAT |
| | | GAAGTTTTAAATCAATCTAAAGTATATATGAGTAAACTTGGTCTGA |
| | | CAGTTACCAATGCTTAATCAGTGAGGCACCTATCTCAGCGATCTGT |
| | | CTATTTCGTTCATCCATAGTTGCCTGACTCCCCGTCGTGTAGATAA |
| | | CTACGATACGGGAGGGCTTACCATCTGGCCCCAGTGCTGCAATGAT |
| | | ACCGCGAGACCCACGCTCACCGGCTCCAGATTTATCAGCAATAAAC |
| | | CAGCCAGCCGGAAGGGCCGAGCGCAGAAGTGGTCCTGCAACTTTAT |
| | | CCGCCTCCATCCAGTCTATTAATTGTTGCCGGGAAGCTAGAGTAAG |
| | | TAGTTCGCCAGTTAATAGTTTGCGCAACGTTGTTGCCATTGCTACA |
| | | GGCATCGTGGTGTCACGCTCGTCGTTTGGTATGGCTTCATTCAGCT |
| | | CCGGTTCCCAACGATCAAGGCGAGTTACATGATCCCCCATGTTGTG |
| | | CAAAAAAGCGGTTAGCTCCTTCGGTCCTCCGATCGTTGTCAGAAGT |
| | | AAGTTGGCCGCAGTGTTATCACTCATGGTTATGGCAGCACTGCATA |
| | | ATTCTCTTACTGTCATGCCATCCGTAAGATGCTTTTCTGTGACTGG |
| | | TGAGTACTCAACCAAGTCATTCTGAGAATAGTGTATGCGGCGACCG |
| | | AGTTGCTCTTGCCCGGCGTCAATACGGGATAATACCGCGCCACATA |
| | | GCAGAACTTTAAAAGTGCTCATCATTGGAAAACGTTCTTCGGGGCG |
| | | AAAACTCTCAAGGATCTTACCGCTGTTGAGATCCAGTTCGATGTAA |
| | | CCCACTCGTGCACCCAACTGATCTTCAGCATCTTTTACTTTCACCA |
| | | GCGTTTCTGGGTGAGCAAAAACAGGAAGGCAAAATGCCGCAAAAAA |
| | | GGGAATAAGGGCGACACGGAAATGTTGAATACTCATACTCTTCCTT |
| | | TTTCAATATTATTGAAGCATTTATCAGGGTTATTGTCTCATGAGCG |
| | | GATACATATTTGAATGTATTTAGAAAAATAAACAAATAGGGGTTCC |
| | | GCGCACATTTCCCCGAAAAGTGCCACCTGAC |
| 117 | Cas12f1- | MAKNTITKTLKLRIVRPYNSAEVEKIVADEKNNREKIALEKNKDKV |
| | Sso7d | KEACSKHLKVAAYCTTQVERNACLFCKARKLDDKFYQKLRGQFPDA |
| | (amino | VFWQEISEIFRQLQKQAAEIYNQSLIELYYEIFIKGKGIANASSVE |
| | acid) | HYLSDVCYTRAAELFKNAAIASGLRSKIKSNFRLKELKNMKSGLPT |
| | | TKSDNFPIPLVKQKGGQYTGFEISNHNSDFIIKIPFGRWQVKKEID |
| | | KYRPWEKFDFEQVQKSPKPISLLLSTQRRKRNKGWSKDEGTEAEIK |
| | | KVMNGDYQTSYIEVKRGSKIGEKSAWMLNLSIDVPKIDKGVDPSII |
| | | GGIDVGVKSPLVCAINNAFSRYSISDNDLFHFNKKMFARRRILLKK |
| | | NRHKRAGHGAKNKLKPITILTEKSERFRKKLIERWACEIADFFIKN |
| | | KVGTVQMENLESMKRKEDSYFNIRLRGFWPYAEMQNKIEFKLKQYG |
| | | IEIRKVAPNNTSKTCSKCGHLNNYFNFEYRKKNKFPHFKCEKCNFK |
| | | ENADYNAALNISNPKLKSTKEEPGSGMATVKFKYKGEEKEVDISKI |
| | | KKVWRVGKMISFTYDEGGGKTGRGAVSEKDAPKELLQMLEKQKK |
| 118 | Cas12f1- | ATGGCCAAGAACACAATTACAAAGACACTGAAGCTGAGGATCGTGA |
| | Sso7d | GACCATACAACAGCGCTGAGGTCGAGAAGATTGTGGCTGATGAAAA |
| | (DNA) | GAACAACAGGGAAAAGATCGCCCTCGAGAAGAACAAGGATAAGGTG |
| | | AAGGAGGCCTGCTCTAAGCACCTGAAAGTGGCCGCCTACTGCACCA |
| | | CACAGGTGGAGAGGAACGCCTGTCTGTTTTGTAAAGCTCGGAAGCT |
| | | GGATGATAAGTTTTACCAGAAGCTGCGGGGCCAGTTCCCCGATGCC |
| | | GTCTTTTGGCAGGAGATTAGCGAGATCTTCAGACAGCTGCAGAAGC |
| | | AGGCCGCCGAGATCTACAACCAGAGCCTGATCGAGCTCTACTACGA |
| | | GATCTTCATCAAGGGCAAGGGCATTGCCAACGCCTCCTCCGTGGAG |
| | | CACTACCTGAGCGACGTGTGCTACACAAGAGCCGCCGAGCTCTTTA |
| | | AGAACGCCGCTATCGCTTCCGGGCTGAGGAGCAAGATTAAGAGTAA |
| | | CTTCCGGCTCAAGGAGCTGAAGAACATGAAGAGCGGCCTGCCCACT |
| | | ACAAAGAGCGACAACTTCCCAATTCCACTGGTGAAGCAGAAGGGGG |
| | | GCCAGTACACAGGGTTCGAGATTTCCAACCACAACAGCGACTTTAT |
| | | TATTAAGATCCCCTTTGGCAGGTGGCAGGTCAAGAAGGAGATTGAC |
| | | AAGTACAGGCCCTGGGAGAAGTTTGATTTCGAGCAGGTGCAGAAGA |
| | | GCCCCAAGCCTATTTCCCTGCTGCTGTCCACACAGCGGCGGAAGAG |
| | | GAACAAGGGGTGGTCTAAGGATGAGGGGACCGAGGCCGAGATTAAG |
| | | AAAGTGATGAACGGCGACTACCAGACAAGCTACATCGAGGTCAAGC |
| | | GGGGCAGTAAGATTGGCGAGAAGAGCGCCTGGATGCTGAACCTGAG |
| | | CATTGACGTGCCAAAGATTGATAAGGGCGTGGATCCCAGCATCATC |
| | | GGAGGGATCGATGTGGGGGTCAAGAGCCCCCTCGTGTGCGCCATCA |
| | | ACAACGCCTTCAGCAGGTACAGCATCTCCGATAACGACCTGTTCCA |
| | | CTTTAACAAGAAGATGTTCGCCCGGCGGAGGATTTTGCTCAAGAAG |
| | | AACCGGCACAAGCGGGCCGGACACGGGGCCAAGAACAAGCTCAAGC |
| | | CCATCACTATCCTGACCGAGAAGAGCGAGAGGTTCAGGAAGAAGCT |
| | | CATCGAGAGATGGGCCTGCGAGATCGCCGATTTCTTTATTAAGAAC |
| | | AAGGTCGGAACAGTGCAGATGGAGAACCTCGAGAGCATGAAGAGGA |
| | | AGGAGGATTCCTACTTCAACATTCGGCTGAGGGGGTTCTGGCCCTA |
| | | CGCTGAGATGCAGAACAAGATTGAGTTTAAGCTGAAGCAGTACGGG |
| | | ATTGAGATCCGGAAGGTGGCCCCCAACAACACCAGCAAGACCTGCA |
| | | GCAAGTGCGGGCACCTCAACAACTACTTCAACTTCGAGTACCGGAA |
| | | GAAGAACAAGTTCCCACACTTCAAGTGCGAGAAGTGCAACTTTAAG |
| | | GAGAACGCCGATTACAACGCCGCCCTGAACATCAGCAACCCTAAGC |
| | | TGAAGAGCACTAAGGAGGAGCCCggatccggaATGGCTACAGTGAA |
| | | GTTTAAGTATAAAGGGGAAGAGAAAGAGGTGGACATCAGTAAAATC |
| | | AAGAAAGTGTGGCGCGTAGGTAAGATGATCAGCTTTACGTACGATG |
| | | AAGGAGGAGGAAAGACTGGCAGAGGAGCCGTTTCAGAAAAGGACGC |
| | | TCCAAAGGAATTGTTGCAGATGCTCGAGAAGCAGAAGAAG |
| 119 | SpCas9- | MDKKYSIGLDIGTNSVGWAVITDEYKVPSKKFKVLGNTDRHSIKKN |
| | Sso7d | LIGALLFDSGETAEATRLKRTARRRYTRRKNRICYLQEIFSNEMAK |
| | (amino | VDDSFFHRLEESFLVEEDKKHERHPIFGNIVDEVAYHEKYPTIYHL |
| | acid) | RKKLVDSTDKADLRLIYLALAHMIKFRGHFLIEGDLNPDNSDVDKL |
| | | FIQLVQTYNQLFEENPINASGVDAKAILSARLSKSRRLENLIAQLP |
| | | GEKKNGLFGNLIALSLGLTPNFKSNFDLAEDAKLQLSKDTYDDDLD |
| | | NLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQI |
| | | HLGELHAILRRQEDFYPFLKDNREKIEKILTFRIPYYVGPLARGNS |
| | | RFAWMTRKSEETITPWNFEEVVDKGASAQSFIERMTNFDKNLPNEK |
| | | VLPKHSLLYEYFTVYNELTKVKYVTEGMRKPAFLSGEQKKAIVDLL |
| | | FKTNRKVTVKQLKEDYFKKIECFDSVEISGVEDRFNASLGTYHDLL |
| | | KIIKDKDFLDNEENEDILEDIVLTLTLFEDREMIEERLKTYAHLFD |
| | | DKVMKQLKRRRYTGWGRLSRKLINGIRDKQSGKTILDFLKSDGFAN |
| | | RNFMQLIHDDSLTFKEDIQKAQVSGQGDSLHEHIANLAGSPAIKKG |
| | | ILQTVKVVDELVKVMGRHKPENIVIEMARENQTTQKGQKNSRERMK |
| | | RIEEGIKELGSQILKEHPVENTQLQNEKLYLYYLQNGRDMYVDQEL |
| | | DINRLSDYDVDHIVPQSFLKDDSIDNKVLTRSDKNRGKSDNVPSEE |
| | | VVKKMKNYWRQLLNAKLITQRKFDNLTKAERGGLSELDKAGFIKRQ |
| | | LVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIR |
| | | KRPLIETNGETGEIVWDKGRDFATVRKVLSMPQVNIVKKTEVQTGG |
| | | FSKESILPKRNSDKLIARKKDWDPKKYGGFDSPTVAYSVLVVAKVE |
| | | KGKSKKLKSVKELLGITIMERSSFEKNPIDFLEAKGYKEVKKDLII |
| | | KLPKYSLFELENGRKRMLASAGELQKGNELALPSKYVNFLYLASHY |
| | | EKLKGSPEDNEQKQLFVEQHKHYLDEIIEQISEFSKRVILADANLD |
| | | KVLSAYNKHRDKPIREQAENIIHLFTLTNLGAPAAFKYFDTTIDRK |
| | | RYTSTKEVLDATLIHQSITGLYETRIDLSQLGGDGSGMATVKFKYK |
| | | GEEKEVDISKIKKVWRVGKMISFTYDEGGGKTGRGAVSEKDAPKEL |
| | | LQMLEKQKK |
| 120 | SpCas9- | atggacaagaagtacagcatcggcctggacatcggcaccaactctg |
| | Sso7d | tgggctgggccgtgatcaccgacgagtacaaggtgcccagcaagaa |
| | (DNA) | attcaaggtgctgggcaacaccgaccggcacagcatcaagaagaac |
| | | ctgatcggagccctgctgttcgacagcggcgaaacagccgaggcca |
| | | cccggctgaagagaaccgccagaagaagatacaccagacggaagaa |
| | | ccggatctgctatctgcaagagatcttcagcaacgagatggccaag |
| | | gtggacgacagcttcttccacagactggaagagtccttcctggtgg |
| | | aagaggataagaagcacgagcggcaccccatcttcggcaacatcgt |
| | | ggacgaggtggcctaccacgagaagtaccccaccatctaccacctg |
| | | agaaagaaactggtggacagcaccgacaaggccgacctgcggctga |
| | | tctatctggccctggcccacatgatcaagttccggggccacttcct |
| | | gatcgagggcgacctgaaccccgacaacagcgacgtggacaagctg |
| | | ttcatccagctggtgcagacctacaaccagctgttcgaggaaaacc |
| | | ccatcaacgccagcggcgtggacgccaaggccatcctgtctgccag |
| | | actgagcaagagcagacggctggaaaatctgatcgcccagctgccc |
| | | ggcgagaagaagaatggcctgttcggaaacctgattgccctgagcc |
| | | tgggcctgacccccaacttcaagagcaacttcgacctggccgagga |
| | | tgccaaactgcagctgagcaaggacacctacgacgacgacctggac |
| | | aacctgctggcccagatcggcgaccagtacgccgacctgtttctgg |
| | | ccgccaagaacctgtccgacgccatcctgctgagcgacatcctgag |
| | | agtgaacaccgagatcaccaaggcccccctgagcgcctctatgatc |
| | | aagagatacgacgagcaccaccaggacctgaccctgctgaaagctc |
| | | tcgtgcggcagcagctgcctgagaagtacaaagagattttcttcga |
| | | ccagagcaagaacggctacgccggctacattgacggcggagccagc |
| | | caggaagagttctacaagttcatcaagcccatcctggaaaagatgg |
| | | acggcaccgaggaactgctcgtgaagctgaacagagaggacctgct |
| | | gcggaagcagcggaccttcgacaacggcagcatcccccaccagatc |
| | | cacctgggagagctgcacgccattctgcggcggcaggaagattttt |
| | | acccattcctgaaggacaaccgggaaaagatcgagaagatcctgac |
| | | cttccgcatcccctactacgtgggccctctggccaggggaaacagc |
| | | agattcgcctggatgaccagaaagagcgaggaaaccatcaccccct |
| | | ggaacttcgaggaagtggtggacaagggcgcttccgcccagagctt |
| | | catcgagcggatgaccaacttcgataagaacctgcccaacgagaag |
| | | gtgctgcccaagcacagcctgctgtacgagtacttcaccgtgtata |
| | | acgagctgaccaaagtgaaatacgtgaccgagggaatgagaaagcc |
| | | cgccttcctgagcggcgagcagaaaaaggccatcgtggacctgctg |
| | | ttcaagaccaaccggaaagtgaccgtgaagcagctgaaagaggact |
| | | acttcaagaaaatcgagtgcttcgactccgtggaaatctccggcgt |
| | | ggaagatcggttcaacgcctccctgggcacataccacgatctgctg |
| | | aaaattatcaaggacaaggacttcctggacaatgaggaaaacgagg |
| | | acattctggaagatatcgtgctgaccctgacactgtttgaggacag |
| | | agagatgatcgaggaacggctgaaaacctatgcccacctgttcgac |
| | | gacaaagtgatgaagcagctgaagcggcggagatacaccggctggg |
| | | gcaggctgagccggaagctgatcaacggcatccgggacaagcagtc |
| | | cggcaagacaatcctggatttcctgaagtccgacggcttcgccaac |
| | | agaaacttcatgcagctgatccacgacgacagcctgacctttaaag |
| | | aggacatccagaaagcccaggtgtccggccagggcgatagcctgca |
| | | cgagcacattgccaatctggccggcagccccgccattaagaagggc |
| | | atcctgcagacagtgaaggtggtggacgagctcgtgaaagtgatgg |
| | | gccggcacaagcccgagaacatcgtgatcgaaatggccagagagaa |
| | | ccagaccacccagaagggacagaagaacagccgcgagagaatgaag |
| | | cggatcgaagagggcatcaaagagctgggcagccagatcctgaaag |
| | | aacaccccgtggaaaacacccagctgcagaacgagaagctgtacct |
| | | gtactacctgcagaatggggggatatgtacgtggaccaggaactgg |
| | | acatcaaccggctgtccgactacgatgtggaccatatcgtgcctca |
| | | gagctttctgaaggacgactccatcgacaacaaggtgctgaccaga |
| | | agcgacaagaaccggggcaagagcgacaacgtgccctccgaagagg |
| | | tcgtgaagaagatgaagaactactggcggcagctgctgaacgccaa |
| | | gctgattacccagagaaagttcgacaatctgaccaaggccgagaga |
| | | ggcggcctgagcgaactggataaggccggcttcatcaagagacagc |
| | | tggtggaaacccggcagatcacaaagcacgtggcacagatcctgga |
| | | ctcccggatgaacactaagtacgacgagaatgacaagctgatccgg |
| | | gaagtgaaagtgatcaccctgaagtccaagctggtgtccgatttcc |
| | | ggaaggatttccagttttacaaagtgcgcgagatcaacaactacca |
| | | ccacgcccacgacgcctacctgaacgccgtcgtgggaaccgccctg |
| | | atcaaaaagtaccctaagctggaaagcgagttcgtgtacggcgact |
| | | acaaggtgtacgacgtgcggaagatgatcgccaagagcgagcagga |
| | | aatcggcaaggctaccgccaagtacttcttctacagcaacatcatg |
| | | aactttttcaagaccgagattaccctggccaacggcgagatccgga |
| | | agcggcctctgatcgagacaaacggcgaaaccggggagatcgtgtg |
| | | ggataagggccgggattttgccaccgtgcggaaagtgctgagcatg |
| | | ccccaagtgaatatcgtgaaaaagaccgaggtgcagacaggcggct |
| | | tcagcaaagagtctatcctgcccaagaggaacagcgataagctgat |
| | | cgccagaaagaaggactgggaccctaagaagtacggcggcttcgac |
| | | agccccaccgtggcctattctgtgctggtggtggccaaagtggaaa |
| | | agggcaagtccaagaaactgaagagtgtgaaagagctgctggggat |
| | | caccatcatggaaagaagcagcttcgagaagaatcccatcgacttt |
| | | ctggaagccaagggctacaaagaagtgaaaaaggacctgatcatca |
| | | agctgcctaagtactccctgttcgagctggaaaacggccggaagag |
| | | aatgctggcctctgccggcgaactgcagaagggaaacgaactggcc |
| | | ctgccctccaaatatgtgaacttcctgtacctggccagccactatg |
| | | agaagctgaagggctcccccgaggataatgagcagaaacagctgtt |
| | | tgtggaacagcacaagcactacctggacgagatcatcgagcagatc |
| | | agcgagttctccaagagagtgatcctggccgacgctaatctggaca |
| | | aagtgctgtccgcctacaacaagcaccgggataagcccatcagaga |
| | | gcaggccgagaatatcatccacctgtttaccctgaccaatctggga |
| | | gcccctgccgccttcaagtactttgacaccaccatcgaccggaaga |
| | | ggtacaccagcaccaaagaggtgctggacgccaccctgatccacca |
| | | gagcatcaccggcctgtacgagacacggatcgacctgtctcagctg |
| | | ggaggcgacggatccggaATGGCTACAGTGAAGTTTAAGTATAAAG |
| | | GGGAAGAGAAAGAGGTGGACATCAGTAAAATCAAGAAAGTGTGGCG |
| | | CGTAGGTAAGATGATCAGCTTTACGTACGATGAAGGAGGAGGAAAG |
| | | ACTGGCAGAGGAGCCGTTTCAGAAAAGGACGCTCCAAAGGAATTGT |
| | | TGCAGATGCTCGAGAAGCAGAAGAAG |
| 121 | SaCas9- | MKRNYILGLDIGITSVGYGIIDYETRDVIDAGVRLFKEANVENNEG |
| | Sso7d | RRSKRGARRLKRRRRHRIQRVKKLLFDYNLLTDHSELSGINPYEAR |
| | (amino | VKGLSQKLSEEEFSAALLHLAKRRGVHNVNEVEEDTGNELSTKEQI |
| | acid) | SRNSKALEEKYVAELQLERLKKDGEVRGSINRFKTSDYVKEAKQLL |
| | | KVQKAYHQLDQSFIDTYIDLLETRRTYYEGPGEGSPFGWKDIKEWY |
| | | EMLMGHCTYFPEELRSVKYAYNADLYNALNDLNNLVITRDENEKLE |
| | | YYEKFQIIENVFKQKKKPTLKQIAKEILVNEEDIKGYRVTSTGKPE |
| | | FTNLKVYHDIKDITARKEIIENAELLDQIAKILTIYQSSEDIQEEL |
| | | TNLNSELTQEEIEQISNLKGYTGTHNLSLKAINLILDELWHTNDNQ |
| | | IAIFNRLKLVPKKVDLSQQKEIPTTLVDDFILSPVVKRSFIQSIKV |
| | | INAIIKKYGLPNDIIIELAREKNSKDAQKMINEMQKRNRQTNERIE |
| | | EIIRTTGKENAKYLIEKIKLHDMQEGKCLYSLEAIPLEDLLNNPFN |
| | | YEVDHIIPRSVSFDNSFNNKVLVKQEENSKKGNRTPFQYLSSSDSK |
| | | ISYETFKKHILNLAKGKGRISKTKKEYLLEERDINRFSVQKDFINR |
| | | NLVDTRYATRGLMNLLRSYFRVNNLDVKVKSINGGFTSFLRRKWKF |
| | | KKERNKGYKHHAEDALIIANADFIFKEWKKLDKAKKVMENQMFEEK |
| | | QAESMPEIETEQEYKEIFITPHQIKHIKDFKDYKYSHRVDKKPNRE |
| | | LINDTLYSTRKDDKGNTLIVNNLNGLYDKDNDKLKKLINKSPEKLL |
| | | MYHHDPQTYQKLKLIMEQYGDEKNPLYKYYEETGNYLTKYSKKDNG |
| | | PVIKKIKYYGNKLNAHLDITDDYPNSRNKVVKLSLKPYRFDVYLDN |
| | | GVYKFVTVKNLDVIKKENYYEVNSKCYEEAKKLKKISNQAEFIASF |
| | | YNNDLIKINGELYRVIGVNNDLLNRIEVNMIDITYREYLENMNDKR |
| | | PPRIIKTIASKTQSIKKYSTDILGNLYEVKSKKHPQIIKKGGSGMA |
| | | TVKFKYKGEEKEVDISKIKKVWRVGKMISFTYDEGGGKTGRGAVSE |
| | | KDAPKELLQMLEKQKK |
| 122 | SaCas9- | atgaagcggaactacatcctgggcctggacatcggcatcaccagcg |
| | Sso7d | tgggctacggcatcatcgactacgagacacgggacgtgatcgatgc |
| | (DNA) | cggcgtgcggctgttcaaagaggccaacgtggaaaacaacgagggc |
| | | aggcggagcaagagaggcgccagaaggctgaagcggcggaggcggc |
| | | atagaatccagagagtgaagaagctgctgttcgactacaacctgct |
| | | gaccgaccacagcgagctgagcggcatcaacccctacgaggccaga |
| | | gtgaagggcctgagccagaagctgagcgaggaagagttctctgccg |
| | | ccctgctgcacctggccaagagaagaggcgtgcacaacgtgaacga |
| | | ggtggaagaggacaccggcaacgagctgtccaccaaagagcagatc |
| | | agccggaacagcaaggccctggaagagaaatacgtggccgaactgc |
| | | agctggaacggctgaagaaagacggcgaagtgcggggcagcatcaa |
| | | cagattcaagaccagcgactacgtgaaagaagccaaacagctgctg |
| | | aaggtgcagaaggcctaccaccagctggaccagagcttcatcgaca |
| | | cctacatcgacctgctggaaacccggcggacctactatgagggacc |
| | | tggcgagggcagccccttcggctggaaggacatcaaagaatggtac |
| | | gagatgctgatgggccactgcacctacttccccgaggaactgcgga |
| | | gcgtgaagtacgcctacaacgccgacctgtacaacgccctgaacga |
| | | cctgaacaatctcgtgatcaccagggacgagaacgagaagctggaa |
| | | tattacgagaagttccagatcatcgagaacgtgttcaagcagaaga |
| | | agaagcccaccctgaagcagatcgccaaagaaatcctcgtgaacga |
| | | agaggatattaagggctacagagtgaccagcaccggcaagcccgag |
| | | ttcaccaacctgaaggtgtaccacgacatcaaggacattaccgccc |
| | | ggaaagagattattgagaacgccgagctgctggatcagattgccaa |
| | | gatcctgaccatctaccagagcagcgaggacatccaggaagaactg |
| | | accaatctgaactccgagctgacccaggaagagatcgagcagatct |
| | | ctaatctgaagggctataccggcacccacaacctgagcctgaaggc |
| | | catcaacctgatcctggacgagctgtggcacaccaacgacaaccag |
| | | atcgctatcttcaaccggctgaagctggtgcccaagaaggtggacc |
| | | tgtcccagcagaaagagatccccaccaccctggtggacgacttcat |
| | | cctgagccccgtcgtgaagagaagcttcatccagagcatcaaagtg |
| | | atcaacgccatcatcaagaagtacggcctgcccaacgacatcatta |
| | | tcgagctggcccgcgagaagaactccaaggacgcccagaaaatgat |
| | | caacgagatgcagaagcggaaccggcagaccaacgagcggatcgag |
| | | gaaatcatccggaccaccggcaaagagaacgccaagtacctgatcg |
| | | agaagatcaagctgcacgacatgcaggaaggcaagtgcctgtacag |
| | | cctggaagccatccctctggaagatctgctgaacaaccccttcaac |
| | | tatgaggtggaccacatcatccccagaagcgtgtccttcgacaaca |
| | | gcttcaacaacaaggtgctcgtgaagcaggaagaaaacagcaagaa |
| | | gggcaaccggaccccattccagtacctgagcagcagcgacagcaag |
| | | atcagctacgaaaccttcaagaagcacatcctgaatctggccaagg |
| | | gcaagggcagaatcagcaagaccaagaaagagtatctgctggaaga |
| | | acgggacatcaacaggttctccgtgcagaaagacttcatcaaccgg |
| | | aacctggtggataccagatacgccaccagaggcctgatgaacctgc |
| | | tgcggagctacttcagagtgaacaacctggacgtgaaagtgaagtc |
| | | catcaatggcggcttcaccagctttctgcggcggaagtggaagttt |
| | | aagaaagagcggaacaaggggtacaagcaccacgccgaggacgccc |
| | | tgatcattgccaacgccgatttcatcttcaaagagtggaagaaact |
| | | ggacaaggccaaaaaagtgatggaaaaccagatgttcgaggaaaag |
| | | caggccgagagcatgcccgagatcgaaaccgagcaggagtacaaag |
| | | agatcttcatcaccccccaccagatcaagcacattaaggacttcaa |
| | | ggactacaagtacagccaccgggtggacaagaagcctaatagagag |
| | | ctgattaacgacaccctgtactccacccggaaggacgacaagggca |
| | | acaccctgatcgtgaacaatctgaacggcctgtacgacaaggacaa |
| | | tgacaagctgaaaaagctgatcaacaagagccccgaaaagctgctg |
| | | atgtaccaccacgacccccagacctaccagaaactgaagctgatta |
| | | tggaacagtacggcgacgagaagaatcccctgtacaagtactacga |
| | | ggaaaccgggaactacctgaccaagtactccaaaaaggacaacggc |
| | | cccgtgatcaagaagattaagtattacggcaacaaactgaacgccc |
| | | atctggacatcaccgacgactaccccaacagcagaaacaaggtcgt |
| | | gaagctgtccctgaagccctacagattcgacgtgtacctggacaat |
| | | ggcgtgtacaagttcgtgaccgtgaagaatctggatgtgatcaaaa |
| | | aagaaaactactacgaagtgaatagcaagtgctatgaggaagctaa |
| | | gaagctgaagaagatcagcaaccaggccgagtttatcgcctccttc |
| | | tacaacaacgatctgatcaagatcaacggcgagctgtatagagtga |
| | | tcggcgtgaacaacgacctgctgaaccggatcgaagtgaacatgat |
| | | cgacatcacctaccgcgagtacctggaaaacatgaacgacaagagg |
| | | ccccccaggatcattaagacaatcgcctccaagacccagagcatta |
| | | agaagtacagcacagacattctgggcaacctgtatgaagtgaaatc |
| | | taagaagcaccctcagatcatcaaaaagggcggatccggaATGGCT |
| | | ACAGTGAAGTTTAAGTATAAAGGGGAAGAGAAAGAGGTGGACATCA |
| | | GTAAAATCAAGAAAGTGTGGCGCGTAGGTAAGATGATCAGCTTTAC |
| | | GTACGATGAAGGAGGAGGAAAGACTGGCAGAGGAGCCGTTTCAGAA |
| | | AAGGACGCTCCAAAGGAATTGTTGCAGATGCTCGAGAAGCAGAAGA |
| | | AG |
| 123 | Cas12f1- | MAKNTITKTLKLRIVRPYNSAEVEKIVADEKNNREKIALEKNKDKV |
| | Aho7c | KEACSKHLKVAAYCTTQVERNACLFCKARKLDDKFYQKLRGQFPDA |
| | (amino | VFWQEISEIFRQLQKQAAEIYNQSLIELYYEIFIKGKGIANASSVE |
| | acid) | HYLSDVCYTRAAELFKNAAIASGLRSKIKSNFRLKELKNMKSGLPT |
| | | TKSDNFPIPLVKQKGGQYTGFEISNHNSDFIIKIPFGRWQVKKEID |
| | | KYRPWEKFDFEQVQKSPKPISLLLSTQRRKRNKGWSKDEGTEAEIK |
| | | KVMNGDYQTSYIEVKRGSKIGEKSAWMLNLSIDVPKIDKGVDPSII |
| | | GGIDVGVKSPLVCAINNAFSRYSISDNDLFHFNKKMFARRRILLKK |
| | | NRHKRAGHGAKNKLKPITILTEKSERFRKKLIERWACEIADFFIKN |
| | | KVGTVQMENLESMKRKEDSYFNIRLRGFWPYAEMQNKIEFKLKQYG |
| | | IEIRKVAPNNTSKTCSKCGHLNNYFNFEYRKKNKFPHFKCEKCNFK |
| | | ENADYNAALNISNPKLKSTKEEPGSGMATKVKFKYKGEEKEVDISK |
| | | IKKVWRVGKMISFTYDDNGKTGRGAVSEKDAPKELLEKLK |
| 124 | Cas12f1- | ATGGCCAAGAACACAATTACAAAGACACTGAAGCTGAGGATCGTGA |
| | Aho7c | GACCATACAACAGCGCTGAGGTCGAGAAGATTGTGGCTGATGAAAA |
| | | GAACAACAGGGAAAAGATCGCCCTCGAGACCACACAGGTGGAGAGG |
| | | AACGCCTGTCTGTTTTGTAAAGCTCGGAAGCTGGATGATAAGTTTT |
| | | ACCAGAAGCTGCGGGGCCAGTTCCCCGATGCCGTCTTTTGGCAGGA |
| | | GATTAGCGAGATCTTCAGACAGCTGCAGAAGCAGGCCGCCGAGATC |
| | | TACAACCAGAGCCTGATCGAGCTCTACTACGAGATCTTCATCAAGG |
| | | GCAAGGGCATTGCCAACGCCTCCTCCGTGGAGCACTACCTGAGCGA |
| | | CGTGTGCTACACAAGAGCCGCCGAGCTCTTTAAGAACGCCGCTATC |
| | | GCTTCCGGGCTGAGGAGCAAGATTAAGAGTAACTTCCGGCTCAAGG |
| | | AGCTGAAGAACATGAAGAGCGGCCTGCCCACTACAAAGAGCGACAA |
| | | CTTCCCAATTCCACTGGTGAAGCAGAAGGGGGGCCAGTACACAGGG |
| | | TTCGAGATTTCCAACCACAACAGCGACTTTATTATTAAGATCCCCT |
| | | TTGGCAGGTGGCAGGTCAAGAAGGAGATTGACAAGTACAGGCCCTG |
| | | GGAGAAGTTTGATTTCGAGCAGGTGCAGAAGAGCCCCAAGCCTATT |
| | | TCCCTGCTGCTGTCCACACAGCGGCGGAAGAGGAACAAGGGGTGGT |
| | | CTAAGGATGAGGGGACCGAGGCCGAGATTAAGAAAGTGATGAACGG |
| | | CGACTACCAGACAAGCTACATCGAGGTCAAGCGGGGCAGTAAGATT |
| | | GGCGAGAAGAGCGCCTGGATGCTGAACCTGAGCATTGACGTGCCAA |
| | | AGATTGATAAGGGCGTGGATCCCAGCATCATCGGAGGGATCGATGT |
| | | GGGGGTCAAGAGCCCCCTCGTGTGCGCCATCAACAACGCCTTCAGC |
| | | AGGTACAGCATCTCCGATAACGACCTGTTCCACTTTAACAAGAAGA |
| | | TGTTCGCCCGGCGGAGGATTTTGCTCAAGAAGAACCGGCACAAGCG |
| | | GGCCGGACACGGGGCCAAGAACAAGCTCAAGCCCATCACTATCCTG |
| | | ACCGAGAAGAGCGAGAGGTTCAGGAAGAAGCTCATCGAGAGATGGG |
| | | CCTGCGAGATCGCCGATTTCTTTATTAAGAACAAGGTCGGAACAGT |
| | | GCAGATGGAGAACCTCGAGAGCATGAAGAGGAAGGAGGATTCCTAC |
| | | TTCAACATTCGGCTGAGGGGGTTCTGGCCCTACGCTGAGATGCAGA |
| | | ACAAGATTGAGTTTAAGCTGAAGCAGTACGGGATTGAGATCCGGAA |
| | | GGTGGCCCCCAACAACACCAGCAAGACCTGCAGCAAGTGCGGGCAC |
| | | CTCAACAACTACTTCAACTTCGAGTACCGGAAGAAGAACAAGTTCC |
| | | CACACTTCAAGTGCGAGAAGTGCAACTTTAAGGAGAACGCCGATTA |
| | | CAACGCCGCCCTGAACATCAGCAACCCTAAGCTGAAGAGCACTAAG |
| | | GAGGAGCCCggatccggaATGGCGACTAAAGTGAAGTTCAAATATA |
| | | AGGGTGAAGAAAAGGAAGTAGATATATCCAAGATCAAGAAGGTATG |
| | | GCGGGTTGGAAAAATGATCAGCTTTACCTATGACGACAACGGAAAG |
| | | ACTGGTAGGGGTGCCGTATCTGAAAAAGATGCCCCCAAAGAATTGT |
| | | TGGAAAAACTCAAG |
| 125 | Linker 3 | GGGGS |

What is claimed:

1. A fusion protein comprising (a) a Cas enzyme or the fragment thereof and (b) a DNA-binding protein or a DNA-binding fragment thereof.

2. The fusion protein of claim 1, wherein the DNA-binding protein or DNA-binding fragment thereof is linked to the Cas enzyme or its fragment via one or more chemical bonds, by a linker, or a combination thereof.

3. The fusion protein of claim 2, wherein the DNA-binding protein or DNA-binding fragment thereof is linked to the Cas enzyme or its fragment via one or more covalent chemical bonds.

4. The fusion protein of claim 2, wherein the DNA-binding protein or DNA-binding fragment thereof is linked to the Cas enzyme or the fragment thereof by a linker.

5. The fusion protein of claim 4, wherein the linker is a flexible linker, a rigid linker, or any combination thereof.

6. The fusion protein of claim 5, wherein the linker is selected from the group consisting of (G)ₙ, (G₄S)ₙ, (GSG)ₙ (SEQ ID NO: 125), (GGGGS)ₙ (SEQ ID NO: 58), (EAAK)ₙ (SEQ ID NO: 59), or (XP)ₙ, wherein n is an integer between 1 and 35 and X is any amino acid.

7. The fusion protein of any one of the preceding claims, wherein the DNA-binding protein or DNA-binding fragment thereof is located upstream of the Cas enzyme or its fragment, downstream of the Cas enzyme or its fragment, or a combination thereof.

8. The fusion protein of claim 7, wherein the DNA-binding protein or DNA-binding fragment thereof is linked to the Cas enzyme or its fragment at the N-terminus, C-terminus, or a combination thereof.

9. The fusion protein of any one of the preceding claims, wherein the fusion protein further comprises at least one nuclear localization signal (NLS), at least one cell penetrating peptide, or a combination thereof.

10. The fusion protein of claim 9, wherein the fusion protein comprises a single NLS.

11. The fusion protein of claim 9, wherein the fusion protein comprises multiple NLS.

12. The fusion protein of claims 9-11, wherein the at least one NLS or the at least one cell penetrating peptide is linked to the fusion protein via one or more chemical bonds, by a linker, or a combination thereof.

13. The fusion protein of claim 12, wherein the at least one NLS or the at least one cell penetrating peptide is linked to the fusion protein via one or more covalent chemical bonds.

14. The fusion protein of claim 13, wherein the at least one NLS or the at least one cell penetrating peptide is linked to the fusion protein by a linker.

15. The fusion protein of claim 14, wherein the linker is a flexible linker, a rigid linker, or any combination thereof.

16. The fusion protein of claim 15, wherein the linker is selected from the group consisting of (G)ₙ, (G₄S)ₙ (SEQ ID NO: 125), (GSG)ₙ, (GGGGS)ₙ (SEQ ID NO: 58), (EAAK)ₙ (SEQ ID NO: 59), or (XP)ₙ, wherein n is an integer between 1 and 35 and X is any amino acid.

17. The fusion protein of claims 9-16, wherein the NLS is linked to the N-terminus of the Cas enzyme or its fragment, the C-terminus of the Cas enzyme or its fragment, the N-terminus of the DNA-binding protein or DNA-binding fragment thereof, the C-terminus of the DNA-binding protein or DNA-binding fragment thereof, or a combination thereof.

18. The fusion protein of claim 17, wherein the NLS is selected from the group consisting of SEQ ID NOs: 34-52.

19. The fusion protein of claims 1-18, wherein the DNA-binding protein or a DNA-binding fragment thereof is a 7 kDa DNA-binding protein, a zinc finger protein, or a transcription activator-like effector protein, or a DNA-binding fragment thereof.

20. The fusion protein of claims 1-18, wherein the DNA-binding protein or a DNA-binding fragment thereof is a 7 kDa DNA-binding protein, a zinc finger protein, or a transcription activator-like effector protein, or a DNA-binding fragment thereof.

21. The fusion protein of claim 20, wherein the 7 kDa DNA-binding protein, or DNA-binding fragment thereof, is selected from the group consisting of Sac7d, Sac7e, Mse7, Mcu7, Aho7a, Aho7b, Aho7c, Sto7, Ssh7b, Sis7a, Sis7b, Ssh7a, Sso7d.

22. The fusion protein of claim 21, wherein the 7 kDa DNA-binding protein, or DNA-binding fragment thereof, is Sso7d or Aho7c.

23. The fusion protein of claim 22, wherein the 7 kDa DNA-binding protein comprises an amino acid sequence at least 70%, at least 80%, at least 90%, at least 95%, or 100% identical to an amino acid sequence of SEQ ID NO: 31 or 33.

24. The fusion protein of any one of the preceding claims, wherein the fusion protein has an editing efficiency that is increased compared to a same Cas enzyme or its fragment, lacking the DNA-binding protein or DNA-binding fragment thereof.

25. The fusion protein of claim 24, wherein the editing efficiency of the fusion protein is increased of at least 1-fold, 2-fold, 3-fold, 4-fold, 5-fold, 6-fold, 7-fold, 8-fold, 9-fold, or 10-fold.

26. The fusion protein of any one of the preceding claims, wherein the Cas enzyme is a type II Cas enzyme or a type V Cas enzyme.

27. The fusion protein of claim 26, wherein the Cas enzyme is a Cas9, or a Cas12 enzyme.

28. The fusion protein of claim 27, wherein the Cas enzyme is selected from the group consisting of SpCas9, SaCas9, NmeCas9, Cas12f1, AsCas12f1, CasMINI, and SpCas12f1.

29. The fusion protein of claim 28, wherein the Cas enzyme is SpCas9 or SaCas9.

30. The fusion protein of claim 29, wherein the Cas enzyme or its fragment comprises an amino acid sequence at least 70%, at least 80%, at least 90%, at least 95%, or 100% identical to an amino acid sequence of SEQ ID NO: 25, 27, 110, or 111.

31. The fusion protein of claim 28, wherein the Cas enzyme is Cas12f1 or SpCas12f1.

32. The fusion protein of claim 31, wherein the Cas enzyme or its fragment comprises an amino acid sequence at least 70%, at least 80%, at least 90%, at least 95%, or 100% identical to an amino acid sequence of SEQ ID NO: 29 or 112.

33. An expression cassette comprising a polynucleotide comprising a nucleotide sequence encoding a fusion protein, wherein the fusion protein comprises (1) a Cas enzyme or its fragment and (2) a DNA-binding protein or a DNA-binding fragment thereof.

34. The expression cassette of claim 33, wherein nucleotide sequence comprises a first portion encoding the Cas enzyme or its fragment and a second portion encoding the DNA-binding protein or DNA-binding fragment thereof.

35. The expression cassette of claim 34, wherein the first nucleotide sequence portion is located upstream of the second nucleotide sequence portion.

36. The expression cassette of claim 35, wherein the DNA-binding protein or DNA-binding fragment thereof is located downstream of the Cas enzyme.

37. The expression cassette of claim 36, wherein the DNA-binding protein or DNA-binding fragment thereof is linked to the Cas enzyme at the C-terminus.

38. The expression cassette of claim 34, wherein the first nucleotide sequence portion is localized downstream of the second nucleotide sequence portion.

39. The expression cassette of claim 38, wherein the DNA-binding protein or DNA-binding fragment thereof is located upstream of the Cas enzyme.

40. The expression cassette of claim 39, wherein the DNA-binding protein or DNA-binding fragment thereof is linked to the Cas enzyme or its fragment at the N-terminus.

41. The expression cassette of any one of the preceding claims, wherein the DNA-binding protein or DNA-binding fragment thereof is linked to the Cas enzyme or its fragment via one or more chemical bonds, by a linker, or a combination thereof.

42. The expression cassette of claim 41, wherein the DNA-binding protein or DNA-binding fragment thereof is linked to the Cas enzyme or its fragment via one or more covalent chemical bonds.

43. The expression cassette of claim 41, wherein the DNA-binding protein or DNA-binding fragment thereof is linked to the Cas enzyme or its fragment by a linker.

44. The expression cassette of claim 43, wherein the linker is a flexible linker, a rigid linker, or any combination thereof.

45. The expression cassette of claim 44, wherein the linker is selected from the group consisting of (G)ₙ, (G₄S)ₙ (SEQ ID NO: 125), (GSG)ₙ, (GGGGS)ₙ (SEQ ID NO: 58), (EAAK)ₙ (SEQ ID NO: 59), or (XP)ₙ, wherein n is an integer between 1 and 35 and X is any amino acid.

46. The expression cassette of any one of the preceding claims, wherein the polynucleotide further comprises a nucleotide sequence encoding at least one nuclear localization signal (NLS), at least one cell penetrating peptide (CPP), or a combination thereof.

47. The expression cassette of claim 46, wherein the polynucleotide comprises a single nucleotide sequence encoding a single NLS.

48. The expression cassette of claim 46, wherein the polynucleotide comprises multiple nucleotide sequences encoding a multiple NLS.

49. The expression cassette of claims 46-48, wherein the NLS is selected from the group consisting of SEQ ID NOs: 34-52.

50. The expression cassette of claims 46-49, wherein the at least one NLS or the at least one cell penetrating peptide is linked to the fusion protein via one or more chemical bonds, by a linker, or a combination thereof.

51. The expression cassette of claim 50, wherein the at least one NLS or the at least one cell penetrating peptide is linked to the fusion protein via one or more covalent chemical bonds.

52. The expression cassette of claim 50, wherein the at least one NLS or the at least one cell penetrating peptide is linked to the fusion protein by a linker.

53. The expression cassette of claim 52, wherein the linker is a flexible linker, a rigid linker, or any combination thereof.

54. The expression cassette of claim 53, wherein the linker is selected from the group consisting of (G)ₙ, (G₄S)ₙ (SEQ ID NO: 125), (GSG)ₙ, (GGGGS)ₙ (SEQ ID NO: 58), (EAAK)ₙ (SEQ ID NO: 59), or (XP)ₙ, wherein n is an integer between 1 and 35 and X is any amino acid.

55. The expression cassette of claims 33-54, wherein the nucleotide sequence encoding the NLS or the CPP is located downstream of the Cas enzyme or its fragment, upstream of the Cas enzyme or its fragment, downstream of the DNA-binding protein or DNA-binding fragment thereof, upstream of the DNA-binding protein or DNA-binding fragment thereof, or a combination thereof.

56. The expression cassette of claims 33-55, wherein the NLS or the CPP is linked to the N-terminus of the Cas enzyme or its fragment, the C-terminus of the Cas enzyme or its fragment, the N-terminus of the DNA-binding protein or DNA-binding fragment thereof, the C-terminus of the DNA-binding protein or DNA-binding fragment thereof, or a combination thereof.

57. The expression cassette of any one of the preceding claims, wherein the polynucleotide further comprises a nucleotide sequence encoding a molecular tag, a selectable marker, a self-cleaving peptide or any combination thereof.

58. The expression cassette of claim 57, wherein the nucleotide sequence encoding the self-cleaving peptide is located upstream of the nucleotide sequence encoding the Cas enzyme or its fragment, upstream of the nucleotide sequence encoding the DNA-binding protein or DNA-binding fragment thereof, upstream of the nucleotide sequence encoding the molecular tag, and downstream of the nucleotide sequence encoding the selectable marker.

59. The expression cassette of claim 57, wherein the nucleotide sequence encoding the self-cleaving peptide is located downstream of the nucleotide sequence encoding the Cas enzyme or its fragment, downstream of the nucleotide sequence encoding the DNA-binding protein or DNA-binding fragment thereof, downstream of the nucleotide sequence encoding the molecular tag, and upstream of the nucleotide sequence encoding the selectable marker.

60. The expression cassette of claims 57-59, wherein the molecular tag is selected from the group consisting of CBP, FLAG, GST, HA, HBH, MBP, Myc, polyHis, S-tag, SUMO, TAP, TRX, or V5.

61. The expression cassette of claims 57-60, wherein the selectable marker is an ampicillin, a streptomycin, a gentamycin, a kanamycin, a hygromycin, a bialaphos herbicide, a sulfonamide resistance conferring selectable marker.

62. The expression cassette of claims 57-61, wherein the self-cleaving peptide is selected from the group consisting of P2A, E2A, F2A, T2A, or furin cleavage site.

63. The expression cassette of claims 57-62, wherein the polynucleotide is translated into a polypeptide comprising the Cas enzyme or its fragment, the DNA-binding protein or DNA-binding fragment thereof, the self-cleaving peptide, the selectable marker, and optionally the NLS, the CPP, the linker, and the molecular tag.

64. The expression cassette of claim 63, wherein the self-cleaving peptide undergoes cleavage upon translation.

65. The expression cassette of claim 64, wherein the cleavage of the self-cleaving peptide produces a first product polypeptide comprising the Cas enzyme or its fragment, the DNA-binding protein or DNA-binding fragment thereof, and, optionally, portion of the self-cleaving peptide, the NLS, the CPP, the linker, and the molecular tag, and a second product polypeptide comprising the selectable marker and optionally portion of the self-cleaving peptide.

66. The expression cassette of claims 33-65, wherein the DNA-binding protein or a DNA-binding fragment thereof is a 7 kDa DNA-binding protein, a zinc finger protein, or a transcription activator-like effector protein, or a DNA-binding fragment thereof.

67. The expression cassette of claims 33-65, wherein the DNA-binding protein or a DNA-binding fragment thereof is a 7 kDa DNA-binding protein, a zinc finger protein, or a transcription activator-like effector protein, or a DNA-binding fragment thereof.

68. The expression cassette of claim 67, wherein the 7 kDa DNA-binding protein, or DNA-binding fragment thereof, is selected from the group consisting of Sac7d, Sac7e, Mse7, Mcu7, Aho7a, Aho7b, Aho7c, Sto7, Ssh7b, Sis7a, Sis7b, Ssh7a, Sso7d.

69. The expression cassette of claim 68, wherein the 7 kDa DNA-binding protein, or DNA-binding fragment thereof, is Sso7d or Aho7c.

70. The expression cassette of claim 69, wherein the 7 kDa DNA-binding protein comprises an amino acid sequence at least 70%, at least 80%, at least 90%, at least 95%, or 100% identical to an amino acid sequence of SEQ ID NO: 31 or 33.

71. The expression cassette of claims 33-70, wherein the fusion protein has an editing efficiency that is increased compared to a same Cas enzyme or its fragment, lacking the DNA-binding protein or a DNA-binding fragment thereof.

72. The expression cassette of claim 71 wherein the editing efficiency of the fusion protein is increased of at least 1-fold, 2-fold, 3-fold, 4-fold, 5-fold, 6-fold, 7-fold, 8-fold, 9-fold, or 10-fold.

73. The expression cassette of any one of the preceding claims, wherein the polynucleotide further comprises a nucleotide sequence encoding a promoter, an enhancer, a silencer, a polyA tail, a poly adenylation signal, a 3′-UTR, a 5′-UTR, an intronic sequence, or any combination thereof.

74. The expression cassette of claim 73, wherein the promoter is a constitutive promoter, a regulatable promoter, an ubiquitous promoter, or a tissue-specific promoter.

75. The expression cassette of claim 74, wherein the promoter is an UbC promoter or a CBh promoter.

76. The expression cassette of claim 75, wherein the promoter comprises a nucleotide sequence at least 70%, at least 80%, at least 90%, at least 95%, or 100% identical to an amino acid sequence of SEQ ID NO: 53 or 54.

77. The expression cassette of claims 33-76, wherein the nucleotide sequence of the polynucleotide is codon optimized for translation in a mammalian cell.

78. The expression cassette of claims 33-77, wherein the Cas enzyme is a type II Cas enzyme or a type V Cas enzyme.

79. The expression cassette of claim 78, wherein the Cas enzyme is a Cas9, or a Cas12 enzyme.

80. The expression cassette of claim 79, wherein the Cas enzyme is selected from the group consisting of SpCas9, SaCas9, NmeCas9, Cas12f1, AsCas12f1, CasMINI, and SpCas12f1.

81. The expression cassette of claim 80, wherein the Cas enzyme is SpCas9 or SaCas9.

82. The expression cassette of claim 81, wherein the Cas enzyme or its fragment comprises an amino acid sequence at least 70%, at least 80%, at least 90%, at least 95%, or 100% identical to an amino acid sequence of SEQ ID NO: 25, 27, 110 or 111.

83. The expression cassette of claim 82, wherein the polynucleotide comprises a nucleotide sequence least 70%, at least 80%, at least 90%, at least 95%, or 100% identical to a nucleotide sequence of SEQ ID NO: 24, 26, 113 or 114.

84. The expression cassette of claims 33-83, wherein the expression cassette encodes an amino acid sequence at least 70%, at least 80%, at least 90%, at least 95%, or 100% identical to an amino acid sequence of SEQ ID NO: 16, 18, 119, or 121.

85. The expression cassette of claim 84, wherein the expression cassette comprises a nucleotide sequence at least 70%, at least 80%, at least 90%, at least 95%, or 100% identical to a nucleotide sequence of SEQ ID NO: 15, 17, 120, or 122.

86. The expression cassette of claim 80, wherein the Cas enzyme is Cas12f1 or SpCas12f1.

87. The expression cassette of claim 86, wherein the Cas enzyme comprises an amino acid sequence at least 70%, at least 80%, at least 90%, at least 95%, or 100% identical to an amino acid sequence of SEQ ID NO: 29 or 112.

88. The expression cassette of claim 87, wherein the polynucleotide comprises a nucleotide sequence least 70%, at least 80%, at least 90%, at least 95%, or 100% identical to a nucleotide sequence of SEQ ID NO: 28 or 115.

89. The expression cassette of claim 33-80 or 86-88 wherein the expression cassette encodes an amino acid sequence at least 70%, at least 80%, at least 90%, at least 95%, or 100% identical to an amino acid sequence of SEQ ID NO: 11, 20, 117, or 123.

90. The expression cassette of claim 89, wherein the expression cassette comprises a nucleotide sequence at least 70%, at least 80%, at least 90%, at least 95%, or 100% identical to a nucleotide sequence of SEQ ID NO: 10, 19, 118, or 124.

91. A vector comprising an expression cassette of any one of claims 33-90.

92. The vector of claim 91, wherein the vector further comprises a backbone nucleotide sequence.

93. The vector of claim 92, wherein the backbone nucleotide sequence encodes a prokaryotic origin of replication, a multiple cloning site, a resistance conferring selectable marker, a promoter, an enhancer, a silencer, a polyA tail, a poly adenylation signal, a 3′-UTR, a 5′-UTR, an intronic sequence, or any combination thereof.

94. The vector of claims 91-93, wherein the vector comprises an expression cassette of any one of claims 32-83.

95. The vector of claims 91-93, wherein the vector comprises an expression cassette of any one of claim 32-78 or 84-88.

96. The vector of claim 95, wherein the vector comprises a nucleotide sequence at least 70%, at least 80%, at least 90%, at least 95%, or 100% identical to a nucleotide sequence of SEQ ID NO: 12, 14, or 116.

97. A composition for cleaving a target nucleic acid sequence in a cell comprising the fusion protein of claims 1-32, the expression cassette of claims 33-90, or the vector of claims 91-96 and at least one guide RNA, at least one vector encoding the at least one guide RNA, or any combination thereof.

98. The composition of claim 97, wherein the Cas enzyme or its fragment, and the guide RNA (gRNA) are capable of forming a Cas enzyme/gRNA or Cas enzyme fragment/gRNA complex in the cell that introduces a double stranded cleavage at the target nucleic acid sequence.

99. The composition of claim 98, wherein the double stranded cleavage is repaired by a cell DNA damage repair machinery.

100. The composition of claim 99, wherein the cell DNA damage repair machinery repairs the double stranded cleavage via nonhomologous end-joining (NHEJ).

101. The composition of claims 97-100, wherein the composition further comprises a repair template and wherein, optionally, wherein the cell DNA damage repair machinery repairs the double stranded cleavage via high-fidelity homology-directed repair (HDR).

102. The composition of claim 101, wherein the repair template is a double-stranded DNA targeting constructs, a single-stranded DNA oligonucleotides (ssODNs), or a combination thereof, and comprises homology arms flanking the double stranded cleavage.

103. The composition of claims 97-102, wherein the guide RNA is chemically synthesized, is transcribed from the vector encoding the guide RNA in vitro before introduction into the cell, or is transcribed from the vector encoding the guide RNA in vivo in the cell, wherein the vector encoding the guide RNA and the vector of any one of claims 89-94, are the same or are different.

104. The composition of claims 97-103, wherein the composition comprises the fusion protein of claims 1-29, the expression cassette of claims 32-83, or the vector of claim 89-92.

105. The composition of claim 104 wherein the guide RNA comprises a crRNA portion and a tracrRNA portion.

106. The composition of claim 105, wherein the crRNA portion and the tracrRNA portion are fused.

107. The composition of claims 105-106, wherein the target nucleic acid sequence comprises a region that is at least 80%, at least 90%, at least 95%, or 100% complementary to the crRNA portion of the guide RNA.

108. The composition of any one of claims 104-107, wherein the target nucleic acid sequence further comprises a protospacer adjacent motif (PAM).

109. The composition of claim 107, wherein the PAM comprises a nucleotide sequence of 5′-NGG, 5′-NGGNG, 5′-NNAGAAW, or 5′-ACAY.

110. The composition of claim 97-103, wherein the composition comprises the fusion protein of claim 1-27 or 30-31, the expression cassette of claims 32-78, 84-88, or the vector of claim 89-91 or 93-94.

111. The composition of claim 109, wherein the guide RNA comprises a crRNA.

112. The composition of claim 110, wherein the target nucleic acid sequence comprises a region that is at least 80%, at least 90%, at least 95%, or 100% complementary to the crRNA.

113. The composition of claims 110-112, wherein the target nucleic acid sequence further comprises a protospacer adjacent motif (PAM).

114. The composition of claim 113, wherein the PAM is a 5′ T-rich sequence.

115. The composition of claim 114, wherein the PAM comprises a nucleotide sequence of ′-NTTC, 5′-GTTT, 5′-GTTC, 5′-AATC, 5′-ACTC, 5′-GGTC, or 5′-GCTC.

116. A method of editing a genome in a cell comprising contacting the cell with the composition of any one of claims 99-115.

117. The method of claim 116, wherein contacting the cell with the composition results in editing the target nucleic acid sequence.

118. The method of claim 117, wherein editing the target nucleic acid region comprises introducing an indel or a large deletion in the target nucleic acid sequence.

119. A method of editing a genome in a cell comprising contacting the cell with the composition of any one of claims 101-115.

120. The method of claim 119, wherein contacting the cell with the composition results in editing the target nuclei acid sequence.

121. The method of claim 120, wherein editing the target nuclei acid region comprises introducing an exogenous sequence in the repair template in the target nucleic acid sequence.

122. The method of claims 116-121, wherein the genome of the cell is edited with an efficiency which is increased compared to the efficiency at which the genome of the cell is edited when the cell is contacted with a composition comprising a same Cas enzyme, a same guide RNA, and, optionally, a same repair template, but not comprising a DNA-binding protein or a DNA-binding fragment thereof.

123. The method of claim 122, wherein the efficiency is increased of at least 1-fold, 2-fold, 3-fold, 4-fold, 5-fold, 6-fold, 7-fold, 8-fold, 9-fold, or 10-fold.

124. The method of any one of the preceding claims, wherein the cell is in a subject.

125. The method of claim 124, wherein the subject is a human.

126. The method of claim 124 or 125, wherein the subject suffers from a genetic disease.

127. The method of claim 126, wherein the genetic disease is selected from the group consisting of achondroplasia, hereditary hemochromatosis, Down Syndrome, hereditary spherocytosis, Tay-Sachs Disease, Usher syndrome, hereditary fructose intolerance, hemophilia, muscular dystrophy, DMD, DM1, DM2, Dilated Cardiomyopathy, TTN-DCM, Huntington's disease, polygenic disorders, breast cancer, ovarian cancer, Parkinson's disease, Bardet-Biedl syndrome, Prader-Willi syndrome, diabetes, heart disease, arthritis, motor neuron disease, albinism, Cri-du-Chat syndrome, cystic fibrosis, fragile X syndrome, galactosemia, Huntington's disease, Jackson-Weiss syndrome, Klinefelter syndrome, Krabbe disease, Langer-Giedion syndrome, Lesch-Nyhan syndrome, Marfan syndrome, myotonic dystrophy, Nail-Patella syndrome, neurofibromatosis, Noonan syndrome, triple X syndrome, osteogenesis imperfecta, Patau syndrome, phenylketonuria, porphyria, retinoblastoma, Rett syndrome, sickle cell disease, Turner syndrome, Usher syndrome, Von Hippel-Lindau syndrome, Waardenburg syndrome, Wilson's disease, xeroderma pigmentosum, XXXX syndrome, or YY syndrome can be characterized by an impaired production of a protein, or by a defective splicing. A hereditary disease such as for example: achondroplasia, hereditary hemochromatosis, Down Syndrome, hereditary spherocytosis, Tay-Sachs Disease, Usher syndrome, hereditary fructose intolerance, hemophilia, muscular dystrophy, Duchenne muscular dystrophy or DMD, polygenic disorders, breast cancer, ovarian cancer, Parkinson's disease, Bardet-Biedl syndrome, Prader-Willi syndrome, diabetes, heart disease, arthritis, motor neuron disease, albinism, Cri-du-Chat syndrome, cystic fibrosis, fragile X syndrome, galactosemia, Huntington's disease, Jackson-Weiss syndrome, Klinefelter syndrome, Krabbe disease, Langer-Giedion syndrome. Lesch-Nyhan syndrome, Marfan syndrome, myotonic dystrophy, Nail-Patella syndrome, neurofibromatosis, Noonan syndrome, triple X syndrome, osteogenesis imperfecta, Patau syndrome, phenylketonuria, porphyria, retinoblastoma, Rett syndrome, sickle cell disease, Turner syndrome, Usher syndrome, Von Hippel-Lindau syndrome, Waardenburg syndrome, Wilson's disease, xeroderma pigmentosum, XXXX syndrome, and YY syndrome.

128. The method of claim 127, wherein the genetic disease is a muscular dystrophy.

129. The method of claim 128, wherein the muscular dystrophy is Duchenne muscular dystrophy.

130. A pharmaceutical formulation comprising the composition of claims 97-115 or a pharmaceutically acceptable salt thereof, and at least one pharmaceutically acceptable excipient or adjuvant.

131. A method of treating a subject comprising administering to the subject the composition of claims 97-115 or a pharmaceutically acceptable salt thereof, or the pharmaceutical formulation of claim 130.

132. The method of claim 131, wherein the subject is a human.

133. The method of claim 131 or 132, wherein the subject suffers from a genetic disease.

134. The method of claim 133, wherein the genetic disease is selected from the group consisting of achondroplasia, hereditary hemochromatosis, Down Syndrome, hereditary spherocytosis, Tay-Sachs Disease, Usher syndrome, hereditary fructose intolerance, hemophilia, muscular dystrophy (e.g., Duchenne muscular dystrophy or DMD), polygenic disorders, breast cancer, ovarian cancer, Parkinson's disease, Bardet-Biedl syndrome, Prader-Willi syndrome, diabetes, heart disease, arthritis, motor neuron disease, albinism, Cri-du-Chat syndrome, cystic fibrosis, fragile X syndrome, galactosemia, Huntington's disease, Jackson-Weiss syndrome, Klinefelter syndrome, Krabbe disease, Langer-Giedion syndrome, Lesch-Nyhan syndrome, Marfan syndrome, myotonic dystrophy, Nail-Patella syndrome, neurofibromatosis, Noonan syndrome, triple X syndrome, osteogenesis imperfecta, Patau syndrome, phenylketonuria, porphyria, retinoblastoma, Rett syndrome, sickle cell disease, Turner syndrome, Usher syndrome, Von Hippel-Lindau syndrome, Waardenburg syndrome, Wilson's disease, xeroderma pigmentosum, XXXX syndrome, or YY syndrome can be characterized by an impaired production of a protein, or by a defective splicing. A hereditary disease such as for example: achondroplasia, hereditary hemochromatosis, Down Syndrome, hereditary spherocytosis, Tay-Sachs Disease, Usher syndrome, hereditary fructose intolerance, hemophilia, muscular dystrophy, Duchenne muscular dystrophy or DMD, polygenic disorders, breast cancer, ovarian cancer, Parkinson's disease, Bardet-Biedl syndrome, Prader-Willi syndrome, diabetes, heart disease, arthritis, motor neuron disease, albinism, Cri-du-Chat syndrome, cystic fibrosis, fragile X syndrome, galactosemia, Huntington's disease, Jackson-Weiss syndrome, Klinefelter syndrome, Krabbe disease, Langer-Giedion syndrome. Lesch-Nyhan syndrome, Marfan syndrome, myotonic dystrophy, Nail-Patella syndrome, neurofibromatosis, Noonan syndrome, triple X syndrome, osteogenesis imperfecta, Patau syndrome, phenylketonuria, porphyria, retinoblastoma, Rett syndrome, sickle cell disease, Turner syndrome, Usher syndrome, Von Hippel-Lindau syndrome, Waardenburg syndrome, Wilson's disease, xeroderma pigmentosum, XXXX syndrome, and YY syndrome.

135. The method of claim 134, wherein the genetic disease is a muscular dystrophy.

136. The method of claim 135, wherein the muscular dystrophy is Duchenne muscular dystrophy.


SEQUENCE LISTING (125 sequences)

SEQ ID NO: 1 - DNA, 73 bp, synthetic construct
  source 1..73: mol_type = other DNA
aaaaggccggcggccacgaaaaaggccggccaggcaaaaaagaaaaagtacccatacgatgttccagattacg

SEQ ID NO: 2 - DNA, 18 bp, synthetic construct
  source 1..18: mol_type = other DNA
cctccgagagctgcatca

SEQ ID NO: 3 - DNA, 200 bp, synthetic construct
  source 1..200: mol_type = other DNA
cttcttctgcttctcgagcatctgcaacaattcctttggagcgtccttttctgaaacggctcctctgccagtctttcctcctccttcatcgtacgtaaagctgatcatcttacctacgcgccacactttcttgattttactgatgtccacctctttctcttcccctttatacttaaacttcactgtagccattccgcttc

SEQ ID NO: 4 - DNA, 41 bp, synthetic construct
  source 1..41: mol_type = other DNA
agagcactaaggaggagcccggaagcggaatggctacagtg

SEQ ID NO: 5 - DNA, 51 bp, synthetic construct
  source 1..51: mol_type = other DNA
ttcgtggccgccggcctttttccgcttcccttcttctgcttctcgagcatc

SEQ ID NO: 6 - DNA, 22 bp, synthetic construct
  source 1..22: mol_type = other DNA
gccaaagattgataagggcgtg

SEQ ID NO: 7 - DNA, 18 bp, synthetic construct
  source 1..18: mol_type = other DNA
gggctcctccttagtgct

SEQ ID NO: 8 - DNA, 19 bp, synthetic construct
  source 1..19: mol_type = other DNA
aggtcttgcaacgtgacac

SEQ ID NO: 9 - DNA, 12069 bp, synthetic construct
  source 1..12069: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggctagcttaattaacctgcaggcccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgactctaggcccaggcggccttgtacaaaaaagcaccggtaggctgccaccatgccaaagaagaagcggaaggtcggtatccacggagtcccagcagccatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagcccaaaaggccggcggccacgaaaaaggccggccaggcaaaaaagaaaaagtacccatacgatgttccagattacgctggaagcggagagggcaggggaagtcttctaacatgcggggacgtggaggaaaatcccggccccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagacccgaattccagggccaggccggccgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 10 - DNA, 3009 bp, synthetic construct
  source 1..3009: mol_type = other DNA
atgccaaagaagaagcggaaggtcggtatccacggagtcccagcagccatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagcccggatccggaatggctacagtgaagtttaagtataaaggggaagagaaagaggtggacatcagtaaaatcaagaaagtgtggcgcgtaggtaagatgatcagctttacgtacgatgaaggaggaggaaagactggcagaggagccgtttcagaaaaggacgctccaaaggaattgttgcagatgctcgagaagcagaagaagggaagcggaaaaaggccggcggccacgaaaaaggccggccaggcaaaaaagaaaaagtacccatacgatgttccagattacgctggaagcggagagggcaggggaagtcttctaacatgcggggacgtggaggaaaatcccggccccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 11 - protein, 1002 aa, synthetic construct
MPKKKRKVGIHGVPAAMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEPGSGMATVKFKYKGEEKEVDISKIKKVWRVGKMISFTYDEGGGKTGRGAVSEKDAPKELLQMLEKQKKGSGKRPAATKKAGQAKKKKYPYDVPDYAGSGEGRGSLLTCGDVEENPGPMKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 12 - DNA, 12279 bp, synthetic construct
  source 1..12279: mol_type = other DNA
gccgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggctagcttaattaacctgcaggcccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgactctaggcccaggcggccttgtacaaaaaagcaccggtaggctgccaccatgccaaagaagaagcggaaggtcggtatccacggagtcccagcagccatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagcccggatccggaatggctacagtgaagtttaagtataaaggggaagagaaagaggtggacatcagtaaaatcaagaaagtgtggcgcgtaggtaagatgatcagctttacgtacgatgaaggaggaggaaagactggcagaggagccgtttcagaaaaggacgctccaaaggaattgttgcagatgctcgagaagcagaagaagggaagcggaaaaaggccggcggccacgaaaaaggccggccaggcaaaaaagaaaaagtacccatacgatgttccagattacgctggaagcggagagggcaggggaagtcttctaacatgcggggacgtggaggaaaatcccggccccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagacccgaattccagggccaggccg

SEQ ID NO: 13 - DNA, 11564 bp, synthetic construct
  source 1..11564: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggctagcttaattaaccggccgccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggtacgccggtaggctgccaccatgccaaagaagaagcggaaggtcggtatccacggagtcccagcagccatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagcccaaaaggccggcggccacgaaaaaggccggccaggcaaaaaagaaaaagtacccatacgatgttccagattacgctggaagcggagagggcaggggaagtcttctaacatgcggggacgtggaggaaaatcccggccccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagacccgaattccagggccaggccggccgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 14 - DNA, 11774 bp, synthetic construct
  source 1..11774: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggctagcttaattaaccggccgccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggtacgccggtaggctgccaccatgccaaagaagaagcggaaggtcggtatccacggagtcccagcagccatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagcccggatccggaatggctacagtgaagtttaagtataaaggggaagagaaagaggtggacatcagtaaaatcaagaaagtgtggcgcgtaggtaagatgatcagctttacgtacgatgaaggaggaggaaagactggcagaggagccgtttcagaaaaggacgctccaaaggaattgttgcagatgctcgagaagcagaagaagggaagcggaaaaaggccggcggccacgaaaaaggccggccaggcaaaaaagaaaaagtacccatacgatgttccagattacgctggaagcggagagggcaggggaagtcttctaacatgcggggacgtggaggaaaatcccggccccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagacccgaattccagggccaggccggccgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 15 - DNA, 5568 bp, synthetic construct
  source 1..5568: mol_type = other DNA
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacggatccggaatggctacagtgaagtttaagtataaaggggaagagaaagaggtggacatcagtaaaatcaagaaagtgtggcgcgtaggtaagatgatcagctttacgtacgatgaaggaggaggaaagactggcagaggagccgtttcagaaaaggacgctccaaaggaattgttgcagatgctcgagaagcagaagaagggaagcggaaaaaggccggcggccacgaaaaaggccggccaggcaaaaaagaaaaagggaagcggagagggcaggggaagtcttctaacatgcggggacgtggaggaaaatcccggccccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 16 - protein, 1855 aa, synthetic construct
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSGMATVKFKYKGEEKEVDISKIKKVWRVGKMISFTYDEGGGKTGRGAVSEKDAPKELLQMLEKQKKGSGKRPAATKKAGQAKKKKGSGEGRGSLLTCGDVEENPGPMKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 17 - DNA, 4581 bp, synthetic construct
  source 1..4581: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcggatccggaatggctacagtgaagtttaagtataaaggggaagagaaagaggtggacatcagtaaaatcaagaaagtgtggcgcgtaggtaagatgatcagctttacgtacgatgaaggaggaggaaagactggcagaggagccgtttcagaaaaggacgctccaaaggaattgttgcagatgctcgagaagcagaagaagggaagcggaaaaaggccggcggccacgaaaaaggccggccaggcaaaaaagaaaaagtacccatacgatgttccagattacgctggaagcggagagggcaggggaagtcttctaacatgcggggacgtggaggaaaatcccggccccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 18 - protein, 1526 aa, synthetic construct
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGGSGMATVKFKYKGEEKEVDISKIKKVWRVGKMISFTYDEGGGKTGRGAVSEKDAPKELLQMLEKQKKGSGKRPAATKKAGQAKKKKYPYDVPDYAGSGEGRGSLLTCGDVEENPGPMKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 19 - DNA, 2997 bp, synthetic construct
  source 1..2997: mol_type = other DNA
atgccaaagaagaagcggaaggtcggtatccacggagtcccagcagccatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagcccggatccggaatggcgactaaagtgaagttcaaatataagggtgaagaaaaggaagtagatatatccaagatcaagaaggtatggcgggttggaaaaatgatcagctttacctatgacgacaacggaaagactggtaggggtgccgtatctgaaaaagatgcccccaaagaattgttggaaaaactcaagggaagcggaaaaaggccggcggccacgaaaaaggccggccaggcaaaaaagaaaaagtacccatacgatgttccagattacgctggaagcggagagggcaggggaagtcttctaacatgcggggacgtggaggaaaatcccggccccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 20 - protein, 998 aa, synthetic construct
MPKKKRKVGIHGVPAAMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEPGSGMATKVKFKYKGEEKEVDISKIKKVWRVGKMISFTYDDNGKTGRGAVSEKDAPKELLEKLKGSGKRPAATKKAGQAKKKKYPYDVPDYAGSGEGRGSLLTCGDVEENPGPMKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 21 - DNA, 11163 bp, synthetic construct
  source 1..11163: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggctagcttaattaagtaaaacgacggccagtgagcgcgcgtaatacgactcactatagggcgaattgggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaccgcttcaccgagtgaaggtgggctgcttgcatcagcctaatgtcgagaagtgctttcttcggaaagtaaccctcgaaacaaagaaaggaatgcaacggagacggacgtctctttttattttttgcggccgcccgcggtggagctccagcttttgttccctttagtgagggttaattgcgcgcttggcgtaatcatggtcatagctgtttcctgttaattaacctgcaggcccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgactctaggcccaggcggccggatccccgggtaccggtcgccaccatggatagcactgagaacgtcatcaagcccttcatgcgcttcaaggtgcacatggagggctccgtgaacggccacgagttcgagatcgagggcgagggcgagggcaagccctacgagggcacccagaccgccaagctgcaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctctctgcaggacggcaccttcatctaccacgtgaagttcatcggcgtgaacttcccctccgacggccccgtaatgcagaagaagactctgggctgggagccctccaccgagcgcctgtacccccgcgacggcgtgctgaagggcgagatccacaaggcgctgaagctgaagggcggcggccactacctggtggagttcaagtcaatctacatggccaagaagcccgtgaagctgcccggctactactacgtggactccaagctggacatcacctcccacaacgaggactacaccgtggtggagcagtacgagcgcgccgaggcccgccaccacctgttccagactcacggtatggacgaattgtacaagcacgacgaattggagggcagaggaagtcttctaacatgcggtgacgtggaggagaatcccggccctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtgtcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatctagaattcggccaggccggccgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 22 - DNA, 19 bp, synthetic construct
  source 1..19: mol_type = other DNA
gtaaaacgacggccagtga

SEQ ID NO: 23 - RNA, 60 nt, synthetic construct
  source 1..60: mol_type = other RNA
cgtctcaaaaaataaaannnnnnnnnnnnnnnnnnnngttgcattcctttctttgtttcg

SEQ ID NO: 24 - DNA, 5358 bp, synthetic construct
  source 1..5358: mol_type = other DNA
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagggaagcggagagggcaggggaagtcttctaacatgcggggacgtggaggaaaatcccggccccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 25 - protein, 1785 aa, synthetic construct
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKGSGEGRGSLLTCGDVEENPGPMKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 26 - DNA, 4371 bp, synthetic construct
  source 1..4371: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtacccatacgatgttccagattacgctggaagcggagagggcaggggaagtcttctaacatgcggggacgtggaggaaaatcccggccccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 27 - protein, 1456 aa, synthetic construct
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKYPYDVPDYAGSGEGRGSLLTCGDVEENPGPMKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 28 - DNA, 2799 bp, synthetic construct
  source 1..2799: mol_type = other DNA
atgccaaagaagaagcggaaggtcggtatccacggagtcccagcagccatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagcccaaaaggccggcggccacgaaaaaggccggccaggcaaaaaagaaaaagtacccatacgatgttccagattacgctggaagcggagagggcaggggaagtcttctaacatgcggggacgtggaggaaaatcccggccccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 29 - protein, 932 aa, synthetic construct
MPKKKRKVGIHGVPAAMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEPKRPAATKKAGQAKKKKYPYDVPDYAGSGEGRGSLLTCGDVEENPGPMKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 30 - DNA, 192 bp, synthetic construct
  source 1..192: mol_type = other DNA
atggctacagtgaagtttaagtataaaggggaagagaaagaggtggacatcagtaaaatcaagaaagtgtggcgcgtaggtaagatgatcagctttacgtacgatgaaggaggaggaaagactggcagaggagccgtttcagaaaaggacgctccaaaggaattgttgcagatgctcgagaagcagaagaag

SEQ ID NO: 31 - protein, 64 aa, synthetic construct
MATVKFKYKGEEKEVDISKIKKVWRVGKMISFTYDEGGGKTGRGAVSEKDAPKELLQMLEKQKK

SEQ ID NO: 32 - DNA, 180 bp, synthetic construct
  source 1..180: mol_type = other DNA
atggcgactaaagtgaagttcaaatataagggtgaagaaaaggaagtagatatatccaagatcaagaaggtatggcgggttggaaaaatgatcagctttacctatgacgacaacggaaagactggtaggggtgccgtatctgaaaaagatgcccccaaagaattgttggaaaaactcaag

SEQ ID NO: 33 - protein, 60 aa, synthetic construct
MATKVKFKYKGEEKEVDISKIKKVWRVGKMISFTYDDNGKTGRGAVSEKDAPKELLEKLK

SEQ ID NO: 34 - protein, 6 aa, synthetic construct
PKLKRQ

SEQ ID NO: 35 - protein, 4 aa, synthetic construct
RPRK

SEQ ID NO: 36 - protein, 8 aa, synthetic construct
RRARRPRG

SEQ ID NO: 37 - protein, 21 aa, synthetic construct
GKRKLITSEEERSPAKRGRKS

SEQ ID NO: 38 - protein, 22 aa, synthetic construct
KGKKGRTQKEKKAARARSKGKN

SEQ ID NO: 39 - protein, 28 aa, synthetic construct
RKRCAAGVGGGPAGCPAPGSTPLKKPRR

SEQ ID NO: 40 - protein, 36 aa, synthetic construct
RKPVTAQERQREREEKRRRRQERAKEREKRRQERER

SEQ ID NO: 41 - protein, 27 aa, synthetic construct
RSGGNHRRNGRGGRGGYNRRNNGYHPY

SEQ ID NO: 42 - protein, 27 aa, synthetic construct
TLLLRETMNNLGVSDHAVLSRKTPQPY

SEQ ID NO: 43 - protein, 18 aa, synthetic construct
PGKMDKGEHRQERRDRPY

SEQ ID NO: 44 - protein, 21 aa, synthetic construct
GKKKKGKPGKRREQRKKKRRT

SEQ ID NO: 45 - protein, 27 aa, synthetic construct
SANKVTKNKSNSSPYLNKRKGKPGPDS

SEQ ID NO: 46 - protein, 43 aa, synthetic construct
VHSHKKKKIPTSPTFTTPKTLTLRRQPKYPRKSAPRRNKLDHY

SEQ ID NO: 47 - protein, 10 aa, synthetic construct
RKHKTNRKPR

SEQ ID NO: 48 - protein, 8 aa, synthetic construct
NRRAKAKR

SEQ ID NO: 49 - protein, 6 aa, synthetic construct
RNKKKK

SEQ ID NO: 50 - protein, 5 aa, synthetic construct
RKVIK

SEQ ID NO: 51 - protein, 7 aa, synthetic construct
PKKKRKV

SEQ ID NO: 52 - protein, 16 aa, synthetic construct
KRPAATKKAGQAKKKK

SEQ ID NO: 53 - DNA, 1212 bp, synthetic construct
  source 1..1212: mol_type = other DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 54 - DNA, 778 bp, synthetic construct
  source 1..778: mol_type = other DNA
ccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 55 - protein, 9 aa, synthetic construct
YPYDVPDYA

SEQ ID NO: 56 - protein, 18 aa, synthetic construct
EGRGSLLTCGDVEENPGP

SEQ ID NO: 57 - protein, 341 aa, synthetic construct
MKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 58 - protein, 5 aa, synthetic construct
  VARIANT 1..5: note = wherein n is any integer, such as an integer in the rage of 1-35
  REGION 1..5: note = Linker
GGGGS

SEQ ID NO: 59 - protein, 4 aa, synthetic construct
  REGION 1..4: note = Linker
  VARIANT 1..4: note = wherein n is any integer, such as an integer in the rage of 1-35
EAAK

SEQ ID NO: 60 - DNA, 55 bp, synthetic construct
  source 1..55: mol_type = other DNA
acactctttccctacacgacgctcttccgatctccgcttttataagggcactgat

SEQ ID NO: 61 - DNA, 50 bp, synthetic construct
  source 1..50: mol_type = other DNA
acactctttccctacacgacgctcttccgatctctttgtggaactcgcaa

SEQ ID NO: 62 - DNA, 56 bp, synthetic construct
  source 1..56: mol_type = other DNA
acactctttccctacacgacgctcttccgatctcagcttgtggataagaaaggatt

SEQ ID NO: 63 - DNA, 57 bp, synthetic construct
  source 1..57: mol_type = other DNA
acactctttccctacacgacgctcttccgatctgttggacactgtagagtaagtcag

SEQ ID NO: 64 - DNA, 58 bp, synthetic construct
  source 1..58: mol_type = other DNA
acactctttccctacacgacgctcttccgatctgggaataatcgaaatgaaagcaggt

SEQ ID NO: 65 - DNA, 52 bp, synthetic construct
  source 1..52: mol_type = other DNA
acactctttccctacacgacgctcttccgatctcaaaatctgtgggctcctg

SEQ ID NO: 66 - DNA, 55 bp, synthetic construct; identical to SEQ ID NO: 60
  source 1..55: mol_type = other DNA

SEQ ID NO: 67 - DNA, 56 bp, synthetic construct
  source 1..56: mol_type = other DNA
acactctttccctacacgacgctcttccgatctgatataagttcccactttgagct

SEQ ID NO: 68 - DNA, 53 bp, synthetic construct
  source 1..53: mol_type = other DNA
acactctttccctacacgacgctcttccgatctcgctatggaactatgctagc

SEQ ID NO: 69 - DNA, 60 bp, synthetic construct
  source 1..60: mol_type = other DNA
acactctttccctacacgacgctcttccgatctcaattacagtgaaacatgtttctctct

SEQ ID NO: 70 - DNA, 58 bp, synthetic construct
  source 1..58: mol_type = other DNA
acactctttccctacacgacgctcttccgatctagtatctcacaatctagaaagcaca

SEQ ID NO: 71 - DNA, 53 bp, synthetic construct
  source 1..53: mol_type = other DNA
acactctttccctacacgacgctcttccgatctttgacatgtggactgaaacc

SEQ ID NO: 72 - DNA, 55 bp, synthetic construct
  source 1..55: mol_type = other DNA
acactctttccctacacgacgctcttccgatctcagtctctggcattcctttata

SEQ ID NO: 73 - DNA, 57 bp, synthetic construct
  source 1..57: mol_type = other DNA
acactctttccctacacgacgctcttccgatctacagtgaacatatgtgctaacaaa

SEQ ID NO: 74 - DNA, 54 bp, synthetic construct
  source 1..54: mol_type = other DNA
acactctttccctacacgacgctcttccgatctagggatttatctgggaaaaca

SEQ ID NO: 75 - DNA, 57 bp, synthetic construct
  source 1..57: mol_type = other DNA
acactctttccctacacgacgctcttccgatctggtgctttggaattataatcctca

SEQ ID NO: 76 - DNA, 55 bp, synthetic construct
  source 1..55: mol_type = other DNA
acactctttccctacacgacgctcttccgatcttatcacagtcaacactagaggc

SEQ ID NO: 77 - DNA, 58 bp, synthetic construct
  source 1..58: mol_type = other DNA
acactctttccctacacgacgctcttccgatctgagaattcatcatggattccactag

SEQ ID NO: 78 - DNA, 57 bp, synthetic construct
  source 1..57: mol_type = other DNA
acactctttccctacacgacgctcttccgatctgccttaaagtcttctcctctttca

SEQ ID NO: 79 - DNA, 52 bp, synthetic construct
  source 1..52: mol_type = other DNA
acactctttccctacacgacgctcttccgatcttgactccctgaggaaatca

SEQ ID NO: 80 - DNA, 56 bp, synthetic construct
  source 1..56: mol_type = other DNA
acactctttccctacacgacgctcttccgatctccatatgccaaaagagtctatca

SEQ ID NO: 81 - DNA, 55 bp, synthetic construct
  source 1..55: mol_type = other DNA
acactctttccctacacgacgctcttccgatcttgcagaatggatgttgtgttag

SEQ ID NO: 82 - DNA, 53 bp, synthetic construct
  source 1..53: mol_type = other DNA
acactctttccctacacgacgctcttccgatctgccagaacacacaaaacatt

SEQ ID NO: 83 - DNA, 58 bp, synthetic construct
  source 1..58: mol_type = other DNA
acactctttccctacacgacgctcttccgatctacgtaatagtctacagaccacatat

SEQ ID NO: 84 - DNA, 55 bp, synthetic construct
  source 1..55: mol_type = other DNA
gtgactggagttcagacgtgtgctcttccgatcttcaagtcatgcactggatgtg

SEQ ID NO: 85 - DNA, 51 bp, synthetic construct
  source 1..51: mol_type = other DNA
gtgactggagttcagacgtgtgctcttccgatctgatggcagagctaacag

SEQ ID NO: 86 - DNA, 57 bp, synthetic construct
  source 1..57: mol_type = other DNA
gtgactggagttcagacgtgtgctcttccgatctgatgagacctctgagaagaaaag

SEQ ID NO: 87 - DNA, 59 bp, synthetic construct
  source 1..59: mol_type = other DNA
gtgactggagttcagacgtgtgctcttccgatctccttgcttactgcttattgctagta

SEQ ID NO: 88 - DNA, 60 bp, synthetic construct
  source 1..60: mol_type = other DNA
gtgactggagttcagacgtgtgctcttccgatctgggtgctttaacaacataagtctgat

SEQ ID NO: 89 - DNA, 53 bp, synthetic construct
  source 1..53: mol_type = other DNA
gtgactggagttcagacgtgtgctcttccgatctggagtcacacgccattttc

SEQ ID NO: 90 - DNA, 55 bp, synthetic construct; identical to SEQ ID NO: 84
  source 1..55: mol_type = other DNA

SEQ ID NO: 91 - DNA, 57 bp, synthetic construct
  source 1..57: mol_type = other DNA
gtgactggagttcagacgtgtgctcttccgatctgaaataatcccattgtacgttga

SEQ ID NO: 92 - DNA, 55 bp, synthetic construct
  source 1..55: mol_type = other DNA
gtgactggagttcagacgtgtgctcttccgatctcttttgctagactcagctgtg

SEQ ID NO: 93 - DNA, 60 bp, synthetic construct
  source 1..60: mol_type = other DNA
gtgactggagttcagacgtgtgctcttccgatctgcagttatccttggtgaaaaatgata

SEQ ID NO: 94 - DNA, 60 bp, synthetic construct
  source 1..60: mol_type = other DNA
gtgactggagttcagacgtgtgctcttccgatctcaattcaaatggtcttttggttacag

SEQ ID NO: 95 - DNA, 56 bp, synthetic construct
  source 1..56: mol_type = other DNA
gtgactggagttcagacgtgtgctcttccgatctgcagattgtttccattgcattc

SEQ ID NO: 96 - DNA, 54 bp, synthetic construct
  source 1..54: mol_type = other DNA
gtgactggagttcagacgtgtgctcttccgatcttagtctgcattgctctatgg

SEQ ID NO: 97 - DNA, 57 bp, synthetic construct
  source 1..57: mol_type = other DNA
gtgactggagttcagacgtgtgctcttccgatctccctagttttagtctaaaccagt

SEQ ID NO: 98 - DNA, 55 bp, synthetic construct
  source 1..55: mol_type = other DNA
gtgactggagttcagacgtgtgctcttccgatctaaccaggcactgttaaaagat

SEQ ID NO: 99 - DNA, 58 bp, synthetic construct
  source 1..58: mol_type = other DNA
gtgactggagttcagacgtgtgctcttccgatctcacaacgtgtagacctgaaataaa

SEQ ID NO: 100 - DNA, 55 bp, synthetic construct
  source 1..55: mol_type = other DNA
gtgactggagttcagacgtgtgctcttccgatctggcaccattccaaagaataca

SEQ ID NO: 101 - DNA, 60 bp, synthetic construct
  source 1..60: mol_type = other DNA
gtgactggagttcagacgtgtgctcttccgatctgtaattttgtctaaaagcctaaaatg

SEQ ID NO: 102 - DNA, 57 bp, synthetic construct
  source 1..57: mol_type = other DNA
gtgactggagttcagacgtgtgctcttccgatcttacatttgattggcatttcctcc

SEQ ID NO: 103 - DNA, 54 bp, synthetic construct
  source 1..54: mol_type = other DNA
gtgactggagttcagacgtgtgctcttccgatcttgaatgaacaaagctgagct

SEQ ID NO: 104 - DNA, 57 bp, synthetic construct
  source 1..57: mol_type = other DNA
gtgactggagttcagacgtgtgctcttccgatctctcagtatctccatttcacagtg

SEQ ID NO: 105 - DNA, 57 bp, synthetic construct
  source 1..57: mol_type = other DNA
gtgactggagttcagacgtgtgctcttccgatctgtttgatgaacattttgagccct

SEQ ID NO: 106 - DNA, 53 bp, synthetic construct
  source 1..53: mol_type = other DNA
gtgactggagttcagacgtgtgctcttccgatctcagagtggtgtttcctagg

SEQ ID NO: 107 - DNA, 60 bp, synthetic construct
  source 1..60: mol_type = other DNA
gtgactggagttcagacgtgtgctcttccgatcttaaagctactccttgtcctgaaattc

SEQ ID NO: 108 - DNA, 20 bp, synthetic construct
  source 1..20: mol_type = other DNA
acagagctaagacaagaacg

SEQ ID NO: 109 - DNA, 20 bp, synthetic construct
  source 1..20: mol_type = other DNA
ggcttgaaagctgttacaag

SEQ ID NO: 110 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 111 - protein, 1052 aa, synthetic construct
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 112 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 113 - DNA, 4101 bp, synthetic construct
  source 1..4101: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 114 - DNA, 3156 bp, synthetic construct
  source 1..3156: mol_type = other DNA
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 115 - DNA, 1587 bp, synthetic construct
  source 1..1587: mol_type = other DNA
atggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagccc

SEQ ID NO: 116 - DNA, 12267 bp, synthetic construct
  source 1..12267: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggctagcttaattaacctgcaggcccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgactctaggcccaggcggccttgtacaaaaaagcaccggtaggctgccaccatgccaaagaagaagcggaaggtcggtatccacggagtcccagcagccatggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagcccggatccggaatggcgactaaagtgaagttcaaatataagggtgaagaaaaggaagtagatatatccaagatcaagaaggtatggcgggttggaaaaatgatcagctttacctatgacgacaacggaaagactggtaggggtgccgtatctgaaaaagatgcccccaaagaattgttggaaaaactcaagggaagcggaaaaaggccggcggccacgaaaaaggccggccaggcaaaaaagaaaaagtacccatacgatgttccagattacgctggaagcggagagggcaggggaagtcttctaacatgcggggacgtggaggaaaatcccggccccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagacccgaattccagggccaggccggccgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 117 - protein, 596 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEPGSGMATVKFKYKGEEKEVDISKIKKVWRVGKMISFTYDEGGGKTGRGAVSEKDAPKELLQMLEKQKK

SEQ ID NO: 118 - DNA, 1788 bp, synthetic construct
  source 1..1788: mol_type = other DNA
atggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagcccggatccggaatggctacagtgaagtttaagtataaaggggaagagaaagaggtggacatcagtaaaatcaagaaagtgtggcgcgtaggtaagatgatcagctttacgtacgatgaaggaggaggaaagactggcagaggagccgtttcagaaaaggacgctccaaaggaattgttgcagatgctcgagaagcagaagaag

SEQ ID NO: 119 - protein, 1434 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSGMATVKFKYKGEEKEVDISKIKKVWRVGKMISFTYDEGGGKTGRGAVSEKDAPKELLQMLEKQKK

SEQ ID NO: 120 - DNA, 4302 bp, synthetic construct
  source 1..4302: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacggatccggaatggctacagtgaagtttaagtataaaggggaagagaaagaggtggacatcagtaaaatcaagaaagtgtggcgcgtaggtaagatgatcagctttacgtacgatgaaggaggaggaaagactggcagaggagccgtttcagaaaaggacgctccaaaggaattgttgcagatgctcgagaagcagaagaag

SEQ ID NO: 121 - protein, 1119 aa, synthetic construct
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGGSGMATVKFKYKGEEKEVDISKIKKVWRVGKMISFTYDEGGGKTGRGAVSEKDAPKELLQMLEKQKK

SEQ ID NO: 122 - DNA, 3357 bp, synthetic construct
  source 1..3357: mol_type = other DNA
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcggatccggaatggctacagtgaagtttaagtataaaggggaagagaaagaggtggacatcagtaaaatcaagaaagtgtggcgcgtaggtaagatgatcagctttacgtacgatgaaggaggaggaaagactggcagaggagccgtttcagaaaaggacgctccaaaggaattgttgcagatgctcgagaagcagaagaag

SEQ ID NO: 123 - protein, 592 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEPGSGMATKVKFKYKGEEKEVDISKIKKVWRVGKMISFTYDDNGKTGRGAVSEKDAPKELLEKLK

SEQ ID NO: 124 - DNA, 1776 bp, synthetic construct
  source 1..1776: mol_type = other DNA
atggccaagaacacaattacaaagacactgaagctgaggatcgtgagaccatacaacagcgctgaggtcgagaagattgtggctgatgaaaagaacaacagggaaaagatcgccctcgagaagaacaaggataaggtgaaggaggcctgctctaagcacctgaaagtggccgcctactgcaccacacaggtggagaggaacgcctgtctgttttgtaaagctcggaagctggatgataagttttaccagaagctgcggggccagttccccgatgccgtcttttggcaggagattagcgagatcttcagacagctgcagaagcaggccgccgagatctacaaccagagcctgatcgagctctactacgagatcttcatcaagggcaagggcattgccaacgcctcctccgtggagcactacctgagcgacgtgtgctacacaagagccgccgagctctttaagaacgccgctatcgcttccgggctgaggagcaagattaagagtaacttccggctcaaggagctgaagaacatgaagagcggcctgcccactacaaagagcgacaacttcccaattccactggtgaagcagaaggggggccagtacacagggttcgagatttccaaccacaacagcgactttattattaagatcccctttggcaggtggcaggtcaagaaggagattgacaagtacaggccctgggagaagtttgatttcgagcaggtgcagaagagccccaagcctatttccctgctgctgtccacacagcggcggaagaggaacaaggggtggtctaaggatgaggggaccgaggccgagattaagaaagtgatgaacggcgactaccagacaagctacatcgaggtcaagcggggcagtaagattggcgagaagagcgcctggatgctgaacctgagcattgacgtgccaaagattgataagggcgtggatcccagcatcatcggagggatcgatgtgggggtcaagagccccctcgtgtgcgccatcaacaacgccttcagcaggtacagcatctccgataacgacctgttccactttaacaagaagatgttcgcccggcggaggattttgctcaagaagaaccggcacaagcgggccggacacggggccaagaacaagctcaagcccatcactatcctgaccgagaagagcgagaggttcaggaagaagctcatcgagagatgggcctgcgagatcgccgatttctttattaagaacaaggtcggaacagtgcagatggagaacctcgagagcatgaagaggaaggaggattcctacttcaacattcggctgagggggttctggccctacgctgagatgcagaacaagattgagtttaagctgaagcagtacgggattgagatccggaaggtggcccccaacaacaccagcaagacctgcagcaagtgcgggcacctcaacaactacttcaacttcgagtaccggaagaagaacaagttcccacacttcaagtgcgagaagtgcaactttaaggagaacgccgattacaacgccgccctgaacatcagcaaccctaagctgaagagcactaaggaggagcccggatccggaatggcgactaaagtgaagttcaaatataagggtgaagaaaaggaagtagatatatccaagatcaagaaggtatggcgggttggaaaaatgatcagctttacctatgacgacaacggaaagactggtaggggtgccgtatctgaaaaagatgcccccaaagaattgttggaaaaactcaag

SEQ ID NO: 125 - protein, 5 aa, synthetic construct
  REGION 1..5: note = Linker
GGGGS